(19) United States
(12) Patent Application Publication
(10) Pub. No.: US 2004/0009527 A1
(43) Pub. Date: Jan. 15, 2004

(54) Common ligand mimics: pseudothiohydantoins

(76) Inventors: Qing Dong, San Diego, CA (US); Fabrice Pierre, La Jolla, CA (US); Hengyuan Lang, San Diego, CA (US); Lin Yu, San Diego, CA (US); Mark Hansen, San Diego, CA (US); Daniel S. Sem, San Diego, CA (US); Maurizio Pellecchia, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/099,136

(22) Filed: Mar. 15, 2002

(51) Int. Cl. G01N 33/53; C07D 417/02; C07D 277/38; G01N 33/543

(52) U.S. Cl. 435/7.1; 548/184; 546/269.7; 436/518

(57) ABSTRACT

The present invention provides common ligand mimics that act as common ligands for a receptor family. The present invention also provides bi-ligands containing these common ligand mimics. Bi-ligands of the invention provide enhanced affinity and/or selectivity of ligand binding to a receptor or receptor family through the synergistic action of the common ligand mimic and specificity ligand that compose the bi-ligand. The present invention also provides combinatorial libraries containing the common ligand mimics and bi-ligands of the invention. Further, the present invention provides methods for manufacturing the common ligand mimics and bi-ligands of the invention and methods for assaying the combinatorial libraries of the invention.

### BACKGROUND OF THE INVENTION

1. Field of the Invention

The present invention relates generally to receptor/ligand interactions and to combinatorial libraries of ligand compounds. The present invention also relates to the manufacture of psudothiohydantoin compounds and combinatorial libraries containing such compounds.

2. Background Information

Two general approaches have traditionally been used for drug discovery: screening for lead compounds and structure-based drug design. Both of these approaches are laborious and time-consuming and often produce compounds that lack the desired affinity or specificity.

Screening for lead compounds involves generating a pool of candidate compounds, often using combinatorial chemistry approaches in which compounds are synthesized by combining chemical groups to generate a large number of diverse candidate compounds that bind to the target or that inhibit binding to the target. The candidate compounds are screened with a drug target of interest to identify lead compounds that bind to the target or inhibit binding to the target. However, the screening process to identify a lead compound can be laborious and time consuming.

Structure-based drug design is an alternative approach to identifying candidate drugs. Structure-based drug design uses three-dimensional structural data, of the drug target as a template to model compounds that bind to the drug target and alter its activity. The compounds identified as potential candidate drugs using structural modeling are used as lead compounds for the development of candidate drugs that exhibit a desired activity toward the drug target.

Identifying compounds using structure-based drug design can be advantageous when compared to the screening approach in that modifications to the compound can often be predicted by modeling studies. However, obtaining structures of relevant drug targets and of drug targets complexed with test compounds is extremely time-consuming and laborious, often taking years to accomplish. The long time period required to obtain structural information useful for developing candidate drugs is particularly limiting with regard to the growing number of newly discovered genes, which are potential drug targets, identified in genomics studies.

Despite the time-consuming and laborious nature of these approaches to drug discovery, both screening for lead compounds and structure-based drug design have led to the identification of a number of useful drugs, such as receptor agonists and antagonists. However, many of the drugs identified by these approaches have unwanted toxicity or side effects. Therefore, there is a need in the art for drugs that have high specificity and reduced toxicity. For example, in addition to binding to the drug target in a pathogenic organism or cancer cell, in some cases the drug also binds to an analogous protein in the patient being treated with the drug, which can result in toxic or unwanted side effects. Therefore, drugs that have high affinity and specificity for a target are particularly useful because administration of a more specific drug at lower dosages will minimize toxicity and side effects.

In addition to drug toxicity and side effects, a number of drugs that were previously highly effective for treating certain diseases have become less effective during prolonged clinical use due to the development of resistance. Drug resistance has become increasingly problematic, particularly with regard to administration of antibiotics. A number of pathogenic organisms have become resistant to several drugs due to prolonged clinical use and, in some cases, have become almost totally resistant to currently available drugs. Furthermore, certain types of cancer develop resistance to cancer therapeutic agents. Therefore, drugs that are retractile to the development of resistance would be particularly desirable for treatment of a variety of diseases.

One approach to developing such drugs is to find compounds that bind to a target protein such as a receptor or enzyme. When such a target protein has two adjacent binding sites, it is especially useful to find “bi-ligand” drugs that can bind at both sites simultaneously. However, the rapid identification of bi-ligand drugs having the optimum combination of affinity and specificity has been difficult. Bi-ligand candidate drugs have been identified using rational drug design, but previous methods are time-consuming and require a precise knowledge of structural features of the receptor. Recent advances in nuclear magnetic spectroscopy (NMR) have allowed the determination of the three-dimensional interactions between a ligand and a receptor in a few instances. However, these efforts have been limited by the size of the receptor and can take years to map and analyze the complete structure of the complexes of receptor and ligand.

Thus, there exists a need for compounds that bind to multiple members of a receptor family. There is also a need for receptor bi-ligands containing such compounds coupled to ligands having a high specificity for the receptor.

There is a further need in the art for methods of preparing such compounds and bi-ligands. There is also a need in the art for methods of preparing combinatorial libraries of the bi-ligands and methods of screening these libraries to find bi-ligands that interact with a drug target with improved affinity and/or specificity. The present invention satisfies these needs and provides related advantages as well.

### SUMMARY OF THE INVENTION

The present invention provides compounds that function as mimics to a natural common ligand for a receptor family. These compounds interact with a conserved binding site on multiple receptors within the receptor family.

In one aspect, the present invention provides compounds that are common ligand mimics for NAD. NAD is a natural common ligand for many oxidoreductases. Thus, compounds of the invention that are common ligand mimics for NAD interact selectively with conserved sites on oxidoreductases.

In one embodiment, the present invention provides compounds of Formula I,

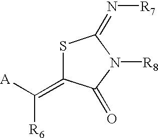

wherein A is an aromatic carbocyclic or heterocyclic ring containing 5, 6, or 7 members and having from 0 to 3 heterocyclic atoms selected from the group consisting of oxygen, nitrogen, and sulfur. A is optionally substituted with from one to five substituents which each independently are hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

In another embodiment, the invention provides compounds of Formula II,

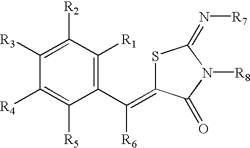

wherein R₁ to R₆ each independently are H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

In still another embodiment, the invention provides compounds of Formula III,

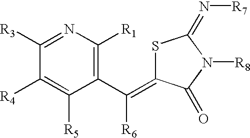

wherein R₁, R₃, R₄, R₅, and R₆ each independently are hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₃, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀ and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

In a second aspect, the present invention provides methods for preparing compounds of Formula I, II, and III. These methods generally comprise reaction of pseudothiohydantoin with a carboxybenzaldehyde, pyridine carboxyaldehyde, or pyrimidine carboxyaldehyde.

In a third aspect, the present invention provides bi-ligands containing a common ligand mimic and a specificity ligand, which interact with distinct sites on a receptor. In one embodiment, the present invention provides bi-ligands that are the reaction products of compounds of Formula I with specificity ligands. In another embodiment, the invention provides bi-ligands containing the reaction products of compounds of Formula II with specificity ligands. In yet another embodiment, the invention provides bi-ligands that are reaction products of compounds of Formula III and specificity ligands. In yet another aspect, the invention provides methods for preparing bi-ligands that are reaction products of the common ligand mimics of general Formulas I, II, and III and a pyridine dicarboxylate specificity ligand.

The present invention further provides combinatorial libraries containing one or more common ligand variants of the compounds of the invention. In one embodiment, the combinatorial libraries of the invention contain one or more common ligand variants of the compounds of Formula I. In other embodiments, the combinatorial libraries of the invention contain one or more common ligand variants of the compounds of Formula II or Formula III.

The present invention also provides combinatorial libraries comprised of one or more bi-ligands that are reaction products of common ligand mimics and specificity ligands. In one embodiment, such combinatorial libraries contain one or more bi-ligands that are the reaction product of compounds of Formula I and specificity ligands. In another embodiment, such combinatorial libraries contain one or more bi-ligands that are the reaction product of compounds of Formula II and specificity ligands. In still another embodiment, such combinatorial libraries contain one or more bi-ligands that are the reaction product of compounds of Formula III and specificity ligands.

The present invention also provides methods for producing and screening combinatorial libraries of bi-ligands for binding to a receptor and families of such receptors.

### BRIEF DESCRIPTION OF THE DRAWINGS

FIG. 1 shows Scheme 1 for the synthesis of pseudothiohydantoin compounds of Formula I. FIG. 1a provides the general reaction scheme for compounds of Formula I, while FIGS. Ib and Ic show the reaction scheme for production of compounds of Formulas II and III, respectively where R₁ to R₅ each are H, OH, COOH, OAlkyl, OAc, COOAlkyl, CN, NO₂, NH₂, or NHAc. The reaction steps are as follows: pseudothiohydantoin is mixed with a carboxy benzaldehyde, or a pyridine carboxyaldehyde or pyrimidine carboxyaldehyde. The mixture is heated for a period of time.

FIG. 2 shows Scheme 2 for the synthesis of bi-ligands containing pseudothiohydantoin common ligand mimics and pyridine dicarboxylate specificity ligands.

FIG. 3 shows a reaction scheme for modification of substituents attached to the common ligand mimics of the invention.

FIGS. 4a-c show various reaction schemes by which combinatorial libraries of the present invention can be made. FIG. 4a shows the reaction scheme for reaction of common ligand mimics of the present invention having a carboxylic acid group with an amine in the presence of hydroxybenzotriazole (HOBt). FIG. 4b shows the reaction of common ligand mimics of the invention having an amine terminal amide substituent with a carboxylic acid in the presence of HOBt. FIG. 4c shows the reaction scheme for reaction of common ligand mimics of the invention having an amine terminal amide substituent with an isocyanate or thioisocyanate.

FIG. 5 shows the results of a oxidoreductase enzymatic panel study of selected pseudothiohydantoin compounds of the invention.

FIG. 6 shows the results of an enzymatic panel study of selected pseudothiohydantoin compounds of the invention.

FIG. 7 shows the results of a oxidoreductase assay of selected bi-ligands of the invention.

FIGS. 8a-c show the names and corresponding structures for exemplified pseudothiohydantoin common ligand mimics of the invention.

FIG. 9 shows examples of bi-ligands of the invention.

### DETAILED DESCRIPTION OF THE INVENTION

The present invention is directed to bi-ligands and the development of combinatorial libraries associated with these bi-ligands. The invention advantageously can be used to develop bi-ligands that bind to two distinct sites on a receptor, a common site and a specificity site. Tailoring of the two portions of the bi-ligand provides optimal binding characteristics. These optimal binding characteristics provide increased diversity within a library, while simultaneously focusing the library on a particular receptor family or a particular member of a receptor family. The two portions of the bi-ligand, a common ligand mimic and a specificity ligand act synergistically to provide higher affinity and/or specificity than either ligand alone.

The technology of the present invention can be applied across receptor families or can be used to screen for specific members of a family. For example, the present invention can be used to screen libraries for common ligand mimics that bind to any oxidoreductase. Alternatively, the present invention can be used to screen for a particular oxidoreductase that will bind a particular specificity ligand.

The present invention provides common ligand mimics that bind selectively to a conserved site on a receptor. The compounds advantageously can be used to develop combinatorial libraries of bi-ligands more efficiently than conventional methods. The present invention takes advantage of NMR spectroscopy to identify the interactions between the common ligand mimic and the receptor, which allows for improved tailoring of the ligand to the receptor.

The present invention also provides bi-ligands containing these common ligand mimics. The bi-ligands of the invention contain a common ligand mimic coupled to a specificity ligand. These bi-ligands provide the ability to tailor the affinity and/or specificity of the ligands to the binding sites on the receptor.

The present invention further provides combinatorial libraries containing bi-ligands of the invention as well as formation of such libraries from the common ligand mimics of the invention. These libraries provide an enhanced number of bi-ligands that bind multiple members of a receptor family than is provided with standard combinatorial techniques due to specific positioning of the specificity ligand on the common ligand mimic. Optimal positioning of the specificity ligand can be determined through NMR studies of the receptor and the common ligand mimic to be employed.

The present invention also provides methods for the preparation of pseudothiohydantoin compounds useful as common ligand mimics in the present invention and methods for the preparation of bi-ligands containing these common ligand mimics. In general, such methods involve reaction of pseudothiohydantoin with a carboxybenzaldehyde, pyridine carboxyaldehyde, or pyrimidine carboxyaldehyde. The present invention also provides methods for modification of the common ligand mimics to form additional common ligand mimics having different bi-ligand directing/binding substituents. The common ligand mimics can be used to create bi-ligands having improved affinity, improved specificity, or both. These and other aspects of the invention are described below.

The present invention provides common ligand mimics. As used herein, the term “ligand” refers to a molecule that can selectively bind to a receptor. The term “selectively” means that the binding interaction is detectable over non-specific interactions as measured by a quantifiable assay. A ligand can be essentially any type of molecule such as an amino acid, peptide, polypeptide, nucleic acid, carbohydrate, lipid, or small organic compound. The term ligand refers both to a molecule capable of binding to a receptor and to a portion of such a molecule, if that portion of a molecule is capable of binding to a receptor. For example, a bi-ligand, which contains a common ligand and specificity ligand, is considered a ligand, as would the common ligand and specificity ligand portions since they can bind to a conserved site and specificity site, respectively. As used herein, the term “ligand” excludes a single atom, for example, a metal atom. Derivatives, analogues, and mimetic compounds also are included within the definition of this term. These derivatives, analogues and mimetic compounds include those containing metals or other inorganic molecules, so long as the metal or inorganic molecule is covalently attached to the ligand in such a manner that the dissociation constant of the metal from the ligand is less than 10-14 M. A ligand can be multi-partite, comprising multiple ligands capable of binding to different sites on one or more receptors, such as a bi-ligand. The ligand components of a multi-partite ligand can be joined together directly, for example, through functional groups on the individual ligand components or can be joined together indirectly, for example, through an expansion linker.

As used herein, the term “common ligand” refers to a ligand that binds to a conserved site on receptors in a receptor family. A “natural common ligand” refers to a ligand that is found in nature and binds to a common site on receptors in a receptor family. As used herein, a “common ligand mimic (CLM)” refers to a common ligand that has structural and/or functional similarities to a natural common ligand but is not naturally occurring. Thus, a common ligand mimic can be a modified natural common ligand, for example, an analogue or derivative of a natural common ligand. A common ligand mimic also can be a synthetic compound or a portion of a synthetic compound that is structurally similar to a natural common ligand.

As used herein, a “common ligand variant” refers to a derivative of a common ligand. A common ligand variant has structural and/or functional similarities to a parent common ligand. A common ligand variant differs from another variant, including the parent common ligand, by at least one atom. For example, as with NAD and NADH, the reduced and oxidized forms differ by an atom and are therefore considered to be variants of each other. A common ligand variant includes reactive forms of a common ligand mimic, such as an anion or cation of the common ligand mimic. As used herein, the term “reactive form” refers to a form of a compound that can react with another compound to form a chemical bond, such as an ionic or covalent bond. For example, where the common ligand mimic is an acid of the form ROOH or an ester of the form ROOR′, the common ligand variant can be ROO⁻.

As used herein, the term “conserved site” on a receptor refers to a site that has structural and/or functional characteristics common to members of a receptor family. A conserved site contains amino acid residues sufficient for activity and/or function of the receptor that are accessible to binding of a natural common ligand. For example, the amino acid residues sufficient for activity and/or function of a receptor that is an enzyme can be amino acid residues in a substrate binding site of the enzyme. Also, the conserved site in an enzyme that binds a cofactor or coenzyme can be amino acid residues that bind the cofactor or coenzyme.

As used herein, the term “receptor” refers to a polypeptide that is capable of selectively binding a ligand. The function or activity of a receptor can be enzymatic activity or ligand binding. Receptors can include, for example, enzymes such as kinases, dehydrogenases, oxidoreductases, GTPases, carboxyl transferases, acyl transferases, decarboxylases, transaminases, racemases, methyl transferases, formyl transferases, and α-ketodecarboxylases.

Furthermore, the receptor can be a functional fragment or modified form of the entire polypeptide so long as the receptor exhibits selective binding to a ligand. A functional fragment of a receptor is a fragment exhibiting binding to a common ligand and a specificity ligand. As used herein, the term “enzyme” refers to a molecule that carries out a catalytic reaction by converting a substrate to a product.

Enzymes can be classified based on Enzyme Commission (EC) nomenclature recommended by the Nomenclature Committee of the International Union of Biochemistry and Molecular Biology (IUBMB) (see, for example, www.expasy.ch/sprot/enzyme.html) (which is incorporated herein by reference). For example, oxidoreductases are classified as oxidoreductases acting on the CH—OH group of donors with NAD⁺ or NADP⁺ as an acceptor (EC 1.1.1); oxidoreductases acting on the aldehyde or oxo group of donors with NAD⁺ or NADP⁺ as an acceptor (EC 1.2.1); oxidoreductases acting on the CH—CH group of donors with NAD⁺ or NADP⁺ as an acceptor (EC 1.3.1); oxidoreductases acting on the CH—NH₂ group of donors with NAD⁺ or NADP⁺ as an acceptor (EC 1.4.1); oxidoreductases acting on the CH—NH group of donors with NAD⁺ or NADP⁺ as an acceptor (EC 1.5.1); oxidoreductases acting on NADH or NADPH (EC 1.6); and oxidoreductases acting on NADH or NADPH with NAD⁺ or NADP⁺ as an acceptor (EC 1.6.1).

Additional oxidoreductases include oxidoreductases acting on a sulfur group of donors with NAD⁺ or NADP⁺ as an acceptor (EC 1.8.1); oxidoreductases acting on diphenols and related substances as donors with NAD⁺ or NADP⁺ as an acceptor (EC 1.10.1); oxidoreductases acting on hydrogen as donor with NAD⁺ or NADP⁺ as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NAD⁺ or NADP⁺ as an acceptor (EC 1.16.1); oxidoreductases acting on —CH₂ groups with NAD⁺ or NADP⁺ as an acceptor (EC 1.17.1) ; and oxidoreductases acting on reduced ferredoxin as donor, with NAD⁺ or NADP⁺ as an acceptor (EC 1.18.1).

Enzymes can also bind coenzymes or cofactors such as nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, and tetrahydrofolate or other cofactors or substrates such as ATP, GTP and S-adenosyl methionine (SAM). In addition, enzymes that bind newly identified cofactors or enzymes can also be receptors.

As used herein, the term “receptor family” refers to a group of two or more receptors that share a common, recognizable amino acid motif. A motif in a related family of receptors occurs because certain amino acid residues, or residues having similar chemical characteristics, are required for the structure, function and/or activity of the receptor and are, therefore, conserved between members of the receptor family. Methods of identifying related members of a receptor family are well known to those skilled in the art and include sequence alignment algorithms and identification of conserved patterns or motifs in a group of polypeptides, which are described in more detail below. Members of a receptor family also can be identified by determination of binding to a common ligand.

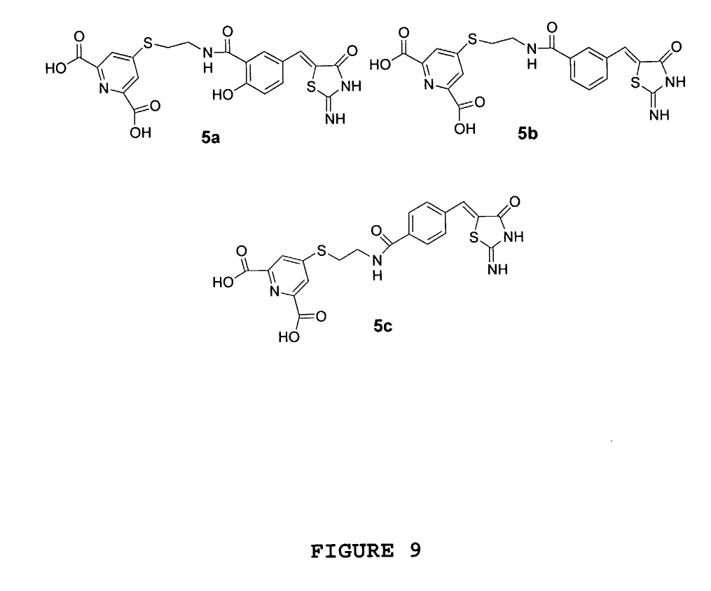

In another aspect, the present invention provides bi-ligands that contain a common ligand mimic as described above and a specificity ligand. As used herein, the term “bi-ligand” refers to a ligand comprising two ligands that bind to independent sites on a receptor. One of the ligands of a bi-ligand is a specificity ligand capable of binding to a site that is specific for a given member of a receptor family when joined to a common ligand. The second ligand of a bi-ligand is a common ligand mimic that binds to a conserved site in a receptor family. The common ligand mimic and specificity ligand are bonded together. Bonding of the two ligands can be direct or indirect, such as through a linking molecule or group. A depiction of exemplary bi-ligands is shown in FIG. 9.

As used herein the term “specificity” refers to the ability of a ligand to differentially bind to one receptor over another receptor in the same receptor family. The differential binding of a particular ligand to a receptor is measurably higher than the binding of the ligand to at least one other receptor in the same receptor family. A ligand having specificity for a receptor refers to a ligand exhibiting specific binding that is at least two-fold higher for one receptor over another receptor in the same receptor family.

As used herein, the term “specificity ligand” refers to a ligand that binds to a specificity site on a receptor. A specificity ligand can bind to a specificity site as an isolated molecule or can bind to a specificity site when attached to a common ligand, as in a bi-ligand. When a specificity ligand is part of a bi-ligand, the specificity ligand can bind to a specificity site that is proximal to a conserved site on a receptor.

As used herein, the term “specificity site” refers to a site on a receptor that provides the binding site for a ligand exhibiting specificity for a receptor. A specificity site on a receptor imparts molecular properties that distinguish the receptor from other receptors in the same receptor family. For example, if the receptor is an enzyme, the specificity site can be a substrate binding site that distinguishes two members of a receptor family which exhibit substrate specificity. A substrate specificity site can be exploited as a potential binding site for the identification of a ligand that has specificity for one receptor over another member of the same receptor family. A specificity site is distinct from the common ligand binding site in that the natural common ligand does not bind to the specificity site.

As used herein, the term “linker” refers to a chemical group that can be attached to either the common ligand or the specificity ligand of a bi-ligand. The invention provides the functional groups through which the common ligand mimic and the specificity ligand are directly bound to one another. The linker can be a simple functional group, such as COOH, NH₂, OH, or the like. Alternatively, the linker can be a complex chemical group containing one or more unsaturation, one or more substituent, and/or one or more heterocyclic atom. Nonlimiting examples of complex linkers are depicted in Tables 4 to 10.

The present invention provides common ligand mimics that are common mimics of NAD and combinatorial libraries containing these common ligand mimics. For example, in one embodiment, compounds of the invention are ligands for conserved sites on dehydrogenases and reductases. Examples of such receptors include, but are not limited to, HMG CoA reductase (HMGCoAR), inosine-5′-monophosphate dehydrogenase (IMPDH), 1-deoxy-D-xylulose-5-phosphate reductase (DOXPR), dihydrodipicolinate reductase (DHPR), dihydrofolate reductase (DHPR), 3-isopropylmalate (IPMDH), glyceraldehyde-3-phosphate dehydrogenase (GAPDH), aldose reductase (AR), alcohol dehydrogenase (ADH), and lactate dehydrogenase (LDH), and enoyl ACP reductase.

The present invention also provides compounds and combinatorial libraries of compounds of the formula:

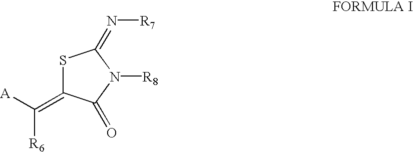

wherein A is an aromatic carbocyclic or heterocyclic ring containing 5, 6, or 7 members and having from 0 to 3 heterocyclic atoms selected from the group consisting of oxygen, nitrogen, and sulfur. A is optionally substituted with from one to five substituents which each independently are hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

As used herein, “alkyl” means a carbon chain having from one to twenty carbon atoms. The alkyl group of the present invention can be straight chain or branched. It can be unsubstituted or can be substituted. When substituted, the alkyl group can have up to ten substituent groups, such as COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X where R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

Additionally, the alkyl group present in the compounds of the invention, whether substituted or unsubstituted, can have one or more of its carbon atoms replaced by a heterocyclic atom, such as an oxygen, nitrogen, or sulfur atom. For example, alkyl as used herein includes groups such as (OCH₂CH₂)n or (OCH₂CH₂CH₂)ₙ, where n has a value such that there are twenty or less carbon atoms in the alkyl group. Similar compounds having alkyl groups containing a nitrogen or sulfur atom are also encompassed by the present invention.

As used herein “alkenyl” means an unsaturated alkyl groups as defined above, where the unsaturation is in the form of a double bond. The alkenyl groups of the present invention can have one or more unsaturations. Nonlimiting examples of such groups include CH═CH₂, CH₂CH₂CH═CHCH₂CH₃, and CH₂CH═CHCH₃. As used herein “alkynyl” means an unsaturated alkyl group as defined above, where the unsaturation is in the form of a triple bond. Alkynyl groups of the present invention can include one or more unsaturations. Nonlimiting examples of such groups include C≡CH, CH₂CH₂C≡CCH₂CH₃, and CH₂C≡CCH₃.

The compounds of the present invention can include compounds in which R₁ to R₆ each independently are complex substituents containing one or more unsaturation, one or more substituent, and/or one or more heterocyclic atom. These complex substituents are also referred to herein as “linkers” or “expansion linkers.” Nonlimiting examples of complex substituents that can be used in the present invention are presented in Tables 4 to 10.

As used herein, “aromatic group” refers to a group that has a planar ring with 4n+2 pi-electrons, where in is a positive integer. The term “aryl” as used herein denotes a nonheterocyclic aromatic compound or group, for example, a benzene ring or naphthalene ring.

As used herein, “heterocyclic group” or “heterocycle” refers to an aromatic compound or group containing one or more heterocyclic atom. Nonlimiting examples of heterocyclic atoms that can be present in the heterocyclic groups of the invention include nitrogen, oxygen and sulfur. In general, heterocycles of the present invention will have from five to seven atoms and can be substituted or unsubstituted. When substituted, substituents include, for example, those groups provided for R₁ to R₁₀. Nonlimiting examples of heterocyclic groups of the invention include pyroles, pyrazoles, imidazoles, pyridines, pyrimidines, pyridzaines, pyrazines, triazines, furans, oxazoles, thiazoles, thiophenes, diazoles, triazoles, tetrazoles, oxadiazoles, thiodiazoles, and fused heterocyclic rings, for example, indoles, benzofurans, benzothiophenes, benzoimidazoles, benzodiazoles, benzotriazoles, and quinolines.

As used herein, the variable “X” indicates a halogen atom. Halogens suitable for use in the present invention include chlorine, fluorine, iodine, and bromine, with bromine being particularly useful. As used herein, “Ac” denotes an acyl group. Suitable acyl groups can have, for example, an alkyl, alkenyl, alkynyl, aromatic, or heterocyclic group as defined above attached to the carbonyl group.

A in Formula I is an aromatic ring. For example, A can be an aromatic carbocyclic ring, such as a benzene ring, or a heterocyclic ring, such as a pyridine ring. A can have from five to seven members. When A is a heterocyclic ring, it can have from one to three heterocyclic atoms. Nonlimiting examples of such heterocyclic atoms include oxygen, nitrogen, and sulfur. A includes, but is not limited to, the heterocyclic groups provided above. A can be substituted with one or multiple substituents. Variation in the substitution provides compounds that allow for addition of a specificity ligand to directed sites on A. Direction of the specificity ligand improves the ease and efficiency of manufacture of combinatorial libraries containing bi-ligands having the common ligand mimic bound to a specificity ligand.

In one embodiment, A contains only one nonhydrogen substituent. In such instances, A can be substituted for example, with the following groups: hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X where R₉, R₁₀, and R₁₁ each independently are hydrogen, alkyl, alkenyl, alkynyl, aryl, or heterocycle or where R₉ and R₁₀ together with the nitrogen atom to which they are attached can be joined to form a heterocyclic ring. For example, A can be substituted with an OH group, a COOH group, a CN group, or a OMe group.

In another embodiment, A can be substituted with two or more nonhydrogen substituents. In such instances, the substituent groups can be the same or different. For example, A can be substituted with two hydroxy groups, or with one hydroxy group and one COOH group. Alternatively, A can be substituted with a hydroxy group and a nitro group. Any combination of the above listed substituents, including complex substituents such as those listed in Tables 4 to 10, is contemplated by the present invention. Similarly, where compounds of the invention contain three or more substituents any combination of the above listed substituents is encompassed by the invention.

Likewise, the substituent R₆ attached to the carbon atom between A and the thiohydantoin ring can be either hydrogen or a substituent other than hydrogen. Where R₆ is a substituent other than hydrogen, it can be alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, HPO₄, H₂PO₃, H₂PO₂, HPO₃R₁₁, PO₂R₁₀R₁₁, CN, or X, where R₉, R₁₀, and R₁₁ are as defined in Formula I. When R₆ contains an active hydroxy group, it also can be present in the form of an ether or ester, for example, an alkyl ether or alkyl ester. Thus, the invention encompasses compounds in which R₆ can be an OAlkyl group or a COOAlkyl group. The present invention further encompasses compounds in which R₆ is a complex substituent such as those provided in Tables 4 to 10.

In one aspect, the invention provides compounds in which all of the substituents attached to A are not hydrogen. In other words, the invention includes compounds in which A is substituted with at least at one substituent other than hydrogen.

Compounds having complex substituents are encompassed by the invention. The following formulas are representative of such compounds. In each of the formula, any combination of the variables listed can exist. Nonlimiting examples of pseudothiohydantoin compounds corresponding to formulas Ia to Ik are provided in Tables 4 to 10. However, it is understood that the invention also encompasses similar compounds in accordance with formulas IIa to IIk and IIIa to IIIk. The compounds represented in Tables 4 to 10 are only examples of compounds of the invention and are not intended to be all-inclusive. One having ordinary skill in the art would readily recognize other compounds within the scope of formulas I, II, and III that are also part of the invention.

In one embodiment, the invention provides compounds and combinatorial libraries of compounds having formula Ia

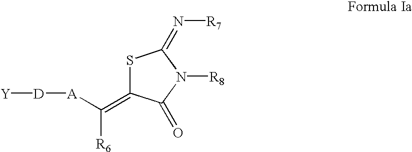

wherein D is alkylene, alkenylene, alkynylene, aryl or heterocycle. Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula I.

As used herein, the terms “alkylene,” “alkenylene,” and “alkynylene” refer to alkyl, alkenyl, and alkynyl groups as defined above in which one additional atom has been removed such that the group is divalent. Nonlimiting examples of such groups include —CH₂CH₂CH₂—, —CH₂CH═CHCH₂—, and —CH₂C≡CCH₂—.

In a second embodiment, the invention provides compounds and combinatorial libraries of compounds having formula Ib

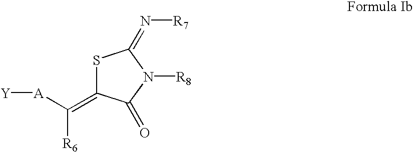

wherein Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula I.

In the following formulas, the variable E can be present or absent. When present, E is defined as provided. When E is absent, the atom immediately distal to E is attached directly to the phenyl ring.

In a third embodiment, the invention provides compounds and combinatorial libraries of compounds having formula Ic

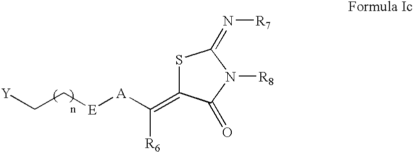

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula I.

In another embodiment, the invention provides compounds and combinatorial libraries of compounds having formula Id

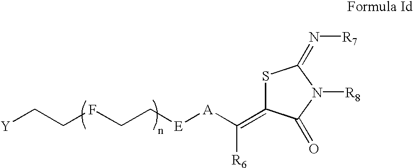

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula I.

In another embodiment, the invention provides compounds and combinatorial libraries of compounds having formula Ie

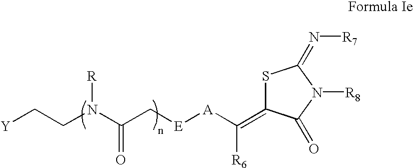

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula I.

In another embodiment, the invention provides compounds and combinatorial libraries of compounds having formula If

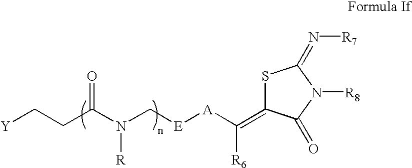

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula I.

In another embodiment, the invention provides compounds and combinatorial libraries of compounds having formula Ig

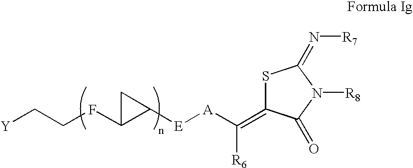

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula I.

In yet another embodiment, the invention provides compounds and combinatorial libraries of compound having formula Ih

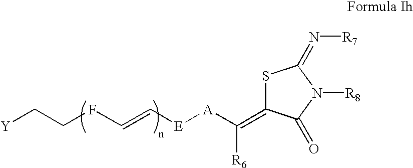

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is CR₁₀R₁₁, CONR₁₁, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula I.

In a further embodiment, the invention provides compounds and combinatorial libraries of compounds having formula Ii

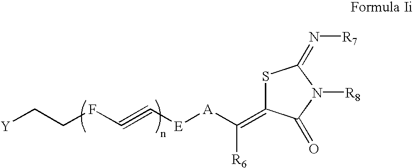

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is CR₁₀R₁₁, CONR₁₁, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula I.

In another embodiment, the invention provides compounds and combinatorial libraries of compounds having formula Ij

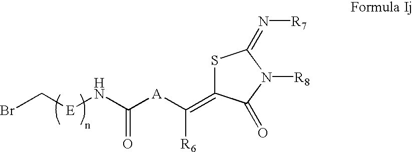

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula I.

In yet another embodiment, the invention provides compounds and combinatorial libraries of compounds having formula Ik

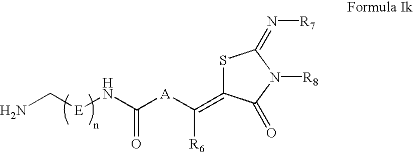

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula I.

In a further embodiment, the invention provides compounds and combinatorial libraries of compounds having formula Il

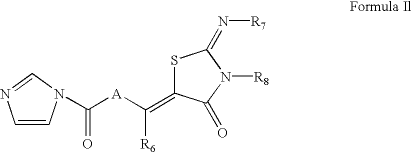

wherein R₆ to R₈ are as defined above for Formula I.

In one aspect, the invention provides compounds and combinatorial libraries of compounds having the formula:

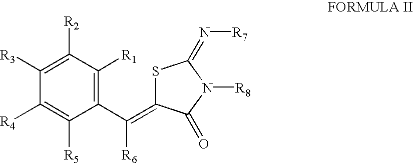

wherein R₁ to R₆ each independently are H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring. The compounds of Formula II are compounds of Formula I in which A is a 6-member aromatic carbocyclic ring, i.e. a benzene ring. However, it is understood by those skilled in the art that the present invention also encompasses compounds containing other five, six, and seven aromatic rings. For convenience, the invention is further described in terms of compounds of Formula II. However, the invention is not limited to such compounds, but includes similar compounds containing other aromatic rings.

In one embodiment of the invention, only one of the substituents on the phenyl ring is a substituent other than hydrogen. For example, R₁ to R₅ is a substituent other than hydrogen. In such instances, R₁ to R₅ independently can be, H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X where R₉, R₁₀, and R₁₁ are defined above for Formula II. For example, R₁ to R₅ each independently can be an amide, a halogen, a hydroxy group, an alkoxy group, an acid group, a nitrile, or a nitro group. When compounds of the invention contain an active hydroxy group, they also can be present in the form of an ether or ester, for example, an alkyl ether or alkyl ester. Thus, the invention encompasses compounds in which R₁ to R₅ can be an OAlkyl group or a COOAlkyl group. Non-limiting examples of OAlkyl groups include OMe (OCH₃), OEt (OCH₂CH₃), OPr (OCH₂CH₂CH₃), and the like. Non-limiting examples of COOAlkyl groups include COOMe, COOEt, COOPr, COOBu, COO-tBu, and the like.

In another embodiment, two or more of R₁ to R₅ are substituents other than hydrogen. In such instances, the substituent groups can be the same or different. For example, the phenyl ring of the compounds can be substituted with two hydroxy groups. Alternatively, the phenyl ring of the compounds can be substituted with an OH group and one of a COOH group, a nitro group, or an alkoxy group. Any combination of the above listed substituents for R₁ to R₅ is contemplated by the present invention. Similarly, where the compounds of the invention contain three or more substituents any combination of R₁ to R₅ is encompassed by the invention.

Likewise, the substituent R₆ attached to the carbon atom between the phenyl and the thiohydantoin rings can be either hydrogen or a substituent other than hydrogen. Where R₆ is a substituent other than hydrogen, it can be alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, HPO₄, H₂PO₃, H₂PO₂, HPO₃R₁₁, PO₂R₁₀R₁₁, CN, or X, where R₉, R₁₀, and R₁₁ are as defined in Formula III. When R₆ contains an active hydroxy group, it also can be present in the form of an ether or ester, for example, an alkyl ether or alkyl ester. Thus, the invention encompasses compounds in which R₆ can be an OAlkyl group or a COOAlkyl group. The present invention further encompasses compounds in which R₆ is a complex substituent such as those provided in Tables 4 to 10.

In one aspect, the invention provides compounds in which not all of R₁ to R₆ are hydrogen. In other words, the invention includes compounds in which at least one of R₁ to R₆ is a substituent other than hydrogen.

In one embodiment, the invention provides compounds and combinatorial libraries of compounds having formula IIa

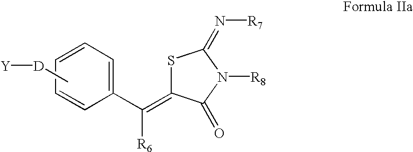

wherein D is alkylene, alkenylene, alkynylene, aryl or heterocycle. Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula II.

In a second embodiment, the invention provides compounds and combinatorial libraries of compounds having formula IIb

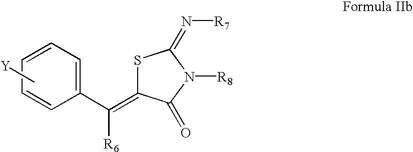

wherein Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula II.

In the following formulas, the variable E can be present or absent. When present, E is defined as provided. When E is absent, the atom immediately distal to E is attached directly to the phenyl ring.

In a third embodiment, the invention provides compounds and combinatorial libraries of compounds having formula IIc

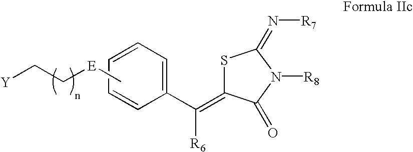

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula II.

In another embodiment, the invention provides compounds and combinatorial libraries of compounds having formula IId

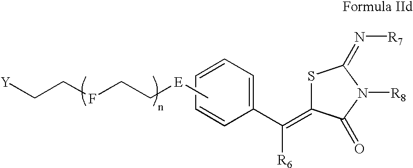

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula II.

In another embodiment, the invention provides compounds and combinatorial libraries of compounds having formula IIe

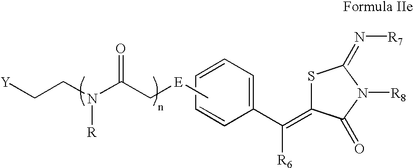

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula II.

In another embodiment, the invention provides compounds and combinatorial libraries of compounds having formula IIf

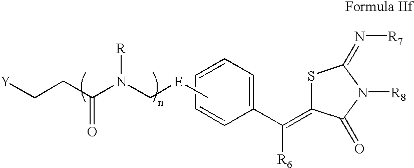

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula II.

In another embodiment, the invention provides compounds and combinatorial libraries of compounds having formula IIg

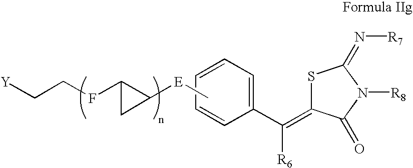

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula II.

In yet another embodiment, the invention provides compounds and combinatorial libraries of compound having formula IIh

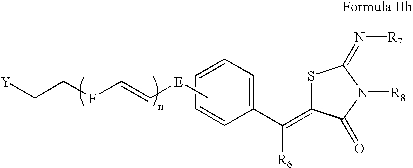

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is CR₁₀R₁₁, CONR₁₁, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula II.

In a further embodiment, the invention provides compounds and combinatorial libraries of compounds having formula IIi

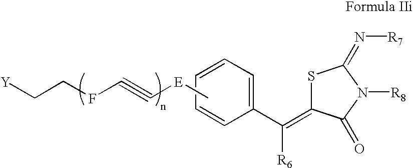

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is CR₁₀R₁₁, CONR₁₁, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula II.

In another embodiment, the invention provides compounds and combinatorial libraries of compounds having formula IIj

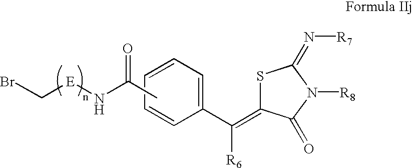

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula II.

In yet another embodiment, the invention provides compounds and combinatorial libraries of compounds having formula IIk

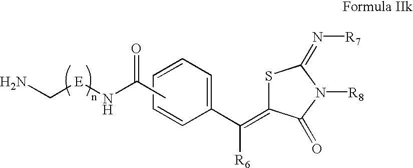

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula II.

In a further embodiment, the invention provides compounds and combinatorial libraries of compounds having formula IIl

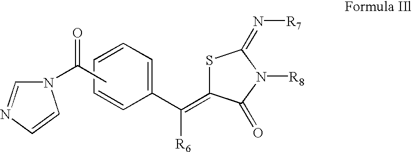

wherein R₆ to R₈ are as defined above for Formula II.

Exemplified compounds of Formula II include, but are not limited to, 5-(4-hydroxy-3-nitro-benzylidene)-2-imino-thiazolidin-4-one; 5-(3-hydroxy-4-nitro-benzylidene)-2-imino-thiazolidin-4-one; 5-(3,4-dihydroxy-benzylidene)-2-imino-thiazolidin-4-one; 3-(2-imino-4-oxo-thiazolidin-5-ylidenemethyl)-benzoic acid; 4-(2-imino-4-oxo-thiazolidin-5-ylidenemethyl)-benzoic acid; 5-(4-hydroxy-3-methoxy-benzylidene)-2-imino-thiazolidin-4-one; 5-(3-hydroxy-4-methoxy-benzylidene)-2-imino-thiazolidin-4-one; 2-hydroxy-5-(2-imino-4-oxo-thiazolidin-5-ylidenemethyl)-benzoic acid; 3-(2-imino-4-oxo-thiazolidin-5-ylidenemethyl)-benzonitrile; 2-imino-5-(3-nitro-benzylidene)-thiazolidin-4-one; 2-(2-imino-4-oxo-thiazolidin-5-ylidenemethyl)-benzoic acid; N-[4-(2-imino-4-oxo-thiazolidin-5-ylidenemethyl)-phenyl]-acetamide; and 5-(2,5-dihydroxy-benzylidene)-2-imino-thiazolidin-4-one.

In another aspect, the invention provides

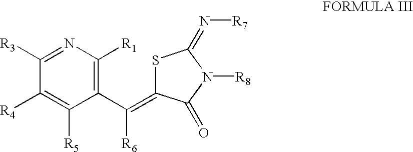

wherein R₁, R₃, R₄, R₅, and R₆ each independently are hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

Compounds of Formula III are compounds of Formula III in which A is a 6-membered aromatic heterocyclic ring containing 5 carbon atoms and 1 nitrogen atom. It is appreciated by those skilled in the art that the present invention encompasses compounds of the general Formula III in which the nitrogen atom on the pyridine ring is at any position relative to the thiohydantoin ring. Such compounds include all manner of combinations for R₁ to R₆ as discussed above with regard to compounds of Formula I. An exemplified compound of this formula is 2-imino-5-pyridin-3-ylmethylene-thiazolidin-4-one.

The present invention encompasses compounds of Formula III in which the nitrogen atom is located at any position on the pyridine ring in relation to the thiohydantoin ring. The present invention also encompasses compounds of Formula III containing heterocyclic rings other than a pyridine ring. Such heterocyclic rings include those having from five to seven ring atoms where from one to three of the ring atoms is a heterocyclic atom, for example, nitrogen, oxygen, or sulfur. Where the heterocyclic ring contains more than one heterocyclic atom, the heterocyclic atoms can be the same or different. Examples of such heterocyclic rings include, but are not limited to, pyroles, pyrazoles, imidazoles, pyridines, pyrimidines, pyridazines, pyrazines, triazines, furans, oxazoles, thiazoles, thiophenes, and quinolines.

The heterocyclic rings of the compounds of Formula III can be unsubstituted or substituted. When substituted, suitable substituents include, but are not limited to hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring. For convenience, the invention is further described in terms of compounds of Formula III. However, the invention is not limited to such compounds, but includes similar compounds containing other heterocyclic rings.

In one embodiment of the invention, only one of R₁ to R₅ is a substituent other than hydrogen. In such instances, R₁ to R₅ independently can be, H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X where R₉, R₁₀, and R₁₁ are as defined above for Formula II. For example, R₁ to R₅ each independently can be an amide, a halogen, a hydroxy group, an alkoxy group, an acid group, a nitrile, or a nitro group. When compounds of the invention contain an active hydroxy group, they also can be present in the form of an ether or ester, for example, an alkyl ether or alkyl ester. Thus, the invention encompasses compounds in which R₁ to R₅ can be an OAlkyl group or a COOAlkyl group. Non-limiting examples of OAlkyl groups include OMe (OCH₃), OEt (OCH₂CH₃), OPr (OCH₂CH₂CH₃), and the like. Non-limiting examples of COOAlkyl groups include COOMe, COOEt, COOPr, COOBu, COO-tBu, and the like.

In another embodiment, two or more of R₁ to R₅ are substituents other than hydrogen. In such instances, the substituent groups-can be the same or different. For example, the phenyl ring of the compounds can be substituted with two hydroxy groups. Alternatively, the phenyl ring of the compounds can be substituted with an OH group and one of a COOH group, a nitro group, or an alkoxy group. Any combination of the above listed substituents for R₁ to R₅ is contemplated by the present invention. Similarly, where the compounds of the invention contain three or more substituents any combination of R₁ to R₅ is encompassed by the invention.

Likewise, the substituent R₆ attached to the carbon atom between the phenyl and the thiohydantoin rings can be either hydrogen or a substituent other than hydrogen. Where R₆ is a substituent other than hydrogen, it can be alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, NR₁₀COR₁₁, N₃, NO₂, PH₃, PH₂R₁₁, HPO₄, H₂PO₃, H₂PO₂, HPO₃R₁₁, PO₂R₁₀R₁₁, CN, or X, where R₉, R₁₀, and R₁₁ are as defined in Formula II. When R₆ contains an active hydroxy group, it also can be present in the form of an ether or ester, for example, an alkyl ether or alkyl ester. Thus, the invention encompasses compounds in which R₆ can be an OAlkyl group or a COOAlkyl group. The present invention further encompasses compounds in which R₆ is a complex substituent such as those provided in Tables 4 to 10.

In one aspect, the invention provides compounds in which not all of R₁ to R₆ are hydrogen. In other words, the invention includes compounds in which at least one of R₁ to R₆ is a substituent other than hydrogen.

In one embodiment, the invention provides compounds and combinatorial libraries of compounds having formula IIIa

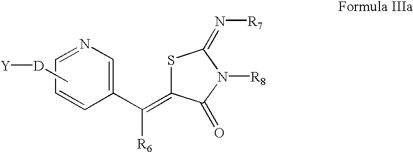

wherein D is alkylene, alkenylene, alkynylene, aryl or heterocycle. Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula III.

In a second embodiment, the invention provides compounds and combinatorial libraries of compounds having formula IIIb

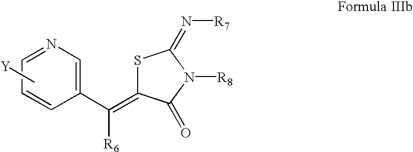

wherein Y is OH, NHR₁₁, SH, COOH, SO₂OH, X, CN, COR₁₁, N₃, CONH₂, CONHR₁₁, C≡CH, or CH═CH₂. R₆ to R₈ are as defined above for Formula III.

In the following formulas, the variable E can be present or absent. When present, E is defined as provided. When E is absent, the atom immediately distal to E is attached directly to the phenyl ring.

In a third embodiment, the invention provides compounds and combinatorial libraries of compounds having formula IIIc

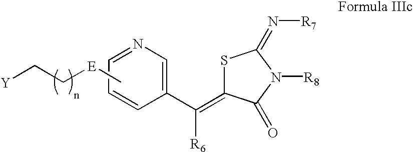

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula III.

In another embodiment, the invention provides compounds and combinatorial libraries of compounds having formula IIId

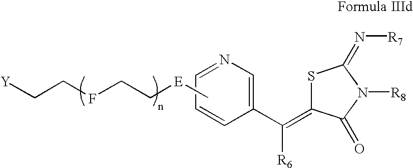

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula III.

In another embodiment, the invention provides compounds and combinatorial libraries of compounds having formula IIIe

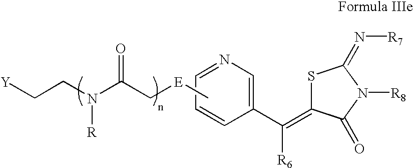

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula III.

In another embodiment, the invention provides compounds and combinatorial libraries of compounds having formula IIIf

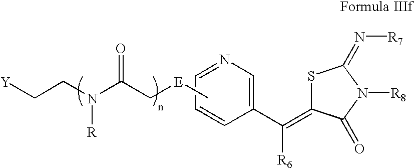

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R is alkyl, alkenyl, alkynyl, aryl or heterocycle; and R₆ to R₈ are as defined above for Formula III.

In another embodiment, the invention provides compounds and combinatorial libraries of compounds having formula IIIg

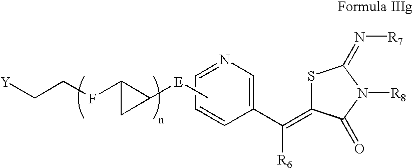

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula III.

In yet another embodiment, the invention provides compounds and combinatorial libraries of compound having formula IIIh

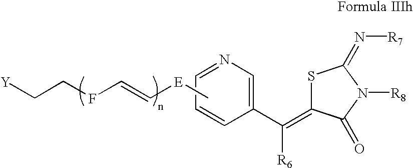

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula III.

In a further embodiment, the invention provides compounds and combinatorial libraries of compounds having formula IIIi

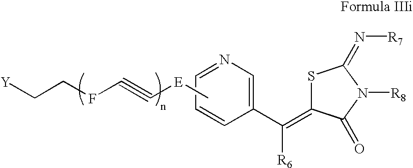

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH. Each F independently is O, S, NR₁₁, CR₁₀R₁₁, CONR₁₁, NR₁₀CONR₁₁, NR₁₀CNHNR₁₁, NR₁₁COO, C≡C, or CH═CH. Y is OH, NHR₁₁, SH, COOH, SO₂OH, X, CN, COR₁₁, N₃, CONH₂, CONHR₁₁, C≡CH, or CH═CH₂; and n is an integer between 0 and 5, inclusive. R₆ to R₈ are as defined above for Formula III.

In another embodiment, the invention provides compounds and combinatorial libraries of compounds having formula IIIj

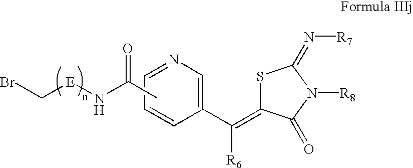

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula III.

In yet another embodiment, the invention provides compounds and combinatorial libraries of compounds having formula IIIk

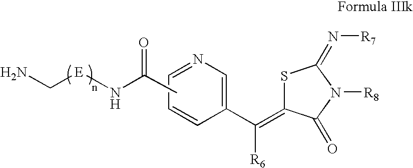

wherein E is O, S, NR₁₁, CR₁₀R₁₁, CONR₁₁, SO₂NR₁₁, NR₁₀CONR₁₁, NR₁₀CNHNR₁₁, NR₁₁COO, C≡C, or CH═CH, and n is an integer between 0 and 5, inclusive. R₆ to R₈ are as defined above for Formula III.

In a further embodiment, the invention provides compounds and combinatorial libraries of compounds having formula IIIl

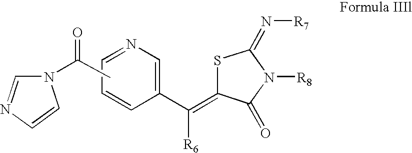

wherein R₆ to R₈ are as defined above for Formula III.

One or more of the compounds of the invention, even within a given library, can be present as a salt. The term “salt” encompasses those salts that form within the carboxylate anions and amine nitrogens and includes salts formed with the organic and inorganic anions and cations discussed below. Furthermore, the term includes salts that form by standard acid-based reactions with basic groups (such as amino groups) and organic or inorganic acids. Such acids include, hydrochloric, hydrofluoric, trifluoroacetic, sulfuric, phosphoric, acetic, succinic, citric, lactic, maleic, fumaric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the sodium, potassium, barium, aluminum, and calcium); ammonium and mono-, di-, and tri-alkyl amines, such as trimethylamine, cyclohexylamine; and the organic cations, such as dibenzylammonium, bis(2-hydroxyethyl)ammonium, and like cations. See for example “Pharmaceutical Salts,” Berge et al., J. Pharm. Sci., 66:1-19 (1977), which is incorporated herein by reference. Other cations encompassed by the above term include the protonated form of procaine, quinine, and N-methylglucosamine, and the protonated forms of basic amino acids such as glycine, ornithine, histidine, phenylglycine, lysine, and arginine. Furthermore, any zwitterionic form of the instant compounds formed by a carboxylic acid and an amino group is referred to by this term. For example, a cation for a carboxylate anion will exist when a position is substituted by a (quaternary ammonium)methyl group.

The compounds of the invention can also exist as solvates and hydrates. Thus, these compounds may crystallize with, for example, waters of hydration, or one, a number of, or any fraction thereof, of molecules of the mother liquor solvent. The solvates and hydrates of such compounds are included within the scope of this invention.

One or more compounds of the invention, even when in a library, can be in the biologically active ester form. Such as the non-toxic, metabolically-labile, ester-form. Such esters induce increased blood levels and prolong efficacy of the corresponding nonesterified forms of the compounds. Ester groups which can be used include the lower alkoxymethyl groups, for example, methoxymethyl, ethoxymethyl, isopropoxymethyl and the like; the —(C₁-C₁₂)alkoxyethyl groups, for example, methoxyethyl, ethoxyethyl, propoxyethyl, isopropoxyethyl and the like; the —(C₁-C₁₀)alkylthiomethyl groups, for example, methylthiomethyl, ethylthiomethyl, iso-propylmethyl and the like; and the acyloxymethyl groups, for example, pivaloyloxymethyl, pivaloyloxyethyl, acetoxymethyl, and acetoxyethyl. Salts, solvates, hydrates, biologically active esters of the compounds of the invention are common ligand variants of the compounds as defined above.

In another aspect, the present invention provides bi-ligands that contain a common ligand mimic as described above and a specificity ligand. In the bi-ligands of the invention, the common ligand mimic and the specificity ligand can be attached directly or indirectly. The common ligand mimic and specificity ligand are attached via a covalent bond formed from the reaction of one or more functional groups on the common ligand mimic with one or more functional groups on the specificity ligand. Direct attachment of the individual ligands in the bi-ligand can occur through reaction of simple functional groups on the ligands. Indirect attachment of the individual ligands in the bi-ligand can occur through a linker molecule. Such linkers include those provided in Tables 4 to 10. These linkers bind to each of the common ligand mimic and the specificity ligand through functional groups on the linker and the individual ligands. Some of the common ligand mimics of the present invention having substituents that include linker molecules, e.g. the common ligand mimics of Tables 4 to 10. Tailoring of the specific type and length of the linker attaching the common ligand mimic and specificity ligand allows tailoring of the bi-ligand to optimize binding of the common ligand mimic to a conservative site on the receptor and binding of the specificity ligand to a specificity site on the receptor.

The present invention provides specificity ligands that are specific for NAD receptors and combinatorial libraries containing these specificity ligands. For example, in one embodiment, compounds of the invention are ligands for specificity sites on dehydrogenases and reductases like those described above.

In another embodiment of the present invention, the protected specificity ligand is a compound having formula

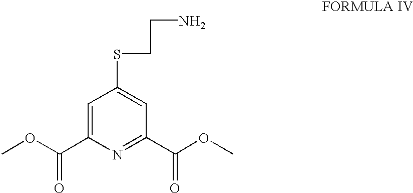

Specificity ligands, such as that of Formula IV can also exist as salts, or in other reactive forms and can be reacted with the common ligand mimics of the invention to provide bi-ligands of the invention.

Bi-ligands of the invention can be bi-ligands for any receptor. In one embodiment, the bi-ligand is a bi-ligand that binds a dehydrogenase or reductase. In another embodiment, bi-ligands of the present invention comprise a pseudothiohydantoin compound as a common ligand mimic and a specificity ligand. For example, bi-ligands of the invention can contain a common ligand mimic of Formula I coupled to a specificity ligand. Alternatively, bi-ligands of the invention can contain a common ligand mimic of Formula II or Formula III coupled to a specificity ligand. The specificity ligand can be any specificity ligand, for example a ligand that binds to a specificity site on an oxidoreductase. In such an embodiment, the specificity ligand can be a pyridine dicarboxylate. Examples of particular bi-ligands that fall within the invention are provided in FIG. 9.

The compounds of the present invention can be produced by any feasible method. For example, the compounds of the present invention can be produced by the following methods. Generally, these methods include reaction of pseudothiohydantoin with a compound such as a carboxybenzaldehyde, pyridine carboxyaldehyde, or pyrimidine carboxyaldehyde. Tailoring of the methods of the invention to produce a particular compound within the scope of the invention is within the level of skill of the ordinary artisan.

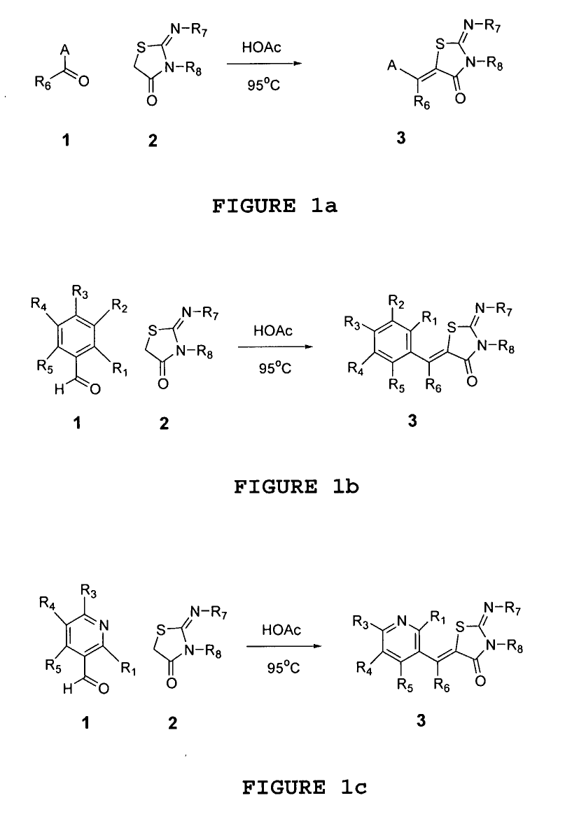

In one aspect, as shown in FIGS. 1a, 1b, and 1c, the present invention provides a method for the manufacture of pseudothiohydantoin compounds. In such a method, pseudothiohydantoin is mixed with a compound such as a carboxybenzaldehyde, pyridine carboxyaldehyde, or pyrimidine carboxyaldehyde. The mixture is heated at a temperature of about 60 to 120° C. for a period of about 1 to 24 hours. For example, the mixture can be heated to a temperature of about 95° C. for a period of about 8 hours. The reaction mixture then can be cooled.

The product can be washed with a mixture of water and ethyl acetate. If desired, the product can be purified by any conventional means.

In one embodiment, pseudothiohydantoin is reacted with 4-carboxybenzaldehyde at a temperature of about 95° C. for 8 hours to produce 4-(2-imino-4-oxo-thiazolidin-5-ylidenemethyl)-benzoic acid.

The methods of the present invention now will be described in terms of specific embodiments for the preparation of a compound of formula I

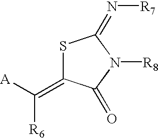

wherein A is an aromatic carbocyclic or heterocyclic ring containing 5, 6, or 7 members and having from 0 to 3 heterocyclic atoms selected from the group consisting of oxygen, nitrogen, and sulfur. A is optionally substituted with from one to five substituents which each independently are hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, or X. R₇ and R₈ each independently are hydrogen, OH, NH₂, alkyl, alkenyl, alkynyl, aryl, or heterocycle, or R₇ and R₈ can be attached indirectly through an alkylene, alkenylene, or alkynylene chain to form a heterocyclic ring fused to the thiohydantoin ring. R₉, R₁₀, and R₁₁ each independently are hydrogen, alkyl, alkenyl, alkynyl, aryl, or heterocycle, or R₉ and R₁₀ together with the nitrogen atom to which they are attached can be joined to form a heterocyclic ring.

The method includes forming a mixture of a pseudothiohydantoin and a carboxybenzaldehyde, carboxypyridine, or carboxypyrimidine. For example, a pseudothiohydantoin and 4-carboxybenzaldehyde can be reacted. The mixture is then heated to a temperature of about 60 to 120° C., for example 95° C., for a period of about 1 to 24 hours, for example 8 hours. The pseudothiohydantoin product can be washed with a mixture of water and ethyl acetate.

Bi-ligands of the present invention can be produced by any feasible method. For example, the compounds of the present invention can be produced by the following methods. These methods are exemplified using a common ligand mimic or Formula II and a pyridine dicarboxylate specificity ligand. However, one having ordinary skill in the art will appreciate that variations in such methods can be employed to produce bi-ligands having other common ligand mimics or other specificity ligands and that such compounds and methods are within the scope of the present invention.

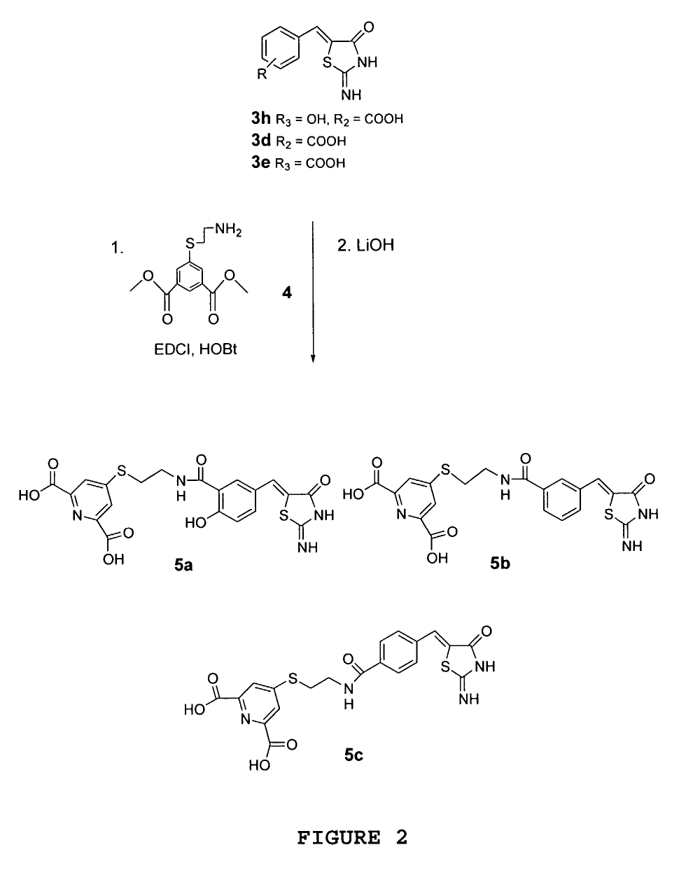

As shown in FIG. 2, a common ligand mimic of the invention, such as a pseudothiohydantoin compound of Formula II can be reacted with a pyridine dicarboxylate compound in a solvent in the presence of HOBt H₂O. Suitable solvents include dimethylformamide, tetrahydrofuran, dimethyl ether, and dichloromethane. For example, the reaction of dicarboxylic acid and pyridine can be performed in dimethylformamide with the addition of hydrated HOBt.H₂O. Triethylamine and 1-dimethylaminopropyl-3-ethyl-carbodiimide (EDCI) are then added to the mixture. The reaction is then stirred at room temperature for a period of about 2 to 50 hours. For example, the reaction can be stirred at room temperature for a period of about two days.

The reaction precipitate is collected and washed in a mixture of solvent and hydrochloric acid. Then, the recovered solid can be suspended in a mixture of alcohol and water, such as a methanol and water mixture. This solution is stirred at room temperature for a period of about 1 to 24 hours until it is homogenous. The solution is then precipitated, for example, with aqueous 2N HCl. The resulting precipitated product can then be filtered, washed with water, and dried.

As used herein, a “combinatorial library” is an intentionally created collection of differing molecules that can be prepared by the means provided below or otherwise and screened for biological activity in a variety of formats (e.g., libraries of soluble molecules, libraries of compounds attached to resin beads, silica chips or other solid supports). A “combinatorial library,” as defined above, involves successive rounds of chemical syntheses based on a common starting structure. The combinatorial libraries can be screened in any variety of assays, such as those detailed below as well as others useful for assessing their biological activity. The combinatorial libraries will generally have at least one active compound and are generally prepared such that the compounds are in equimolar quantities.

Compounds described in previous work that are not taught as part of a collection of compounds or not taught as intended for use as part of such a collection are not part of a “combinatorial library” of the invention. In addition, compounds that are in an unintentional or undesired mixture are not part of a “combinatorial library” of the invention.

The present invention provides combinatorial libraries containing two or more compounds. The present invention also provides combinatorial libraries containing three, four, or five or more compounds. The present invention further provides combinatorial libraries that can contain ten or more compounds, for example, fifty or more compounds. If desired, the combinatorial libraries of the invention can contain 100,000 or more, or even 1,000,000 or more, compounds.

In one embodiment, the present invention provides combinatorial libraries containing common ligand variants of compounds of Formula I. These common ligand variants are active forms of the compounds of Formula I that are capable of binding to a specificity ligand to form a bi-ligand. For example, where one of the substituents, e.g. R₁ to R₅, is a COOH or COOAlkyl group, the common ligand variant can be a compound containing the group COO⁻. Common ligand variants of the invention include common ligand mimics in which the substituents on the compounds are complex ligands such as those attached to the compounds listed in Tables 4 to 10. Compounds of formulas II and III can similarly be used to prepare combinatorial libraries of the present invention.

In another embodiment, the present invention provides combinatorial libraries containing bi-ligands of the invention. The bi-ligands are the reaction product of a common ligand mimic and a specificity ligand which interact with distinct sites on a single receptor. For example, the common ligand mimic can be one or more common ligand mimic for NAD that binds to a conserved site on a dehydrogenase, like ADH. In such a bi-ligand, the specificity ligand is one or more ligands that bind a specificity site on ADH.

Such combinatorial libraries can contain bi-ligands having a single common ligand mimic bonded to multiple specificity ligands. Alternatively, the combinatorial libraries can contain bi-ligands having a single specificity ligand bonded to multiple common ligand mimics. In another aspect, the combinatorial libraries can contain multiple common ligand mimics and multiple specificity ligands for one or more receptors.

The use of a common ligand mimic of the invention to produce the combinatorial library allows generation of combinatorial libraries having improved affinity and/or specificity. Selection and tailoring of the substituents on the common ligand mimic also allows for production of combinatorial libraries in a more efficient manner than heretofore possible.

Bi-ligand libraries of the invention can be prepared in a variety of different ways. For example, two methods employing a resin, such as HOBt resin, carbodiimide resin, or DIEA (diisopropyldiisoamine) resin can be used to form bi-ligand libraries. In one such method, bi-ligand libraries can be prepared via direct coupling of amines to common ligand mimics of the invention having a carboxylic acid group.

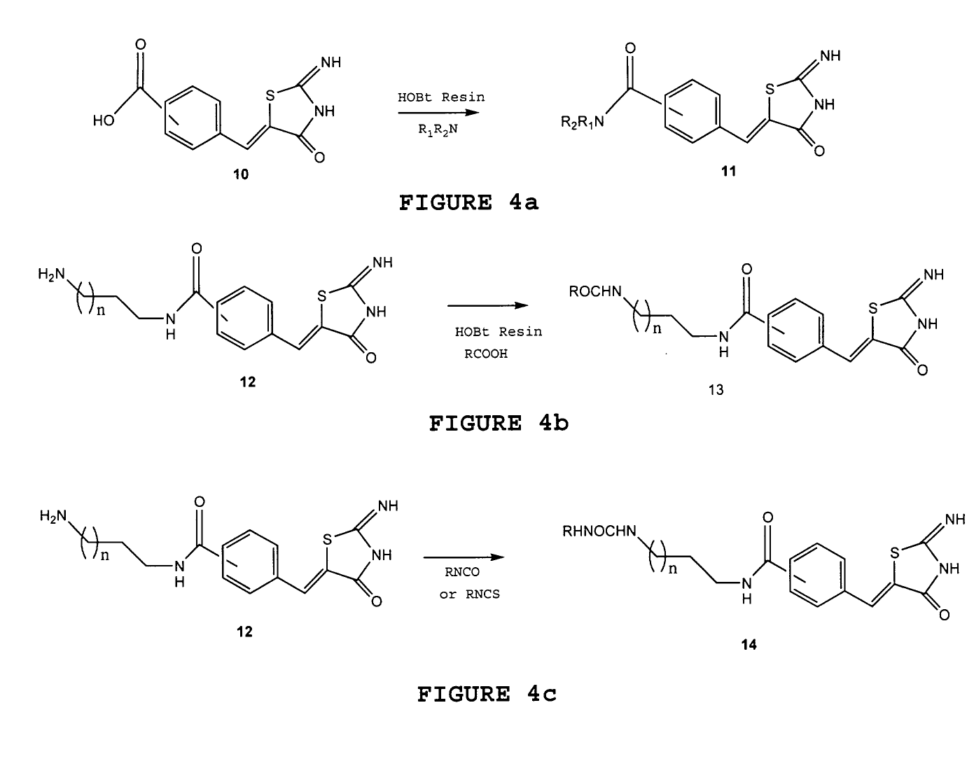

As shown in FIG. 4a, bi-ligand libraries can be prepared in the following manner. HOBt resin is swelled in a dry solvent, such as dry DMF, and added to a solution of a common ligand mimic of the invention that is dissolved in a solvent, such as a mixture of DMF and DIC. The solution is shaken at room temperature overnight and then washed with 3× dry DMF and 3× dry THF.

The resin is added to a solution of an amine in a mixed solvent, for example dry THF/DMF. The mixture is shaken again at room temperature overnight. The resin then can be filtered and washed with solvent, and the filtrate can be collected and vacuum dried to provide bi-ligands of the invention. Nonlimiting examples of amines useful for the preparation of bi-ligand libraries include those in Table 1.

TABLE 1

| | | |
|---|---|---|
| cyclopropylamine | Nipecotamide | 3-chloro-p-anisidine |
| isopropylamine | N-butylamine | 5-amino-l-napthol |
| N,N-diethyl-N′- | 2-(2-aminoethyl)-1- | 2-amino-5,6-dimethyl- |
| methylethylenediamine | methylpyrrolidine | benzimidazole |
| N-(3-aminopropyl)-N- | 2-(aminomethyl)-1- | N,N-diethyl-p- |
| methylaniline | ethylpyrrolidine | phenylenediamine |
| hydroxylamine | N-(2-aminoethyl)- | 1-(2-pyridyl) |
| hydrochloride | piperidine | piperazine |
| 4-amino-1,2,4- | 4-(2-aminoethyl) | 3,5- |
| triazole | morpholine | dimethoxybenzylamine |
| N-methylallylamine | Propylamine | Pyrrolidine |
| 3-pyrroline | 3-aminobenzamide | 1-phenylpiperazine |
| diethylamine | ethyl 3-aminobutyrate | 4-butoxyaniline |
| isobutylamine | 5-aminoindan | Cyclopentylamine |
| 1-(3-aminopropyl) | trans-2- | 2,4-dimethoxy |
| pyrrolidine | phenylcyclopropylamine | benzylamine |
| N-methylpropylamine | 3-phenyl-1-propylamine | 4-pentylaniline |
| sec-butylamine | beta-methylphenethylamine | ethyl 4-aminobutyrate |
| 2-methoxyethylamine | N-methylphenethylamine | 1-cyclohexylpiperazine |
| cyclobutylamine | p-isopropylaniline | 4-piperidinopiperidine |
| 2,3-dimethoxybenzylamine | 2-amino-5-trifluoromethyl-1,3,4- | 2-amino-5- |
| | thiadiazole | chlorobenzoxazole |
| ethyl 4-amino-1- | N,N-dimethyl-1,4- | 2-(aminomethyl) |
| piperidinecarboxylate | phenylenediamine | benzimidazole |
| morpholine | N-(4- | 2-aminobiphenyl |
| | pyridylmethyl)ethylamine | |
| 1-ethylpropylamine | 4-aminobenzamide | 3-aminobiphenyl |
| neopentylamine | 3,4-(methylenedioxy)- | N-undecylamine |
| | aniline | |
| N-ethylisopropylamine | 4-hydroxybenzamide | Piperidine |
| N-methylbutylamine | 6-aminonicotinamide | 4-cyclohexylaniline |
| 2-amino-1- | 4-fluorophenethylamine | 2-(trifluoromethyl) |
| methyloxypropane | hydrochloride | benzylamine |
| 3-methoxypropylamine | 3-amino-4-methylbenzyl | 2,4-dimethyl-6-aminophenol |
| | alcohol | |
| thiazolidine | 3-methoxybenzylamine | 2,4-dichlorobenzylamine |
| 3-amino-1,2,4-triazine | 4-ethoxyaniline | 3,4-dichlorobenzylamine |
| furfurylamine | 4-methoxy-2-methylaniline | 4-aminoquinaldine |
| diallylamine | 4-methoxybenzylamine | 4-(methylthio)aniline |
| 2-methylpiperidine | m-phenetidine | 1-benzylpiperazine |
| 3-methylpiperidine | 5-amino-2-methoxyphenol | 4-piperidino aniline |
| 4-methylpiperidine | Tyramine | 4-(trifluoromethoxy)- |
| | | aniline |
| cyclohexylamine | 2-fluorophenethylamine | 4-hexylaniline |
| hexamethyleneimine | 3-fluorophenethylamine | 4-amino-2,6- |
| | | dichlorophenol |
| 1-aminopiperidine | 3-(methylthio)aniline | 4-morpholinoaniline |
| 2-amino-4-methoxy-6- | (3S)-(+)-1-benzyl-3- | N-(2-aminoethyl)-N- |
| methylpyrimidine | aminopyrrolidine | ethyl-m-toluidine |
| tetrahydrofurfurylamine | 1-methylpiperazine | 4-chlorobenzylamine |
| 1,3-dimethylbutylamine | Dipropylamine | 1-(2-furoyl)piperazine |
| 3-chlorobenzylamine | 2-chlorobenzylamine | 1-(2-fluorophenyl) |
| | | piperazine |
| 4-aminomorpholine | 3,3,5- | 1-(4-fluorophenyl) |
| | trimethylcyclohexylamine | piperazine |
| N-(3′-aminopropyl)-2- | 4-aminophenylacetic acid | 2-(3,4-dimethoxyphenyl) |
| pyrrolidinone | ethyl ester | ethylamine |
| 3-dimethylamino | N-acetylethylenediamine | 2-amino-fluorene |
| propylamine | | |
| N-isopropylethylene | 2,4-difluorobenzylamine | 3,4,5-trimethoxyaniline |
| diamine | | |
| o-toluidine | N-phenyl-p-phenylenediamine | 4-aminodiphenylmethane |
| 1-aminonaphthalene | 2,6-difluorobenzylamine | Aminodiphenylmethane |
| 5-amino-1-pentanol | 3,4-difluorobenzylamine | 2,5-difluorobenzylamine |
| 3-ethoxypropylamine | 2-(aminomethyl)-1,3- | 3-phenoxyaniline |
| | dioxolane | |
| 3-(methylthio) | 2-aminonaphthalene | 4-phenoxyaniline |
| propylamine | | |
| benzylamine | p-phenetidine hydrochloride | 1-(3- |
| | | chlorophenyl)piperazine |
| m-toluidine | 8-aminoquinoline | 4-amino-1- |
| | | benzylpiperidine |
| 3-fluoroaniline | N-(3-aminopropyl) | 4-aminohippuric acid |
| | morpholine | |
| p-toluidine | 7-amino-4-methylcoumarin | 2-amino-9-fluorenone |
| 1-amino-5,6,7,8- | 4-piperidone monohydrate | 2-methyl-1-(3- |
| tetrahydronaphthalene | hydrochloride | methylphenyl)piperazine |
| 2-(aminomethyl)pyridine | 2-amino-1- | 3,4,5- |
| | methylbenzimidazole | trimethoxybenzylamine |
| 3-(aminomethyl)pyridine | 4-phenylbutylamine | 2,2-diphenylethylamine |
| 4-(aminomethyl)pyridine | 4-amino-N-methylphthalimide | 3-benzyloxyaniline |
| 1,2,3,4-tetrahydro-1- | 4-(2-aminoethyl)benzene | 4-amino-4′- |
| naphthylamine | sulfonamide | methyldiphenylether |
| 2-amino-4- | N-propylcyclopropane | 1-methyl-3- |
| methylbenzothiazole | methylamine | phenylpropylamine |
| 2-thiophenemethylamine | 4-tert-butylaniline | exo-2-aminonorbornane |
| 2-methylcyclohexylamine | 4′-aminoacetanilide | 1,4-benzodioxan-5-amine |
| 3,5-dimethylpiperidine | N-(4-aminobenzoyl)-beta- | Piperonylamine |
| | alanine | |
| 4-methylcyclohexylamine | methyl 3-amino-benzoate | 5-phenoxy-o-anisidine |
| N-isopropyl-N-phenyl-p- | 2-methoxy-N-phenyl-1,4- | 4-amino-4′- |
| phenylenediamine | phenylenediamine | chlorodiphenylether |
| cyclohexanemethylamine | 2-ethoxybenzylamine | 1-piperonylpiperazine |
| heptamethyleneimine | 2-methoxyphenethylamine | 4-amino-4′- |
| | | methoxystilbene |
| 1-(4- | 4-isopropoxyaniline | Cycloheptylamine |
| nitrophenyl)piperazine | | |
| 1-piperazinecarbox | 4-methoxyphenethylamine | (−)-cis-myrtanylamine |
| aldehyde | | |
| 2-amino-4- | 3,5-dimethoxyaniline | 4-(4-nitrophenoxy)- |
| methylthiazole | | aniline |
| 1,3,3-trimethyl-6- | alpha-(cyanoimino)-3,4- | 4-amino-4′- |
| azabicyclo[3,2,1]octane | dichlorophenethylamine | nitrodiphenylsulfide |
| 1-methylhomopiperazine | 1-ethylpiperazine | 2-amino-7-bromofluorene |
| N-(2-aminoethyl) | 4-tert-butylcyclohexylamine | 2-(3-chlorophenyl) |
| pyrrolidine | | ethylamine |
| 2-amino-5-phenyl-1,3,4- | 2-amino-4,5,6,7- | (1R,2S)-(+)-cis-1-amino- |
| thiadiazole sulfate | tetrahydrobenzo(b) | 2-indanol |
| | thiophene-3-carbonitrile | |
| 1-amino-4- | 2-(4-chlorophenyl) | n-undecylamine |
| methylpiperazine | ethylamine | |
| 2-heptylamine | 1-(3-aminopropyl)-2- | 2,6-dimethylmorpholine |
| | pipecoline | |
| N,N,N′-trimethyl-1,3- | 4-amino-2,2,6,6- | d(+)-alpha- |
| propanediamine | tetramethylpiperidine | methylbenzylamine |
| N-methylhexylamine | ethyl nipecotate | dl-1-amino-2-propanol |
| 1-(3-aminopropyl)-4- | N,N-dimethyl-N′- | dl-alpha- |
| methyl-piperazine | ethylethylenediamine | methylbenzylamine |
| 3-aminobenzyl alcohol | N,N-diethylethylenediamine | o-anisidine |
| (R)-(+)-2-amino-3- | 2-(furfurylthio) ethylamine | 3-amino-4-methylbenzyl |
| phenylpropanol | | alcohol |
| 2-(2-aminoethyl)-1,3- | 2,3-dimethyl | 3-amino5,5-dimethyl-2- |
| dioxolane | cyclohexylamine | cyclohexen-1-one |
| 6-amino-1-hexanol | N-methyl-b-alaninenitrile | 3-aminophenol |
| 3-isopropoxy | 1-methyl-4- | (R)-(+)-1- |
| propylamine | (methylamino)piperidine | phenylpropylamine |
| 2-methylbenzylamine | 1-amino-2-butanol | 2-piperidineethanol |
| (R)-1-(4-methylphenyl) | 2-amino-2-methyl-1-propanol | 2,3-dimethyl-4- |
| ethylamine | | aminophenol |
| 3-methylbenzylamine | 4-amino-1-butanol | 1-aminoindan |
| 4-methylbenzylamine | 3-(ethylamino)propionitrile | Phenethylamine |
| N-methylbenzylamine | 4-hydroxypiperidine | 3,4-dimethylaniline |
| (+/−)-2-amino-1-butanol | N-(2-hydroxyethyl) | 1-naphthalene |
| | piperazine | methylamine |
| 2-(2-aminoethyl) | S(+)-1-cyclohexyl | 2-aminophenethyl alcohol |
| pyridine | ethylamine | |
| 6-amino-m-cresol | 4-aminophenol | Decylamine |
| m-anisidine | 2-ethylpiperidine | 4-aminophenethyl alcohol |
| p-anisidine | N-methylcyclohexylamine | Diethanolamine |
| methyl 4-aminobenzoate | 3-piperidinemethanol | 2-(methylthio)aniline |
| 5-amino-o-cresol | 2,4-dimethylaniline | 4-amino-2-chlorophenol |
| 4-fluorobenzylamine | 2,5-dimethylaniline | Dibenzylamine |
| 1-(3-aminopropyl)- | 6′-amino-3′,4′(methylene- | 2-(aminomethyl)-5- |
| imidazole | dioxy)acetophenone | methylpyrazine |
| 2-(1-cyclohexenyl) | 3-amino-4-hydroxybenzoic | (R)-(+)-1-(4- |
| ethylamine | acid | methoxyphenyl)ethylamine |
| 2,(2-thienyl)ethylamine | (1R, 2S)-1-amino-2-indanol | 4-ethynylaniline |
| 1-(3,4-dichlorophenyl) | N-(4-amino-2- | 1(−)-2amino-3-phenyl-1- |
| piperazine | chlorophenyl)morpholine | propanol |
| 1-acetylpiperazine | N-benzyl-2-phenylethylamine | 5-tert-butyl-o-anisidine |
| isonipecotamide | 5-phenyl-o-anisidine | 4-amino salicylic acid |
| 2-amino-m-cresol | Cyclooctylamine | 2,4-dimethoxyaniline |
| 2-methoxy-6- | 3-hydroxytyramine | 4-amino-3-hydroxybenzoic |
| methylaniline | hydrobromide | acid |
| 2-aminonorbornane | 2-[2-(aminomethyl) | 1-amino-2- |
| hydrochloride | phenylthio]benzyl alcohol | methylnaphthalene |
| 5-aminoindazole | 2-amino-1,3-propanediol | 3-amino-5-phenylpyrazole |
| 5-aminobenzotriazole | 3-amino-1,2-propanediol | Veratrylamine |
| methyl 4-aminobutyrate | 3-bromobenzylamine | 3-amino-1-phenyl-2- |
| hydrochloride | hydrochloride | pyrazolin-5-one |
| 2-chloro-4,6- | 1-(2-methoxyphenyl) | 5-amino-1-methyl-3- |
| dimethylaniline | piperazine hydrochloride | (thien-2-yl)pyrazole |
| (1S,2S)-(+)-2-amino-1- | 4-benzyloxyaniline | 3,5-bis(trifluoro- |
| phenyl-1,3-propanediol | hydrochloride | methyl)-benzylamine |
| 2-bromobenzylamine | (S)-(+)-2-amino-3- | 3-aminopyrrolidine |
| hydrochloride | cyclohexyl-1-propanol HCl | dihydrochloride |
| N-(4-methoxyphenyl)-p- | | 2-piperidinemethanol |
| phenylenediamine | | |
| hydrochloride | | |

In another of such methods, bi-ligand libraries can be prepared by reacting carboxylic acids to common ligand mimics of the present invention having an amine or amide containing substituent.

As shown in FIG. 4b, bi-ligand libraries of the invention can also be prepared in the following manner. HOBt resin is swelled a dry solvent, such as dry DMF, and added to a solution of a carboxylic acid in a solvent, such as a mixture of dry DMF and DIC. The solution is shaken at room temperature overnight and then washed with 3× dry DMF and 1× dry THF. The resin is added to a solution of a common ligand mimic of the invention in a mixed solvent, for example dry THF/DMF. The solution is again shaken at room temperature overnight. The resin then can be filtered and washed with solvent, followed by collection and vacuum drying of the filtrate to provide bi-ligands of the invention. Nonlimiting examples of carboxylic acids useful for the preparation of bi-ligand libraries include those in Table 2.

TABLE 2

| | | |
|---|---|---|
| acetic acid | 5-Bromonicotinic acid | 4-Chlorobenzoic acid |
| 4-Chloro-3-nitrobenzoic | 4-(3-Hydroxyphenoxy) | 4-Biphenylcarboxylic |
| acid | benzoic Acid | acid |
| N-Acetylglycine | 3,5-Dihydroxybenzoic acid | 2-Bromobenzoic acid |
| Propionic acid | 2,4-Dihydroxybenzoic acid | 3-Bromobenzoic acid |
| Crotonic acid | 2,3-Dihydroxybenzoic acid | 4-Bromobenzoic acid |
| 4-pentenoic acid | 2-Chloro-5-nitrobenzoic | 4-Phenoxybenzoic acid |
| | acid | |
| methacrylic acid | 6-Mercaptonicotinic acid | 4-Mercaptobenzoic acid |
| Pyruvic acid | Cyclohexanepropionic acid | Acrylic acid |
| 3-Hydroxy-2-methyl-4- | 1-(4-Chlorophenyl)-1- | 4-Hydroxy-3-(morpholino- |
| quinolinecarboxylic | cyclopropanecarboxylic acid | mehtyl)benzoic acid |
| acid | | |
| n-butyric acid | 3-Chlorobenzoic acid | Isobutyric acid |
| methoxyacetic acid | 2-Chlorobenzoic acid | 3-Indolebutyric acid |
| mercaptoacetic acid | 5-Nitro-2-furoic acid | 2,6-Difluorobenzoic acid |
| 2,3-Difluorobenzoic acid | 6-Chloronicotinic acid | Ethoxyacetic acid |
| trans-2,3- | 1,4-Dihydroxy-2-napthoic | 3,7-Dihydroxy-2-napthoic |
| dimethylacrylic acid | acid | acid |
| Cyclobutanecarboxylic | 2-methylcyclopropane | 2-Chloro-4-nitrobenzoic acid |
| acid | carboxylic acid | |
| cyclopropanecarboxylic | 4-(4-Hydroxyphenoxy) | 9H-Fluorene-9-carboxylic |
| acid | benzoic Acid | acid |
| 2-ketobutyric acid | 3,5-Difluorobenzoic acid | Pentafluorobenzoic acid |
| Isovaleric acid | 2,4-Difluorobenzoic acid | Indole-5-carboxylic acid |
| Trimethylacetic acid | 3,4,5-Trimethoxybenzoic | 3-Nitrobenzoic acid |
| 99% | acid | |
| 3-methoxypropionic acid | Indole-2-carboxylic acid | 3-Phenoxybenzoic acid |
| 3-Hydroxybutyric acid | 2-benzofurancarboxylic acid | 4-Phenylbutyric acid |
| 4,8-Dihydroxyquinoline- | 2,3,4-Trimethoxybenzoic | 3-(3,4-Dimethoxyphenyl) |
| 2-carboxylic acid | acid | propionic acid |
| (Methylthio)acetic acid | indazole-3-carboxylic acid | 3-chloropropionic acid |
| Pyrrole-2-carboxylic acid | Benzotriazole-5-carboxylic | 3-bromo-4-methylbenzoic |
| | acid | acid |
| 4-Aminobenzoic acid | Indoline-2-carboxylic acid | 3-Bromophenylacetic acid |
| 5-Acetylsalicylic acid | Pentafluoropropionic acid | 4-bromophenylacetic acid |
| 2-Furoic acid | 4-acetylbenzoic acid | 2-Iodobenzoic acid |
| Cyclopentanecarboxylic acid | 5-Norbornene-2,3- | 9-Flourenone-2- |
| | dicarboxylic acid | carboxylic acid |
| | monomethyl ester | |
| trans-3-Hexenoic acid | 3-(5-Nitro-2-furyl)acrylic | xanthene-9-carboxylic |
| 97% | Acid | acid |
| Piperonylic acid | 4-Carboxyphenylboronic acid | 3-Benzoylbenzoic acid |
| 2-tetrahydrofuroic acid | 4-Dimethylaminobenzoic acid | 4-benzoylbenzoic acid |
| 2-Phenoxybenzoic acid | 3-Dimethylaminobenzoic acid | 2-Butynoic acid |
| Tetrahydro-3-furoic | 3-Methoxyphenylacetic acid | 2-Hydroxyisobutyric acid |
| acid | | |
| hexanoic acid | 4-Ethoxybenzoic acid | 2,4-Hexadienoic acid |
| 2-Ethylbutyric acid | 4-methoxyphenylacetic acid | (Ethylthio)acetic acid |
| DL-3-Methylvaleric | (alpha,alpha,alpha-tetra- | 1-Cyclohexene-1- |
| acid, 97% | fluoro-p-tolyl)acetic acid | carboxylic acid |
| Tert-Butylacetic acid, | 1,4-Benzodioxan-2- | 2-Phenoxymethylbenzoic |
| 98% | carboxylic acid | Acid |
| 1-Acetylpiperidine-4- | (R)-(−)-5-oxo-2- | 2-hydroxy-2- |
| carboxylic acid | tetrahydro-furancarboxylic | methylbutyric acid |
| | acid | |
| Vanillic acid | 2,6-Dichloronicotinic acid | 3-Allyloxypropionic acid |
| Benzoic acid | 5-Methoxysalicylic acid | 5-Methylhexanoic acid |
| Picolinic acid, 99% | (4-Pyridylthio)acetic acid | 2-Aminonicotinic acid |
| Nicotinic acid | 2-(Methylthio)nicotinic | 6-Methylpicolinic acid |
| | acid | |
| 2-Pyrazinecarboxylic | 1-Methyl-1- | 2-Ethyl-2-hydroxybutyric |
| acid | cyclohexanecarboxylic acid | acid |
| 1-methyl-2- | 2-Hydroxy-6-methylpyridine- | 3-Cyclohexenecarboxylic |
| pyrrolecarboxylic acid | 3-carboxylic acid | acid |
| 1- | (R)-(+)-3-Methylsuccinic | 2-Hydroxyphenylacetic |
| Isoquinolinecarboxylic | acid -1-monomethyl ester | acid |
| acid | | |
| 4-butylbenzoic acid | Quinoline-4-carboxylic acid | 2,6-Dimethylbenzoic acid |
| 2-Thiophenecarboxylic | 1H-Indole-3-acetic acid | Thiophene-3-carboxylic |
| acid | | acid |
| 5-Fluoroindole-2- | 5-Hydroxy-2- | 2-(n-Propylthio) |
| carboxylic acid | indolecarboxylic acid | nicotinic acid |
| (S)-(−)-2-Pyrrolidone- | (R)-(−)-4-Methylglutaric | DL-2-Hydroxy-4- |
| 5-carboxylic acid | acid 1-monomethyl ester | (methylthio)butyric acid |
| Itaconic acid monoethyl | 5-methylisoxazole-4- | 2-Amino-6-fluorobenzoic |
| ester | carboxylic acid | acid |
| m-Toluic acid | 4-Acetamidobenzoic acid | 2-Mercaptonicotinic acid |
| p-Toluic acid | 4-Aminosalicylic acid | 6-Methylnicotinic acid |
| 2-Methylnicotinic acid | 3-Acetamidobenzoic acid | 2,5-Difluorobenzoic acid |
| 3-aminobenzoic acid | Succinamic acid | o-Toluic acid |
| 2-Chloroisonicotinic | 2-(4-Fluorobenzoyl)benzoic | 2-Fluorophenylacetic |
| acid | acid | acid |
| 3-Hydroxybenzoic acid | 3,4-Dimethoxybenzoic acid | 2-Acetylbenzoic acid |
| 4-Hydroxybenzoic acid | 3,5-Dimethoxybenzoic acid | 4-chlorosalicylic acid |
| 2,5-Dimethoxybenzoic | 3-(3,4-Dihydroxyphenyl) | 1-Phenyl-1-cyclopropane |
| acid | propionic acid | carboxylic acid |
| 5-Norbornene-2- | 5-Methyl-2- | 2,5-Dimethylphenylacetic |
| carboxylic acid | pyrazinecarboxylic acid | acid |
| (2-n- | 3-Hydroxy-4-nitrobenzoic | 2,4,6-Trimethylbenzoic |
| Butoxyethoxy)acetic | acid | acid |
| Acid | | |
| 5-Bromofuroic acid | 5-Nitrosalicylic acid | 2-Ethoxybenzoic acid |
| 6-Hydroxynicotinic acid | 4-Chloro-o-anisic acid | Salicylic acid |
| 2-Methoxyphenylacetic | 3-Chloro-4- | 3-Methyl-2- |
| acid | hydroxyphenylacetic acid | thiophenecarboxylic acid |
| 2,4- | trans-4-n-propylcyclohexane | 2-Amino-5-chlorobenzoic |
| Difluorophenylacetic | carboxylic acid | acid |
| acid | | |
| 2-Chloro-6-methyl-3- | 2-Hydroxyquinoline-4- | O-Chlorophenylacetic |
| pyridinecarboxylic acid | carboxylic acid | acid |
| 4-Fluorobenzoic acid | 3-indolepropionic acid | 4-Octyloxybenzoic acid |
| 3-Flurobenzoic acid | 2-Amino-4-chlorobenzoic | 5-Bromofuroic acid |
| | acid | |
| alpha, alpha,alpha- | Alpha,Alpha,Alpha- | Alpha, Alpha, Alpha- |
| trifluoro-p-toluic acid | Trifluoro-o-toluic acid | Trifluoro-m-toluic acid |
| 2-Thiopheneacetic acid | 2,5-Dimethyl-3-furoic acid | (+/−)-Citronellic acid |
| 3-Thiopheneacetic acid | Chromone-2-carboxylic acid | 2-Fluorobenzoic acid |
| 5-Bromo-2,4- | 2-[(4S)-2,2-Dimethyl-5-oxo- | 2,5-Difluorophenylacetic |
| dihydroxybenzoic acid | 1,3-dioxolane-4-yl] acetic | acid |
| monohydrate | acid | |
| (R)-(+)-2- | 3-Hydroxy-2- | 2,4,5-Trifluorobenzoic |
| Benzyloxypropionic acid | quinoxalinecarboxylic acid | acid |
| 4-cyanobenzoic acid | Coumarin-3-carboxylic acid | 2-Chloronicotinic acid |
| 3-Cyanobenzoic acid | 2,4-Dichlorobenzoic acid | 2-Chloro-6-fluorobenzoic |
| | | acid |
| phthalide-3-acetic acid | 2,5-Dichlorobenzoic acid | 3-indoleglyoxylic acid |
| 2,5-Dimethylphenoxy | 5-Methoxyindole-2- | 2,3,4-Trifluorobenzoic |
| acetic acid | carboxylic acid | acid |
| 2,5-Dimethylbenzoic | 2,6-Dichlorobenzoic acid | 4-Isobutylbenzoic acid |
| acid | | |
| 3,4-Dimethylbenzoic | 3,4-Dichlorobenzoic acid | 1-Naphthoic acid |
| acid | | |
| p-Tolylacetic acid | 2,3-Dichlorobenzoic acid | m-Tolylacetic acid |
| 4-acetylphenoxyacetic | 2,4-Dimethylphenoxyacetic | 2,4-Dimethoxybenzoic |
| acid | acid | acid |
| 2,4-Dimethylbenzoic | (−)-2-oxo-4- | 1-Adamantanecarboxylic |
| acid | thiazolidinecarboxylic acid | acid |
| 3,5-Dimethylbenzoic | 2,3-Dimethylphenoxyacetic | 2-Amino-5-nitrobenzoic |
| acid | acid | acid |
| 2-Bromoacrylic acid | 3-Methylhippuric acid | 3,5-Dichlorobenzoic acid |
| 3-(3-pyridyl)propionic | 4-(4-methoxyphenyl)butyric | 2,3-Dimethoxybenzoic |
| acid | acid | acid |
| 1-Hydroxy-2-naphthoic | 2-(4-Hydroxyphenoxy) | 2-(allylthio)nicotinic |
| acid | propionic acid | acid |
| 3-methylsalicylic acid | N,N-dimethylsuccinamic acid | 2-(Ethylthio)nicotinic |
| | | acid |
| P-Anisic acid | 2-Mehtylhippuric acid | 6-bromohexanoic acid |
| o-Anisic acid | 5-Chloroindole-2-carboxylic | Itaconic acid mono-n- |
| | acid | butyl ester |
| 4-Nitrophenoxyacetic | trans-4-n-Butylcyclohexane | 2-(4-Chlorophenyl)-2- |
| acid | carboxylic acid | methylpropionic acid |
| 5-methylsalicylic acid | Rhodanine-N-acetic acid | 2-Chloromandelic acid |
| 6-Hydroxy-1-napthoic | 2-Chloro-4,5- | 2-Biphenylcarboxylic |
| acid | difluorobenzoic acid | acid |
| 3,5-dimethoxy-4- | 2,3,4,5-Tetrafluorobenzoic | 4-Bromo-2-fluorocinnamic |
| methylbenzoic acid | acid | acid |
| 1-Adamantaneacetic acid | 2-Chloro-4- | 1-Naphthaleneacetic acid |
| | fluorophenylacetic acid | |
| Cyclopentylacetic acid | (2,5-Dimethoxyphenyl)acetic | 2-Chloro-4- |
| | acid | fluorocinnamic acid |
| 1-Phenylcyclopentane | 2-(4-Chlorophenoxy)-2- | Cyclohexanecarboxylic |
| carboxylic acid | methylpropionic acid | acid |
| 1-(p-Tolyl)-1- | (2S)-4-(1,3- | 2,6-Dichloro-5- |
| cyclopentanecarboxylic | Dioxoisoindolin-2-yl)-2- | fluoropyridine-3- |
| acid | hydroxy butanoic acid | carboxylic acid |
| 2,6- | (4-Chlorophenylthio) acetic | 3-Hydroxy-7-methoxy-2- |
| Dichlorophenylacetic | acid | naphthoic acid |
| acid | | |
| (−)-Camphanic acid | 2,3-Diphenylpropionic acid | DL-2-Methylbutyric acid |
| 2-Amino-5-bromobenzoic | Beta-(4-Methylbenzyl) | Rhodanine-3-propionic |
| acid | mercaptopropionic acid | acid |
| 2,5-Dimethoxy cinnamic | 2,5-Dichlorophenylthio | trans-2-Methyl-2- |
| acid | glycolic acid | pentenoic acid |
| trans-2-Pentenoic acid | (−)-Camphanic acid | 2-Methyl-3-furoic acid |
| Valeric acid | mono-Ethyl malonate | trans-2-hexenoic acid |
| 3-(2- | 2-Chloro-6- | 4-Benzyloxyphenylacetic |
| benzothiazolylthio) | fluorophenylacetic acid | acid |
| propionic acid | | |
| 2,4,Dichlorophenylacetic | 5-Bromo-2-fluorocinnamic | 4-(4-tert- |
| acid | acid | butylphenyl)benzoic acid |
| (+/−)-2-(6-Methoxy-2- | 2-(carboxymethylthio)-4,6- | 1-Piperidinepropionic |
| naphthyl)propionic acid | dimethylpyridine | acid |
| | monohydrate | |
| 3-Cyclopentylpropionic | (2- | Alpha-Methylcinnamic |
| acid | Benzothiazolylthio)acetic | acid |
| | acid | |
| 2-Ethoxynaphthoic acid | DL-Lactic acid | 2-Methylhexanoic acid |
| trans-3-Furanacrylic | 1-(4-Methoxyphenyl)-1- | 3-Hydroxy-2-pyridine- |
| acid | cyclopentanecarboxylic acid | carboxylic acid |
| 2,3-Dichlorophenoxy | 2,4-Dichlorophenoxy acetic | 3-Mercaptoisobutyric |
| acetic acid | acid | Acid |
| 5-Fluoro-2- | (3,4-Dimethoxyphenyl)acetic | 2-Thiopheneglyoxylic |
| methylbenzoic acid | acid | acid |
| (2-Napthoxy)-acetic | o-Tolylacetic acid | 2-Hydroxyoctanoic acid |
| acid | | |
| Urocanic acid | Hydrocinnamic acid | N-Acetyl-l-proline |
| Dl-Mandelic acid | DL-2-Phenylpropionic acid | N-Methyl-maleamic acid |
| Coumalic acid | 4-(Methylamino)benzoic acid | 3,4-Difluorobenzoic acid |
| 4-Methyl-1-cyclohexane | Tetrahydro-2, 2-dimethyl-5- | DL-2-phenoxypropionic |
| carboxylic acid | oxo-3-furancarboxylic acid | acid |
| m-Anisic acid | 3-Hydroxyphenylacetic acid | Indole-3-carboxylic acid |
| Cyclohexylacetic acid | Phenoxyacetic acid | 3-Fluorocinnamic acid |
| Cycloheptanecarboxylic | 3-Amino-1H-1,2,4-triazole- | 3-Fluoro-4-methylbenzoic |
| acid | 5-carboxylic acid | acid |
| 2-Octynoic acid | trans-Styrylacetic acid | 2-Methylcinnamic acid |
| 2-Propylpentanoic acid | 3-Fluorophenylacetic acid | 4-Acetylbutyric acid |
| 2-Methylheptanoic acid | Furylacrylic acid | Phenylpyruvic acid |
| Octanoic acid | Thiosalicylic acid | mono-Ethyl succinate |
| 3-(2-Thienyl)acrylic | Alpha-Methylhydrocinnamic | Alpha-Fluorocinnamic |
| acid | acid | acid |
| mono-Methyl glutarate | 3-(2-Thienyl)propanoic acid | 3-Phenoxypropionic acid |
| trans-3-(3- | trans-3-(3-Thienyl)acrylic | 3,4-(Methylenedioxy) |
| Pyridyl)acrylic acid | acid | phenylacetic acid |
| 3-Noradamantane | 4-Acetyl-3,5-dimethyl-2- | 3-(2-Hydroxyphenyl) |
| carboxylic acid | pyrrolecarboxylic acid | propionic acid |
| 2-Nitrobenzoic acid | DL-Atrolactic acid | 4-Methylsalicylic acid |
| 4- | 2-Methyl-1H-benzimidazole- | 3-Fluoro-4- |
| (Dimethylamino)butyric | 5-carboxylic acid | methoxybenzoic acid |
| acid hydrochloride | | |
| 3-Chloro-4- | 4-(Dimethylamino) | 3,4-Difluorocinnamic |
| hydroxybenzoic acid | phenylacetic acid | acid |
| DL-3-Phenyllactic acid | 3-Benzoylpropionic acid | Homovanillic acid |
| 2-Methyl-terephthalic | 3-(Diethylamino) propionic | 3-(4-Methylbenzoyl) |
| acid | acid hydrochloride | propionic acid |
| 4-(2-Thienyl)butyric | 3,4-Dihydro-2,2-dimethyl-4- | Cyclohexanepentanoic |
| acid | oxo-2H-pyran-6-carboxylic | acid |
| | acid | |
| Cyclohexanebutyric acid | mono-Methyl phthalate | Undecanoic acid |
| 3-Chlorophenylacetic | 3,5-Difluorophenylacetic | 6-Hydroxy-2-naphthoic |
| acid | acid | acid |
| 3-Benzoylacrylic acid | 4-Amino-2-chlorobenzoic | 3-Indoleacrylic acid |
| | acid | |
| 3-Amino-4-chlorobenzoic | 4-(4-Methylphenyl)butyric | 3-Hydroxy-2-naphthoic |
| acid | acid | acid |
| 3,4- | 3-(4- | 2-Hydroxy-1-naphthoic |
| Difluorophenylacetic | Methoxyphenyl)propionic | acid |
| acid | acid | |
| 2,5-Dimethylphenoxy | trans-3-(4- | 5-Methyl-2-nitrobenzoic |
| acetic acid | Methylbenzoyl)acrylic acid | acid |
| 3-Quinolinecarboxylic | 3-(2- | 3,5-Dimethyl-p-anisic |
| acid | Methoxyphenyl)propionic | acid |
| | acid | |
| Decanoic acid | 2-Naphthoic acid | 4-Benzoylbutyric acid |
| 5-Chlorosalicylic acid | Quinaldic acid | N-Methylhippuric acid |
| 3-(3-Methoxyphenyl) | 5-Nitrothiophene-2- | 4-(Diethylamino) benzoic |
| propionic acid | carboxylic acid | acid |
| 2-Methyl-6-nitrobenzoic | Alpha,Alpha,Alpha-2- | N,N-Dimethyl-1- |
| acid | Tetrafluoro-p-toloic acid | phenylalanine |
| Ibuprofen | 2-Nitrophenylacetic acid | 4-Benzyloxybutyric acid |
| 3-Pyridylacetic acid | 2-Methyl-5-nitrobenzoic | Diethylphosphonoacetic |
| | acid | acid |
| 2-Oxo-6-pentyl-2H- | mono-Methyl cis-5- | 2-Methyl-3-nitrobenzoic |
| pyran-3-carboxylic acid | norbornene -endo-2,3- | acid |
| | dicarboxylate | |
| DL-2-(3-Chlorophenoxy) | 3,5-Dichloro-4- | trans-2-Chloro- |
| propionic acid | hydroxybenzoic acid | fluorocinnamic acid |
| 5-Bromo-2-thiophene | DL-4-Hydroxy-3- | 2-Phenylmercapto |
| carboxylic acid | methoxymandelic acid | methylbenzoic acid |
| 3,4-Diethoxybenzoic | Alpha-Phenyl-o-toluic acid | Diphenylacetic acid |
| acid | | |
| 5-Bromosalicylic Acid | Adipic acid monoethyl ester | Syringic acid |
| 3,5-Dichloroanthranilic | trans-2,4-Dimethoxycinnamic | 4-(4-Hydroxyphenyl) |
| acid | acid | benzoic Acid |
| Alpha-Phenylcinnamic | trans-2,3-dimethoxycinnamic | 3-(Phenylsulfonyl) |
| acid | acid | propionic acid |
| 3,3-Diphenylpropionic | (s)-(−)-2-[(Phenylamino) | 3-(Trifluoromethyl) |
| acid | carbonyloxy]propionic acid | cinnamic acid |
| Cyclohexylphenylacetic | 4-(3-Methyl-5-oxo-2- | 3,4-Dimethoxycinnamic |
| acid | pyrazoline-1-yl)benzoic | acid |
| | acid | |
| 4-(Trifluoromethyl) | Pentafluorophenoxyacetic | Trans-2,4- |
| mandelic acid | acid | Dichlorocinnamic acid |
| 2-Nitrophenylpyruvic | Alpha-Phenylcyclopentane | 3,4-Dichlorophenylacetic |
| acid | acetic acid | acid |
| 4-(Hexyloxy)benzoic | 4-Butoxyphenylacetic acid | 4-Bromocinnamic acid |
| acid | | |
| 7-Hydroxycoumarin-4- | 3-(3,4,5-Trimethoxyphenyl) | 2-Chloro-5- |
| acetic acid | propionic acid | (methylthio)benzoic acid |
| 1,3-dioxo-2- | 3,4,5-Trimethoxy | 3-Bromo-4-fluorocinnamic |
| isoindolineacetic acid | phenylacetic acid | acid |
| Anthracene-9-carboxylic | p-Bromophenoxyacetic acid | N-Carbobenzyloxy-L- |
| acid | | proline |
| (Phenylthio)acetic acid | 4-Butoxyphenylacetic acid | 3-Phenylbutyric acid |
| Acridine-9-carboxylic | 4-Benzyloxybenzoic acid | 3,4,5-Triethoxybenzoic |
| acid hydrate | | acid |
| 7-Chloro-4-hydroxy-3- | 1,4-dihydro-1-ehtyl-7- | |
| quinolinecarboxylic | methyl-4-oxo-1, | |
| acid | 8-naphthyridine- | |
| | 3-carboxylic acid | |
| gamma-Oxo-(1,1′- | 2-Ethoxycarbonylamino-3- | 3,5-Di-tert-butyl-4- |
| biphenyl)-4-butanoic | phenyl-propionic acid | hydroxybenzoic acid |
| aicd | | |
| 2-Cyclopentene-1-acetic | 3,4,5-Trimethoxycinnamic | 3-(BOC-amino)benzoic |
| acid | acid | acid |
| 4-Methoxysalicylic acid | 4-Fluorocinnamic acid | 4,5-Dibromo2-furoic acid |
| 2-Hydroxynicotinic acid | 4-Bromo-3,5- | 5-Phenylvaleric acid |
| | dihydroxybenzoic acid | |
| 4-Pentynoic acid | 4-Ethoxybenzoic acid | 4-Acetoxybenzoic acid |
| 3,3-Dimethylacrylic | Dicyclohexylacetic acid | 3-Acetoxybenzoic acid |
| acid | | |
| 4-Methoxy-2- | cis-2- | 4-Methyl-3-nitrobenzoic |
| methylbenzoic acid | (2-Thiophenecarbonyl)-1- | acid |
| | cyclohexanecarboxylic | |
| | acid | |
| 4-Methylvaleric acid | (2-Methylphenoxy)acetic | 4-Isopropoxybenzoic acid |
| | acid | |
| 3,3,3- | (4-Methylphenoxy)acetic | 4-Nitrophenylacetic acid |
| Trifluoropropionic acid | acid | |
| 2-Methyl-1-cyclohexane | 2,2,3,3-Tetramethyl | 3-Methyl-1-cyclohexane |
| carboxylic acid | cyclopropanecarboxylic acid | carboxylic acid |
| 4-Amino-3-nitrobenzoic | 5-Methyl-2- | 4-Methoxyphenoxyacetic |
| acid | thiophenecarboxylic acid | acid |
| 3-Methoxysalicylic acid | 4-Fluorophenylacetic acid | 2-Phenoxybutyric acid |
| 3,5-Dimethoxy-4- | (R)-(−)-2,2-Dimethyl-5- | 4-Hydroxymandelic acid |
| hydroxycinnamic acid | oxo-1,3-dioxolane-4-acetic | monohydrate |
| | acid | |
| (2-Methoxyphenoxyl) | 2,2-Dichloro-1-methylcyclo- | 4-Hydroxyphenylacetic |
| acetic acid | propanecarboxylic acid | acid |
| 2-Ethylbenzoic acid | 4-Fluorophenoxyacetic acid | 4-tert-Butylbenzoic acid |
| 5-Fluoro-2- | (R)-(−)-2-(4-Hydroxy | 2,6-Dimethoxynicotinic |
| methoxybenzoic acid | phenoxy)-propionic acid | acid |
| 2-Carboxyethyl | 4-Hydroxy-3-nitrobenzoic | 3,4-Difluorohydro |
| phosphonic acid | acid | cinnamic acid |
| 4-Hydroxy-3-methoxy | 3-Chloro-2-methylbenzoic | 2-Chloro-4-fluorobenzoic |
| benzoic acid | acid | cinnamic acid |
| 4-Fluoro-3- | 2-Chloro-6-methylnicotinic | 4-Chlorophenoxyacetic |
| methylbenzoic acid | acid | acid |
| 3-Fluoro-2- | 2,2-Bis(hydroxymethyl) | 5-Chloro-2- |
| methylbenzoic acid | butyric acid | methoxybenzoic acid |
| 5-Amino-4-methyl- | (2,2-Dimethyl-5-[2,5- | (Alpha, Alpha, Alpha- |
| cyclohexa-1,5-diene- | dimethylphenoxy]-pentanoic | Trifluoro-m-tolyl)acetic |
| 1,4-dicarboxylic acid | acid) | acid |
| 4-Methoxycyclohexane | 1-Methylindole-3-carboxylic | (R)-(−)-3- |
| carboxylic acid | acid | Chloromandelic acid |
| 4-Propylbenzoic acid | 4-Chlorophenylacetic acid | 4-Bromomandelic acid |
| 2-Methoxy-4- | 4-Oxo-4H-1-benzopyran-2- | 2-Mercapto-4-methyl-5- |
| (methylthio) -benzoic | carboxylic acid | thiazoleacetic acid |
| acid | | |
| 2-(Trifluoromethyl) | 4-Methoxy-3-nitrobenzoic | 3,4-Dichlorocinnamic |
| cinnamic acid | acid | acid |
| 3-Methylcyclohexane | 4-Methoxy-2- | 5-Methoxy-2-methyl-3- |
| carboxylic acid | quinolinecarboxylic acid | indoleacetic acid |
| 2-(4-Nitrophenyl) | 4-(4-Methoxyphenyl)butyric | 4-Carboxybenzene |
| propionic acid | acid | sulfonamide |
| 2-Hydroxy-5-(1H-pyrrol- | 3-Chloro-4- | 4-Chloro-2-nitrobenzoic |
| 1-yl)-benzoic acid | hydroxyphenylacetic acid | acid |
| 2-Methyl-3-indoleacetic | 2-Fluoro- | 4-Amino-5-chloro-2- |
| acid | 3(trifluoromethyl)-benzoic | methoxybenzoic acid |
| | acid | |
| 4-Chloro-2- | 2-(2-Nitrophenoxy)acetic | 3-Acetoxy-2- |
| fluorocinnamic acid | acid | methylbenzoic acid |
| 2,4,6-Trichlorobenzoic | 3,4-Dichlorophenoxyacetic | 2-Bibenzylcarboxylic |
| acid | acid | acid |
| 2-Chloro-5- | (S)-(+)-6-Methoxy-alpha- | 4-(3,4-Dimethoxyphenyl)- |
| (trifluoromethyl)benzoic | methyl-2-naphthalenacetic | butyric acid |
| acid | acid | |
| 4-Ethylbiphenyl-4′- | 2-Bromo-5-methoxybenzoic | 5-Bromo-2-chlorobenzoic |
| carboxylic acid | acid | acid |
| 3,5-Dinitro-p-toluic | 1-Methyl-2- | 1-Methyl-3-indoleacetic |
| acid | nitroterephthalate | acid |
| 4-Pentylbenzoic acid | 4-n-Heptyloxybenzoic acid | 4-Biphenylacetic acid |

Alternatively, bi-ligand libraries of the invention can be built through the direct reaction of isocyanates or thioisocyanates using a combination of solid phase chemistry and solution phase chemistry.

As shown in FIG. 4c, bi-ligand libraries of the invention can further be prepared in the following manner. A solution of an isocyanate or thioisocyanate and a common ligand mimic of the invention is formed in a solvent, such as DMSO. The isocyanate and common ligand mimic are allowed to react overnight, followed by the addition of aminomethylated polystyrene Resin (NovaBiochem, Cat. No. 01-64-0383). This mixture is then shaken at room temperature for a period of time, for example about 4 hours. The resin then can be filtered and dried under reduced pressure to yield the desired product. Nonlimiting examples of isocyanates and thioisocyanates are provided in Table 3.

TABLE 3

| | |
|---|---|
| allyl isocyanate | 3-chloro-4-methylphenyl isocyanate |
| N-propyl isocyanate | 1-naphthyl isocyanate |
| pentyl isocyanate | 3-chloro-4-fluorophenyl isocyanate |
| phenyl isocyanate | 2,6-diethylphenyl isocyanate |
| m-tolyl isocyanate | 1-adamantyl isocyanate |
| p-tolyl isocyanate | 2-methyl-4-nitrophenyl isocyanate |
| o-tolyl isocyanate | 2-methyl-5-nitrophenyl isocyanate |
| benzyl isocyanate | 2-methyl-3-nitrophenyl isocyanate |
| 4-fluorophenyl isocyanate | 4-methyl-2-nitrophenyl isocyanate |
| heptyl isocyanate | 4-methyl-3-nitrophenyl isocyanate |
| 3-cyanophenyl isocyanate | 2,4-dimethoxyphenyl isocyanate |
| 2,6-dimethylphenyl isocyanate | 2,5-dimethoxyphenyl isocyanate |
| 2-ethylphenyl isocyanate | 2-fluoro-5-nitrophenyl isocyanate |
| 2,5-dimethylphenyl isocyanate | 4-fluoro-3-nitrophenyl isocyanate |
| 2,4-dimethylphenyl isocyanate | 5-chloro-2-methoxyphenyl isocyanate |
| 3,4-dimethylphenyl isocyanate | ethyl-6-isocyanatohexanoate |
| 4-ethylphenyl isocyanate | 4-(trifluoromethyl)phenyl isocyanate |
| 3-ethylphenyl isocyanate | 3-(trifluoromethyl)phenyl isocyanate |
| 2,3-dimethylphenyl isocyanate | 2-(trifluoromethyl)phenyl isocyanate |
| 2-methoxyphenyl isocyanate | 3,4-dichlorophenyl isocyanate |
| 3-methoxyphenyl isocyanate | 2,4-dichlorophenyl isocyanate |
| 4-methoxyphenyl isocyanate | 3,5-dichlorophenyl isocyanate |
| 5-chloro-3-methylphenyl | 2,3-dichlorophenyl isocyanate |
| isocyanate | |
| 2-chlorophenyl isocyanate | trichloroacetyl isocyanate |
| 3-chlorophenyl isocyanate | ethyl-4-isocyanatobenzoate |
| 2,4-difluorophenyl isocyanate | Isopropyl isocyanate |
| 3,4-difluorophenyl isocyanate | Butyl isocyanate |
| 2,6-difluorophenyl isocyanate | cyclopentyl isocyanate |
| butyl isocyanatoacetate | cyclohexyl isocyanate |
| trans-2-phenylcyclopropyl | o-tolyl isocyanate |
| isocyanate | |
| trichloromethyl isocyanate | 3-fluorophenyl isocyanate |
| 3-acetylphenyl isocyanate | 2-fluorophenyl isocyanate |
| 4-acetylphenyl isocyanate | ethyl 3-isocyanatopropionate |
| 2-isopropylphenyl isocyanate | 4-methylbenzyl isocyanate |
| 2-ethyl-6-methylphenyl isocyanate | phenethyl isocyanate |
| 2,4,6-trimethylphenyl isocyanate | 3-fluorobenzyl isocyanate |
| 4-ethoxyphenyl isocyanate | 4-fluorobenzyl isocyanate |
| 2-methoxy-5-methylphenyl | 3-fluoro-4-methylphenyl isocyanate |
| isocyanate | |
| 2-ethoxyphenyl isocyanate | 2,4-difluorophenyl isocyanate |
| 4-methoxy-2-methylphenyl | 3,4-difluorophenyl isocyanate |
| isocyanate | |
| 4-methoxybenzyl isocyanate | 2,6-difluorophenyl isocyanate |
| 2-nitrophenyl isocyanate | 3,5-difluorophenyl isocyanate |
| 4-nitrophenyl isocyanate | octyl isocyanate |
| 3-nitrophenyl isocyanate | 1,1,3,3-tetramethylbutyl isocyanate |
| 4-(methylthio)phenyl isocyanate | trans-2-phenylcyclopropyl isocyanate |
| 2-(methylthio)phenyl isocyanate | trichloromethyl isocyanate |
| 5-chloro-2-methylphenyl | 4-isopropylphenyl isocyanate |
| isocyanate | |
| 4-chloro-2-methylphenyl | propyl isothiocyanate |
| isocyanate | |
| 2-isopropyl-6-methylphenyl | 3,4-(methylenedioxy)phenyl |
| isocyanate | isocyanate |
| 2-chloro-6-methylphenyl | 2-chloro-5-methylphenyl isocyanate |
| isocyanate | |
| 3-chloro-2-methylphenyl | 2-chlorobenzyl isocyanate |
| isocyanate | |
| isobutyl isothiocyanate | 3-chloro-4-fluorophenyl isocyanate |
| tert-butyl isothiocyanate | 2,6-diethylphenyl isocyanate |
| N-butyl isothiocyanate | 4-N-butylphenyl isocyanate |
| 2-methoxyethyl isothiocyanate | methyl-4-isocyanato-benzoate |
| N-amyl isothiocyanate | 3-carbomethoxyphenyl isocyanate |
| 3-methoxypropyl isothiocyanate | methyl-2-isocyanatobenzoate |
| phenyl isothiocyanate | 1-adamantyl isocyanate |
| cyclohexyl isothiocyanate | 2-methyl-4-nitrophenyl isocyanate |
| 2-tetrahydrofurfuryl isothiocyanate | 2-methyl-5-nitrophenyl isocyanate |
| o-tolyl isothiocyanate | 2-methyl-3-nitrophenyl isocyanate |
| benzyl isothiocyanate | 4-methyl-2-nitrophenyl isocyanate |
| m-tolyl isothiocyanate | 4-methyl-3-nitrophenyl isocyanate |
| 4-fluorophenyl isothiocyanate | diethoxyphosphinyl isocyanate |
| 2-fluorophenyl isothiocyanate | 2,4-dimethoxyphenyl isocyanate |
| 3-fluorophenyl isothiocyanate | 2,5-dimethoxyphenyl isocyanate |
| heptyl isothiocyanate | 3,4-dimethoxyphenyl isocyanate |
| ethyl 3-isothiocyanatopropionate | 2-fluoro-5-nitrophenyl isocyanate |
| ethyl 2-isothiocyanatopropionate | 4-fluoro-3-nitrophenyl isocyanate |
| 4-cyanophenyl isothiocyanate | benzenesulphonyl isocyanate |
| 2-ethylphenyl isothiocyanate | 5-chloro-2-methoxyphenyl isocyanate |
| 2,6-dimethylphenyl isothiocyanate | 3-chloro-4-methoxyphenyl isocyanate |
| 2-phenylethyl isothiocyanate | ethyl-6-isocyanatohexanoate |
| 2,4-dimethylphenyl isothiocyanate | 4-(trifluoromethyl)phenyl isocyanate |
| 4-methylbenzyl isothiocyanate | 3-(trifluoromethyl)phenyl isocyanate |
| 2-phenylethyl isothiocyanate | 2-(trifluoromethyl)phenyl isocyanate |
| 3-methoxyphenyl isothiocyanate | 2-(trifluoromethyl)phenyl isocyanate |
| 2-methoxyphenyl isothiocyanate | 3,4-dichlorophenyl isocyanate |
| 4-methoxyphenyl isothiocyanate | 2,6-dichlorophenyl isocyanate |
| 4-chlorophenyl isothiocyanate | 2,4-dichlorophenyl isocyanate |
| 2-chlorophenyl isothiocyanate | 2,5-dichlorophenyl isocyanate |
| 3-chlorophenyl isothiocyanate | 3,5-dichlorophenyl isocyanate |
| 2,4-difluorophenyl isothiocyanate | 2,3-dichlorophenyl isocyanate |
| 2-morpholinoethyl isothiocyanate | trichloroacetyl isocyanate |
| 3-acetylphenyl isothiocyanate | 2-ethyl-6-isopropylphenyl isocyanate |
| 4-isopropylphenyl isothiocyanate | ethyl-3-isocyanatobenzoate |
| 2-isopropylphenyl isothiocyanate | ethyl-4-isocyanatobenzoate |
| 4-(dimethylamino)phenyl | 2-isopropyl-6-methylphenyl |
| isothiocyanate | isocyanate |
| 4-ethoxyphenyl isothiocyanate | ethyl-2-isocyanatobenzoate |
| 4-methoxybenzyl isothiocyanate | 4-butoxyphenyl isocyanate |
| 3-nitrophenyl isothiocyanate | 2-methoxy-5-nitrophenyl isocyanate |
| 4-nitrophenyl isothiocyanate | 2-biphenylylisocyanate |
| 2-(methylthio)phenyl | 4-biphenyl isocyanate |
| isothiocyanate | |
| 3-(methylthio)phenyl | p-toluenesulphonyl isocyanate |
| isothiocyanate | |
| 4-(methylthio)phenyl | o-toluenesulphonyl isocyanate |
| isothiocyanate | |
| 1-naphthyl isothiocyanate | undecyl isocyanate |
| 2-chlorobenzyl isothiocyanate | 2-bromophenyl isocyanate |
| 4-chlorobenzyl isothiocyanate | 3-bromophenyl isocyanate |
| 3-chloro-4-methylphenyl | 4,5-dimethyl-2-nitrophenyl |
| isothiocyanate | isocyanate |
| 4-chloro-2-methylphenyl | 5-chloro-2-methylphenyl |
| isothiocyanate | isothiocyanate |
| 4-bromophenyl isocyanate | 2-chloro-4-nitrophenyl isocyanate |
| 3-morpholinopropyl isothiocyanate | 2-chloro-5-nitrophenyl isocyanate |
| 4-N-butylphenyl isothiocyanate | 4-chloro-2-nitrophenyl isocyanate |
| allyl isothiocyanate | ethyl isothiocyanate |
| 2-methoxycarbonylphenyl | 2-chloro-6-methylphenyl |
| isothiocyanate | isothiocyanate |
| 1-adamantyl isothiocyanate | isopropyl isothiocyanate |
| 4-methyl-2-nitrophenyl | 4-chloro-3-nitrophenyl |
| isothiocyanate | isothiocyanate |
| 3,4-dimethoxyphenyl | 3-bromophenyl isothiocyanate |
| isothiocyanate | |
| 2,5-dimethoxyphenyl | 2-bromophenyl isothiocyanate |
| isothiocyanate | |
| 2,4-dimethoxyphenyl | 2,6-diisopropylphenyl isothiocyanate |
| isothiocyanate | |
| 5-chloro-2-methoxyphenyl | 2-(3,4-dimethoxyphenyl)ethyl |
| isothiocyanate | isothiocyanate |
| 2-(trifluoromethyl)phenyl | 4-bromo-2-methylphenyl |
| isothiocyanate | isothiocyanate |
| 4-(trifluoromethyl)phenyl | 2-bromo-4-methylphenyl |
| isothiocyanate | isothiocyanate |
| 2,6-dichlorophenyl isothiocyanate | cyclododecyl isothiocyanate |
| 2,3-dichlorophenyl isothiocyanate | 4-phenylazophenyl |
| | isothiocyanate1111 |
| 3,5-dichlorophenyl isothiocyanate | 4-diethylaminophenyl isothiocyanate |
| 4-methoxy-2-nitrophenyl | |
| isothiocyanate | |

The present invention is based on the development of bi-ligands that bind to two independent sites on a receptor. The combination of two ligands into a single molecule allows both ligands to simultaneously bind to the receptor and thus can provide synergistically higher affinity than either ligand alone (Dempsey and Snell, Biochemistry 2:1414-1419 (1963); and Radzicka and Wolfenden, Methods Enzymol. 249:284-303 (1995), each of which is incorporated herein by reference). The generation of libraries of bi-ligands focused for binding to a receptor family or a particular receptor in a receptor family has been described previously (see WO 99/60404, which is incorporated herein by reference). The common ligand mimics of the present invention allow for increased diversity of bi-ligand libraries while simultaneously preserving the ability to focus a library for binding to a receptor family.

As described previously (see WO 99/60404), when developing bi-ligands having binding activity for a receptor family, it is generally desirable to use a common ligand having relatively modest binding activity, for example, mM to μM binding activity. This binding activity is increased when combined with a specificity ligand.

The common ligand mimic can be modified through the addition of substituents, which can also be called expansion linkers. Substitution of the common ligand mimic allows for tailoring of the bi-ligand by directing the attachment location of the specificity ligand on the common ligand mimic. Tailoring of the bi-ligand in this manner provides optimal binding of the common ligand mimic to the conserved site on the receptor and of the specificity ligand to the specificity site on the same receptor. Through such tailoring, libraries having improved diversity and improved receptor binding can be produced. The bi-ligands contained in such libraries also exhibit improved affinity and/or specificity.

A number of formats for generating combinatorial libraries are well known in the art, for example soluble libraries, compounds attached to resin beads, silica chips or other solid supports. As an example, the “split resin approach” may be used, as described in U.S. Pat. No. 5,010,175 to Rutter and in Gallop et al., J. Med. Chem., 37:1233-1251 (1994), incorporated by reference herein.

Methods for generating libraries of bi-ligands having diversity at the specificity ligand position have been described previously (see WO 99/60404, WO 00/75364, and U.S. Pat. No. 6,333,149 which issued Dec. 25, 2001). A library of bi-ligands is generated so that the binding affinity of the common ligand mimic and the specificity ligand can synergistically contribute to the binding interactions of the bi-ligand with a receptor having the respective conserved site and specificity site. Thus, the bi-ligands are generated with the specificity ligand and common ligand mimic oriented so that they can simultaneously bind to the specificity site and conserved site, respectively, of a receptor.

The present invention also provides methods of screening combinatorial libraries of bi-ligands comprising one or more common ligand mimic bound to a variety of specificity ligands and identification of bi-ligands having binding activity for the receptor. Thus, the present invention provides methods for generating a library of bi-ligands suitable for screening a particular member of a receptor family as well as other members of a receptor family.

Development of combinatorial libraries of bi-ligands of the invention begins with selection of a receptor family. Methods for determining that two receptors are in the same family, and thus constitute a receptor family, are well known in the art. For example, one method for determining if two receptors are related is BLAST, Basic Local Alignment Search Tool, available on the National Center for Biotechnology Information web page (www.ncbi.nlm.gov/BLAST/) (which is incorporated herein by reference) and modified BLAST protocols. A second resource for identifying members of a receptor family is PROSITE, available at ExPASy (www.expasy.ch/sprot/prosite.html) (which is incorporated herein by reference). A third resource for identifying members of a receptor family is Structural Classification of Proteins (SCOP) available at SCOP (scop.mrc-lmb.cam.ac.uk/scop/) (which is incorporated herein by reference).

Once a receptor family has been identified, the next step in development of bi-ligands involves determining whether there is a natural common ligand that binds at least two members of the receptor family, and preferably to several or most members of the receptor family. In some cases, a natural common ligand for the identified receptor family is already known. For example, it is known that dehydrogenases bind to dinucleotides such as NAD or NADP. Therefore, NAD or NADP are natural common ligands to a number of dehydrogenase family members. Similarly, all kinases bind ATP, and, thus, ATP is a natural common ligand to kinases.

After a receptor family has been selected, at least two receptors in the receptor family are selected as receptors for identifying useful common ligand mimics. Selection criteria depend upon the specific use of the bi-ligands to be produced. Once common ligand mimics are identified, these compounds are screened for binding affinity to the receptor family.

Those common ligand mimics having the most desirable binding activity then can be modified by adding substituents that are useful for the attachment and orientation of a specificity ligand. For example, in the present invention, thiohydantoins and psudohydantoins were determined to be common ligand mimics for NAD. These compounds can be modified, for example, by the addition of substituents to the phenyl or heterocyclic ring attached to the thiohydantoin ring. For example, the phenyl or heterocyclic ring can be substituted with a COOH group, two hydroxy groups, a hydroxy and a nitro group, or an NHAc group. These groups provide attachment points for the specificity ligand. Substituents added to the phenyl or heterocyclic ring can also act as blocking groups to prevent attachment of a specificity ligand at a particular site or can act to orient the specificity ligand in a particular manner to improve binding of the bi-ligand to the receptor.

Methods of screening for common ligand mimics and bi-ligands containing the common ligand mimics are well known in the art. For example, a receptor can be incubated in the presence of a known ligand and one or more potential common ligand mimics. In some cases, the natural common ligand has an intrinsic property that is useful for detecting whether the natural common ligand is bound. For example, the natural common ligand for dehydrogenases, NAD, has intrinsic fluorescence. Therefore, increased fluorescence in the presence of potential common ligand mimics due to displacement of NAD can be used to detect competition for binding of NAD to a target NAD binding receptor (Li and Lin, Eur. J. Biochem. 235:180-186 (1996); and Ambroziak and Pietruszko, Biochemistry 28:5367-5373 (1989), each of which is incorporated herein by reference).

In other cases, when the natural common ligand does not have an intrinsic property useful for detecting ligand binding, the known ligand can be labeled with a detectable moiety. For example, the natural common ligand for kinases, ATP, can be radiolabeled with ³²P, and the displacement of radioactive ATP from an ATP binding receptor in the presence of potential common ligand mimics can be used to detect additional common ligand mimics. Any detectable moiety, for example a radioactive or fluorescent label, can be added to the known ligand so long as the labeled known ligand can bind to a receptor having a conserved site. Similarly, a radioactive or fluorescent moiety can be added to NAD or a derivative thereof to facilitate screening of the NAD common ligand mimics and/or bi-ligands of the invention.

The pool of potential common ligand mimics screened for competitive binding with a natural common ligand can be a broad range of compounds of various structures. However, the pool of potential ligands can also be focused on compounds that are more likely to bind to a conserved site in a receptor family. For example, a pool of candidate common ligand mimics can be chosen based on structural similarities to the natural common ligand.

Thiohydantoin compounds and pseudothiohydantoin compounds were identified as common ligand mimics of NAD by first determining the three-dimensional structure of NAD, the natural common ligand, and searching commercially available databases of commercially available molecules such as the Available Chemicals Directory (MDL Information Systems, Inc.; San Leandro, Calif.) to identify potential common ligands having similar shape or electrochemical properties to NAD. Methods for identifying molecules having similar structure are well known in the art and are commercially available (Doucet and Weber, in Computer-Aided Molecular Design: Theory and Applications, Academic Press, San Diego, Calif. (1996), which is incorporated herein by reference; software is available from Molecular Simulations, Inc., San Diego, Calif.). Furthermore, if structural information is available for the conserved site in the receptor, particularly with a known ligand bound, compounds that fit the conserved site can be identified through computational methods (Blundell, Nature 384 Supp:23-26 (1996), which is incorporated herein by reference). These methods also can be used to screen for specificity ligands and bi-ligands of the invention.

Once a library of bi-ligands is generated, the library can be screened for binding activity to a receptor in a corresponding receptor family. Methods of screening for binding activity that are well known in the art can be used to test for binding activity.

The common ligand mimics and bi-ligands of the present invention can be screened, for example, by the following methods. Screening can be performed through kinetic assays that evaluate the ability of the common ligand mimic or bi-ligand to react with the receptor. For example, where the receptor is a reductase or dehydrogenase for which NAD is a natural common ligand, compounds of the invention can be assayed for their ability to oxidize NADH or NADPH or for their ability to reduce NAD⁺. Such assays are described more fully in Examples 23 through 25.

### EXAMPLES

Starting materials were obtained from commercial suppliers and used without further purification. ¹H NMR spectra were acquired on a Bruker Avance 300 spectrometer at 300 MHz for ¹H NMR and 75 MHz for ¹³C NMR. Chemical shifts are recorded in parts per million (δ) relative to TMS (δ=0.0 ppm) for ¹H or to the residual signal of deuterated solvents (chloroform, δ=7.25 ppm for ¹H; δ=77.0 ppm for ¹³C). Coupling constant J is reported in Hz. Chromatography was performed on silica gel with ethyl acetate/hexane as eluant unless otherwise noted. Mass spectra were recorded on LCQ from Finnigan.

### Example 1

Preparation of 4-(2-imino-4-oxo-thiazolidin-5-ylidenemethyl)-benzoic acid (compound 3e)

This example describes the synthesis of 4-(2-imino-4-oxo-thiazolidin-5-ylidenemethyl)-benzoic acid according to the reaction scheme shown in FIG. 1. Compound numbers correspond to those in the figure. This procedure is the general procedure for preparation of the compounds of the invention.

Pseudothiohydantoin-(compound 2, 116 mg, 1 mmol) and 4-carboxybenzaldehyde (1 mmol) were suspended in acetic acid (3 ml). The mixture was heated at 95° C. for 8 hours and then cooled to room temperature. The solid product was collected and washed with a combination of water and ethyl acetate to give 4-(2-imino-4-oxo-thiazolidin-5-ylidenemethyl)-benzoic acid as a solid (compound 3e, 215 mg, 0.89 mmol, 89%)

### Example 2

Preparation of 5-(4-hydroxy-3-nitro-benzylidene)-2-imino-thiazolidin-4-one (compound 3a)

The compound 5-(4-hydroxy-3-nitro-benzylidene)-2-imino-thiazolidin-4-one was prepared from 4-hydroxy-3-nitrobenzaldehyde following the procedure in Example 1 at a yield of 79%. NMR analysis of the compound provided the following:

¹H NMR (300 MHz, DMSO-d₆): δ7.25 (d, J=8.4, 1H), 7.57 (s, 1H), 7.76 (d, J=8.4, 1H), 8.10 (s, 1H), 9.18 (s, 1H).

### Example 3

Preparation of 5-(3-hydroxy-4-nitro-benzylidene)-2-imino-thiazolidin-4-one (compound 3b)

The compound 5-(3-hydroxy-4-nitro-benzylidene)-2-imino-thiazolidin-4-one was prepared from 3-hydroxy-4-nitrobenzaldehyde following the procedure in Example 1 at a yield of 71%. NMR analysis of the compound provided the following:

¹H NMR (300 MHz, DMSO-d₆): δ7.17 (d, J=8.7, 1H), 7.30 (s, 1H), 7.54 (s, 1H), 8.33 (d, J=8.7, 1H), 9.34 (s, 1H). MS: m/z 266 (M+1)

### Example 4

Preparation of 5-(3,4-dihydroxy-benzylidene)-2-imino-thiazolidin-4-one (compound 3c)

The compound 5-(3,4-dihydroxy-benzylidene)-2-imino-thiazolidin-4-one was prepared from 3,4-dihydroxy-benzaldehyde following the procedure in Example 1 at a yield of 68%. NMR analysis of the compound provided the following:

¹H NMR (300 MHz, DMSO-d₆): δ6.82-6.92 (m, 2H), 6.97 (s, 1H), 7.41 (s, 1H)

### Example 5

Preparation of 3-(2-imino-4-oxo-thiazolidin-5-ylidenemethyl)-benzoic acid (compound 3d)

The compound 3-(2-imino-4-oxo-thiazolidin-5-ylidenemethyl)-benzoic acid was prepared from 3-carboxybenzaldehyde following the procedure in Example 1 at a yield of 81%. NMR analysis of the compound provided the following:

¹H NMR (300 MHz, DMSO-d₆): δ7.61-7.66 (m, 1H), 7.66 (s, 1H), 7.84-7.86 (m, 1H), 7.95-7.98 (m, 1H), 8.17 (s, 1H).

### Example 6

Preparation of 4-(2-imino-4-oxo-thiazolidin-5-ylidenemethyl)-benzoic acid (compound 3e)

The compound 4-(2-imino-4-oxo-thiazolidin-5-ylidenemethyl)-benzoic acid was prepared from 4-carboxybenzaldehyde following the procedure in Example 1 at a yield of 89%. NMR analysis of the compound provided the following:

¹H NMR (300 MHz, DMSO-d₆): δ7.64-7.70 (m, 2H) ; 7.70 (s, 1H), 8.03-8.05 (m, 2H).

### Example 7

Preparation of 5-(4-hydroxy-3-methoxy-benzylidene)-2-imino-thiazolidin-4-one (compound 3f)

The compound 5-(4-hydroxy-3-methoxy-benzylidene)-2-imino-thiazolidin-4-one was prepared from 4-hydroxy-3-methoxybenzaldehyde following the procedure in Example 1 at a yield of 72%. NMR analysis of the compound provided the following:

¹H NMR (300 MHz, DMSO-d₆): δ6.89-6.91 (m, 1H), 7.02-7.05 (m, 1H), 7.15 (s, 1H), 7.52 (s, 1H). MS: m/z=251 (M+1).

### Example 8

Preparation of 5-(3-hydroxy-4-methoxy-benzylidene)-2-imino-thiazolidin-4-one (compound 3g)

The compound 5-(3-hydroxy-4-methoxy-benzylidene)-2-imino-thiazolidin-4-one was prepared from 3-hydroxy-4-methoxybenzaldehyde following the procedure in Example 1 at a yield of 64%. NMR analysis of the compound provided the following:

¹H NMR (300 MHz, DMSO-d₆): δ7.00-7.04 (m, 2H), 7.04 (s, 1H), 7.44 (s, 1H).

### Example 9

Preparation of 2-hydroxy-5-(2-imino-4-oxo-thiazolidin-5-ylidenemethyl)-benzoic acid (compound 3h)

The compound 2-hydroxy-5-(2-imino-4-oxo-thiazolidin-5-ylidenemethyl)-benzoic acid was prepared from 5-formylsalicylic acid following the procedure in Example 1 at a yield of 72%. NMR analysis of the compound provided the following:

¹H NMR (300 MHz, DMSO-d₆): δ7.08 (d, J=8.4, 1H), 7.56 (s, 1H), 7.76 (d, J=8.4, 1H), 8.04 (s, 1H), 9.11 (s, 1H). MS: m/z=265 (M+1).

### Example 10

Preparation of 3-(2-imino-4-oxo-thiazolidin-5-ylidenemethyl)-benzonitrile (compound 3i)

The 3-(2-imino-4-oxo-thiazolidin-5-ylidenemethyl)-benzonitrile was prepared from 3-cyanobenzaldehyde following the procedure in Example 1 at a yield of 73%. NMR analysis of the compound provided the following:

¹H NMR (300 MHz, DMSO-d₆): δ7.63 (s, 1H), 7.70-7.75 (m, 1H), 7.86-7.89 (m, 2H), 8.02 (s, 1H), 9.27 (s, 1H). MS: m/z 230 (M+1).

### Example 11

Preparation of 2-imino-5-(3-nitro-benzylidene)-thiazolidin-4-one (compound 3j)

The compound 2-imino-5-(3-nitro-benzylidene)-thiazolidin-4-one was prepared from 3-nitrobenzaldehyde following the procedure in Example 1 at a yield of 70%. NMR analysis of the compound provided the following:

¹H NMR (300 MHz, DMSO-d₆): δ7.74 (s, 1H), 7.77-7.83 (m, 1H), 8.03-8.05 (m, 1H), 8.24-8.27 (m, 1H), 8.41 (s, 1H), 9.29 (s, 1H).

### Example 12

Preparation of 2-(2-imino-4-oxo-thiazolidin-5-ylidenemethyl)-benzoic acid (compound 3k)

The 2-(2-imino-4-oxo-thiazolidin-5-ylidenemethyl)-benzoic acid was prepared from 2-carboxybenzaldehyde following the procedure in Example 1 at a yield of 69%. NMR analysis of the compound provided the following:

¹H NMR (300 MHz, DMSO-d₆): δ7.52-7.70 (m, 3H), 7.93-7.95 (m, 1H), 8.16 (s, 1H), 9.12 (s, 1H) ; MS: m/z 249 (M+1).

### Example 13

Preparation of N-[4-(2-imino-4-oxo-thiazolidin-5-ylidenemethyl)-phenyl]-acetamide (compound 31)

The compound N-[4-(2-imino-4-oxo-thiazolidin-5-ylidenemethyl)-phenyl]-acetamide was prepared from 4-acetamidobenzaldehyde following the procedure in Example 1 at a yield of 81%. NMR analysis of the compound provided the following:

¹H NMR (300 MHz, DMSO-d₆): δ7.50 (d, 2H), 7.52 (s, 1H) 8.72 (d, 2H), 9.12 (s, 1H). MS: m/z 262 (M+1).

### Example 14

Preparation of 2-imino-5-pyridin-3-ylmethylene-thiazolidin-4-one (compound 3m)

The 2-imino-5-pyridin-3-ylmethylene-thiazolidin-4-one was prepared from 3-pyridinecarboxaldehyde following the procedure in Example 1 at a yield of 77%. NMR analysis of the compound provided the following:

1H NMR (300 MHz, DMSO-d₆): δ7.53-7.57 (m, 1H), 7.63 (s, 1H), 7.92-7.95, (m, 1H), 8.57-8.59 (m, 1H), 8.81 (s, 1H), 9.27 (s, 1H); MS: m/z 206 (M+1).

### Example 15

Preparation of 5-(2,5-dihydroxy-benzylidene)-2-imino-thiazolidin-4-one (compound 3n)

The compound 5-(2,5-dihydroxy-benzylidene)-2-imino-thiazolidin-4-one was prepared from 2,5-dihydroxybenzaldehyde following the procedure in Example 1 at a yield of 75%. NMR analysis of the compound provided the following:

¹H NMR (300 MHz, DMSO-d₆): δ6.99-7.05 (m, 1H), 7.05 (s, 1H), 7.28-7.31 (m, 1H), 8.05 (s, 1H), 9.75 (s, 1H).

### Example 16

Preparation of 4-{2-[2-hydroxy-5-(2-imino-4-oxo-thiazolidin-5-ylidenemethyl)-benzoylamino]-ethylsulfanyl}-pyridine-2,6-dicarboxylic acid (compound 5a)

This example describes the synthesis of bi-ligands of the invention following the reaction scheme show in FIG. 9. Compound numbers correspond to those in the figure.

The compound 4-amino-pyridine-2,6-dicarboxylic acid dimethyl ester (compound 4, free base, 77 mg, 0.284 mmol), 2-hydroxy-5-(2-imino-4-oxo-thiazolidin-5-ylidenemethyl)-benzoic acid (compound 3 h, 75 mg, 0.284 mmol) and HOBt.H₂O (52 mg, 0.340 mmol) were dissolved in DMF (1 ml). Triethylamine (47 μl, 0.0338 mmol) and ethylene dichloride (EDCl, 72 mg, 0.375 mmol) were added to the mixture which was then stirred at room temperature for 17 hours. The resulting precipitate (39 mg) was collected on a funnel and washed with a mixture of DMF and aqueous 2N HCl.

Next, 37 mg of the solid was suspended in a mixture of MeOH (0.5 ml) and water (0.5 ml), followed by the addition of LiOH (12 mg, 0.50 mmol). The solution was then stirred at room temperature for 2 hours until homogenous. The compound was precipitated with aqueous 2N HCl. The product was filtered, dried, and isolated to give 4-{2-[2-hydroxy-5-(2-imino-4-oxo-thiazolidin-5-ylidenemethyl)-benzoylamino]-ethylsulfanyl}-pyridine-2,6-dicarboxylic acid as a yellow solid (compound 5a, 26.2 mg, 20%).

¹H NMR (300 MHz, DMSO-d₆): δ3.44 (m, 2H), 3.65 (m, 2H) 7.05 (d, J=8.6, 1H), 7.57 (d, J=7.1, 1H), 7.49 (s, 1H), 8.07 (s, 3H), 9.12 (br.s., 1H), 9.40 (br.s., 1H); MS m/z 489 (M+1).

### Example 17

Preparation of 4-{2-[3-(2-imino-4-oxo-thiazolidin-5-ylidenemethyl)-benzoylamino]-ethylsulfanyl}-pyridine-2,6-dicarboxylic acid (compound 5b)

This example describes the synthesis of bi-ligands of the invention following the reaction scheme shown in FIG. 9. Compound numbers correspond to those in the figure.

The compound 4-amino-pyridine-2,6-dicarboxylic acid dimethyl ester (compound 4, free base, 88 mg, 0.326 mmol), pseudothiohydantoin (compound 3d, 81 mg, 0.326 mmol) and HOBt.H₂O (60 mg, 0.392 mmol) were suspended in DMF (2 ml). Triethylamine (54 μl, 0.388 mmol) and EDCl (75 mg, 0.391 mmol) were added to the suspension, followed by stirring at room temperature for 2.5 days.

The resulting precipitate (41 mg) was collected on a funnel and washed with a mixture of DMF and aqueous 0.5N HCl. The crude compound (37.3 mg) was the suspended in a mixture of water (0.5 ml) and MeOH (0.5 ml). LiOH (16 mg, 0.668 mmol) was added to the mixture, which was stirred at room temperature for 1.5 hours until homogenous. The, the mixture was acidified with aqueous 2N HCl. The resulting precipitate was collected, washed with water, and dried to give 4-{2-[3-(2-imino-4-oxo-thiazolidin-5-ylidenemethyl)-benzoylamino]-ethylsulfanyl}-pyridine-2,6-dicarboxylic acid as a pale yellow powder (compound 5b, 32.5 mg, 92%).

¹H NMR (300 MHz, DMSO-d₆) δ3.43 (m, 2H), 3.60 (m, 2H), 7.59 (t, J=7.7, 1H), 7.62 (s, 1H), 7.73 (d, J=7.7, 1H), 7.84 (d, J=7.6, 1H), 8.05 (s, 1H), 8.07 (s, 2H), 8.91 (br. t., J=5.0, 1H), 9.32 (br.s., 1H); MS m/z 385 (M+H+CO₂).

### Example 18

Preparation of 4-{2-[4-(2-imino-4-oxo-thiazolidin-5-ylidenemethyl)-benzoylamino]-ethylsulfanyl}-pyridine-2,6-dicarboxylic acid (compound 5c)

This example describes the synthesis of bi-ligands of the invention following the reaction scheme shown in FIG. 9. Compound numbers correspond to the numbers in the figure.

The compound 4-amino-pyridine-2,6-dicarboxylic acid dimethyl ester (compound 4, free base, 75 mg, 0.277 mmol), 4-(2-imino-4-oxo-thiazolidin-5-ylidenemethyl)-benzoic acid (compound 3e, 83 mg, 0.334 mmol) and HOBt.H₂O (61 mg, 0.398 mmol) were dissolved in DMF (2 ml). Triethylamine (0.14 ml, 1.01 mmol) and ethylene dichloride EDCl (76 mg, 0.396 mmol) were added to the mixture which was then stirred at room temperature for 2 days. The resulting pale yellow precipitate (94 mg) was filtered and washed with aqueous 2N HCl.

Next, 78 mg of the solid was suspended in a mixture of MeOH (0.5 ml) and water (0.5 ml), followed by the addition of LiOH (26 mg, 0.96 mmol). The solution was then stirred at room temperature for 2.5 hours. The mixture was acidified with aqueous 2N HCl, and the product collected on a funnel. The remaining triethylamine (about 20%) was eliminated by subjecting the product to ultrasound for 30 minutes in aqueous HCl. The product was filter to provide 4-{2-[4-(2-imino-4-oxo-thiazolidin-5-ylidenemethyl)-benzoylamino]-ethylsulfanyl}-pyridine-2,6-dicarboxylic acid as a yellow powder (compound 5c, 41 mg, 32%).

¹H NMR (300 MHz, DMSO-d₆): δ3.58 (t, J=5.5, 2H) and one signal overlapped by water, 7.63 (s, 1H), 7.64 (d, J=9.7, 2H), 7.92 (d, J=8.1, 2H), 8.07 (s, 2H), 8.88 (br.t., J=5.1, 1H), 9.26 (br.s., 1H), and 9.53 (br.s., 1H); MS m/z 473 (M+1).

### Example 19

Preparation of Common Ligand Mimics Having Amide Linkers

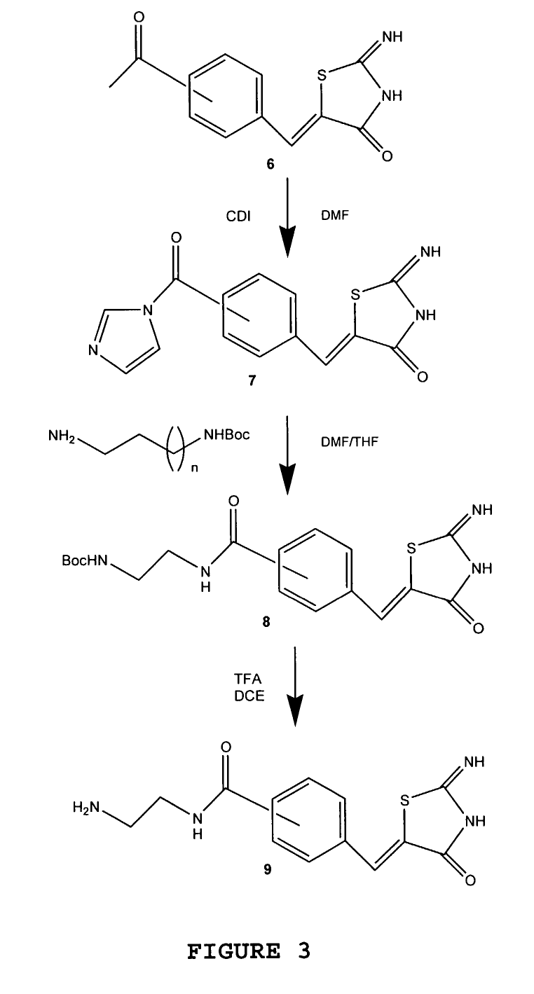

This example describes the synthesis of common ligand mimics of the invention containing a linker group following the reaction scheme shown in FIG. 3. Compound numbers correspond to the numbers in the figure.

In a 500 ml round-bottom flask, compound 6 is dissolved in dry DMF by heating. The solution is cooled to a temperature of 40 to 50° C. THF (ca 150 ml) and 1,1′-carbonyldiimidazole (4.5 g) are added to the solution. After shaking for 20 minutes, the flask is capped and refrigerated overnight at −10° C. The precipitate is collected by filtration and washed with THF to provide intermediate compound 7.

A mixture of dry DMF (30 ml) and dry THF (80 ml) is prepared in a 250 ml flask. Intermediate compound 7 is added to the mixture. Boc protected diamines (1.2 eq) are added to the mixture which then is heated at a temperature of 65° C. for a period of 1 hour. By this time, the undissolved solid has dissolved, and a clear solution is obtained. The solvent then is evaporated under reduced pressure to provide compound 8.

A solution of 50% trifluoacetic acid in dichloroethane (100 ml) is added compound 8 and reacted for 10 minutes. Extra solvent is evaporated, resulting in a yellow solid. The yellow solid is then dissolved in 40 to 50 ml of DMF by heating. The solution is cooled to room temperature, and a Na₂CO₃ solution (150-200 ml, 5%) is added. When a yellow precipitate forms, it is filtered. Otherwise, more DMF solvent is evaporated, and more water is added. The yellow solid, compound 9, is washed with a mixture of water and MeOH and then dried to provide 5 to 5.5 g of product compound 9.

Examples of compounds, which can be produced by the methods described in Example 19, include those in Tables 4 to 10.

TABLE 4

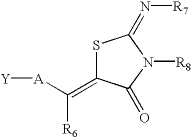

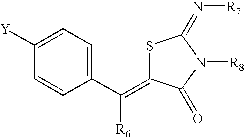

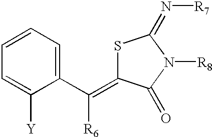

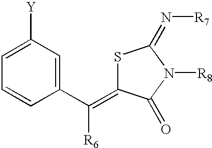

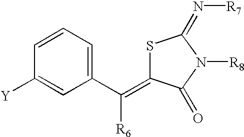

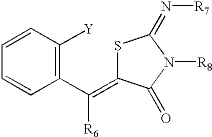

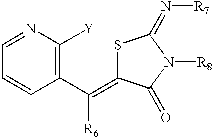

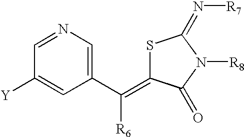

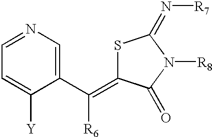

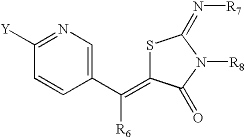

| | Y |
|---|---|
| 1 | OH |
| 1 | SH |
| 1 | COOH |
| 1 | SO₂H |
| 1 | Cl |
| 1 | Br |
| 1 | I |
| 1 | F |
| 1 | CN |
| 1 | N₃ |
| 1 | CONH₂ |
| 1 | CH═CH₂ |
| 1 | C≡CH |
| 1 | NH₂ |
| 1 | NHR |
| 1 | COH |
| 1 | COR |
| 2 | OH |
| 2 | SH |
| 2 | COOH |
| 2 | SO₂H |
| 2 | Cl |
| 2 | Br |
| 2 | I |
| 2 | F |
| 2 | CN |
| 2 | N₃ |
| 2 | CONH₂ |
| 2 | CH═CH₂ |
| 2 | C≡CH |
| 2 | NH₂ |
| 2 | NHR |
| 2 | COH |
| 2 | COR |
| 3 | OH |
| 3 | SH |
| 3 | COOH |
| 3 | SO₂H |
| 3 | Cl |
| 3 | Br |
| 3 | I |
| 3 | F |
| 3 | CN |
| 3 | N₃ |
| 3 | CONH₂ |
| 3 | CH═CH₂ |
| 3 | C≡CH |
| 3 | NH₂ |
| 3 | NHR |
| 3 | COH |
| 3 | COR |
| 4 | OH |
| 4 | SH |
| 4 | COOH |
| 4 | SO₂H |
| 4 | Cl |
| 4 | Br |
| 4 | I |
| 4 | F |
| 4 | CN |
| 4 | N₃ |
| 4 | CONH₂ |
| 4 | CH═CH₂ |
| 4 | C≡CH |
| 4 | NH₂ |
| 4 | NHR |
| 4 | COH |
| 4 | COR |
| 5 | OH |
| 5 | SH |
| 5 | COOH |
| 5 | SO₂H |
| 5 | Cl |
| 5 | Br |
| 5 | I |
| 5 | F |
| 5 | CN |
| 5 | N₃ |
| 5 | CONH₂ |
| 5 | CH═CH₂ |
| 5 | C≡CH |
| 5 | NH₂ |
| 5 | NHR |
| 5 | COH |
| 5 | COR |

R = alkyl, alkenyl, alkynyl, aryl, or heterocycle

TABLE 5

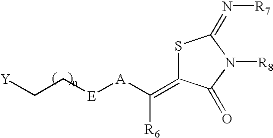

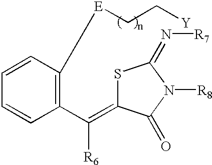

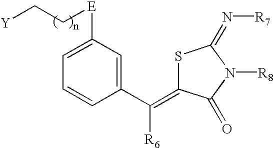

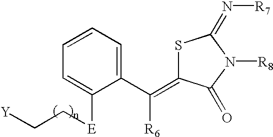

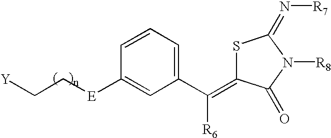

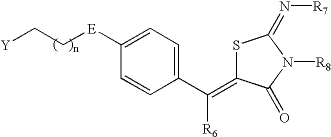

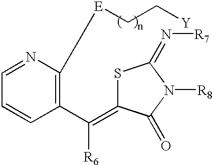

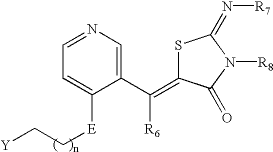

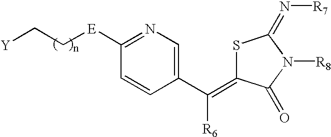

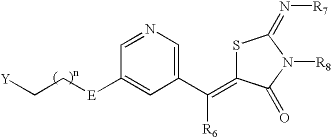

| n | E | Y |
|---|---|---|
| 0 | O | OH |
| 0 | O | SH |
| 0 | O | COOH |
| 0 | O | SO₂H |
| 0 | O | Cl |
| 0 | O | Br |
| 0 | O | I |
| 0 | O | F |
| 0 | O | CN |
| 0 | O | N₃ |
| 0 | O | CONH₂ |
| 0 | O | CH═CH₂ |
| 0 | O | C≡CH |
| 0 | O | NH₂ |
| 0 | O | NHR |
| 0 | O | COH |
| 0 | O | COR |
| 0 | CH₂ | OH |
| 0 | CH₂ | SH |
| 0 | CH₂ | COOH |
| 0 | CH₂ | SO₂H |
| 0 | CH₂ | Cl |
| 0 | CH₂ | Br |
| 0 | CH₂ | I |
| 0 | CH₂ | F |
| 0 | CH₂ | CN |
| 0 | CH₂ | N₃ |
| 0 | CH₂ | CONH₂ |
| 0 | CH₂ | CH═CH₂ |
| 0 | CH₂ | C≡CH |
| 0 | CH₂ | NH₂ |
| 0 | CH₂ | NHR |
| 0 | CH₂ | COH |
| 0 | CH₂ | COR |
| 0 | SO₂NH | OH |
| 0 | SO₂NH | SH |
| 0 | SO₂NH | COOH |
| 0 | SO₂NH | SO₂H |
| 0 | SO₂NH | Cl |
| 0 | SO₂NH | Br |
| 0 | SO₂NH | I |
| 0 | SO₂NH | FN |
| 0 | SO₂NH | CN |
| 0 | SO₂NH | N₃ |
| 0 | SO₂NH | CONH₂ |
| 0 | SO₂NH | CH≡CH₂ |
| 0 | SO₂NH | C≡CH |
| 0 | SO₂NH | NH_{R} |
| 0 | SO₂NH | NHR |
| 0 | SO₂NH | COH |
| O | SO₂NH | COR |
| 0 | NHCNHNH | OH |
| 0 | NHCNHNH | SH |
| 0 | NHCNHNH | COOH |
| 0 | NHCNHNH | SO₂H |
| 0 | NHCNHNH | Cl |
| 0 | NHCNHNH | Br |
| 0 | NHCNHNH | I |
| 0 | NHCNHNH | F |
| 0 | NHCNHNH | CN |
| 0 | NHCNHNH | N₃ |
| 0 | NHCNHNH | CONH₂ |
| 0 | NHCNHNH | CH═CH₂ |
| 0 | NHCNHNH | CC≡H |
| 0 | NHCNHNH | NH₂ |
| 0 | NHCNHNH | NHR |
| 0 | NHCNHNH | COH |
| 0 | NHCNHNH | COR |
| 0 | C≡C | OH |
| 0 | C≡C | SH |
| 0 | C≡C | COOH |
| 0 | C≡C | SO₂H |
| 0 | C≡C | Cl |
| 0 | C≡C | Br |
| 0 | C≡C | I |
| 0 | C≡C | F |
| 0 | C≡C | CN |
| 0 | C≡C | N₃ |
| 0 | C≡C | CONH₂ |
| 0 | C≡C | CH═CH₂ |
| 0 | C≡C | C≡CH |
| 0 | C≡C | NH₂ |
| 0 | C≡C | NHR |
| 0 | C≡C | COH |
| 0 | C≡C | COR |
| 1 | NH | OH |
| 1 | NH | SH |
| 1 | NH | COOH |
| 1 | NH | SO₂H |
| 1 | NH | Cl |
| 1 | NH | Br |
| 1 | NH | I |
| 1 | NH | F |
| 1 | NH | CN |
| 1 | NH | N₃ |
| 1 | NH | CONH₂ |
| 1 | NH | CH═CH₂ |
| 1 | NH | C≡CH |
| 1 | NH | NH₂ |
| 1 | NH | NHR |
| 1 | NH | COH |
| 1 | NH | COR |
| 1 | CONH | OH |
| 1 | CONH | SH |
| 1 | CONH | COOH |
| 1 | CONH | SO₂H |
| 1 | CONH | Cl |
| 1 | CONH | Br |
| 1 | CONH | I |
| 1 | CONH | F |
| 1 | CONH | CN |
| 1 | CONH | N₃ |
| 1 | CONH | CONH₂ |
| 1 | CONH | CH═CH₂ |
| 1 | CONH | C≡CH |
| 1 | CONH | NH₂ |
| 1 | CONH | NHR |
| 1 | CONH | COH |
| 1 | CONH | COR |
| 1 | NHCONH | OH |
| 1 | NHCONH | SH |
| 1 | NHCONH | COOH |
| 1 | NHCONH | SO₂H |
| 1 | NHCONH | Cl |
| 1 | NHCONH | Br |
| 1 | NHCONH | I |
| 1 | NHCONH | F |
| 1 | NHCONH | CN |
| 1 | NHCONH | N₃ |
| 1 | NHCONH | CONH₂ |
| 1 | NHCONH | CH═CH₂ |
| 1 | NHCONH | C≡CH |
| 1 | NHCONH | NH₂ |
| 1 | NHCONH | NHR |
| 1 | NHCONH | COH |
| 1 | NHCONH | COR |
| 1 | NHCOO | OH |
| 1 | NHCOO | SH |
| 1 | NHCOO | COOH |
| 1 | NHCOO | SO₂H |
| 1 | NHCOO | Cl |
| 1 | NHCOO | Br |
| 1 | NHCOO | I |
| 1 | NHCOO | F |
| 1 | NHCOO | CN |
| 1 | NHCOO | N₃ |
| 1 | NHCOO | CONH₂ |
| 1 | NHCOO | CH═CH₂ |
| 1 | NHCOO | C≡CH |
| 1 | NHCOO | NH₂ |
| 1 | NHCOO | NHR |
| 1 | NHCOO | COH |
| 1 | NHCOO | COR |
| 2 | O | OH |
| 2 | O | SH |
| 2 | O | COOH |
| 2 | O | SO₂H |
| 2 | O | Cl |
| 2 | O | Br |
| 2 | O | I |
| 2 | O | F |
| 2 | O | CN |
| 2 | O | N₃ |
| 2 | O | CONH₂ |
| 2 | O | CH═CH₂ |
| 2 | O | C≡CH |
| 2 | O | NH₂ |
| 2 | O | NHR |
| 2 | O | COH |
| 2 | O | COR |
| 2 | CH₂ | OH |
| 2 | CH₂ | SH |
| 2 | CH₂ | COOH |
| 2 | CH₂ | SO₂H |
| 2 | CH₂ | Cl |
| 2 | CH₂ | Br |
| 2 | CH₂ | I |
| 2 | CH₂ | F |
| 2 | CH₂ | CN |
| 2 | CH₂ | N₃ |
| 2 | CH₂ | CONH₂ |
| 2 | CH₂ | CH═CH₂ |
| 2 | CH₂ | C≡CH |
| 2 | CH₂ | NH₂ |
| 2 | CH₂ | NHR |
| 2 | CH₂ | COH |
| 2 | CH₂ | COR |
| 2 | SO₂NH | OH |
| 2 | SO₂NH | SH |
| 2 | SO₂NH | COOH |
| 2 | SO₂NH | SO₂H |
| 2 | SO₂NH | Cl |
| 2 | SO₂NH | Br |
| 2 | SO₂NH | I |
| 2 | SO₂NH | F |
| 2 | SO₂NH | CN |
| 2 | SO₂NH | N₃ |
| 2 | SO₂NH | CONH₂ |
| 2 | SO₂NH | CH═CH₂ |
| 2 | SO₂NH | C≡CH |
| 2 | SO₂NH | NH₂ |
| 2 | SO₂NH | NHR |
| 2 | SO₂NH | COH |
| 2 | SO₂NH | COR |
| 2 | NHCNHNH | OH |
| 2 | NHCNHNH | SH |
| 2 | NHCNHNH | COOH |
| 2 | NHCNHNH | SO₂H |
| 2 | NHCNHNH | Cl |
| 2 | NHCNHNH | Br |
| 2 | NHCNHNH | I |
| 2 | NHCNHNH | F |
| 2 | NHCNHNH | CN |
| 2 | NHCNHNH | N₃ |
| 2 | NHCNHNH | CONH₂ |
| 2 | NHCNHNH | CH═CH₂ |
| 2 | NHCNHNH | CCH |
| 2 | NHCNHNH | NH₂ |
| 2 | NHCNHNH | NHR |
| 2 | NHCNHNH | COH |
| 2 | NHCNHNH | COR |
| 2 | C≡C | OH |
| 2 | C≡C | SH |
| 2 | C≡C | COOH |
| 2 | C≡C | SO₂H |
| 2 | C≡C | Cl |
| 2 | C≡C | Br |
| 2 | C≡C | I |
| 2 | C≡C | F |
| 2 | C≡C | CN |
| 2 | C≡C | N₃ |
| 2 | C≡C | CONH₂ |
| 2 | C≡C | CH═CH₂ |
| 2 | C≡C | C≡CH |
| 2 | C≡C | NH₂ |
| 2 | C≡C | NHR |
| 2 | C≡C | COH |
| 2 | C≡C | COR |
| 3 | NH | OH |
| 3 | NH | SH |
| 3 | NH | COOH |
| 3 | NH | SO₂H |
| 3 | NH | Cl |
| 3 | NH | Br |
| 3 | NH | I |
| 3 | NH | F |
| 3 | NH | CN |
| 3 | NH | N₃ |
| 3 | NH | CONH₂ |
| 3 | NH | CH═CH₂ |
| 3 | NH | C≡CH |
| 3 | NH | NH₂ |
| 3 | NH | NHR |
| 3 | NH | COH |
| 3 | NH | COR |
| 3 | CONH | OH |
| 3 | CONH | SH |
| 3 | CONH | COOH |
| 3 | CONH | SO₂H |
| 3 | CONH | Cl |
| 3 | CONH | Br |
| 3 | CONH | I |
| 3 | CONH | F |
| 3 | CONH | CN |
| 3 | CONH | N₃ |
| 3 | CONH | CONH₂ |
| 3 | CONH | CH═CH₂ |
| 3 | CONH | C≡CH |
| 3 | CONH | NH₂ |
| 3 | CONH | NHR |
| 3 | CONH | COH |
| 3 | CONH | COR |
| 3 | NHCONH | OH |
| 3 | NHCONH | SH |
| 3 | NHCONH | COOH |
| 3 | NHCONH | SO₂H |
| 3 | NHCONH | Cl |
| 3 | NHCONH | Br |
| 3 | NHCONH | I |
| 3 | NHCONH | F |
| 3 | NHCONH | CN |
| 3 | NHCONH | N₃ |
| 3 | NHCONH | CONH₂ |
| 3 | NHCONH | CH═CH₂ |
| 3 | NHCONH | C≡CH |
| 3 | NHCONH | NH₂ |
| 3 | NHCONH | NHR |
| 3 | NHCONH | COH |
| 3 | NHCONH | COR |
| 3 | NHCOO | OH |
| 3 | NHCOO | SH |
| 3 | NHCOO | COOH |
| 3 | NHCOO | SO₂H |
| 3 | NHCOO | Cl |
| 3 | NHCOO | Br |
| 3 | NHCOO | I |
| 3 | NHCOO | F |
| 3 | NHCOO | CN |
| 3 | NHCOO | N₃ |
| 3 | NHCOO | CONH₂ |
| 3 | NHCOO | CH═CH₂ |
| 3 | NHCOO | C≡CH |
| 3 | NHCOO | NH₂ |
| 3 | NHCOO | NHR |
| 3 | NHCOO | COH |
| 3 | NHCOO | COR |
| 4 | O | OH |
| 4 | O | SH |
| 4 | O | COOH |
| 4 | O | SO₂H |
| 4 | O | Cl |
| 4 | O | Br |
| 4 | O | I |
| 4 | O | F |
| 4 | O | CN |
| 4 | O | N₃ |
| 4 | O | CONH₂ |
| 4 | O | CH═CH₂ |
| 4 | O | C≡CH |
| 4 | O | NH₂ |
| 4 | O | NHR |
| 4 | O | COH |
| 4 | O | COR |
| 4 | CH₂ | OH |
| 4 | CH₂ | SH |
| 4 | CH₂ | COOH |
| 4 | CH₂ | SO₂H |
| 4 | CH₂ | Cl |
| 4 | CH₂ | Br |
| 4 | CH₂ | I |
| 4 | CH₂ | F |
| 4 | CH₂ | CN |
| 4 | CH₂ | N₃ |
| 4 | CH₂ | CONH₂ |
| 4 | CH₂ | CH═CH₂ |
| 4 | CH₂ | C≡CH |
| 4 | CH₂ | NH₂ |
| 4 | CH₂ | NHR |
| 4 | CH₂ | COH |
| 4 | CH₂ | COR |
| 4 | SO₂NH | OH |
| 4 | SO₂NH | SH |
| 4 | SO₂NH | COOH |
| 4 | SO₂NH | SO₂H |
| 4 | SO₂NH | Cl |
| 4 | SO₂NH | Br |
| 4 | SO₂NH | I |
| 4 | SO₂NH | F |
| 4 | SO₂NH | CN |
| 4 | SO₂NH | N₃ |
| 4 | SO₂NH | CONH₂ |
| 4 | SO₂NH | CH═CH₂ |
| 4 | SO₂NH | C≡CH |
| 4 | SO₂NH | NH₂ |
| 4 | SO₂NH | NHR |
| 4 | SO₂NH | COH |
| 4 | SO₂NH | COR |
| 4 | NHCNHNH | OH |
| 4 | NHCNHNH | SH |
| 4 | NHCNHNH | COOH |
| 4 | NHCNHNH | SO₂H |
| 4 | NHCNHNH | Cl |
| 4 | NHCNHNH | Br |
| 4 | NHCNHNH | I |
| 4 | NHCNHNH | F |
| 4 | NHCNHNH | CN |
| 4 | NHCNHNH | N₃ |
| 4 | NHCNHNH | CONH₂ |
| 4 | NHCNHNH | CH═CH₂ |
| 4 | NHCNHNH | C≡CH |
| 4 | NHCNHNH | NH₂ |
| 4 | NHCNHNH | NHR |
| 4 | NHCNHNH | COH |
| 4 | NHCNHNH | COR |
| 4 | C≡C | OH |
| 4 | C≡C | SH |
| 4 | C≡C | COOH |
| 4 | C≡C | SO₂H |
| 4 | C≡C | Cl |
| 4 | C≡C | Br |
| 4 | C≡C | I |
| 4 | C≡C | F |
| 4 | C≡C | CN |
| 4 | C≡C | N₃ |
| 4 | C≡C | CONH₂ |
| 4 | C≡C | CH═CH₂ |
| 4 | C≡C | C≡CH |
| 4 | C≡C | NH₂ |
| 4 | C≡C | NHR |
| 4 | C≡C | COH |
| 4 | C≡C | COR |
| 5 | NH | OH |
| 5 | NH | SH |
| 5 | NH | COOH |
| 5 | NH | SO₂H |
| 5 | NH | Cl |
| 5 | NH | Br |
| 5 | NH | I |
| 5 | NH | F |
| 5 | NH | CN |
| 5 | NH | N₃ |
| 5 | NH | CONH₂ |
| 5 | NH | CH═CH₂ |
| 5 | NH | C≡CH |
| 5 | NH | NH₂ |
| 5 | NH | NHR |
| 5 | NH | COH |
| 5 | NH | COR |
| 5 | CONH | OH |
| 5 | CONH | SH |
| 5 | CONH | COOH |
| 5 | CONH | SO₂H |
| 5 | CONH | Cl |
| 5 | CONH | Br |
| 5 | CONH | I |
| 5 | CONH | F |
| 5 | CONH | CN |
| 5 | CONH | N₃ |
| 5 | CONH | CONH₂ |
| 5 | CONH | CH═CH₂ |
| 5 | CONH | C≡CH |
| 5 | CONH | NH₂ |
| 5 | CONH | NHR |
| 5 | CONH | COH |
| 5 | CONH | COR |
| 5 | NHCONH | OH |
| 5 | NHCONH | SH |
| 5 | NHCONH | COOH |
| 5 | NHCONH | SO₂H |
| 5 | NHCONH | Cl |
| 5 | NHCONH | Br |
| 5 | NHCONH | I |
| 5 | NHCONH | F |
| 5 | NHCONH | CN |
| 5 | NHCONH | N₃ |
| 5 | NHCONH | CONH₂ |
| 5 | NHCONH | CH═CH₂ |
| 5 | NHCONH | C≡CH |
| 5 | NHCONH | NH₂ |
| 5 | NHCONH | NHR |
| 5 | NHCONH | COH |
| 5 | NHCONH | COR |
| 5 | NRCNHNR | OH |
| 5 | NRCNHNR | SH |
| 5 | NRCNHNR | COOH |
| 5 | NRCNHNR | SO₂H |
| 5 | NRCNHNR | Cl |
| 5 | NRCNHNR | Br |
| 5 | NRCNHNR | I |
| 5 | NRCNHNR | F |
| 5 | NRCNHNR | CN |
| 5 | NRCNHNR | N₃ |
| 5 | NRCNHNR | CONH₂ |
| 5 | NRCNHNR | CH═CH₂ |
| 5 | NRCNHNR | C≡CH |
| 5 | NRCNHNR | NH₂ |
| 5 | NRCNHNR | NHR |
| 5 | NRCNHNR | COH |
| 5 | NRCNHNR | COR |
| 5 | CH₂═CH₂ | OH |
| 5 | CH₂═CH₂ | SH |
| 5 | CH₂═CH₂ | COOH |
| 5 | CH₂═CH₂ | SO₂H |
| 5 | CH₂═CH₂ | Cl |
| 0 | S | OH |
| 0 | S | SH |
| 0 | S | COOH |
| 0 | S | SO₂H |
| 0 | S | Cl |
| 0 | S | Br |
| 0 | S | I |
| 0 | S | F |
| 0 | S | CN |
| 0 | S | N₃ |
| 0 | S | CONH₂ |
| 0 | S | CH═CH₂ |
| 0 | S | C≡CH |
| 0 | S | NH₂ |
| 0 | S | NHR |
| 0 | S | COH |
| 0 | S | COR |
| 0 | COR₁R₂ | OH |
| 0 | COR₁R₂ | SH |
| 0 | COR₁R₂ | COOH |
| 0 | COR₁R₂ | SO₂H |
| 0 | COR₁R₂ | Cl |
| 0 | COR₁R₂ | Br |
| 0 | COR₁R₂ | I |
| 0 | COR₁R₂ | F |
| 0 | COR₁R₂ | CN |
| 0 | COR₁R₂ | N₃ |
| 0 | COR₁R₂ | CONH₂ |
| 0 | COR₁R₂ | CH═CH₂ |
| 0 | COR₁R₂ | C≡CH |
| 0 | COR₁R₂ | NH₂ |
| 0 | COR₁R₂ | NHR |
| 0 | COR₁R₂ | COH |
| 0 | COR₁R₂ | COR |
| 0 | SO₂NR | OH |
| 0 | SO₂NR | SH |
| 0 | SO₂NR | COOH |
| 0 | SO₂NR | SO₂H |
| 0 | SO₂NR | Cl |
| 0 | SO₂NR | Br |
| 0 | SO₂NR | I |
| 0 | SO₂NR | F |
| 0 | SO₂NR | CN |
| 0 | SO₂NR | N₃ |
| 0 | SO₂NR | CONH₂ |
| 0 | SO₂NR | CH═CH₂ |
| 0 | SO₂NR | C≡CH |
| 0 | SO₂NR | NH₂ |
| 0 | SO₂NR | NHR |
| 0 | SO₂NR | COH |
| 0 | SO₂NR | COR |
| 0 | NRCNHNR | OH |
| 0 | NRCNHNR | SH |
| 0 | NRCNHNR | COOH |
| 0 | NRCNHNR | SO₂H |
| 0 | NRCNHNR | Cl |
| 0 | NRCNHNR | Br |
| 0 | NRCNHNR | I |
| 0 | NRCNHNR | F |
| 0 | NRCNHNR | CN |
| 0 | NRCNHNR | N₃ |
| 0 | NRCNHNR | CONH₂ |
| 0 | NRCNHNR | CH═CH₂ |
| 0 | NRCNHNR | C≡CH |
| 0 | NRCNHNR | NH₂ |
| 0 | NRCNHNR | NHR |
| 0 | NRCNHNR | COH |
| 0 | NRCNHNR | COR |
| 0 | CH₂═CH₂ | OH |
| 0 | CH₂═CH₂ | SH |
| 0 | CH₂═CH₂ | COOH |
| 0 | CH₂═CH₂ | SO₂H |
| 0 | CH₂═CH₂ | Cl |
| 0 | CH₂═CH₂ | Br |
| 0 | CH₂═CH₂ | I |
| 0 | CH₂═CH₂ | F |
| 0 | CH₂═CH₂ | CN |
| 0 | CH₂═CH₂ | N₃ |
| 0 | CH₂═CH₂ | CONH₂ |
| 0 | CH₂═CH₂ | CH═CH₂ |
| 0 | CH₂═CH₂ | C≡CH |
| 0 | CH₂═CH₂ | NH₂ |
| 0 | CH₂═CH₂ | NHR |
| 0 | CH₂═CH₂ | COH |
| 0 | CH₂═CH₂ | COR |
| 1 | NR | OH |
| 1 | NR | SH |
| 1 | NR | COOH |
| 1 | NR | SO₂H |
| 1 | NR | Cl |
| 1 | NR | Br |
| 1 | NR | I |
| 1 | NR | F |
| 1 | NR | CN |
| 1 | NR | N₃ |
| 1 | NR | CONH₂ |
| 1 | NR | CH═CH₂ |
| 1 | NR | C≡CH |
| 1 | NR | NH₂ |
| 1 | NR | NHR |
| 1 | NR | COH |
| 1 | NR | COR |
| 1 | CONR | OH |
| 1 | CONR | SH |
| 1 | CONR | COOH |
| 1 | CONR | SO₂H |
| 1 | CONR | Cl |
| 1 | CONR | Br |
| 1 | CONR | I |
| 1 | CONR | F |
| 1 | CONR | CN |
| 1 | CONR | N₃ |
| 1 | CONR | CONH₂ |
| 1 | CONR | CH═CH₂ |
| 1 | CONR | C≡CH |
| 1 | CONR | NH₂ |
| 1 | CONR | NHR |
| 1 | CONR | COH |
| 1 | CONR | COR |
| 1 | NRCONR | OH |
| 1 | NRCONR | SH |
| 1 | NRCONR | COOH |
| 1 | NRCONR | SO₂H |
| 1 | NRCONR | Cl |
| 1 | NRCONR | Br |
| 1 | NRCONR | I |
| 1 | NRCONR | F |
| 1 | NRCONR | CN |
| 1 | NRCONR | N₃ |
| 1 | NRCONR | CONH₂ |
| 1 | NRCONR | CH═CH₂ |
| 1 | NRCONR | C≡CH |
| 1 | NRCONR | NH₂ |
| 1 | NRCONR | NHR |
| 1 | NRCONR | COH |
| 1 | NRCONR | COR |
| 1 | NRCOO | OH |
| 1 | NRCOO | SH |
| 1 | NRCOO | COOH |
| 1 | NRCOO | SO₂H |
| 1 | NRCOO | Cl |
| 1 | NRCOO | Br |
| 1 | NRCOO | I |
| 1 | NRCOO | F |
| 1 | NRCOO | CN |
| 1 | NRCOO | N₃ |
| 1 | NRCOO | CONH₂ |
| 1 | NRCOO | CH═CH₂ |
| 1 | NRCOO | C≡CH |
| 1 | NRCOO | NH₂ |
| 1 | NRCOO | NHR |
| 1 | NRCOO | COH |
| 1 | NRCOO | COR |
| 2 | S | OH |
| 2 | S | SH |
| 2 | S | COOH |
| 2 | S | SO₂H |
| 2 | S | Cl |
| 2 | S | Br |
| 2 | S | I |
| 2 | S | F |
| 2 | S | CN |
| 2 | S | N₃ |
| 2 | S | CONH₂ |
| 2 | S | CH═CH₂ |
| 2 | S | C≡CH |
| 2 | S | NH₂ |
| 2 | S | NHR |
| 2 | S | COH |
| 2 | S | COR |
| 2 | COR₁R₂ | OH |
| 2 | COR₁R₂ | SH |
| 2 | COR₁R₂ | COOH |
| 2 | COR₁R₂ | SO₂H |
| 2 | COR₁R₂ | Cl |
| 2 | COR₁R₂ | Br |
| 2 | COR₁R₂ | I |
| 2 | COR₁R₂ | F |
| 2 | COR₁R₂ | CN |
| 2 | COR₁R₂ | N₃ |
| 2 | COR₁R₂ | CONH₂ |
| 2 | COR₁R₂ | CH═CH₂ |
| 2 | COR₁R₂ | C≡CH |
| 2 | COR₁R₂ | NH₂ |
| 2 | COR₁R₂ | NHR |
| 2 | COR₁R₂ | COH |
| 2 | COR₁R₂ | COR |
| 2 | SO₂NR | OH |
| 2 | SO₂NR | SH |
| 2 | SO₂NR | COOH |
| 2 | SO₂NR | SO₂H |
| 2 | SO₂NR | Cl |
| 2 | SO₂NR | Br |
| 2 | SO₂NR | I |
| 2 | SO₂NR | F |
| 2 | SO₂NR | CN |
| 2 | SO₂NR | N₃ |
| 2 | SO₂NR | CONH₂ |
| 2 | SO₂NR | CH═CH₂ |
| 2 | SO₂NR | CCH |
| 2 | SO₂NR | NH₂ |
| 2 | SO₂NR | NHR |
| 2 | SO₂NR | COH |
| 2 | SO₂NR | COR |
| 2 | NRCNHNR | OH |
| 2 | NRCNHNR | SH |
| 2 | NRCNHNR | COOH |
| 2 | NRCNHNR | SO₂H |
| 2 | NRCNHNR | Cl |
| 2 | NRCNHNR | Br |
| 2 | NRCNHNR | I |
| 2 | NRCNHNR | F |
| 2 | NRCNHNR | CN |
| 2 | NRCNHNR | N₃ |
| 2 | NRCNHNR | CONH₂ |
| 2 | NRCNHNR | CH═CH₂ |
| 2 | NRCNHNR | C≡CH |
| 2 | NRCNHNR | NH₂ |
| 2 | NRCNHNR | NHR |
| 2 | NRCNHNR | COH |
| 2 | NRCNHNR | COR |
| 2 | CH₂═CH₂ | OH |
| 2 | CH₂═CH₂ | SH |
| 2 | CH₂═CH₂ | COOH |
| 2 | CH₂═CH₂ | SO₂H |
| 2 | CH₂═CH₂ | Cl |
| 2 | CH₂═CH₂ | Br |
| 2 | CH₂═CH₂ | I |
| 2 | CH₂═CH₂ | F |
| 2 | CH₂═CH₂ | CN |
| 2 | CH₂═CH₂ | N₃ |
| 2 | CH₂═CH₂ | CONH₂ |
| 2 | CH₂═CH₂ | CH═CH₂ |
| 2 | CH₂═CH₂ | C≡CH |
| 2 | CH₂═CH₂ | NH₂ |
| 2 | CH₂═CH₂ | NHR |
| 2 | CH₂═CH₂ | COH |
| 2 | CH₂═CH₂ | COR |
| 3 | NR | OH |
| 3 | NR | SH |
| 3 | NR | COOH |
| 3 | NR | SO₂H |
| 3 | NR | Cl |
| 3 | NR | Br |
| 3 | NR | I |
| 3 | NR | F |
| 3 | NR | CN |
| 3 | NR | N₃ |
| 3 | NR | CONH₂ |
| 3 | NR | CH═CH₂ |
| 3 | NR | C≡CH |
| 3 | NR | NH₂ |
| 3 | NR | NHR |
| 3 | NR | COH |
| 3 | NR | COR |
| 3 | CONR | OH |
| 3 | CONR | SH |
| 3 | CONR | COOH |
| 3 | CONR | SO₂H |
| 3 | CONR | Cl |
| 3 | CONR | Br |
| 3 | CONR | I |
| 3 | CONR | F |
| 3 | CONR | CN |
| 3 | CONR | N₃ |
| 3 | CONR | CONH₂ |
| 3 | CONR | CH═CH₂ |
| 3 | CONR | C≡CH |
| 3 | CONR | NH₂ |
| 3 | CONR | NHR |
| 3 | CONR | COH |
| 3 | CONR | COR |
| 3 | NRCONR | OH |
| 3 | NRCONR | SH |
| 3 | NRCONR | COOH |
| 3 | NRCONR | SO₂H |
| 3 | NRCONR | Cl |
| 3 | NRCONR | Br |
| 3 | NRCONR | I |
| 3 | NRCONR | F |
| 3 | NRCONR | CN |
| 3 | NRCONR | N₃ |
| 3 | NRCONR | CONH₂ |
| 3 | NRCONR | CH═CH₂ |
| 3 | NRCONR | C≡CH |
| 3 | NRCONR | NH₂ |
| 3 | NRCONR | NHR |
| 3 | NRCONR | COH |
| 3 | NRCONR | COR |
| 3 | NRCOO | OH |
| 3 | NRCOO | SH |
| 3 | NRCOO | COOH |
| 3 | NRCOO | SO₂H |
| 3 | NRCOO | Cl |
| 3 | NRCOO | Br |
| 3 | NRCOO | I |
| 3 | NRCOO | F |
| 3 | NRCOO | CN |
| 3 | NRCOO | N₃ |
| 3 | NRCOO | CONH₂ |
| 3 | NRCOO | CH═CH₂ |
| 3 | NRCOO | C≡CH |
| 3 | NRCOO | NH₂ |
| 3 | NRCOO | NHR |
| 3 | NRCOO | COH |
| 3 | NRCOO | COR |
| 4 | S | OH |
| 4 | S | SH |
| 4 | S | COOH |
| 4 | S | SO₂H |
| 4 | S | Cl |
| 4 | S | Br |
| 4 | S | I |
| 4 | S | F |
| 4 | S | CN |
| 4 | S | N₃ |
| 4 | S | CONH₂ |
| 4 | S | CH═CH₂ |
| 4 | S | C≡CH |
| 4 | S | NH₂ |
| 4 | S | NHR |
| 4 | S | COH |
| 4 | S | COR |
| 4 | COR₁R₂ | OH |
| 4 | COR₁R₂ | SH |
| 4 | COR₁R₂ | COOH |
| 4 | COR₁R₂ | SO₂H |
| 4 | COR₁R₂ | Cl |
| 4 | COR₁R₂ | Br |
| 4 | COR₁R₂ | I |
| 4 | COR₁R₂ | F |
| 4 | COR₁R₂ | CN |
| 4 | COR₁R₂ | N₃ |
| 4 | COR₁R₂ | CONH₂ |
| 4 | COR₁R₂ | CH═CH₂ |
| 4 | COR₁R₂ | C≡CH |
| 4 | COR₁R₂ | NH₂ |
| 4 | COR₁R₂ | NHR |
| 4 | COR₁R₂ | COH |
| 4 | COR₁R₂ | COR |
| 4 | SO₂NR | OH |
| 4 | SO₂NR | SH |
| 4 | SO₂NR | COOH |
| 4 | SO₂NR | SO₂H |
| 4 | SO₂NR | Cl |
| 4 | SO₂NR | Br |
| 4 | SO₂NR | I |
| 4 | SO₂NR | F |
| 4 | SO₂NR | CN |
| 4 | SO₂NR | N₃ |
| 4 | SO₂NR | CONH₂ |
| 4 | SO₂NR | CH═CH₂ |
| 4 | SO₂NR | C≡CH |
| 4 | SO₂NR | NH₂ |
| 4 | SO₂NR | NHR |
| 4 | SO₂NR | COH |
| 4 | SO₂NR | COR |
| 4 | NRCNHNR | OH |
| 4 | NRCNHNR | SH |
| 4 | NRCNHNR | COOH |
| 4 | NRCNHNR | SO₂H |
| 4 | NRCNHNR | Cl |
| 4 | NRCNHNR | Br |
| 4 | NRCNHNR | I |
| 4 | NRCNHNR | F |
| 4 | NRCNHNR | CN |
| 4 | NRCNHNR | N₃ |
| 4 | NRCNHNR | CONH₂ |
| 4 | NRCNHNR | CH═CH₂ |
| 4 | NRCNHNR | C≡CH |
| 4 | NRCNHNR | NH₂ |
| 4 | NRCNHNR | NHR |
| 4 | NRCNHNR | COH |
| 4 | NRCNHNR | COR |
| 4 | CH₂═CH₂ | OH |
| 4 | CH₂═CH₂ | SH |
| 4 | CH₂═CH₂ | COOH |
| 4 | CH₂═CH₂ | SO₂H |
| 4 | CH₂═CH₂ | Cl |
| 4 | CH₂═CH₂ | Br |
| 4 | CH₂═CH₂ | I |
| 4 | CH₂═CH₂ | F |
| 4 | CH₂═CH₂ | CN |
| 4 | CH₂═CH₂ | N₃ |
| 4 | CH₂═CH₂ | CONH₂ |
| 4 | CH₂═CH₂ | CH═CH₂ |
| 4 | CH₂═CH₂ | C≡CH |
| 4 | CH₂═CH₂ | NH₂ |
| 4 | CH₂═CH₂ | NHR |
| 4 | CH₂═CH₂ | COH |
| 4 | CH₂═CH₂ | COR |
| 5 | NR | OH |
| 5 | NR | SH |
| 5 | NR | COOH |
| 5 | NR | SO₂H |
| 5 | NR | Cl |
| 5 | NR | Br |
| 5 | NR | I |
| 5 | NR | F |
| 5 | NR | CN |
| 5 | NR | N₃ |
| 5 | NR | CONH₂ |
| 5 | NR | CH═CH₂ |
| 5 | NR | C≡CH |
| 5 | NR | NH₂ |
| 5 | NR | NHR |
| 5 | NR | COH |
| 5 | NR | COR |
| 5 | CONR | OH |
| 5 | CONR | SH |
| 5 | CONR | COOH |
| 5 | CONR | SO₂H |
| 5 | CONR | Cl |
| 5 | CONR | Br |
| 5 | CONR | I |
| 5 | CONR | F |
| 5 | CONR | CN |
| 5 | CONR | N₃ |
| 5 | CONR | CONH₂ |
| 5 | CONR | CH═CH₂ |
| 5 | CONR | C≡CH |
| 5 | CONR | NH₂ |
| 5 | CONR | NHR |
| 5 | CONR | COH |
| 5 | CONR | COR |
| 5 | NRCONR | OH |
| 5 | NRCONR | SH |
| 5 | NRCONR | COOH |
| 5 | NRCONR | SO₂H |
| 5 | NRCONR | Cl |
| 5 | NRCONR | Br |
| 5 | NRCONR | I |
| 5 | NRCONR | F |
| 5 | NRCONR | CN |
| 5 | NRCONR | N₃ |
| 5 | NRCONR | CONH₂ |
| 5 | NRCONR | CH═CH₂ |
| 5 | NRCONR | C≡CH |
| 5 | NRCONR | NH₂ |
| 5 | NRCONR | NHR |
| 5 | NRCONR | COH |
| 5 | NRCONR | COR |
| 5 | NHCOO | OH |
| 5 | NHCOO | SH |
| 5 | NHCOO | COOH |
| 5 | NHCOO | SO₂H |
| 5 | NHCOO | Cl |
| 5 | NHCOO | Br |
| 5 | NHCOO | I |
| 5 | NHCOO | F |
| 5 | NHCOO | CN |
| 5 | NHCOO | N₃ |
| 5 | NHCOO | CONH₂ |
| 5 | NHCOO | CH═CH₂ |
| 5 | NHCOO | C≡CH |
| 5 | NHCOO | NH₂ |
| 5 | NHCOO | NHR |
| 5 | NHCOO | COH |
| 5 | NHCOO | COR |
| 5 | CH₂═CH₂ | Br |
| 5 | CH₂═CH₂ | I |
| 5 | CH₂═CH₂ | F |
| 5 | CH₂═CH₂ | CN |
| 0 | NR | OH |
| 0 | NR | SH |
| 0 | NR | COOH |
| 0 | NR | SO₂H |
| 0 | NR | Cl |
| 0 | NR | Br |
| 0 | NR | I |
| 0 | NR | F |
| 0 | NR | CN |
| 0 | NR | N₃ |
| 0 | NR | CONH₂ |
| 0 | NR | CH═CH₂ |
| 0 | NR | C≡CH |
| 0 | NR | NH₂ |
| 0 | NR | NHR |
| 0 | NR | COH |
| 0 | NR | COR |
| 0 | CONR | OH |
| 0 | CONR | SH |
| 0 | CONR | COOH |
| 0 | CONR | SO₂H |
| 0 | CONR | Cl |
| 0 | CONR | Br |
| 0 | CONR | I |
| 0 | CONR | F |
| 0 | CONR | CN |
| 0 | CONR | N₃ |
| 0 | CONR | CONH₂ |
| 0 | CONR | CH═CH₂ |
| 0 | CONR | C≡CH |
| 0 | CONR | NH₂ |
| 0 | CONR | NHR |
| 0 | CONR | COH |
| 0 | CONR | COR |
| 0 | NRCONR | OH |
| 0 | NRCONR | SH |
| 0 | NRCONR | COOH |
| 0 | NRCONR | SO₂H |
| 0 | NRCONR | Cl |
| 0 | NRCONR | Br |
| 0 | NRCONR | I |
| 0 | NRCONR | F |
| 0 | NRCONR | CN |
| 0 | NRCONR | N₃ |
| 0 | NRCONR | CONH₂ |
| 0 | NRCONR | CH═CH₂ |
| 0 | NRCONR | C≡CH |
| 0 | NRCONR | NH₂ |
| 0 | NRCONR | NHR |
| 0 | NRCONR | COH |
| 0 | NRCONR | COR |
| 0 | NRCOO | OH |
| 0 | NRCOO | SH |
| 0 | NRCOO | COOH |
| 0 | NRCOO | SO₂H |
| 0 | NRCOO | Cl |
| 0 | NRCOO | Br |
| 0 | NRCOO | I |
| 0 | NRCOO | F |
| 0 | NRCOO | CN |
| 0 | NRCOO | N₃ |
| 0 | NRCOO | CONH₂ |
| 0 | NRCOO | CH═CH₂ |
| 0 | NRCOO | C≡CH |
| 0 | NRCOO | NH₂ |
| 0 | NRCOO | NHR |
| 0 | NRCOO | COH |
| 0 | NRCOO | COR |
| 1 | S | OH |
| 1 | S | SH |
| 1 | S | COOH |
| 1 | S | SO₂H |
| 1 | S | Cl |
| 1 | S | Br |
| 1 | S | I |
| 1 | S | F |
| 1 | S | CN |
| 1 | S | N₃ |
| 1 | S | CONH₂ |
| 1 | S | CH═CH₂ |
| 1 | S | C≡CH |
| 1 | S | NH₂ |
| 1 | S | NHR |
| 1 | S | COH |
| 1 | S | COR |
| 1 | COR₁R₂ | OH |
| 1 | COR₁R₂ | SH |
| 1 | COR₁R₂ | COOH |
| 1 | COR₁R₂ | SO₂H |
| 1 | COR₁R₂ | Cl |
| 1 | COR₁R₂ | Br |
| 1 | COR₁R₂ | I |
| 1 | COR₁R₂ | F |
| 1 | COR₁R₂ | CN |
| 1 | COR₁R₂ | N₃ |
| 1 | COR₁R₂ | CONH₂ |
| 1 | COR₁R₂ | CH═CH₂ |
| 1 | COR₁R₂ | C≡CH |
| 1 | COR₁R₂ | NH₂ |
| 1 | COR₁R₂ | NHR |
| 1 | COR₁R₂ | COH |
| 1 | COR₁R₂ | COR |
| 1 | SO₂NR | OH |
| 1 | SO₂NR | SH |
| 1 | SO₂NR | COOH |
| 1 | SO₂NR | SO₂H |
| 1 | SO₂NR | Cl |
| 1 | SO₂NR | Br |
| 1 | SO₂NR | I |
| 1 | SO₂NR | F |
| 1 | SO₂NR | CN |
| 1 | SO₂NR | N₃ |
| 1 | SO₂NR | CONH₂ |
| 1 | SO₂NR | CH═CH₂ |
| 1 | SO₂NR | C≡CH |
| 1 | SO₂NR | NH₂ |
| 1 | SO₂NR | NHR |
| 1 | SO₂NR | COH |
| 1 | SO₂NR | COR |
| 1 | NRCNHNR | OH |
| 1 | NRCNHNR | SH |
| 1 | NRCNHNR | COOH |
| 1 | NRCNHNR | SO₂H |
| 1 | NRCNHNR | Cl |
| 1 | NRCNHNR | Br |
| 1 | NRCNHNR | I |
| 1 | NRCNHNR | F |
| 1 | NRCNHNR | CN |
| 1 | NRCNHNR | N₃ |
| 1 | NRCNHNR | CONH₂ |
| 1 | NRCNHNR | CH═CH₂ |
| 1 | NRCNHNR | C≡CH |
| 1 | NRCNHNR | NH₂ |
| 1 | NRCNHNR | NHR |
| 1 | NRCNHNR | COH |
| 1 | NRCNHNR | COR |
| 1 | CH═CH₂ | OH |
| 1 | CH═CH₂ | SH |
| 1 | CH═CH₂ | COOH |
| 1 | CH═CH₂ | SO₂H |
| 1 | CH═CH₂ | Cl |
| 1 | CH═CH₂ | Br |
| 1 | CH═CH₂ | I |
| 1 | CH═CH₂ | F |
| 1 | CH═CH₂ | CN |
| 1 | CH═CH₂ | N₃ |
| 1 | CH═CH₂ | CONH₂ |
| 1 | CH═CH₂ | CH═CH₂ |
| 1 | CH═CH₂ | CCH |
| 1 | CH═CH₂ | NH₂ |
| 1 | CH═CH₂ | NHR |
| 1 | CH═CH₂ | COH |
| 1 | CH═CH₂ | COR |
| 2 | NR | OH |
| 2 | NR | SH |
| 2 | NR | COOH |
| 2 | NR | SO₂H |
| 2 | NR | Cl |
| 2 | NR | Br |
| 2 | NR | I |
| 2 | NR | F |
| 2 | NR | CN |
| 2 | NR | N₃ |
| 2 | NR | CONH₂ |
| 2 | NR | CH═CH₂ |
| 2 | NR | C≡CH |
| 2 | NR | NH₂ |
| 2 | NR | NHR |
| 2 | NR | COH |
| 2 | NR | COR |
| 2 | CONR | OH |
| 2 | CONR | SH |
| 2 | CONR | COOH |
| 2 | CONR | SO₂H |
| 2 | CONR | Cl |
| 2 | CONR | Br |
| 2 | CONR | I |
| 2 | CONR | F |
| 2 | CONR | CN |
| 2 | CONR | N₃ |
| 2 | CONR | CONH₂ |
| 2 | CONR | CH═CH₂ |
| 2 | CONR | C≡CH |
| 2 | CONR | NH₂ |
| 2 | CONR | NHR |
| 2 | CONR | COH |
| 2 | CONR | COR |
| 2 | NRCONR | OH |
| 2 | NRCONR | SH |
| 2 | NRCONR | COOH |
| 2 | NRCONR | SO₂H |
| 2 | NRCONR | Cl |
| 2 | NRCONR | Br |
| 2 | NRCONR | I |
| 2 | NRCONR | F |
| 2 | NRCONR | CN |
| 2 | NRCONR | N₃ |
| 2 | NRCONR | CONH₂ |
| 2 | NRCONR | CH═CH₂ |
| 2 | NRCONR | CCH |
| 2 | NRCONR | NH₂ |
| 2 | NRCONR | NHR |
| 2 | NRCONR | COH |
| 2 | NRCONR | COR |
| 2 | NRCOO | OH |
| 2 | NRCOO | SH |
| 2 | NRCOO | COOH |
| 2 | NRCOO | SO₂H |
| 2 | NRCOO | Cl |
| 2 | NRCOO | Br |
| 2 | NRCOO | I |
| 2 | NRCOO | F |
| 2 | NRCOO | CN |
| 2 | NRCOO | N₃ |
| 2 | NRCOO | CONH₂ |
| 2 | NRCOO | CH═CH₂ |
| 2 | NRCOO | C≡CH |
| 2 | NRCOO | NH₂ |
| 2 | NRCOO | NHR |
| 2 | NRCOO | COH |
| 2 | NRCOO | COR |
| 3 | S | OH |
| 3 | S | SH |
| 3 | S | COOH |
| 3 | S | SO₂H |
| 3 | S | Cl |
| 3 | S | Br |
| 3 | S | I |
| 3 | S | F |
| 3 | S | CN |
| 3 | S | N₃ |
| 3 | S | CONH₂ |
| 3 | S | CH═CH₂ |
| 3 | S | C≡CH |
| 3 | S | NH₂ |
| 3 | S | NHR |
| 3 | S | COH |
| 3 | S | COR |
| 3 | COR₁R₂ | OH |
| 3 | COR₁R₂ | SH |
| 3 | COR₁R₂ | COOH |
| 3 | COR₁R₂ | SO₂H |
| 3 | COR₁R₂ | Cl |
| 3 | COR₁R₂ | Br |
| 3 | COR₁R₂ | I |
| 3 | COR₁R₂ | F |
| 3 | COR₁R₂ | CN |
| 3 | COR₁R₂ | N₃ |
| 3 | COR₁R₂ | CONH₂ |
| 3 | COR₁R₂ | CH═CH₂ |
| 3 | COR₁R₂ | C≡CH |
| 3 | COR₁R₂ | NH₂ |
| 3 | COR₁R₂ | NHR |
| 3 | COR₁R₂ | COH |
| 3 | COR₁R₂ | COR |
| 3 | SO₂NR | OH |
| 3 | SO₂NR | SH |
| 3 | SO₂NR | COOH |
| 3 | SO₂NR | SO₂H |
| 3 | SO₂NR | Cl |
| 3 | SO₂NR | Br |
| 3 | SO₂NR | I |
| 3 | SO₂NR | F |
| 3 | SO₂NR | CN |
| 3 | SO₂NR | N₃ |
| 3 | SO₂NR | CONH₂ |
| 3 | SO₂NR | CH═CH₂ |
| 3 | SO₂NR | C≡CH |
| 3 | SO₂NR | NH₂ |
| 3 | SO₂NR | NHR |
| 3 | SO₂NR | COH |
| 3 | SO₂NR | COR |
| 3 | NRCNHNR | OH |
| 3 | NRCNHNR | SH |
| 3 | NRCNHNR | COOH |
| 3 | NRCNHNR | SO₂H |
| 3 | NRCNHNR | Cl |
| 3 | NRCNHNR | Br |
| 3 | NRCNHNR | I |
| 3 | NRCNHNR | F |
| 3 | NRCNHNR | CN |
| 3 | NRCNHNR | N₃ |
| 3 | NRCNHNR | CONH₂ |
| 3 | NRCNHNR | CH═CH₂ |
| 3 | NRCNHNR | C≡CH |
| 3 | NRCNHNR | NH₂ |
| 3 | NRCNHNR | NHR |
| 3 | NRCNHNR | COH |
| 3 | NRCNHNR | COR |
| 3 | CH₂═CH₂ | OH |
| 3 | CH₂═CH₂ | SH |
| 3 | CH₂═CH₂ | COOH |
| 3 | CH₂═CH₂ | SO₂H |
| 3 | CH₂═CH₂ | Cl |
| 3 | CH₂═CH₂ | Br |
| 3 | CH₂═CH₂ | I |
| 3 | CH₂═CH₂ | F |
| 3 | CH₂═CH₂ | CN |
| 3 | CH₂═CH₂ | N₃ |
| 3 | CH₂═CH₂ | CONH₂ |
| 3 | CH₂═CH₂ | CH═CH₂ |
| 3 | CH₂═CH₂ | C≡CH |
| 3 | CH₂═CH₂ | NH₂ |
| 3 | CH₂═CH₂ | NHR |
| 3 | CH₂═CH₂ | COH |
| 3 | CH₂═CH₂ | COR |
| 4 | NR | OH |
| 4 | NR | SH |
| 4 | NR | COOH |
| 4 | NR | SO₂H |
| 4 | NR | Cl |
| 4 | NR | Br |
| 4 | NR | I |
| 4 | NR | F |
| 4 | NR | CN |
| 4 | NR | N₃ |
| 4 | NR | CONH₂ |
| 4 | NR | CH═CH₂ |
| 4 | NR | C≡CH |
| 4 | NR | NH₂ |
| 4 | NR | NHR |
| 4 | NR | COH |
| 4 | NR | COR |
| 4 | CONR | OH |
| 4 | CONR | SH |
| 4 | CONR | COOH |
| 4 | CONR | SO₂H |
| 4 | CONR | Cl |
| 4 | CONR | Br |
| 4 | CONR | I |
| 4 | CONR | F |
| 4 | CONR | CN |
| 4 | CONR | N₃ |
| 4 | CONR | CONH₂ |
| 4 | CONR | CH═CH₂ |
| 4 | CONR | C≡CH |
| 4 | CONR | NH₂ |
| 4 | CONR | NHR |
| 4 | CONR | COH |
| 4 | CONR | COR |
| 4 | NRCONR | OH |
| 4 | NRCONR | SH |
| 4 | NRCONR | COOH |
| 4 | NRCONR | SO₂H |
| 4 | NRCONR | Cl |
| 4 | NRCONR | Br |
| 4 | NRCONR | I |
| 4 | NRCONR | F |
| 4 | NRCONR | CN |
| 4 | NRCONR | N₃ |
| 4 | NRCONR | CONH₂ |
| 4 | NRCONR | CH═CH₂ |
| 4 | NRCONR | C≡CH |
| 4 | NRCONR | NH₂ |
| 4 | NRCONR | NHR |
| 4 | NRCONR | COH |
| 4 | NRCONR | COR |
| 4 | NRCOO | OH |
| 4 | NRCOO | SH |
| 4 | NRCOO | COOH |
| 4 | NRCOO | SO₂H |
| 4 | NRCOO | Cl |
| 4 | NRCOO | Br |
| 4 | NRCOO | I |
| 4 | NRCOO | F |
| 4 | NRCOO | CN |
| 4 | NRCOO | N₃ |
| 4 | NRCOO | CONH₂ |
| 4 | NRCOO | CH═CH₂ |
| 4 | NRCOO | C≡CH |
| 4 | NRCOO | NH₂ |
| 4 | NRCOO | NHR |
| 4 | NRCOO | COH |
| 4 | NRCOO | COR |
| 5 | S | OH |
| 5 | S | SH |
| 5 | S | COOH |
| 5 | S | SO₂H |
| 5 | S | Cl |
| 5 | S | Br |
| 5 | S | I |
| 5 | S | F |
| 5 | S | CN |
| 5 | S | N₃ |
| 5 | S | CONH₂ |
| 5 | S | CH═CH₂ |
| 5 | S | C≡CH |
| 5 | S | NH₂ |
| 5 | S | NHR |
| 5 | S | COH |
| 5 | S | COR |
| 5 | COR₁R₂ | OH |
| 5 | COR₁R₂ | SH |
| 5 | COR₁R₂ | COOH |
| 5 | COR₁R₂ | SO₂H |
| 5 | COR₁R₂ | Cl |
| 5 | COR₁R₂ | Br |
| 5 | COR₁R₂ | I |
| 5 | COR₁R₂ | F |
| 5 | COR₁R₂ | CN |
| 5 | COR₁R₂ | N₃ |
| 5 | COR₁R₂ | CONH₂ |
| 5 | COR₁R₂ | CH═CH₂ |
| 5 | COR₁R₂ | C≡CH |
| 5 | COR₁R₂ | NH₂ |
| 5 | COR₁R₂ | NHR |
| 5 | COR₁R₂ | COH |
| 5 | COR₁R₂ | COR |
| 5 | SO₂NR | OH |
| 5 | SO₂NR | SH |
| 5 | SO₂NR | COOH |
| 5 | SO₂NR | SO₂H |
| 5 | SO₂NR | Cl |
| 5 | SO₂NR | Br |
| 5 | SO₂NR | I |
| 5 | SO₂NR | F |
| 5 | SO₂NR | CN |
| 5 | SO₂NR | N₃ |
| 5 | SO₂NR | CONH₂ |
| 5 | SO₂NR | CH═CH₂ |
| 5 | SO₂NR | C≡CH |
| 5 | SO₂NR | NH₂ |
| 5 | SO₂NR | NHR |
| 5 | SO₂NR | COH |
| 5 | SO₂NR | COR |
| 5 | NRCNHNR | OH |
| 5 | NRCNHNR | SH |
| 5 | NRCNHNR | COOH |
| 5 | NRCNHNR | SO₂H |
| 5 | NRCNHNR | Cl |
| 5 | NRCNHNR | Br |
| 5 | NRCNHNR | I |
| 5 | NRCNHNR | F |
| 5 | NRCNHNR | CN |
| 5 | NRCNHNR | N₃ |
| 5 | NRCNHNR | CONH₂ |
| 5 | NRCNHNR | CH═CH₂ |
| 5 | NRCNHNR | C≡CH |
| 5 | NRCNHNR | NH₂ |
| 5 | NRCNHNR | NHR |
| 5 | NRCNHNR | COH |
| 5 | NRCNHNR | COR |
| 5 | C≡C | OH |
| 5 | C≡C | SH |
| 5 | C≡C | COOH |
| 5 | C≡C | SO₂H |
| 5 | C≡C | Cl |
| 5 | C≡C | Br |
| 5 | C≡C | I |
| 5 | C≡C | F |
| 5 | C≡C | CN |
| 5 | C≡C | N₃ |
| 5 | C≡C | CONH₂ |
| 5 | C≡C | CH═CH₂ |
| 5 | C≡C | C≡CH |
| 5 | C≡C | NH₂ |
| 5 | C≡C | NHR |
| 5 | C≡C | COH |
| 5 | C≡C | COR |
| 5 | CH₂═CH₂ | NH₂ |
| 5 | CH₂═CH₂ | NHR |
| 5 | CH₂═CH₂ | COH |
| 5 | CH₂═CH₂ | COR |
| N | E | Y |
| 0 | NH | OH |
| 0 | NH | SH |
| 0 | NH | COOH |
| 0 | NH | SO₂H |
| 0 | NH | Cl |
| 0 | NH | Br |
| 0 | NH | I |
| 0 | NH | F |
| 0 | NH | CN |
| 0 | NH | N₃ |
| 0 | NH | CONH₂ |
| 0 | NH | CH═CH₂ |
| 0 | NH | C≡CH |
| 0 | NH | NH₂ |
| 0 | NH | NHR |
| 0 | NH | COH |
| 0 | NH | COR |
| 0 | CONH | OH |
| 0 | CONH | SH |
| 0 | CONH | COOH |
| 0 | CONH | SO₂H |
| 0 | CONH | Cl |
| 0 | CONH | Br |
| 0 | CONH | I |
| 0 | CONH | F |
| 0 | CONH | CN |
| 0 | CONH | N₃ |
| 0 | CONH | CONH₂ |
| 0 | CONH | CH═CH₂ |
| 0 | CONH | C≡CH |
| 0 | CONH | NH₂ |
| 0 | CONH | NHR |
| 0 | CONH | COH |
| 0 | CONH | COR |
| 0 | NHCONH | OH |
| 0 | NHCONH | SH |
| 0 | NHCONH | COOH |
| 0 | NHCONH | SO₂H |
| 0 | NHCONH | Cl |
| 0 | NHCONH | Br |
| 0 | NHCONH | I |
| 0 | NHCONH | F |
| 0 | NHCONH | CN |
| 0 | NHCONH | N₃ |
| 0 | NHCONH | CONH₂ |
| 0 | NHCONH | CH═CH₂ |
| 0 | NHCONH | C≡CH |
| 0 | NHCONH | NH₂ |
| 0 | NHCONH | NHR |
| 0 | NHCONH | COH |
| 0 | NHCONH | COR |
| 0 | NHCOO | OH |
| 0 | NHCOO | SH |
| 0 | NHCOO | COOH |
| 0 | NHCOO | SO₂H |
| 0 | NHCOO | Cl |
| 0 | NHCOO | Br |
| 0 | NHCOO | I |
| 0 | NHCOO | F |
| 0 | NHCOO | CN |
| 0 | NHCOO | N₃ |
| 0 | NHCOO | CONH₂ |
| 0 | NHCOO | CH═CH₂ |
| 0 | NHCOO | C≡CH |
| 0 | NHCOO | NH₂ |
| 0 | NHCOO | NHR |
| 0 | NHCOO | COH |
| 0 | NHCOO | COR |
| 1 | O | OH |
| 1 | O | SH |
| 1 | O | COOH |
| 1 | O | SO₂H |
| 1 | O | Cl |
| 1 | O | Br |
| 1 | O | I |
| 1 | O | F |
| 1 | O | CN |
| 1 | O | N₃ |
| 1 | O | CONH₂ |
| 1 | O | CH═CH₂ |
| 1 | O | C≡CH |
| 1 | O | NH₂ |
| 1 | O | NHR |
| 1 | O | COH |
| 1 | O | COR |
| 1 | CH₂ | OH |
| 1 | CH₂ | SH |
| 1 | CH₂ | COOH |
| 1 | CH₂ | SO₂H |
| 1 | CH₂ | Cl |
| 1 | CH₂ | Br |
| 1 | CH₂ | I |
| 1 | CH₂ | F |
| 1 | CH₂ | CN |
| 1 | CH₂ | N₃ |
| 1 | CH₂ | CONH₂ |
| 1 | CH₂ | CH═CH₂ |
| 1 | CH₂ | C≡CH |
| 1 | CH₂ | NH₂ |
| 1 | CH₂ | NHR |
| 1 | CH₂ | COH |
| 1 | CH₂ | COR |
| 1 | SO₂NH | OH |
| 1 | SO₂NH | SH |
| 1 | SO₂NH | COOH |
| 1 | SO₂NH | SO₂H |
| 1 | SO₂NH | Cl |
| 1 | SO₂NH | Br |
| 1 | SO₂NH | I |
| 1 | SO₂NH | F |
| 1 | SO₂NH | CN |
| 1 | SO₂NH | N₃ |
| 1 | SO₂NH | CONH₂ |
| 1 | SO₂NH | CH═CH₂ |
| 1 | SO₂NH | C≡CH |
| 1 | SO₂NH | NH₂ |
| 1 | SO₂NH | NHR |
| 1 | SO₂NH | COH |
| 1 | SO₂NH | COR |
| 1 | NHCNHNH | OH |
| 1 | NHCNHNH | SH |
| 1 | NHCNHNH | COOH |
| 1 | NHCNHNH | SO₂H |
| 1 | NHCNHNH | Cl |
| 1 | NHCNHNH | Br |
| 1 | NHCNHNH | I |
| 1 | NHCNHNH | F |
| 1 | NHCNHNH | CN |
| 1 | NHCNHNH | N₃ |
| 1 | NHCNHNH | CONH₂ |
| 1 | NHCNHNH | CH═CH₂ |
| 1 | NHCNHNH | C≡CH |
| 1 | NHCNHNH | NH₂ |
| 1 | NHCNHNH | NHR |
| 1 | NHCNHNH | COH |
| 1 | NHCNHNH | COR |
| 1 | C≡C | OH |
| 1 | C≡C | SH |
| 1 | C≡C | COOH |
| 1 | C≡C | SO₂H |
| 1 | C≡C | Cl |
| 1 | C≡C | Br |
| 1 | C≡C | I |
| 1 | C≡C | F |
| 1 | C≡C | CN |
| 1 | C≡C | N₃ |
| 1 | C≡C | CONH₂ |
| 1 | C≡C | CH═CH₂ |
| 1 | C≡C | CCH |
| 1 | C≡C | NH₂ |
| 1 | C≡C | NHR |
| 1 | C≡C | COH |
| 1 | C≡C | COR |
| 2 | NH | OH |
| 2 | NH | SH |
| 2 | NH | COOH |
| 2 | NH | SO₂H |
| 2 | NH | Cl |
| 2 | NH | Br |
| 2 | NH | I |
| 2 | NH | F |
| 2 | NH | CN |
| 2 | NH | N₃ |
| 2 | NH | CONH₂ |
| 2 | NH | CH═CH₂ |
| 2 | NH | C≡CH |
| 2 | NH | NH₂ |
| 2 | NH | NHR |
| 2 | NH | COH |
| 2 | NH | COR |
| 2 | CONH | OH |
| 2 | CONH | SH |
| 2 | CONH | COOH |
| 2 | CONH | SO₂H |
| 2 | CONH | Cl |
| 2 | CONH | Br |
| 2 | CONH | I |
| 2 | CONH | F |
| 2 | CONH | CN |
| 2 | CONH | N₃ |
| 2 | CONH | CONH₂ |
| 2 | CONH | CH═CH₂ |
| 2 | CONH | C≡CH |
| 2 | CONH | NH₂ |
| 2 | CONH | NHR |
| 2 | CONH | COH |
| 2 | CONH | COR |
| 2 | NHCONH | OH |
| 2 | NHCONH | SH |
| 2 | NHCONH | COOH |
| 2 | NHCONH | SO₂H |
| 2 | NHCONH | Cl |
| 2 | NHCONH | Br |
| 2 | NHCONH | I |
| 2 | NHCONH | F |
| 2 | NHCONH | CN |
| 2 | NHCONH | N₃ |
| 2 | NHCONH | CONH₂ |
| 2 | NHCONH | CH═CH₂ |
| 2 | NHCONH | CCH |
| 2 | NHCONH | NH₂ |
| 2 | NHCONH | NHR |
| 2 | NHCONH | COH |
| 2 | NHCONH | COR |
| 2 | NHCOO | OH |
| 2 | NHCOO | SH |
| 2 | NHCOO | COOH |
| 2 | NHCOO | SO₂H |
| 2 | NHCOO | Cl |
| 2 | NHCOO | Br |
| 2 | NHCOO | I |
| 2 | NHCOO | F |
| 2 | NHCOO | CN |
| 2 | NHCOO | N₃ |
| 2 | NHCOO | CONH₂ |
| 2 | NHCOO | CH═CH₂ |
| 2 | NHCOO | C≡CH |
| 2 | NHCOO | NH₂ |
| 2 | NHCOO | NHR |
| 2 | NHCOO | COH |
| 2 | NHCOO | COR |
| 3 | O | OH |
| 3 | O | SH |
| 3 | O | COOH |
| 3 | O | SO₂H |
| 3 | O | Cl |
| 3 | O | Br |
| 3 | O | I |
| 3 | O | F |
| 3 | O | CN |
| 3 | O | N₃ |
| 3 | O | CONH₂ |
| 3 | O | CH═CH₂ |
| 3 | O | C≡CH |
| 3 | O | NH₂ |
| 3 | O | NHR |
| 3 | O | COH |
| 3 | O | COR |
| 3 | CH₂ | OH |
| 3 | CH₂ | SH |
| 3 | CH₂ | COOH |
| 3 | CH₂ | SO₂H |
| 3 | CH₂ | Cl |
| 3 | CH₂ | Br |
| 3 | CH₂ | I |
| 3 | CH₂ | F |
| 3 | CH₂ | CN |
| 3 | CH₂ | N₃ |
| 3 | CH₂ | CONH₂ |
| 3 | CH₂ | CH═CH₂ |
| 3 | CH₂ | C≡CH |
| 3 | CH₂ | NH₂ |
| 3 | CH₂ | NHR |
| 3 | CH₂ | COH |
| 3 | CH₂ | COR |
| 3 | SO₂NH | OH |
| 3 | SO₂NH | SH |
| 3 | SO₂NH | COOH |
| 3 | SO₂NH | SO₂H |
| 3 | SO₂NH | Cl |
| 3 | SO₂NH | Br |
| 3 | SO₂NH | I |
| 3 | SO₂NH | F |
| 3 | SO₂NH | CN |
| 3 | SO₂NH | N₃ |
| 3 | SO₂NH | CONH₂ |
| 3 | SO₂NH | CH═CH₂ |
| 3 | SO₂NH | C≡CH |
| 3 | SO₂NH | NH₂ |
| 3 | SO₂NH | NHR |
| 23 | SO₂NH | COH |
| 3 | SO₂NH | COR |
| 3 | NHCNHNH | OH |
| 3 | NHCNHNH | SH |
| 3 | NHCNHNH | COOH |
| 3 | NHCNHNH | SO₂H |
| 3 | NHCNHNH | Cl |
| 3 | NHCNHNH | Br |
| 3 | NHCNHNH | I |
| 3 | NHCNHNH | F |
| 3 | NHCNHNH | CN |
| 3 | NHCNHNH | N₃ |
| 3 | NHCNHNH | CONH₂ |
| 3 | NHCNHNH | CH═CH₂ |
| 3 | NHCNHNH | C≡CH |
| 3 | NHCNHNH | NH₂ |
| 3 | NHCNHNH | NHR |
| 3 | NHCNHNH | COH |
| 3 | NHCNHNH | COR |
| 3 | C≡C | OH |
| 3 | C≡C | SH |
| 3 | C≡C | COOH |
| 3 | C≡C | SO₂H |
| 3 | C≡C | Cl |
| 3 | C≡C | Br |
| 3 | C≡C | I |
| 3 | C≡C | F |
| 3 | C≡C | CN |
| 3 | C≡C | N₃ |
| 3 | C≡C | CONH₂ |
| 3 | C≡C | CH═CH₂ |
| 3 | C≡C | C≡CH |
| 3 | C≡C | NH₂ |
| 3 | C≡C | NHR |
| 3 | C≡C | COH |
| 3 | C≡C | COR |
| 4 | NH | OH |
| 4 | NH | SH |
| 4 | NH | COOH |
| 4 | NH | SO₂H |
| 4 | NH | Cl |
| 4 | NH | Br |
| 4 | NH | I |
| 4 | NH | F |
| 4 | NH | CN |
| 4 | NH | N₃ |
| 4 | NH | CONH₂ |
| 4 | NH | CH═CH₂ |
| 4 | NH | C≡CH |
| 4 | NH | NH₂ |
| 4 | NH | NHR |
| 4 | NH | COH |
| 4 | NH | COR |
| 4 | CONH | OH |
| 4 | CONH | SH |
| 4 | CONH | COOH |
| 4 | CONH | SO₂H |
| 4 | CONH | Cl |
| 4 | CONH | Br |
| 4 | CONH | I |
| 4 | CONH | F |
| 4 | CONH | CN |
| 4 | CONH | N₃ |
| 4 | CONH | CONH₂ |
| 4 | CONH | CH═CH₂ |
| 4 | CONH | C≡CH |
| 4 | CONH | NH₂ |
| 4 | CONH | NHR |
| 4 | CONH | COH |
| 4 | CONH | COR |
| 4 | NHCONH | OH |
| 4 | NHCONH | SH |
| 4 | NHCONH | COOH |
| 4 | NHCONH | SO₂H |
| 4 | NHCONH | Cl |
| 4 | NHCONH | Br |
| 4 | NHCONH | I |
| 4 | NHCONH | F |
| 4 | NHCONH | CN |
| 4 | NHCONH | N₃ |
| 4 | NHCONH | CONH₂ |
| 4 | NHCONH | CH═CH₂ |
| 4 | NHCONH | C≡CH |
| 4 | NHCONH | NH₂ |
| 4 | NHCONH | NHR |
| 4 | NHCONH | COH |
| 4 | NHCONH | COR |
| 4 | NHCOO | OH |
| 4 | NHCOO | SH |
| 4 | NHCOO | COOH |
| 4 | NHCOO | SO₂H |
| 4 | NHCOO | Cl |
| 4 | NHCOO | Br |
| 4 | NHCOO | I |
| 4 | NHCOO | F |
| 4 | NHCOO | CN |
| 4 | NHCOO | N₃ |
| 4 | NHCOO | CONH₂ |
| 4 | NHCOO | CH═CH₂ |
| 4 | NHCOO | C≡CH |
| 4 | NHCOO | NH₂ |
| 4 | NHCOO | NHR |
| 4 | NHCOO | COH |
| 4 | NHCOO | COR |
| 5 | O | OH |
| 5 | O | SH |
| 5 | O | COOH |
| 5 | O | SO₂H |
| 5 | O | Cl |
| 5 | O | Br |
| 5 | O | I |
| 5 | O | F |
| 5 | O | CN |
| 5 | O | N₃ |
| 5 | O | CONH₂ |
| 5 | O | CH═CH₂ |
| 5 | O | C≡CH |
| 5 | O | NH₂ |
| 5 | O | NHR |
| 5 | O | COH |
| 5 | O | COR |
| 5 | CH₂ | OH |
| 5 | CH₂ | SH |
| 5 | CH₂ | COOH |
| 5 | CH₂ | SO₂H |
| 5 | CH₂ | Cl |
| 5 | CH₂ | Br |
| 5 | CH₂ | I |
| 5 | CH₂ | F |
| 5 | CH₂ | CN |
| 5 | CH₂ | N₃ |
| 5 | CH₂ | CONH₂ |
| 5 | CH₂ | CH═CH₂ |
| 5 | CH₂ | C≡CH |
| 5 | CH₂ | NH₂ |
| 5 | CH₂ | NHR |
| 5 | CH₂ | COH |
| 5 | CH₂ | COR |
| 5 | SO₂NH | OH |
| 5 | SO₂NH | SH |
| 5 | SO₂NH | COOH |
| 5 | SO₂NH | SO₂H |
| 5 | SO₂NH | Cl |
| 5 | SO₂NH | Br |
| 5 | SO₂NH | I |
| 5 | SO₂NH | F |
| 5 | SO₂NH | CN |
| 5 | SO₂NH | N₃ |
| 5 | SO₂NH | CONH₂ |
| 5 | SO₂NH | CH═CH₂ |
| 5 | SO₂NH | C≡CH |
| 5 | SO₂NH | NH₂ |
| 5 | SO₂NH | NHR |
| 5 | SO₂NH | COH |
| 5 | SO₂NH | COR |
| 5 | NHCNHNH | OH |
| 5 | NHCNHNH | SH |
| 5 | NHCNHNH | COOH |
| 5 | NHCNHNH | SO₂H |
| 5 | NHCNHNH | Cl |
| 5 | NHCNHNH | Br |
| 5 | NHCNHNH | I |
| 5 | NHCNHNH | F |
| 5 | NHCNHNH | CN |
| 5 | NHCNHNH | N₃ |
| 5 | NHCNHNH | CONH₂ |
| 5 | NHCNHNH | CH═CH₂ |
| 5 | NHCNHNH | C≡CH |
| 5 | NHCNHNH | NH₂ |
| 5 | NHCNHNH | NHR |
| 5 | NHCNHNH | COH |
| 5 | NHCNHNH | COR |
| 5 | NRCOO | OH |
| 5 | NRCOO | SH |
| 5 | NRCOO | COOH |
| 5 | NRCOO | SO₂H |
| 5 | NRCOO | Cl |
| 5 | NRCOO | Br |
| 5 | NRCOO | I |
| 5 | NRCOO | F |
| 5 | NRCOO | CN |
| 5 | NRCOO | N₃ |
| 5 | NRCOO | CONH₂ |
| 5 | NRCOO | CH═CH₂ |
| 5 | NRCOO | C≡CH |
| 5 | NRCOO | NH₂ |
| 5 | NRCOO | NHR |
| 5 | NRCOO | COH |
| 5 | NRCOO | COR |
| 5 | CH₂═CH₂ | N₃ |
| 5 | CH₂═CH₂ | CONH₂ |
| 5 | CH₂═CH₂ | CH═CH₂ |
| 5 | CH₂═CH₂ | C≡CH |

R, R₁, and R₂ = H, alkyl, alkenyl, alkynyl, aryl, and heterocycle

TABLE 6

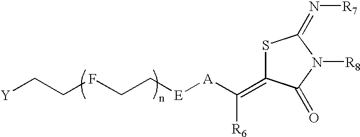

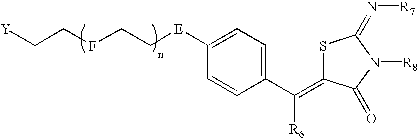

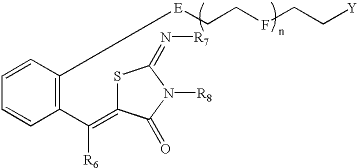

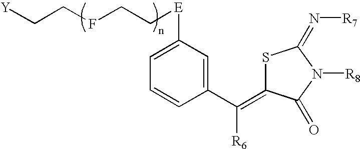

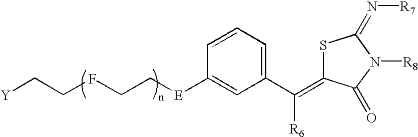

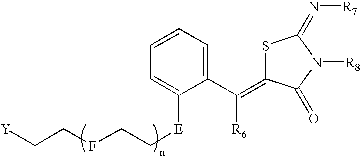

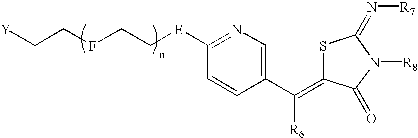

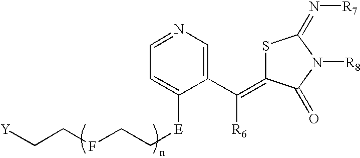

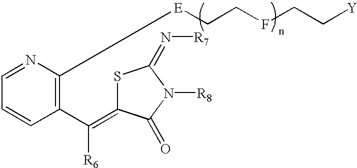

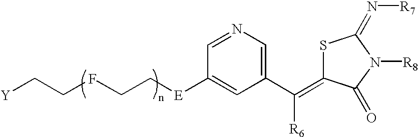

| n | E | F | Y |
|---|---|---|---|
| 0 | O | O | OH |
| 0 | O | O | NH₂ |
| 0 | O | CONR | I |
| 0 | O | NRCONR | COH |
| 0 | O | NRCONR | COR |
| 0 | O | NRCOO | CH═CH₂ |
| 0 | O | CH═CH | NHR |
| 0 | O | CH═CH | COH |
| 0 | S | S | NHR |
| 0 | S | S | COH |
| 0 | S | S | COR |
| 0 | S | CR₁R₂ | COH |
| 0 | S | CR₁R₂ | COR |
| 0 | S | SO₂NR | OH |
| 0 | S | SO₂NR | SO₂H |
| 0 | S | NRCNHNR | CONH₂ |
| 0 | S | NRCNHNR | CH═CH₂ |
| 0 | NR | O | C≡CH |
| 0 | NR | CONR | Cl |
| 0 | NR | CONR | COR |
| 0 | NR | NRCONR | OH |
| 0 | NR | NRCONR | SH |
| 0 | NR | NRCONR | CONH₂ |
| 0 | NR | NRCOO | COR |
| 0 | NR | CH═CH | OH |
| 0 | NR | CH═CH | N₃ |
| 0 | NR | CH═CH | CONH₂ |
| 0 | NR | CH═CH | CH═CH₂ |
| 0 | CR₁R₂ | S | COH |
| 0 | CR₁R₂ | S | COR |
| 0 | CR₁R₂ | CR₁R₂ | SH |
| 0 | CR₁R₂ | CR₁R₂ | COOH |
| 0 | CR₁R₂ | CR₁R₂ | NH₂ |
| 0 | CR₁R₂ | SO₂NR | Cl |
| 0 | CR₁R₂ | SO₂NR | CN |
| 0 | CR₁R₂ | SO₂NR | N₃ |
| 0 | CR₁R₂ | NRCNHNR | NHR |
| 0 | CR₁R₂ | NRCNHNR | COR |
| 0 | CR₁R₂ | C≡C | OH |
| 0 | CR₁R₂ | C≡C | Br |
| 0 | CONR | O | OH |
| 0 | CONR | O | SH |
| 0 | CONR | O | COR |
| 0 | CONR | NR | OH |
| 0 | CONR | NR | COR |
| 0 | CONR | CONR | OH |
| 0 | CONR | CONR | SH |
| 0 | CONR | CONR | COOH |
| 0 | CONR | NRCOO | Br |
| 0 | CONR | NRCOO | CONH₂ |
| 0 | CONR | CH═CH | CONH₂ |
| 0 | CONR | CH═CH | CH═CH₂ |
| 0 | CONR | CH═CH | NH₂ |
| 0 | SO₂NR | S | SH |
| 0 | SO₂NR | S | COOH |
| 0 | SO₂NR | S | F |
| 0 | SO₂NR | CR₁R₂ | CONH₂ |
| 0 | SO₂NR | SO₂NR | F |
| 0 | SO₂NR | SO₂NR | N₃ |
| 0 | SO₂NR | SO₂NR | CH═CH₂ |
| 0 | SO₂NR | NRCNHNR | SH |
| 0 | SO₂NR | NRCNHNR | SO₂H |
| 0 | SO₂NR | NRCNHNR | Cl |
| 0 | SO₂NR | C≡C | NHR |
| 0 | SO₂NR | C≡C | COR |
| 0 | NRCONR | O | OH |
| 0 | NRCONR | O | SH |
| 0 | NRCONR | O | COOH |
| 0 | NRCONR | NR | SO₂H |
| 0 | NRCONR | NR | COH |
| 0 | NRCONR | NR | COR |
| 0 | NRCONR | CONR | F |
| 0 | NRCONR | CONR | CH═CH₂ |
| 0 | NRCONR | CONR | C≡CH |
| 0 | NRCONR | NRCONR | COR |
| 0 | NRCONR | NRCOO | OH |
| 0 | NRCONR | NRCOO | COH |
| 0 | NRCONR | NRCOO | COR |
| 0 | NRCONR | CH═CH | OH |
| 0 | NRCONR | CH═CH | SH |
| 0 | NRCONR | CH═CH | COOH |
| 0 | NRCNHNR | S | C≡CH |
| 0 | NRCNHNR | S | NH₂ |
| 0 | NRCNHNR | S | NHR |
| 0 | NRCNHNR | CR₁R₂ | Br |
| 0 | NRCNHNR | CR₁R₂ | NH₂ |
| 0 | NRCNHNR | CR₁R₂ | NHR |
| 0 | NRCNHNR | SO₂NR | SH |
| 0 | NRCNHNR | SO₂NR | COOH |
| 0 | NRCNHNR | NRCNHNR | CN |
| 0 | NRCNHNR | NRCNHNR | N₃ |
| 0 | NRCNHNR | NRCNHNR | CONH₂ |
| 0 | NRCNHNR | C≡C | SH |
| 0 | NRCNHNR | C≡C | COOH |
| 0 | NRCOO | O | CN |
| 0 | NRCOO | O | N₃ |
| 0 | NRCOO | O | CONH₂ |
| 0 | NRCOO | CONR | CN |
| 0 | NRCOO | CONR | N₃ |
| 0 | NRCOO | NRCONR | COH |
| 0 | NRCOO | NRCONR | COR |
| 0 | NRCOO | NRCOO | OH |
| 0 | NRCOO | NRCOO | SH |
| 0 | NRCOO | CH═CH | F |
| 0 | C≡C | S | COOH |
| 0 | C≡C | S | SO₂H |
| 0 | C≡C | CR₁R₂ | NH₂ |
| 0 | C≡C | CR₁R₂ | NHR |
| 0 | C≡C | CR₁R₂ | COH |
| 0 | C≡C | SO₂NR | COH |
| 0 | C≡C | SO₂NR | COR |
| 0 | C≡C | NRCNHNR | OH |
| 0 | C≡C | NRCNHNR | SO₂H |
| 0 | C≡C | NRCNHNR | Cl |
| 0 | C≡C | C≡C | OH |
| 0 | C≡C | C≡C | CN |
| 0 | CH═CH | O | CH═CH₂ |
| 0 | CH═CH | O | C≡CH |
| 0 | CH═CH | O | COR |
| 0 | CH═CH | NR | OH |
| 0 | CH═CH | NR | SH |
| 0 | CH═CH | NRCONR | COH |
| 0 | CH═CH | NRCONR | COR |
| 0 | CH═CH | NRCOO | SH |
| 0 | CH═CH | NRCOO | NHR |
| 0 | CH═CH | NRCOO | COH |
| 0 | CH═CH | CH═CH | OH |
| 0 | CH═CH | CH═CH | SH |
| 0 | O | S | OH |
| 0 | O | S | NH₂ |
| 0 | O | SO₂NR | I |
| 0 | O | NRCNHNR | COH |
| 0 | O | NRCNHNR | COR |
| 0 | O | C≡C | CH═CH₂ |
| 0 | S | O | NHR |
| 0 | S | O | COH |
| 0 | S | NR | NHR |
| 0 | S | NR | COH |
| 0 | S | NR | COR |
| 0 | S | CONR | COH |
| 0 | S | CONR | COR |
| 0 | S | NRCONR | OH |
| 0 | S | NRCONR | SO₂H |
| 0 | S | NRCOO | CONH₂ |
| 0 | S | NRCOO | CH═CH₂ |
| 0 | NR | S | C≡CH |
| 0 | NR | SO₂NR | Cl |
| 0 | NR | SO₂NR | COR |
| 0 | NR | NRCNHNR | OH |
| 0 | NR | NRCNHNR | SH |
| 0 | NR | NRCNHNR | CONH₂ |
| 0 | NR | | COR |
| 0 | CR₁R₂ | O | OH |
| 0 | CR₁R₂ | O | N₃ |
| 0 | CR₁R₂ | O | CONH₂ |
| 0 | CR₁R₂ | O | CH═CH₂ |
| 0 | CR₁R₂ | NR | COH |
| 0 | CR₁R₂ | NR | COR |
| 0 | CR₁R₂ | CONR | SH |
| 0 | CR₁R₂ | CONR | COOH |
| 0 | CR₁R₂ | CONR | NH₂ |
| 0 | CR₁R₂ | NRCONR | Cl |
| 0 | CR₁R₂ | NRCONR | CN |
| 0 | CR₁R₂ | NRCONR | N₃ |
| 0 | CR₁R₂ | NRCOO | NHR |
| 0 | CR₁R₂ | NRCOO | COR |
| 0 | CR₁R₂ | CH═CH | OH |
| 0 | CR₁R₂ | CH═CH | Br |
| 0 | CONR | S | OH |
| 0 | CONR | S | SH |
| 0 | CONR | S | COR |
| 0 | CONR | CR₁R₂ | OH |
| 0 | CONR | CR₁R₂ | COR |
| 0 | CONR | SO₂NR | OH |
| 0 | CONR | SO₂NR | SH |
| 0 | CONR | SO₂NR | COOH |
| 0 | CONR | C≡C | Br |
| 0 | CONR | C≡C | CONH₂ |
| 0 | SO₂NR | O | CONH₂ |
| 0 | SO₂NR | O | CH═CH₂ |
| 0 | SO₂NR | O | NH₂ |
| 0 | SO₂NR | NR | SH |
| 0 | SO₂NR | NR | COOH |
| 0 | SO₂NR | NR | F |
| 0 | SO₂NR | CONR | CONH₂ |
| 0 | SO₂NR | NRCONR | F |
| 0 | SO₂NR | NRCONR | N₃ |
| 0 | SO₂NR | NRCONR | CH═CH₂ |
| 0 | SO₂NR | NRCOO | SH |
| 0 | SO₂NR | NRCOO | SO₂H |
| 0 | SO₂NR | NRCOO | Cl |
| 0 | SO₂NR | CH═CH | NHR |
| 0 | SO₂NR | CH═CH | COR |
| 0 | NRCONR | S | OH |
| 0 | NRCONR | S | SH |
| 0 | NRCONR | S | COOH |
| 0 | NRCONR | CR₁R₂ | SO₂H |
| 0 | NRCONR | CR₁R₂ | COH |
| 0 | NRCONR | CR₁R₂ | COR |
| 0 | NRCONR | SO₂NR | F |
| 0 | NRCONR | SO₂NR | CH═CH₂ |
| 0 | NRCONR | SO₂NR | C≡CH |
| 0 | NRCONR | NRCNHNR | COR |
| 0 | NRCONR | C≡C | OH |
| 0 | NRCONR | C≡C | COH |
| 0 | NRCONR | | COR |
| 0 | NRCNHNR | O | OH |
| 0 | NRCNHNR | O | SH |
| 0 | NRCNHNR | O | COOH |
| 0 | NRCNHNR | NR | C≡CH |
| 0 | NRCNHNR | NR | NH₂ |
| 0 | NRCNHNR | NR | NHR |
| 0 | NRCNHNR | CONR | Br |
| 0 | NRCNHNR | CONR | NH₂ |
| 0 | NRCNHNR | CONR | NHR |
| 0 | NRCNHNR | NRCONR | SH |
| 0 | NRCNHNR | NRCONR | COOH |
| 0 | NRCNHNR | NRCOO | CN |
| 0 | NRCNHNR | NRCOO | N₃ |
| 0 | NRCNHNR | NRCOO | CONH₂ |
| 0 | NRCNHNR | CH═CH | SH |
| 0 | NRCNHNR | CH═CH | COOH |
| 0 | NRCOO | S | CN |
| 0 | NRCOO | S | N₃ |
| 0 | NRCOO | S | CONH₂ |
| 0 | NRCOO | SO₂NR | CN |
| 0 | NRCOO | SO₂NR | N₃ |
| 0 | NRCOO | NRCNHNR | COH |
| 0 | NRCOO | NRCNHNR | COR |
| 0 | NRCOO | C≡C | OH |
| 0 | NRCOO | C≡C | SH |
| 0 | C≡C | O | F |
| 0 | C≡C | NR | COOH |
| 0 | C≡C | NR | SO₂H |
| 0 | C≡C | CONR | NH₂ |
| 0 | C≡C | CONR | NHR |
| 0 | C≡C | CONR | COH |
| 0 | C≡C | NRCONR | COH |
| 0 | C≡C | NRCONR | COR |
| 0 | C≡C | NRCOO | OH |
| 0 | C≡C | NRCOO | SO₂H |
| 0 | C≡C | NRCOO | Cl |
| 0 | C≡C | CH═CH | OH |
| 0 | C≡C | CH═CH | CN |
| 0 | CH═CH | S | CH═CH₂ |
| 0 | CH═CH | S | C≡CH |
| 0 | CH═CH | S | COR |
| 0 | CH═CH | CR₁R₂ | OH |
| 0 | CH═CH | CR₁R₂ | SH |
| 0 | CH═CH | NRCNHNR | COH |
| 0 | CH═CH | NRCNHNR | COR |
| 0 | CH═CH | C≡C | SH |
| 0 | CH═CH | C≡C | NHR |
| 0 | CH═CH | C≡C | COH |
| 0 | CH═CH | CH═CH | N₃ |
| 0 | CH═CH | CH═CH | CONH₂ |
| 1 | O | O | C≡CH |
| 1 | O | O | NH₂ |
| 1 | O | O | NHR |
| 1 | O | NR | NHR |
| 1 | O | NR | COH |
| 1 | O | CONR | SH |
| 1 | O | CONR | SO₂H |
| 1 | O | NRCONR | OH |
| 1 | O | NRCONR | SH |
| 1 | O | NRCOO | SH |
| 1 | O | NRCOO | COOH |
| 1 | O | CH═CH | OH |
| 1 | O | CH═CH | COH |
| 1 | O | CH═CH | COR |
| 1 | S | S | OH |
| 1 | S | S | CH═CH₂ |
| 1 | S | S | NH₂ |
| 1 | S | CR₁R₂ | Cl |
| 1 | S | CR₁R₂ | Br |
| 1 | S | SO₂NR | Br |
| 1 | S | SO₂NR | COH |
| 1 | S | NRCNHNR | COOH |
| 1 | S | NRCNHNR | F |
| 1 | S | C≡C | OH |
| 1 | S | C≡C | SH |
| 1 | S | C≡C | COOH |
| 1 | S | C≡C | C≡CH |
| 1 | NR | O | SO₂H |
| 1 | NR | O | Cl |
| 1 | NR | O | CN |
| 1 | NR | NR | CONH₂ |
| 1 | NR | NR | CH═CH₂ |
| 1 | NR | CONR | CONH₂ |
| 1 | NR | CONR | COR |
| 1 | NR | NRCONR | NHR |
| 1 | NR | NRCONR | COH |
| 1 | NR | NRCOO | OH |
| 1 | NR | NRCOO | N₃ |
| 1 | NR | NRCOO | CONH₂ |
| 1 | NR | CH═CH | N₃ |
| 1 | NR | CH═CH | CONH₂ |
| 1 | NR | CH═CH | CH═CH₂ |
| 1 | CR₁R₂ | S | Br |
| 1 | CR₁R₂ | S | N₃ |
| 1 | CR₁R₂ | S | NHR |
| 1 | CR₁R₂ | S | COH |
| 1 | CR₁R₂ | CR₁R₂ | SO₂H |
| 1 | CR₁R₂ | SO₂NR | COOH |
| 1 | CR₁R₂ | SO₂NR | SO₂H |
| 1 | CR₁R₂ | NRCNHNR | CN |
| 1 | CR₁R₂ | NRCNHNR | COH |
| 1 | CR₁R₂ | NRCNHNR | COR |
| 1 | CR₁R₂ | C≡C | SH |
| 1 | CR₁R₂ | C≡C | COOH |
| 1 | CONR | O | OH |
| 1 | CONR | O | SH |
| 1 | CONR | O | COOH |
| 1 | CONR | NR | CN |
| 1 | CONR | NR | N₃ |
| 1 | CONR | NR | COH |
| 1 | CONR | NR | COR |
| 1 | CONR | CONR | OH |
| 1 | CONR | CONR | F |
| 1 | CONR | CONR | NHR |
| 1 | CONR | CONR | COR |
| 1 | CONR | NRCONR | OH |
| 1 | CONR | NRCONR | SO₂H |
| 1 | CONR | NRCOO | SH |
| 1 | CONR | NRCOO | COOH |
| 1 | CONR | NRCOO | COH |
| 1 | CONR | CH═CH | Cl |
| 1 | CONR | CH═CH | Br |
| 1 | SO₂NR | S | N₃ |
| 1 | SO₂NR | S | CONH₂ |
| 1 | SO₂NR | S | COR |
| 1 | SO₂NR | CR₁R₂ | SH |
| 1 | SO₂NR | CR₁R₂ | COOH |
| 1 | SO₂NR | SO₂NR | SO₂H |
| 1 | SO₂NR | SO₂NR | Cl |
| 1 | SO₂NR | SO₂NR | Br |
| 1 | SO₂NR | SO₂NR | COH |
| 1 | SO₂NR | NRCNHNR | OH |
| 1 | SO₂NR | NRCNHNR | NH₂ |
| 1 | SO₂NR | C≡C | Br |
| 1 | SO₂NR | C≡C | COR |
| 1 | NRCONR | O | SH |
| 1 | NRCONR | O | NH₂ |
| 1 | NRCONR | NR | Cl |
| 1 | NRCONR | NR | I |
| 1 | NRCONR | CONR | F |
| 1 | NRCONR | CONR | N₃ |
| 1 | NRCONR | NRCONR | OH |
| 1 | NRCONR | NRCONR | COR |
| 1 | NRCONR | NRCOO | OH |
| 1 | NRCONR | NRCOO | COR |
| 1 | NRCONR | CH═CH | OH |
| 1 | NRCONR | CH═CH | COOH |
| 1 | NRCNHNR | S | NH₂ |
| 1 | NRCNHNR | S | NHR |
| 1 | NRCNHNR | S | COH |
| 1 | NRCNHNR | CR₁R₂ | F |
| 1 | NRCNHNR | CR₁R₂ | CN |
| 1 | NRCNHNR | SO₂NR | CN |
| 1 | NRCNHNR | SO₂NR | NHR |
| 1 | NRCNHNR | SO₂NR | COH |
| 1 | NRCNHNR | NRCNHNR | Cl |
| 1 | NRCNHNR | NRCNHNR | Br |
| 1 | NRCNHNR | NRCNHNR | CH═CH₂ |
| 1 | NRCNHNR | C≡C | OH |
| 1 | NRCNHNR | C≡C | SO₂H |
| 1 | NRCNHNR | C≡C | COR |
| 1 | NRCOO | O | F |
| 1 | NRCOO | O | N₃ |
| 1 | NRCOO | O | CONH₂ |
| 1 | NRCOO | NR | OH |
| 1 | NRCOO | NR | SH |
| 1 | NRCOO | NR | I |
| 1 | NRCOO | CONR | OH |
| 1 | NRCOO | CONR | N₃ |
| 1 | NRCOO | CONR | COR |
| 1 | NRCOO | NRCONR | OH |
| 1 | NRCOO | NRCONR | N₃ |
| 1 | NRCOO | NRCOO | SH |
| 1 | NRCOO | NRCOO | CH═CH₂ |
| 1 | NRCOO | CH═CH | I |
| 1 | NRCOO | CH═CH | F |
| 1 | NRCOO | CH═CH | C≡CH |
| 1 | C≡C | S | I |
| 1 | C≡C | S | F |
| 1 | C≡C | S | CH═CH₂ |
| 1 | C≡C | CR₁R₂ | OH |
| 1 | C≡C | CR₁R₂ | SH |
| 1 | C≡C | CR₁R₂ | COOH |
| 1 | C≡C | CR₁R₂ | SO₂H |
| 1 | C≡C | SO₂NR | NHR |
| 1 | C≡C | NRCNHNR | SH |
| 1 | C≡C | NRCNHNR | SO₂H |
| 1 | C≡C | NRCNHNR | COR |
| 1 | C≡C | C≡C | OH |
| 1 | C≡C | C≡C | COH |
| 1 | C≡C | C≡C | COR |
| 1 | CH═CH | O | OH |
| 1 | CH═CH | O | COOH |
| 1 | CH═CH | O | COH |
| 1 | CH═CH | NR | SO₂H |
| 1 | CH═CH | NR | F |
| 1 | CH═CH | NR | COH |
| 1 | CH═CH | CONR | SH |
| 1 | CH═CH | CONR | I |
| 1 | CH═CH | CONR | F |
| 1 | CH═CH | NRCONR | CH═CH₂ |
| 1 | CH═CH | NRCONR | C≡CH |
| 1 | CH═CH | NRCONR | NH₂ |
| 1 | CH═CH | NRCOO | COH |
| 1 | CH═CH | NRCOO | COR |
| 1 | CH═CH | CH═CH | OH |
| 1 | CH═CH | CH═CH | Br |
| 1 | CH═CH | CH═CH | I |
| 1 | O | S | C≡CH |
| 1 | O | S | NH₂ |
| 1 | O | S | NHR |
| 1 | O | CR₁R₂ | NHR |
| 1 | O | CR₁R₂ | COH |
| 1 | O | SO₂NR | SH |
| 1 | O | SO₂NR | SO₂H |
| 1 | O | NRCNHNR | OH |
| 1 | O | NRCNHNR | SH |
| 1 | O | C≡C | SH |
| 1 | O | C≡C | COOH |
| 1 | S | O | OH |
| 1 | S | O | COH |
| 1 | S | O | COR |
| 1 | S | NR | OH |
| 1 | S | NR | CH═CH₂ |
| 1 | S | NR | NH₂ |
| 1 | S | CONR | Cl |
| 1 | S | CONR | Br |
| 1 | S | NRCONR | Br |
| 1 | S | NRCONR | COH |
| 1 | S | NRCOO | COOH |
| 1 | S | NRCOO | F |
| 1 | S | CH═CH | OH |
| 1 | S | CH═CH | SH |
| 1 | S | CH═CH | COOH |
| 1 | S | CH═CH | C≡CH |
| 1 | NR | S | SO₂H |
| 1 | NR | S | Cl |
| 1 | NR | S | CN |
| 1 | NR | CR₁R₂ | CONH₂ |
| 1 | NR | CR₁R₂ | CH═CH₂ |
| 1 | NR | SO₂NR | CONH₂ |
| 1 | NR | SO₂NR | COR |
| 1 | NR | NRCNHNR | NHR |
| 1 | NR | NRCNHNR | COH |
| 1 | NR | C≡C | OH |
| 1 | NR | C≡C | N₃ |
| 1 | NR | C≡C | CONH₂ |
| 1 | CR₁R₂ | O | N₃ |
| 1 | CR₁R₂ | O | CONH₂ |
| 1 | CR₁R₂ | O | CH═CH₂ |
| 1 | CR₁R₂ | NR | Br |
| 1 | CR₁R₂ | NR | N₃ |
| 1 | CR₁R₂ | NR | NHR |
| 1 | CR₁R₂ | NR | COH |
| 1 | CR₁R₂ | CONR | SO₂H |
| 1 | CR₁R₂ | NRCONR | COOH |
| 1 | CR₁R₂ | NRCONR | SO₂H |
| 1 | CR₁R₂ | NRCOO | CN |
| 1 | CR₁R₂ | NRCOO | COH |
| 1 | CR₁R₂ | NRCOO | COR |
| 1 | CR₁R₂ | CH═CH | SH |
| 1 | CR₁R₂ | CH═CH | COOH |
| 1 | CONR | S | OH |
| 1 | CONR | S | SH |
| 1 | CONR | S | COOH |
| 1 | CONR | CR₁R₂ | CN |
| 1 | CONR | CR₁R₂ | N₃ |
| 1 | CONR | CR₁R₂ | COH |
| 1 | CONR | CR₁R₂ | COR |
| 1 | CONR | SO₂NR | OH |
| 1 | CONR | SO₂NR | F |
| 1 | CONR | SO₂NR | NHR |
| 1 | CONR | SO₂NR | COR |
| 1 | CONR | NRCNHNR | OH |
| 1 | CONR | NRCNHNR | SO₂H |
| 1 | CONR | C≡C | SH |
| 1 | CONR | C≡C | COOH |
| 1 | CONR | C≡C | COH |
| 1 | SO₂NR | O | Cl |
| 1 | SO₂NR | O | Br |
| 1 | SO₂NR | NR | N₃ |
| 1 | SO₂NR | NR | CONH₂ |
| 1 | SO₂NR | NR | COR |
| 1 | SO₂NR | CONR | SH |
| 1 | SO₂NR | CONR | COOH |
| 1 | SO₂NR | NRCONR | SO₂H |
| 1 | SO₂NR | NRCONR | Cl |
| 1 | SO₂NR | NRCONR | Br |
| 1 | SO₂NR | NRCONR | COH |
| 1 | SO₂NR | NRCOO | OH |
| 1 | SO₂NR | NRCOO | NH₂ |
| 1 | SO₂NR | CH═CH | Br |
| 1 | SO₂NR | CH═CH | COR |
| 1 | NRCONR | S | SH |
| 1 | NRCONR | S | NH₂ |
| 1 | NRCONR | CR₁R₂ | Cl |
| 1 | NRCONR | CR₁R₂ | I |
| 1 | NRCONR | SO₂NR | F |
| 1 | NRCONR | SO₂NR | N₃ |
| 1 | NRCONR | NRCNHNR | OH |
| 1 | NRCONR | NRCNHNR | COR |
| 1 | NRCONR | C≡C | OH |
| 1 | NRCONR | | COR |
| 1 | NRCNHNR | O | OH |
| 1 | NRCNHNR | O | COOH |
| 1 | NRCNHNR | NR | NH₂ |
| 1 | NRCNHNR | NR | NHR |
| 1 | NRCNHNR | NR | COH |
| 1 | NRCNHNR | CONR | F |
| 1 | NRCNHNR | CONR | CN |
| 1 | NRCNHNR | NRCONR | CN |
| 1 | NRCNHNR | NRCONR | NHR |
| 1 | NRCNHNR | NRCONR | COH |
| 1 | NRCNHNR | NRCOO | Cl |
| 1 | NRCNHNR | NRCOO | Br |
| 1 | NRCNHNR | NRCOO | CH═CH₂ |
| 1 | NRCNHNR | CH═CH | OH |
| 1 | NRCNHNR | CH═CH | SO₂H |
| 1 | NRCNHNR | CH═CH | COR |
| 1 | NRCOO | S | F |
| 1 | NRCOO | S | N₃ |
| 1 | NRCOO | S | CONH₂ |
| 1 | NRCOO | CR₁R₂ | OH |
| 1 | NRCOO | CR₁R₂ | SH |
| 1 | NRCOO | CR₁R₂ | I |
| 1 | NRCOO | SO₂NR | OH |
| 1 | NRCOO | SO₂NR | N₃ |
| 1 | NRCOO | SO₂NR | COR |
| 1 | NRCOO | NRCNHNR | OH |
| 1 | NRCOO | NRCNHNR | N₃ |
| 1 | NRCOO | C≡C | SH |
| 1 | NRCOO | C≡C | CH═CH₂ |
| 1 | C≡C | O | I |
| 1 | C≡C | O | F |
| 1 | C≡C | O | C≡CH |
| 1 | C≡C | NR | I |
| 1 | C≡C | NR | F |
| 1 | C≡C | NR | CH═CH₂ |
| 1 | C≡C | CONR | OH |
| 1 | C≡C | CONR | SH |
| 1 | C≡C | CONR | COOH |
| 1 | C≡C | CONR | SO₂H |
| 1 | C≡C | NRCONR | NHR |
| 1 | C≡C | NRCOO | SH |
| 1 | C≡C | NRCOO | SO₂H |
| 1 | C≡C | NRCOO | COR |
| 1 | C≡C | CH═CH | OH |
| 1 | C≡C | CH═CH | COH |
| 1 | C≡C | CH═CH | COR |
| 1 | CH═CH | S | OH |
| 1 | CH═CH | S | COOH |
| 1 | CH═CH | S | COH |
| 1 | CH═CH | CR₁R₂ | SO₂H |
| 1 | CH═CH | CR₁R₂ | F |
| 1 | CH═CH | CR₁R₂ | COH |
| 1 | CH═CH | SO₂NR | SH |
| 1 | CH═CH | SO₂NR | I |
| 1 | CH═CH | SO₂NR | F |
| 1 | CH═CH | NRCNHNR | CH═CH₂ |
| 1 | CH═CH | NRCNHNR | C≡CH |
| 1 | CH═CH | NRCNHNR | NH₂ |
| 1 | CH═CH | C≡C | COH |
| 1 | CH═CH | C≡C | COR |
| 1 | CH═CH | CH═CH | N₃ |
| 1 | CH═CH | CH═CH | NHR |
| 1 | CH═CH | CH═CH | COH |
| 2 | O | O | F |
| 2 | O | O | CN |
| 2 | O | O | N₃ |
| 2 | O | NR | Br |
| 2 | O | NR | F |
| 2 | O | NR | COR |
| 2 | O | CONR | OH |
| 2 | O | CONR | SH |
| 2 | O | CONR | COOH |
| 2 | O | NRCONR | N₃ |
| 2 | O | NRCONR | CONH₂ |
| 2 | O | NRCOO | Cl |
| 2 | O | NRCOO | CH═CH₂ |
| 2 | O | CH═CH | SH |
| 2 | O | CH═CH | COOH |
| 2 | O | CH═CH | COH |
| 2 | S | S | COOH |
| 2 | S | S | SO₂H |
| 2 | S | S | Cl |
| 2 | S | S | NHR |
| 2 | S | CR₂R₂ | CN |
| 2 | S | CR₂R₂ | C≡CH |
| 2 | S | CR₂R₂ | NH₂ |
| 2 | S | SO₂NR | Cl |
| 2 | S | SO₂NR | Br |
| 2 | S | SO₂NR | N₃ |
| 2 | S | NRCNHNR | Br |
| 2 | S | NRCNHNR | I |
| 2 | S | NRCNHNR | COR |
| 2 | S | C≡C | OH |
| 2 | S | C≡C | SH |
| 2 | S | C≡C | CH═CH₂ |
| 2 | NR | O | C≡CH |
| 2 | NR | O | NH₂ |
| 2 | NR | O | NHR |
| 2 | NR | NR | Br |
| 2 | NR | NR | F |
| 2 | NR | NR | NH₂ |
| 2 | NR | NR | NHR |
| 2 | NR | CONR | CN |
| 2 | NR | CONR | COR |
| 2 | NR | NRCONR | OH |
| 2 | NR | NRCONR | SH |
| 2 | NR | NRCOO | CH═CH₂ |
| 2 | NR | NRCOO | C≡CH |
| 2 | NR | NRCOO | NH₂ |
| 2 | NR | CH═CH | Br |
| 2 | NR | CH═CH | NH₂ |
| 2 | NR | CH═CH | COH |
| 2 | NR | CH═CH | COR |
| 2 | CR₂R₂ | S | OH |
| 2 | CR₂R₂ | S | SH |
| 2 | CR₂R₂ | S | NH₂ |
| 2 | CR₂R₂ | CR₂R₂ | CN |
| 2 | CR₂R₂ | CR₂R₂ | N₃ |
| 2 | CR₂R₂ | CR₂R₂ | CONH₂ |
| 2 | CR₂R₂ | CR₂R₂ | CH═CH₂ |
| 2 | CR₂R₂ | SO₂NR | OH |
| 2 | CR₂R₂ | SO₂NR | Br |
| 2 | CR₂R₂ | SO₂NR | I |
| 2 | CR₂R₂ | SO₂NR | F |
| 2 | CR₂R₂ | NRCNHNR | SH |
| 2 | CR₂R₂ | NRCNHNR | COOH |
| 2 | CR₂R₂ | NRCNHNR | SO₂H |
| 2 | CR₂R₂ | C≡C | Cl |
| 2 | CR₂R₂ | C≡C | NH₂ |
| 2 | CR₂R₂ | C≡C | COH |
| 2 | CONR | O | SO₂H |
| 2 | CONR | O | N₃ |
| 2 | CONR | NR | COOH |
| 2 | CONR | NR | SO₂H |
| 2 | CONR | NR | Cl |
| 2 | CONR | CONR | CH═CH₂ |
| 2 | CONR | CONR | C≡CH |
| 2 | CONR | CONR | NH₂ |
| 2 | CONR | NRCONR | NH₂ |
| 2 | CONR | NRCONR | NHR |
| 2 | CONR | NRCOO | CN |
| 2 | CONR | NRCOO | COR |
| 2 | CONR | CH═CH | OH |
| 2 | CONR | CH═CH | Br |
| 2 | CONR | CH═CH | I |
| 2 | SO₂NR | S | OH |
| 2 | SO₂NR | S | SH |
| 2 | SO₂NR | S | COH |
| 2 | SO₂NR | CR₂R₂ | COOH |
| 2 | SO₂NR | CR₂R₂ | COR |
| 2 | SO₂NR | SO₂NR | OH |
| 2 | SO₂NR | SO₂NR | SH |
| 2 | SO₂NR | SO₂NR | COOH |
| 2 | SO₂NR | NRCNHNR | CH═CH₂ |
| 2 | SO₂NR | NRCNHNR | COH |
| 2 | SO₂NR | NRCNHNR | COR |
| 2 | SO₂NR | C≡C | NHR |
| 2 | SO₂NR | C≡C | COH |
| 2 | NRCONR | O | COOH |
| 2 | NRCONR | O | CONH₂ |
| 2 | NRCONR | O | CH═CH₂ |
| 2 | NRCONR | NR | Cl |
| 2 | NRCONR | NR | Br |
| 2 | NRCONR | CONR | COH |
| 2 | NRCONR | CONR | COR |
| 2 | NRCONR | NRCONR | SH |
| 2 | NRCONR | NRCONR | CN |
| 2 | NRCONR | NRCOO | F |
| 2 | NRCONR | NRCOO | CN |
| 2 | NRCONR | CH═CH | I |
| 2 | NRCONR | CH═CH | F |
| 2 | NRCONR | CH═CH | CN |
| 2 | NRCNHNR | S | F |
| 2 | NRCNHNR | S | COH |
| 2 | NRCNHNR | S | COR |
| 2 | NRCNHNR | CR₂R₂ | COR |
| 2 | NRCNHNR | SO₂NR | OH |
| 2 | NRCNHNR | SO₂NR | N₃ |
| 2 | NRCNHNR | NRCNHNR | CONH₂ |
| 2 | NRCNHNR | NRCNHNR | COH |
| 2 | NRCNHNR | NRCNHNR | COR |
| 2 | NRCNHNR | C≡C | OH |
| 2 | NRCNHNR | C≡C | SH |
| 2 | NRCNHNR | C≡C | NH₂ |
| 2 | NRCOO | O | I |
| 2 | NRCOO | O | C≡CH |
| 2 | NRCOO | O | COR |
| 2 | NRCOO | NR | SH |
| 2 | NRCOO | NR | COOH |
| 2 | NRCOO | CONR | I |
| 2 | NRCOO | CONR | CN |
| 2 | NRCOO | NRCONR | OH |
| 2 | NRCOO | NRCONR | SH |
| 2 | NRCOO | NRCOO | Br |
| 2 | NRCOO | NRCOO | F |
| 2 | NRCOO | NRCOO | N₃ |
| 2 | NRCOO | CH═CH | CN |
| 2 | NRCOO | CH═CH | C≡CH |
| 2 | NRCOO | CH═CH | NH₂ |
| 2 | C≡C | S | COOH |
| 2 | C≡C | S | CONH₂ |
| 2 | C≡C | S | NHR |
| 2 | C≡C | CR₂R₂ | COOH |
| 2 | C≡C | SO₂NR | SH |
| 2 | C≡C | SO₂NR | N₃ |
| 2 | C≡C | SO₂NR | CONH₂ |
| 2 | C≡C | SO₂NR | CH═CH₂ |
| 2 | C≡C | NRCNHNR | I |
| 2 | C≡C | NRCNHNR | F |
| 2 | C≡C | NRCHNHR | NHR |
| 2 | C≡C | C≡C | CH═CH₂ |
| 2 | C≡C | C≡C | C≡CH |
| 2 | CH═CH | O | CONH₂ |
| 2 | CH═CH | O | NHR |
| 2 | CH═CH | O | COR |
| 2 | CH═CH | NR | I |
| 2 | CH═CH | NR | F |
| 2 | CH═CH | NR | CN |
| 2 | CH═CH | NR | CH═CH₂ |
| 2 | CH═CH | CONR | C≡CH |
| 2 | CH═CH | CONR | NH₂ |
| 2 | CH═CH | NRCONR | Cl |
| 2 | CH═CH | NRCONR | N₃ |
| 2 | CH═CH | NRCOO | SH |
| 2 | CH═CH | NRCOO | CONH₂ |
| 2 | CH═CH | NRCOO | CH═CH₂ |
| 2 | CH═CH | NRCOO | C≡CH |
| 2 | CH═CH | CH═CH | SO₂H |
| 2 | CH═CH | CH═CH | Cl |
| 2 | CH═CH | CH═CH | Br |
| 2 | O | S | F |
| 2 | O | S | CN |
| 2 | O | S | N₃ |
| 2 | O | CR₂R₂ | Br |
| 2 | O | CR₂R₂ | F |
| 2 | O | CR₂R₂ | COR |
| 2 | O | SO₂NR | OH |
| 2 | O | SO₂NR | SH |
| 2 | O | SO₂NR | COOH |
| 2 | O | NRCNHNR | N₃ |
| 2 | O | NRCNHNR | CONH₂ |
| 2 | O | C≡C | Cl |
| 2 | O | C≡C | CH═CH₂ |
| 2 | S | O | SH |
| 2 | S | O | COOH |
| 2 | S | O | COH |
| 2 | S | NR | COOH |
| 2 | S | NR | SO₂H |
| 2 | S | NR | Cl |
| 2 | S | NR | NHR |
| 2 | S | CONR | CN |
| 2 | S | CONR | C≡CH |
| 2 | S | CONR | NH₂ |
| 2 | S | NRCONR | Cl |
| 2 | S | NRCONR | Br |
| 2 | S | NRCONR | N₃ |
| 2 | S | NRCOO | Br |
| 2 | S | NRCOO | I |
| 2 | S | NRCOO | COR |
| 2 | S | CH═CH | OH |
| 2 | S | CH═CH | SH |
| 2 | S | CH═CH | CH═CH₂ |
| 2 | NR | S | C≡CH |
| 2 | NR | S | NH₂ |
| 2 | NR | S | NHR |
| 2 | NR | CR₂R₂ | Br |
| 2 | NR | CR₂R₂ | F |
| 2 | NR | CR₂R₂ | NH₂ |
| 2 | NR | CR₂R₂ | NHR |
| 2 | NR | SO₂NR | CN |
| 2 | NR | SO₂NR | COR |
| 2 | NR | NRCNHNR | OH |
| 2 | NR | NRCNHNR | SH |
| 2 | NR | C≡C | CH═CH₂ |
| 2 | NR | C≡C | C≡CH |
| 2 | NR | C≡C | NH₂ |
| 2 | CR₂R₂ | O | Br |
| 2 | CR₂R₂ | OO | NH₂ |
| 2 | CR₂R₂ | O | COH |
| 2 | CR₂R₂ | O | COR |
| 2 | CR₂R₂ | NR | OH |
| 2 | CR₂R₂ | NR | SH |
| 2 | CR₂R₂ | NR | NH₂ |
| 2 | CR₂R₂ | CONR | CN |
| 2 | CR₂R₂ | CONR | N₃ |
| 2 | CR₂R₂ | CONR | CONH₂ |
| 2 | CR₂R₂ | CONR | CH═CH₂ |
| 2 | CR₂R₂ | NRCONR | OH |
| 2 | CR₂R₂ | NRCONR | Br |
| 2 | CR₂R₂ | NRCONR | I |
| 2 | CR₂R₂ | NRCONR | F |
| 2 | CR₂R₂ | NRCOO | SH |
| 2 | CR₂R₂ | NRCOO | COOH |
| 2 | CR₂R₂ | NRCOO | SO₂H |
| 2 | CR₂R₂ | CH═CH | Cl |
| 2 | CR₂R₂ | CH═CH | NH₂ |
| 2 | CR₂R₂ | CH═CH | COH |
| 2 | CONR | S | SO₂H |
| 2 | CONR | S | N₃ |
| 2 | CONR | CR₂R₂ | COOH |
| 2 | CONR | CR₂R₂ | SO₂H |
| 2 | CONR | CR₂R₂ | Cl |
| 2 | CONR | SO₂NR | CH═CH₂ |
| 2 | CONR | SO₂NR | C≡CH |
| 2 | CONR | SO₂NR | NH₂ |
| 2 | CONR | NRCNHNR | NH₂ |
| 2 | CONR | NRCNHNR | NHR |
| 2 | CONR | C≡C | CN |
| 2 | CONR | C≡C | COR |
| 2 | SO₂NR | O | OH |
| 2 | SO₂NR | O | Br |
| 2 | SO₂NR | O | I |
| 2 | SO₂NR | NR | OH |
| 2 | SO₂NR | NR | SH |
| 2 | SO₂NR | NR | COH |
| 2 | SO₂NR | CONR | COOH |
| 2 | SO₂NR | CONR | COR |
| 2 | SO₂NR | NRCONR | OH |
| 2 | SO₂NR | NRCONR | SH |
| 2 | SO₂NR | NRCONR | COOH |
| 2 | SO₂NR | NRCOO | CH═CH₂ |
| 2 | SO₂NR | NRCOO | COH |
| 2 | SO₂NR | NRCOO | COR |
| 2 | SO₂NR | CH═CH | NHR |
| 2 | SO₂NR | CH═CH | COH |
| 2 | NRCONR | S | COOH |
| 2 | NRCONR | S | CONH₂ |
| 2 | NRCONR | S | CH═CH₂ |
| 2 | NRCONR | CR₂R₂ | Cl |
| 2 | NRCONR | CR₂R₂ | Br |
| 2 | NRCONR | SO₂NR | COH |
| 2 | NRCONR | SO₂NR | COR |
| 2 | NRCONR | NRCNHNR | SH |
| 2 | NRCONR | NRCNHNR | CN |
| 2 | NRCONR | C≡C | F |
| 2 | NRCONR | C≡C | CN |
| 2 | NRCNHNR | O | I |
| 2 | NRCNHNR | O | F |
| 2 | NRCNHNR | O | CN |
| 2 | NRCNHNR | NR | F |
| 2 | NRCNHNR | NR | COH |
| 2 | NRCNHNR | NR | COR |
| 2 | NRCNHNR | CONR | COR |
| 2 | NRCNHNR | NRCONR | OH |
| 2 | NRCNHNR | NRCONR | N₃ |
| 2 | NRCNHNR | NRCOO | CONH₂ |
| 2 | NRCNHNR | NRCOO | COH |
| 2 | NRCNHNR | NRCOO | COR |
| 2 | NRCNHNR | CH═CH | OH |
| 2 | NRCNHNR | CH═CH | SH |
| 2 | NRCNHNR | CH═CH | NH₂ |
| 2 | NRCOO | S | I |
| 2 | NRCOO | S | C≡CH |
| 2 | NRCOO | S | COR |
| 2 | NRCOO | CR₂R₂ | SH |
| 2 | NRCOO | CR₂R₂ | COOH |
| 2 | NRCOO | SO₂NR | I |
| 2 | NRCOO | SO₂NR | CN |
| 2 | NRCOO | NRCNHNR | OH |
| 2 | NRCOO | NRCNHNR | SH |
| 2 | NRCOO | C≡C | Br |
| 2 | NRCOO | C≡C | F |
| 2 | NRCOO | C≡C | N₃ |
| 2 | C≡C | O | CN |
| 2 | C≡C | O | C≡CH |
| 2 | C≡C | O | NH₂ |
| 2 | C≡C | NR | COOH |
| 2 | C≡C | NR | CONH₂ |
| 2 | C≡C | NR | NHR |
| 2 | C≡C | CONR | COOH |
| 2 | C≡C | NRCONR | SH |
| 2 | C≡C | NRCONR | N₃ |
| 2 | C≡C | NRCONR | CONH₂ |
| 2 | C≡C | NRCONR | CH═CH₂ |
| 2 | C≡C | NRCOO | I |
| 2 | C≡C | NRCOO | F |
| 2 | C≡C | NRCOO | NHR |
| 2 | C≡C | CH═CH | CH═CH₂ |
| 2 | C≡C | CH═CH | C≡CH |
| 2 | CH═CH | S | CONH₂ |
| 2 | CH═CH | S | NHR |
| 2 | CH═CH | S | COR |
| 2 | CH═CH | CR₂R₂ | I |
| 2 | CH═CH | CR₂R₂ | F |
| 2 | CH═CH | CR₂R₂ | CN |
| 2 | CH═CH | CR₂R₂ | CH═CH₂ |
| 2 | CH═CH | SO₂NR | C≡CH |
| 2 | CH═CH | SO₂NR | NH₂ |
| 2 | CH═CH | NRCNHNR | Cl |
| 2 | CH═CH | NRCNHNR | N₃ |
| 2 | CH═CH | C≡C | SH |
| 2 | CH═CH | C≡C | CONH₂ |
| 2 | CH═CH | C≡C | CH═CH₂ |
| 2 | CH═CH | C≡C | C≡CH |
| 2 | CH═CH | CH═CH | C≡CH |
| 2 | CH═CH | CH═CH | NH₂ |
| 2 | CH═CH | CH═CH | NHR |
| 3 | O | O | Cl |
| 3 | O | O | I |
| 3 | O | NR | CONH₂ |
| 3 | O | NR | CH═CH₂ |
| 3 | O | NR | NH₂ |
| 3 | O | CONR | NH₂ |
| 3 | O | CONR | NHR |
| 3 | O | NRCONR | N₃ |
| 3 | O | NRCONR | CONH₂ |
| 3 | O | NRCOO | SH |
| 3 | O | NRCOO | F |
| 3 | O | NRCOO | N₃ |
| 3 | O | NRCOO | C≡CH |
| 3 | O | NRCOO | NH₂ |
| 3 | O | CH═CH | NH₂ |
| 3 | O | CH═CH | COH |
| 3 | O | CH═CH | COR |
| 3 | S | S | OH |
| 3 | S | S | SH |
| 3 | S | S | NHR |
| 3 | S | S | COH |
| 3 | S | CR₃R₂ | NH₂ |
| 3 | S | SO₂NR | SH |
| 3 | S | SO₂NR | COOH |
| 3 | S | NRCNHNR | I |
| 3 | S | NRCNHNR | CONH₂ |
| 3 | S | NRCNHNR | COR |
| 3 | S | C≡C | OH |
| 3 | S | C≡C | SH |
| 3 | NR | O | CH═CH₂ |
| 3 | NR | O | C≡CH |
| 3 | NR | O | COH |
| 3 | NR | NR | SH |
| 3 | NR | NR | COOH |
| 3 | NR | NR | SO₂H |
| 3 | NR | CONR | NH₂ |
| 3 | NR | CONR | NHR |
| 3 | NR | CONR | COH |
| 3 | NR | NRCONR | COOH |
| 3 | NR | NRCONR | C≡CH |
| 3 | NR | NRCONR | NH₂ |
| 3 | NR | NRCOO | OH |
| 3 | NR | NRCOO | NHR |
| 3 | NR | CH═CH | COOH |
| 3 | NR | CH═CH | I |
| 3 | CR₃R₂ | S | Br |
| 3 | CR₃R₂ | CR₃R₂ | CH═CH₂ |
| 3 | CR₃R₂ | CR₃R₂ | C≡CH |
| 3 | CR₃R₂ | SO₂NR | NH₂ |
| 3 | CR₃R₂ | SO₂NR | NHR |
| 3 | CR₃R₂ | SO₂NR | COH |
| 3 | CR₃R₂ | NRCNHNR | COOH |
| 3 | CR₃R₂ | NRCNHNR | SO₂H |
| 3 | CR₃R₂ | NRCNHNR | COH |
| 3 | CR₃R₂ | C≡C | SO₂H |
| 3 | CR₃R₂ | C≡C | CN |
| 3 | CONR | O | SO₂H |
| 3 | CONR | O | Cl |
| 3 | CONR | O | Br |
| 3 | CONR | NR | N₃ |
| 3 | CONR | NR | CONH₂ |
| 3 | CONR | NR | CH═CH₂ |
| 3 | CONR | CONR | C≡CH |
| 3 | CONR | CONR | NH₂ |
| 3 | CONR | NRCONR | I |
| 3 | CONR | NRCONR | N₃ |
| 3 | CONR | NRCOO | COH |
| 3 | CONR | NRCOO | COR |
| 3 | CONR | CH═CH | OH |
| 3 | CONR | CH═CH | SH |
| 3 | SO₂NR | S | SO₂H |
| 3 | SO₂NR | S | COH |
| 3 | SO₂NR | S | COR |
| 3 | SO₂NR | CR₃R₂ | OH |
| 3 | SO₂NR | CR₃R₂ | SH |
| 3 | SO₂NR | CR₃R₂ | CONH₂ |
| 3 | SO₂NR | CR₃R₂ | CH═CH₂ |
| 3 | SO₂NR | SO₂NR | SH |
| 3 | SO₂NR | SO₂NR | COH |
| 3 | SO₂NR | SO₂NR | COR |
| 3 | SO₂NR | NRCNHNR | OH |
| 3 | SO₂NR | NRCNHNR | SH |
| 3 | SO₂NR | C≡C | CH═CH₂ |
| 3 | SO₂NR | C≡C | NH₂ |
| 3 | SO₂NR | C≡C | NHR |
| 3 | NRCONR | O | Br |
| 3 | NRCONR | O | I |
| 3 | NRCONR | NR | F |
| 3 | NRCONR | NR | CN |
| 3 | NRCONR | CONR | SO₂H |
| 3 | NRCONR | CONR | Cl |
| 3 | NRCONR | NRCONR | SH |
| 3 | NRCONR | NRCONR | CONH₂ |
| 3 | NRCONR | NRCONR | CH═CH₂ |
| 3 | NRCONR | NRCOO | NH₂ |
| 3 | NRCONR | NRCOO | COH |
| 3 | NRCONR | CH═CH | OH |
| 3 | NRCONR | CH═CH | CONH₂ |
| 3 | NRCONR | CH═CH | CH═CH₂ |
| 3 | NRCNHNR | S | SH |
| 3 | NRCNHNR | S | COOH |
| 3 | NRCNHNR | S | SO₂H |
| 3 | NRCNHNR | SO₂NR | Br |
| 3 | NRCNHNR | SO₂NR | C≡CH |
| 3 | NRCNHNR | SO₂NR | NH₂ |
| 3 | NRCNHNR | NRCNHNR | COOH |
| 3 | NRCNHNR | NRCNHNR | SO₂H |
| 3 | NRCNHNR | C≡C | Cl |
| 3 | NRCNHNR | C≡C | Br |
| a3 | NRCOO | O | SH |
| 3 | NRCOO | O | COOH |
| 3 | NRCOO | O | SO₂H |
| 3 | NRCOO | NR | F |
| 3 | NRCOO | NR | CN |
| 3 | NRCOO | NR | COR |
| 3 | NRCOO | CONR | C≡CH |
| 3 | NRCOO | CONR | COH |
| 3 | NRCOO | CONR | COR |
| 3 | NRCOO | NRCONR | OH |
| 3 | NRCOO | NRCONR | COR |
| 3 | NRCOO | NRCOO | Br |
| 3 | NRCOO | CH═CH | CONH₂ |
| 3 | NRCOO | CH═CH | CH═CH₂ |
| 3 | C≡C | S | OH |
| 3 | C≡C | CR₃R₂ | I |
| 3 | C≡C | CR₃R₂ | F |
| 3 | C≡C | CR₃R₂ | NH₂ |
| 3 | C≡C | SO₂NR | N₃ |
| 3 | C≡C | SO₂NR | CONH₂ |
| 3 | C≡C | SO₂NR | CH═CH₂ |
| 3 | C≡C | NRCNHNR | CH═CH₂ |
| 3 | C≡C | NRCNHNR | C≡CH |
| 3 | C≡C | C≡C | I |
| 3 | C≡C | C≡C | C≡CH |
| 3 | C≡C | C≡C | NH₂ |
| 3 | C≡C | C≡C | NHR |
| 3 | CH═CH | O | COOH |
| 3 | CH═CH | O | CN |
| 3 | CH═CH | NR | I |
| 3 | CH═CH | NR | F |
| 3 | CH═CH | CONR | CN |
| 3 | CH═CH | CONR | N₃ |
| 3 | CH═CH | CONR | C≡CH |
| 3 | CH═CH | NRCONR | NHR |
| 3 | CH═CH | NRCOO | Br |
| 3 | CH═CH | NRCOO | I |
| 3 | CH═CH | CH═CH | Cl |
| 3 | O | O | OH |
| 3 | O | O | SH |
| 3 | O | NR | CH═CH₂ |
| 3 | O | NR | C≡CH |
| 3 | O | NR | NH₂ |
| 3 | O | CONR | Br |
| 3 | O | NRCONR | Br |
| 3 | O | NRCONR | CONH₂ |
| 3 | O | NRCOO | COH |
| 3 | O | NRCOO | COR |
| 3 | O | CH═CH | CONH₂ |
| 3 | O | CH═CH | CH═CH₂ |
| 3 | O | CH═CH | C≡CH |
| 3 | S | S | CONH₂ |
| 3 | S | S | CH═CH₂ |
| 3 | S | S | C≡CH |
| 3 | S | S | NH₂ |
| 3 | S | CR₃R₂ | N₃ |
| 3 | S | CR₃R₂ | C≡CH |
| 3 | S | SO₂NR | Br |
| 3 | S | SO₂NR | NHR |
| 3 | S | SO₂NR | COH |
| 3 | S | NRCNHNR | N₃ |
| 3 | S | NRCNHNR | COR |
| 3 | S | C≡C | OH |
| 3 | S | C≡C | SH |
| 3 | S | C≡C | Br |
| 3 | NR | O | SH |
| 3 | NR | O | COOH |
| 3 | NR | O | CONH₂ |
| 3 | NR | O | COR |
| 3 | NR | NR | OH |
| 3 | NR | NR | I |
| 3 | NR | NR | F |
| 3 | NR | CONR | F |
| 3 | NR | CONR | CONH₂ |
| 3 | NR | NRCONR | Br |
| | NR | NRCONR | I |
| 3 | NR | NRCOO | CN |
| 3 | NR | NRCOO | N₃ |
| 3 | NR | NRCOO | CONH₂ |
| 3 | NR | CH═CH | Cl |
| 3 | NR | CH═CH | Br |
| 3 | CR₃R₂ | S | COOH |
| 3 | CR₃R₂ | S | SO₂H |
| 3 | CR₃R₂ | S | Cl |
| 3 | CR₃R₂ | CR₃R₂ | COOH |
| 3 | CR₃R₂ | CR₃R₂ | I |
| 3 | CR₃R₂ | CR₃R₂ | CH═CH₂ |
| 3 | CR₃R₂ | CR₃R₂ | C≡CH |
| 3 | CR₃R₂ | SO₂NR | F |
| 3 | CR₃R₂ | SO₂NR | CH═CH₂ |
| 3 | CR₃R₂ | SO₂NR | C≡CH |
| 3 | CR₃R₂ | SO₂NR | NH₂ |
| 3 | CR₃R₂ | NRCNHNR | OH |
| 3 | CR₃R₂ | NRCNHNR | SH |
| 3 | CR₃R₂ | C≡C | C≡CH |
| 3 | CR₃R₂ | C≡C | NH₂ |
| 3 | CONR | O | SH |
| 3 | CONR | O | COOH |
| 3 | CONR | O | CONH₂ |
| 3 | CONR | NR | I |
| 3 | CONR | NR | F |
| 3 | CONR | CONR | OH |
| 3 | CONR | CONR | SH |
| 3 | CONR | CONR | COOH |
| 3 | CONR | NRCONR | NHR |
| 3 | CONR | NRCONR | COH |
| 3 | CONR | NRCOO | I |
| 3 | CONR | NRCOO | F |
| 3 | CONR | CH═CH | F |
| 3 | CONR | CH═CH | COR |
| 3 | SO₂NR | S | OH |
| 3 | SO₂NR | S | SH |
| 3 | SO₂NR | CR₃R₂ | N₃ |
| 3 | SO₂NR | CR₃R₂ | CONH₂ |
| 3 | SO₂NR | SO₂NR | COOH |
| 3 | SO₂NR | SO₂NR | CN |
| 3 | SO₂NR | SO₂NR | N₃ |
| 3 | SO₂NR | SO₂NR | CONH₂ |
| 3 | SO₂NR | NRCNHNR | CN |
| 3 | SO₂NR | NRCNHNR | CH═CH₂ |
| 3 | SO₂NR | C≡C | SO₂H |
| 3 | SO₂NR | C≡C | Cl |
| 3 | SO₂NR | C≡C | Br |
| 3 | NRCONR | O | C≡CH |
| 3 | NRCONR | O | NH₂ |
| 3 | NRCONR | NR | Cl |
| 3 | NRCONR | NR | Br |
| 3 | NRCONR | NR | CONH₂ |
| 3 | NRCONR | CONR | OH |
| 3 | NRCONR | CONR | F |
| 3 | NRCONR | CONR | CN |
| 3 | NRCONR | NRCONR | CONH₂ |
| 3 | NRCONR | NRCONR | CH═CH₂ |
| 3 | NRCONR | NRCOO | CONH₂ |
| 3 | NRCONR | NRCOO | COH |
| 3 | NRCONR | CH═CH | SO₂H |
| 3 | NRCONR | CH═CH | Cl |
| 3 | NRCONR | CH═CH | F |
| 3 | NRCNHNR | S | OH |
| 3 | NRCNHNR | S | Br |
| 3 | NRCNHNR | CR₃R₂ | OH |
| 3 | NRCNHNR | CR₃R₂ | SH |
| 3 | NRCNHNR | CR₃R₂ | CH═CH₂ |
| 3 | NRCNHNR | SO₂NR | I |
| 3 | NRCNHNR | SO₂NR | NHR |
| 3 | NRCNHNR | SO₂NR | COH |
| 3 | NRCNHNR | SO₂NR | COR |
| 3 | NRCNHNR | NRCNHNR | N₃ |
| 3 | NRCNHNR | NRCNHNR | CONH₂ |
| 3 | NRCNHNR | NRCNHNR | COR |
| 3 | NRCNHNR | C≡C | OH |
| 3 | NRCNHNR | C≡C | COR |
| 3 | NRCOO | O | OH |
| a3 | NRCOO | O | SH |
| 3 | NRCOO | O | COR |
| 3 | NRCOO | NR | OH |
| 3 | NRCOO | NR | SH |
| 3 | NRCOO | NR | COOH |
| 3 | NRCOO | CONR | NH₂ |
| 3 | NRCOO | CONR | NHR |
| 3 | NRCOO | NRCONR | CH═CH₂ |
| 3 | NRCOO | NRCONR | NHR |
| 3 | NRCOO | NRCOO | I |
| 3 | NRCOO | CH═CH | OH |
| 3 | NRCOO | CH═CH | SH |
| 3 | NRCOO | CH═CH | COOH |
| 3 | C≡C | S | C≡CH |
| 3 | C≡C | S | NH₂ |
| 3 | C≡C | S | NHR |
| 3 | C≡C | CR₃R₂ | SO₂H |
| 3 | C≡C | CR₃R₂ | Cl |
| 3 | C≡C | CR₃R₂ | Br |
| 3 | C≡C | SO₂NR | OH |
| 3 | C≡C | SO₂NR | SH |
| 3 | C≡C | SO₂NR | Br |
| 3 | C≡C | NRCNHNR | CONH₂ |
| 3 | C≡C | NRCNHNR | NHR |
| 3 | C≡C | C≡C | C≡CH |
| 3 | C≡C | C≡C | NH₂ |
| 3 | C≡C | C≡C | COR |
| 3 | CH═CH | O | OH |
| 3 | CH═CH | O | SH |
| 3 | CH═CH | O | COOH |
| 3 | CH═CH | O | SO₂H |
| 3 | CH═CH | O | Cl |
| 3 | CH═CH | NR | OH |
| 3 | CH═CH | NR | COOH |
| 3 | CH═CH | NR | F |
| 3 | CH═CH | CONR | NH₂ |
| 3 | CH═CH | CONR | NHR |
| 3 | CH═CH | CONR | COH |
| 3 | CH═CH | CONR | COR |
| 3 | CH═CH | NRCONR | OH |
| 3 | CH═CH | NRCOO | CH═CH₂ |
| 3 | CH═CH | NRCOO | NHR |
| 3 | CH═CH | CH═CH | I |
| 3 | CH═CH | CH═CH | F |
| 3 | CH═CH | CH═CH | CN |
| 3 | O | O | OH |
| 3 | O | O | SH |
| 3 | O | O | COOH |
| 3 | O | NR | CONH₂ |
| 3 | O | NR | CH═CH₂ |
| 3 | O | NR | C≡CH |
| 3 | O | CONR | CONH₂ |
| 3 | O | CONR | CH═CH₂ |
| 3 | O | NRCONR | CONH₂ |
| 3 | O | NRCONR | CH═CH₂ |
| 3 | O | NRCOO | COOH |
| 3 | O | NRCOO | SO₂H |
| 3 | O | NRCOO | Cl |
| 3 | O | CH═CH | SO₂H |
| 3 | O | CH═CH | Cl |
| 3 | O | CH═CH | COR |
| 3 | S | S | OH |
| 3 | S | S | SH |
| 3 | S | S | COOH |
| 3 | S | S | SO₂H |
| 3 | S | CR₃R₂ | CONH₂ |
| 3 | S | CR₃R₂ | CH═CH₂ |
| 3 | S | CR₃R₂ | NHR |
| 3 | S | SO₂NR | NHR |
| 3 | S | SO₂NR | COH |
| 3 | S | SO₂NR | COR |
| 3 | S | NRCNHNR | OH |
| 3 | S | NRCNHNR | NH₂ |
| 3 | S | NRCNHNR | NHR |
| 3 | S | C≡C | I |
| 3 | S | C≡C | NH₂ |
| 3 | NR | O | SO₂H |
| 3 | NR | O | F |
| 3 | NR | O | CN |
| 3 | NR | O | N₃ |
| 3 | NR | O | NH₂ |
| 3 | NR | NR | SH |
| 3 | NR | NR | COOH |
| 3 | NR | CONR | CN |
| 3 | NR | CONR | COR |
| 3 | NR | NRCONR | OH |
| 3 | NR | NRCONR | NHR |
| 3 | NR | NRCOO | SO₂H |
| 3 | NR | NRCOO | C≡CH |
| 3 | NR | NRCOO | NH₂ |
| 3 | NR | NRCOO | NHR |
| 3 | NR | CH═CH | COR |
| 3 | CR₃R₂ | S | OH |
| 3 | CR₃R₂ | S | SH |
| 3 | CR₃R₂ | CR₃R₂ | SO₂H |
| 3 | CR₃R₂ | CR₃R₂ | Cl |
| 3 | CR₃R₂ | SO₂NR | OH |
| 3 | CR₃R₂ | SO₂NR | C≡CH |
| 3 | CR₃R₂ | SO₂NR | NH₂ |
| 3 | CR₃R₂ | SO₂NR | NHR |
| 3 | CR₃R₂ | NRCNHNR | Cl |
| 3 | CR₃R₂ | NRCNHNR | COR |
| 3 | CR₃R₂ | C≡C | Cl |
| 3 | CR₃R₂ | C≡C | Br |
| 3 | CR₃R₂ | C≡C | NHR |
| 3 | CONR | O | COR |
| 3 | CONR | NR | OH |
| 3 | CONR | NR | SH |
| 3 | CONR | NR | C≡CH |
| 3 | CONR | CONR | Br |
| 3 | CONR | CONR | I |
| 3 | CONR | CONR | F |
| 3 | CONR | NRCONR | OH |
| 3 | CONR | NRCOO | COOH |
| 3 | CONR | NRCOO | SO₂H |
| 3 | CONR | NRCOO | F |
| 3 | CONR | CH═CH | Cl |
| 3 | CONR | CH═CH | NHR |
| 3 | SO₂NR | S | OH |
| 3 | SO₂NR | S | SH |
| 3 | SO₂NR | S | NH₂ |
| 3 | SO₂NR | S | NHR |
| 3 | SO₂NR | CR₃R₂ | Cl |
| 3 | SO₂NR | CR₃R₂ | Br |
| 3 | SO₂NR | SO₂NR | Br |
| 3 | SO₂NR | SO₂NR | I |
| 3 | SO₂NR | NRCNHNR | OH |
| 3 | SO₂NR | NRCNHNR | SH |
| 3 | SO₂NR | NRCNHNR | COR |
| 3 | SO₂NR | C≡C | OH |
| 3 | SO₂NR | C≡C | CN |
| 3 | NRCONR | O | I |
| 3 | NRCONR | O | COH |
| 3 | NRCONR | O | COR |
| 3 | NRCONR | NR | OH |
| 3 | NRCONR | NR | SH |
| 3 | NRCONR | CONR | OH |
| 3 | NRCONR | CONR | SH |
| 3 | NRCONR | CONR | SO₂H |
| 3 | NRCONR | NRCONR | I |
| 3 | NRCONR | NRCONR | N₃ |
| 3 | NRCONR | NRCONR | CONH₂ |
| 3 | NRCONR | NRCOO | SH |
| 3 | NRCONR | NRCOO | COOH |
| 3 | NRCONR | CH═CH | CN |
| 3 | NRCONR | CH═CH | N₃ |
| 3 | NRCONR | CH═CH | COR |
| 3 | NRCNHNR | S | OH |
| 3 | NRCNHNR | S | COH |
| 3 | NRCNHNR | S | COR |
| 3 | NRCNHNR | CR₃R₂ | Br |
| 3 | NRCNHNR | CR₃R₂ | N₃ |
| 3 | NRCNHNR | SO₂NR | C≡CH |
| 3 | NRCNHNR | SO₂NR | COH |
| 3 | NRCNHNR | NRCNHNR | NHR |
| 3 | NRCNHNR | NRCNHNR | COH |
| 3 | NRCNHNR | NRCNHNR | COR |
| 3 | NRCNHNR | C≡C | OH |
| 3 | NRCNHNR | C≡C | Br |
| 3 | NRCNHNR | C≡C | I |
| 3 | NRCOO | O | COH |
| 3 | NRCOO | O | COR |
| 3 | NRCOO | NR | CONH₂ |
| 3 | NRCOO | NR | CH═CH₂ |
| 3 | NRCOO | NR | COH |
| 3 | NRCOO | NR | COR |
| 3 | NRCOO | CONR | OH |
| 3 | NRCOO | CONR | Cl |
| 3 | NRCOO | CONR | CONH₂ |
| 3 | NRCOO | NRCONR | Cl |
| 3 | NRCOO | NRCONR | N₃ |
| 3 | NRCOO | NRCONR | CONH₂ |
| 3 | NRCOO | NRCONR | CH═CH₂ |
| 3 | NRCOO | NRCOO | Cl |
| 3 | NRCOO | NRCOO | NH₂ |
| 3 | NRCOO | CH═CH | I |
| 3 | NRCOO | CH═CH | F |
| 3 | C≡C | S | CN |
| 3 | C≡C | S | NHR |
| 3 | C≡C | CR₃R₂ | COOH |
| 3 | C≡C | CR₃R₂ | SO₂H |
| 3 | C≡C | CR₃R₂ | CN |
| 3 | C≡C | SO₂NR | Cl |
| 3 | C≡C | SO₂NR | COR |
| 3 | C≡C | NRCNHNR | OH |
| 3 | C≡C | NRCNHNR | F |
| 3 | C≡C | NRCNHNR | NH₂ |
| 3 | C≡C | C≡C | I |
| 3 | C≡C | C≡C | F |
| 3 | C≡C | C≡C | CN |
| 3 | CH═CH | O | F |
| 3 | CH═CH | O | CN |
| 3 | CH═CH | NR | CONH₂ |
| 3 | CH═CH | NR | CH═CH₂ |
| 3 | CH═CH | NR | C≡CH |
| 3 | CH═CH | NR | NH₂ |
| 3 | CH═CH | CONR | C≡CH |
| 3 | CH═CH | CONR | NH₂ |
| 3 | CH═CH | NRCONR | I |
| 3 | CH═CH | NRCONR | F |
| 3 | CH═CH | NRCOO | OH |
| 3 | CH═CH | NRCOO | COOH |
| 3 | CH═CH | NRCOO | SO₂H |
| 3 | CH═CH | CH═CH | OH |
| 3 | CH═CH | CH═CH | COOH |
| 3 | CH═CH | CH═CH | CN |
| 3 | O | S | Cl |
| 3 | O | S | I |
| 3 | O | CR₃R₂ | CONH₂ |
| 3 | O | CR₃R₂ | CH═CH₂ |
| 3 | O | CR₃R₂ | NH₂ |
| 3 | O | SO₂NR | NH₂ |
| 3 | O | SO₂NR | NHR |
| 3 | O | NRCNHNR | N₃ |
| 3 | O | NRCNHNR | CONH₂ |
| 3 | O | C≡C | SH |
| 3 | O | C≡C | F |
| 3 | O | C≡C | N₃ |
| 3 | O | C≡C | C≡CH |
| 3 | O | C≡C | NH₂ |
| 3 | S | O | NH₂ |
| 3 | S | O | COH |
| 3 | S | O | COR |
| 3 | S | NR | OH |
| 3 | S | NR | SH |
| 3 | S | NR | NHR |
| 3 | S | NR | COH |
| 3 | S | CONR | NH₂ |
| 3 | S | NRCONR | SH |
| 3 | S | NRCONR | COOH |
| 3 | S | NRCOO | I |
| 3 | S | NRCOO | CONH₂ |
| 3 | S | NRCOO | COR |
| 3 | S | CH═CH | OH |
| 3 | S | CH═CH | SH |
| 3 | NR | S | CH═CH₂ |
| 3 | NR | S | C≡CH |
| 3 | NR | S | COH |
| 3 | NR | CR₃R₂ | SH |
| 3 | NR | CR₃R₂ | COOH |
| 3 | NR | CR₃R₂ | SO₂H |
| 3 | NR | SO₂NR | NH₂ |
| 3 | NR | SO₂NR | NHR |
| 3 | NR | SO₂NR | COH |
| 3 | NR | NRCNHNR | COOH |
| 3 | NR | NRCNHNR | C≡CH |
| 3 | NR | NRCNHNR | NH₂ |
| 3 | NR | C≡C | OH |
| 3 | NR | C≡C | NHR |
| 3 | CR₃R₂ | O | COOH |
| 3 | CR₃R₂ | O | I |
| 3 | CR₃R₂ | NR | Br |
| 3 | CR₃R₂ | CONR | CH═CH₂ |
| 3 | CR₃R₂ | CONR | C≡CH |
| 3 | CR₃R₂ | NRCONR | NH₂ |
| 3 | CR₃R₂ | NRCONR | NHR |
| 3 | CR₃R₂ | NRCONR | COH |
| 3 | CR₃R₂ | NRCOO | COOH |
| 3 | CR₃R₂ | NRCOO | SO₂H |
| 3 | CR₃R₂ | NRCOO | COH |
| 3 | CR₃R₂ | CH═CH | SO₂H |
| 3 | CR₃R₂ | CH═CH | CN |
| 3 | CONR | S | SO₂H |
| 3 | CONR | S | Cl |
| 3 | CONR | S | Br |
| 3 | CONR | CR₃R₂ | N₃ |
| 3 | CONR | CR₃R₂ | CONH₂ |
| 3 | CONR | CR₃R₂ | CH═CH₂ |
| 3 | CONR | SO₂NR | C≡CH |
| 3 | CONR | SO₂NR | NH₂ |
| 3 | CONR | NRCNHNR | I |
| 3 | CONR | NRCNHNR | N₃ |
| 3 | CONR | C≡C | COH |
| 3 | CONR | C≡C | COR |
| 3 | SO₂NR | O | OH |
| 3 | SO₂NR | O | SH |
| 3 | SO₂NR | NR | SO₂H |
| 3 | SO₂NR | NR | COH |
| 3 | SO₂NR | NR | COR |
| 3 | SO₂NR | CONR | OH |
| 3 | SO₂NR | CONR | SH |
| 3 | SO₂NR | CONR | CONH₂ |
| 3 | SO₂NR | CONR | CH═CH₂ |
| 3 | SO₂NR | NRCONR | SH |
| 3 | SO₂NR | NRCONR | COH |
| 3 | SO₂NR | NRCONR | COR |
| 3 | SO₂NR | NRCOO | OH |
| 3 | SO₂NR | NRCOO | SH |
| 3 | SO₂NR | CH═CH | CH═CH₂ |
| 3 | SO₂NR | CH═CH | NH₂ |
| 3 | SO₂NR | CH═CH | NHR |
| 3 | NRCONR | S | Br |
| 3 | NRCONR | S | I |
| 3 | NRCONR | CR₃R₂ | F |
| 3 | NRCONR | CR₃R₂ | CN |
| 3 | NRCONR | SO₂NR | SO₂H |
| 3 | NRCONR | SO₂NR | Cl |
| 3 | NRCONR | NRCNHNR | SH |
| 3 | NRCONR | NRCNHNR | CONH₂ |
| 3 | NRCONR | NRCNHNR | CH═CH₂ |
| 3 | NRCONR | C≡C | NH₂ |
| 3 | NRCONR | C≡C | COH |
| 3 | NRCNHNR | O | OH |
| 3 | NRCNHNR | O | CONH₂ |
| 3 | NRCNHNR | O | CH═CH₂ |
| 3 | NRCNHNR | NR | SH |
| 3 | NRCNHNR | NR | COOH |
| 3 | NRCNHNR | NR | SO₂H |
| 3 | NRCNHNR | NRCONR | Br |
| 3 | NRCNHNR | NRCONR | C≡CH |
| 3 | NRCNHNR | NRCONR | NH₂ |
| 3 | NRCNHNR | NRCOO | COOH |
| 3 | NRCNHNR | NRCOO | SO₂H |
| 3 | NRCNHNR | CH═CH | Cl |
| 3 | NRCNHNR | CH═CH | Br |
| 3 | NRCOO | S | SH |
| 3 | NRCOO | S | COOH |
| 3 | NRCOO | S | SO₂H |
| 3 | NRCOO | CR₃R₂ | F |
| 3 | NRCOO | CR₃R₂ | CN |
| 3 | NRCOO | CR₃R₂ | COR |
| 3 | NRCOO | SO₂NR | C≡CH |
| 3 | NRCOO | SO₂NR | COH |
| 3 | NRCOO | SO₂NR | COR |
| 3 | NROCO | NRCNHNR | OH |
| 3 | NRCOO | NRCNHNR | COR |
| 3 | NRCOO | C≡C | Br |
| 3 | C≡C | O | CONH₂ |
| 3 | C≡C | O | CH═CH₂ |
| 3 | C≡C | NR | OH |
| 3 | C≡C | CONR | I |
| 3 | C≡C | CONR | F |
| 3 | C≡C | CONR | NH₂ |
| 3 | C≡C | NRCONR | N₃ |
| 3 | C≡C | NRCONR | CONH₂ |
| 3 | C≡C | NRCONR | CH═CH₂ |
| 3 | C≡C | NRCOO | CH═CH₂ |
| 3 | C≡C | NRCOO | C≡CH |
| 3 | C≡C | CH═CH | I |
| 3 | C≡C | CH═CH | C≡CH |
| 3 | C≡C | CH═CH | NH₂ |
| 3 | C≡C | CH═CH | NHR |
| 3 | CH═CH | S | COOH |
| 3 | CH═CH | S | CN |
| 3 | CH═CH | CR₃R₂ | I |
| 3 | CH═CH | CR₃R₂ | F |
| 3 | CH═CH | SO₂NR | CN |
| 3 | CH═CH | SO₂NR | N₃ |
| 3 | CH═CH | SO₂NR | C≡CH |
| 3 | CH═CH | NRCNHNR | NHR |
| 3 | CH═CH | C≡C | Br |
| 3 | CH═CH | C≡C | I |
| 3 | CH═CH | CH═CH | NH₂ |
| 3 | O | S | OH |
| 3 | O | S | SH |
| 3 | O | CR₃R₂ | CH═CH₂ |
| 3 | O | CR₃R₂ | C≡CH |
| 3 | O | CR₃R₂ | NH₂ |
| 3 | O | SO₂NR | Br |
| 3 | O | NRCNHNR | Br |
| 3 | O | NRCNHNR | CONH₂ |
| 3 | O | C≡C | COH |
| 3 | O | C≡C | COR |
| 3 | S | O | CONH₂ |
| 3 | S | O | CH═CH₂ |
| 3 | S | O | C≡CH |
| 3 | S | NR | CONH₂ |
| 3 | S | NR | CH═CH₂ |
| 3 | S | NR | C≡CH |
| 3 | S | NR | NH₂ |
| 3 | S | CONR | N₃ |
| 3 | S | CONR | C≡CH |
| 3 | S | NRCONR | Br |
| 3 | S | NRCONR | NHR |
| 3 | S | NRCONR | COH |
| 3 | S | NRCOO | N₃ |
| 3 | S | NRCOO | COR |
| 3 | S | CH═CH | OH |
| 3 | S | CH═CH | SH |
| 3 | S | CH═CH | Br |
| 3 | NR | S | SH |
| 3 | NR | S | COOH |
| 3 | NR | S | CONH₂ |
| 3 | NR | S | COR |
| 3 | NR | CR₃R₂ | OH |
| 3 | NR | CR₃R₂ | I |
| 3 | NR | CR₃R₂ | F |
| 3 | NR | SO₂NR | F |
| 3 | NR | SO₂NR | CONH₂ |
| 3 | NR | NRCNHNR | Br |
| 3 | NR | NRCNHNR | I |
| 3 | NR | C≡C | CN |
| 3 | NR | C≡C | N₃ |
| 3 | NR | C≡C | CONH₂ |
| 3 | CR₃R₂ | O | Cl |
| 3 | CR₃R₂ | O | Br |
| 3 | CR₃R₂ | NR | COOH |
| 3 | CR₃R₂ | NR | SO₂H |
| 3 | CR₃R₂ | NR | Cl |
| 3 | CR₃R₂ | CONR | COOH |
| 3 | CR₃R₂ | CONR | I |
| 3 | CR₃R₂ | CONR | CH═CH₂ |
| 3 | CR₃R₂ | CONR | C≡CH |
| 3 | CR₃R₂ | NRCONR | F |
| 3 | CR₃R₂ | NRCONR | CH═CH₂ |
| 3 | CR₃R₂ | NRCONR | C≡CH |
| 3 | CR₃R₂ | NRCONR | NH₂ |
| 3 | CR₃R₂ | NRCOO | OH |
| 3 | CR₃R₂ | NRCOO | SH |
| 3 | CR₃R₂ | CH═CH | C≡CH |
| 3 | CR₃R₂ | CH═CH | NH₂ |
| 3 | CONR | SH | SH |
| 3 | CONR | S | COOH |
| 3 | CONR | S | CONH₂ |
| 3 | CONR | CR₃R₂ | I |
| 3 | CONR | CR₃R₂ | F |
| 3 | CONR | SO₂NR | OH |
| 3 | CONR | SO₂NR | SH |
| 3 | CONR | SO₂NR | COOH |
| 3 | CONR | NRCNHNR | NHR |
| 3 | CONR | NRCNHNR | COH |
| 3 | CONR | C≡C | I |
| 3 | CONR | C≡C | F |
| 3 | SO₂NR | O | F |
| 3 | SO₂NR | O | COR |
| 3 | SO₂NR | NR | OH |
| 3 | SO₂NR | NR | SH |
| 3 | SO₂NR | CONR | N₃ |
| 3 | SO₂NR | CONR | CONH₂ |
| 3 | SO₂NR | NRCONR | COOH |
| 3 | SO₂NR | NRCONR | CN |
| 3 | SO₂NR | NRCONR | N₃ |
| 3 | SO₂NR | NRCONR | CONH₂ |
| 3 | SO₂NR | NRCOO | N |
| 3 | SO₂NR | NRCOO | CH═CH₂ |
| 3 | SO₂NR | CH═CH | SO₂H |
| 3 | SO₂NR | CH═CH | Cl |
| 3 | SO₂NR | CH═CH | Br |
| 3 | NRCONR | S | C≡CH |
| 3 | NRCONR | S | NH₂ |
| 3 | NRCONR | CR₃R₂ | Cl |
| 3 | NRCONR | CR₃R₂ | Br |
| 3 | NRCONR | CR₃R₂ | CONH₂ |
| 3 | NRCONR | SO₂NR | OH |
| 3 | NRCONR | SO₂NR | F |
| 3 | NRCONR | SO₂NR | CN |
| 3 | NRCONR | NRCNHNR | CONH₂ |
| 3 | NRCONR | NRCNHNR | CH═CH₂ |
| 3 | NRCONR | C≡C | CONH₂ |
| 3 | NRCONR | C≡C | COH |
| 3 | NRCNHNR | O | SO₂H |
| 3 | NRCNHNR | O | Cl |
| 3 | NRCNHNR | O | F |
| 3 | NRCNHNR | NR | OH |
| 3 | NRCNHNR | NR | Br |
| 3 | NRCNHNR | CONR | OH |
| 3 | NRCNHNR | CONR | SH |
| 3 | NRCNHNR | CONR | CH═CH₂ |
| 3 | NRCNHNR | NRCONR | I |
| 3 | NRCNHNR | NRCONR | NHR |
| 3 | NRCNHNR | NRCONR | COH |
| 3 | NRCNHNR | NRCONR | COR |
| 3 | NRCNHNR | NRCOO | N₃ |
| 3 | NRCNHNR | NRCOO | CONH₂ |
| 3 | NRCNHNR | NRCOO | COR |
| 3 | NRCNHNR | CH═CH | OH |
| 3 | NRCNHNR | CH═CH | COR |
| 3 | NRCOO | S | OH |
| 3 | NRCOO | S | SH |
| 3 | NRCOO | S | COR |
| 3 | NRCOO | CR₃R₂ | OH |
| 3 | NRCOO | CR₃R₂ | SH |
| 3 | NRCOO | CR₃R₂ | COOH |
| 3 | NRCOO | SO₂NR | NH₂ |
| 3 | NRCOO | SO₂NR | NHR |
| 3 | NROCO | NRCNHNR | CH═CH₂ |
| 3 | NRCOO | NRCNHNR | NHR |
| 3 | NRCOO | C≡C | I |
| 3 | C≡C | O | OH |
| 3 | C≡C | O | SH |
| 3 | C≡C | O | COOH |
| 3 | C≡C | NR | C≡CH |
| 3 | C≡C | NR | NH₂ |
| 3 | C≡C | NR | NHR |
| 3 | C≡C | CONR | SO₂H |
| 3 | C≡C | CONR | Cl |
| 3 | C≡C | CONR | Br |
| 3 | C≡C | NRCONR | OH |
| 3 | C≡C | NRCONR | SH |
| 3 | C≡C | NRCONR | Br |
| 3 | C≡C | NRCOO | CONH₂ |
| 3 | C≡C | NRCOO | NHR |
| 3 | C≡C | CH═CH | C≡CH |
| 3 | C≡C | CH═CH | NH₂ |
| 3 | C≡C | CH═CH | COR |
| 3 | CH═CH | S | OH |
| 3 | CH═CH | S | SH |
| 3 | CH═CH | S | COOH |
| 3 | CH═CH | S | SO₂H |
| 3 | CH═CH | S | Cl |
| 3 | CH═CH | CR₃R₂ | OH |
| 3 | CH═CH | CR₃R₂ | COOH |
| 3 | CH═CH | CR₃R₂ | F |
| 3 | CH═CH | SO₂NR | NH₂ |
| 3 | CH═CH | SO₂NR | NHR |
| 3 | CH═CH | SO₂NR | COH |
| 3 | CH═CH | SO₂NR | COR |
| 3 | CH═CH | NRCNHNR | OH |
| 3 | CH═CH | C≡C | CH═CH₂ |
| 3 | CH═CH | C≡C | NHR |
| 3 | CH═CH | CH═CH | COH |
| 3 | CH═CH | CH═CH | COR |
| 3 | O | S | OH |
| 3 | O | S | SH |
| 3 | O | S | COOH |
| 3 | O | CR₃R₂ | CONH₂ |
| 3 | O | CR₃R₂ | CH═CH₂ |
| 3 | O | CR₃R₂ | C≡CH |
| 3 | O | SO₂NR | CONH₂ |
| 3 | O | SO₂NR | CH═CH₂ |
| 3 | O | NRCNHNR | CONH₂ |
| 3 | O | NRCNHNR | CH═CH₂ |
| 3 | O | C≡C | COOH |
| 3 | O | C≡C | SO₂H |
| 3 | O | C≡C | Cl |
| 3 | S | O | SO₂H |
| 3 | S | O | Cl |
| 3 | S | O | COR |
| 3 | S | NR | OH |
| 3 | S | NR | SH |
| 3 | S | NR | COOH |
| 3 | S | NR | SO₂H |
| 3 | S | CONR | CONH₂ |
| 3 | S | CONR | CH═CH₂ |
| 3 | S | CONR | NHR |
| 3 | S | NRCONR | NHR |
| 3 | S | NRCONR | COH |
| 3 | S | NRCONR | COR |
| 3 | S | NRCOO | OH |
| 3 | S | NRCOO | NH₂ |
| 3 | S | NRCOO | NHR |
| 3 | S | CH═CH | I |
| 3 | S | CH═CH | NH₂ |
| 3 | NR | S | SO₂H |
| 3 | NR | S | F |
| 3 | NR | S | CN |
| 3 | NR | S | N₃ |
| 3 | NR | S | NH₂ |
| 3 | NR | CR₃R₂ | SH |
| 3 | NR | CR₃R₂ | COOH |
| 3 | NR | SO₂NR | CN |
| 3 | NR | SO₂NR | COR |
| 3 | NR | NRCNHNR | OH |
| 3 | NR | NRCNHNR | NHR |
| 3 | NR | C≡C | SO₂H |
| 3 | NR | C≡C | C≡CH |
| 3 | NR | C≡C | NH₂ |
| 3 | NR | C≡C | NHR |
| 3 | CR₃R₂ | O | COR |
| 3 | CR₃R₂ | NR | OH |
| 3 | CR₃R₂ | NR | SH |
| 3 | CR₃R₂ | CONR | SO₂H |
| 3 | CR₃R₂ | CONR | Cl |
| 3 | CR₃R₂ | NRCONR | OH |
| 3 | CR₃R₂ | NRCONR | C≡CH |
| 3 | CR₃R₂ | NRCONR | NH₂ |
| 3 | CR₃R₂ | NRCONR | NHR |
| 3 | CR₃R₂ | NRCOO | Cl |
| 3 | CR₃R₂ | NRCOO | COR |
| 3 | CR₃R₂ | CH═CH | Cl |
| 3 | CR₃R₂ | CH═CH | Br |
| 3 | CR₃R₂ | CH═CH | NHR |
| 3 | CONR | S | COR |
| 3 | CONR | CR₃R₂ | OH |
| 3 | CONR | CR₃R₂ | SH |
| 3 | CONR | CR₃R₂ | C≡CH |
| 3 | CONR | SO₂NR | Br |
| 3 | CONR | SO₂NR | I |
| 3 | CONR | SO₂NR | F |
| 3 | CONR | NRCNHNR | OH |
| 3 | CONR | C≡C | COOH |
| 3 | CONR | C≡C | SO₂H |
| 3 | CONR | C≡C | F |
| 3 | SO₂NR | O | Cl |
| 3 | SO₂NR | O | NHR |
| 3 | SO₂NR | NR | OH |
| 3 | SO₂NR | NR | SH |
| 3 | SO₂NR | NR | NH₂ |
| 3 | SO₂NR | NR | NHR |
| 3 | SO₂NR | CONR | Cl |
| 3 | SO₂NR | CONR | Br |
| 3 | SO₂NR | NRCONR | Br |
| 3 | SO₂NR | NRCONR | I |
| 3 | SO₂NR | NRCOO | OH |
| 3 | SO₂NR | NRCOO | SH |
| 3 | SO₂NR | NRCOO | COR |
| 3 | SO₂NR | CH═CH | OH |
| 3 | SO₂NR | CH═CH | CN |
| 3 | NRCONR | S | I |
| 3 | NRCONR | S | COH |
| 3 | NRCONR | S | COR |
| 3 | NRCONR | CR₃R₂ | OH |
| 3 | NRCONR | CR₃R₂ | SH |
| 3 | NRCONR | SO₂NR | OH |
| 3 | NRCONR | SO₂NR | SH |
| 3 | NRCONR | SO₂NR | SO₂H |
| 3 | NRCONR | NRCNHNR | I |
| 3 | NRCONR | NRCNHNR | N₃ |
| 3 | NRCONR | NRCNHNR | CONH₂ |
| 3 | NRCONR | C≡C | SH |
| 3 | NRCONR | C≡C | COOH |
| 3 | NRCNHNR | O | CN |
| 3 | NRCNHNR | O | N₃ |
| 3 | NRCNHNR | O | COR |
| 3 | NRCNHNR | NR | OH |
| 3 | NRCNHNR | NR | COH |
| 3 | NRCNHNR | NR | COR |
| 3 | NRCNHNR | CONR | Br |
| 3 | NRCNHNR | CONR | N₃ |
| 3 | NRCNHNR | NRCONR | C≡CH |
| 3 | NRCNHNR | NRCONR | COH |
| 3 | NRCNHNR | NRCOO | NHR |
| 3 | NRCNHNR | NRCOO | COH |
| 3 | NRCNHNR | NRCOO | COR |
| 3 | NRCNHNR | CH═CH | OH |
| 3 | NRCNHNR | CH═CH | Br |
| 3 | NRCNHNR | CH═CH | I |
| 3 | NRCOO | S | COH |
| 3 | NRCOO | S | COR |
| 3 | NRCOO | CR₃R₂ | CONH₂ |
| 3 | NRCOO | CR₃R₂ | CH═CH₂ |
| 3 | NRCOO | CR₃R₂ | COH |
| 3 | NRCOO | CR₃R₂ | COR |
| 3 | NRCOO | SO₂NR | OH |
| 3 | NRCOO | SO₂NR | Cl |
| 3 | NRCOO | SO₂NR | CONH₂ |
| 3 | NRCOO | NRCNHNR | Cl |
| 3 | NRCOO | NRCNHNR | N₃ |
| 3 | NRCOO | NRCNHNR | CONH₂ |
| 3 | NRCOO | NRCNHNR | CH═CH₂ |
| 3 | NRCOO | C≡C | Cl |
| 3 | NRCOO | C≡C | NH₂ |
| 3 | C≡C | O | I |
| 3 | C≡C | O | F |
| 3 | C≡C | NR | CN |
| 3 | C≡C | NR | NHR |
| 3 | C≡C | CONR | COOH |
| 3 | C≡C | CONR | SO₂H |
| 3 | C≡C | CONR | CN |
| 3 | C≡C | NRCONR | Cl |
| 3 | C≡C | NRCONR | COR |
| 3 | C≡C | NRCOO | OH |
| 3 | C≡C | NRCOO | F |
| 3 | C≡C | NRCOO | NH₂ |
| 3 | C≡C | CH═CH | I |
| 3 | C≡C | CH═CH | F |
| 3 | C≡C | CH═CH | CN |
| 3 | CH═CH | S | F |
| 3 | CH═CH | S | CN |
| 3 | CH═CH | CR₃R₂ | CONH₂ |
| 3 | CH═CH | CR₃R₂ | CH═CH₂ |
| 3 | CH═CH | CR₃R₂ | C≡CH |
| 3 | CH═CH | CR₃R₂ | NH₂ |
| 3 | CH═CH | SO₂NR | C≡CH |
| 3 | CH═CH | SO₂NR | NH₂ |
| 3 | CH═CH | NRCNHNR | I |
| 3 | CH═CH | NRCNHNR | F |
| 3 | CH═CH | C≡C | OH |
| 3 | CH═CH | C≡C | COOH |
| 3 | CH═CH | C≡C | SO₂H |
| 3 | CH═CH | CH═CH | N₃ |
| 3 | CH═CH | CH═CH | CH═CH₂ |
| 4 | O | O | OH |
| 4 | O | O | SH |
| 4 | O | O | CONH₂ |
| 4 | O | NR | SH |
| 4 | O | NR | Cl |
| 4 | O | NR | NHR |
| 4 | O | CONR | F |
| 4 | O | CONR | CH═CH₂ |
| 4 | O | CONR | COR |
| 4 | O | NRCONR | OH |
| 4 | O | NRCONR | NHR |
| 4 | O | NRCOO | CN |
| 4 | O | NRCOO | NHR |
| 4 | O | CH═CH | Br |
| 4 | O | CH═CH | C≡CH |
| 4 | O | CH═CH | NH₂ |
| 4 | S | S | Br |
| 4 | S | S | N₃ |
| 4 | S | S | NH₂ |
| 4 | S | S | NHR |
| 4 | S | CR₄R₂ | OH |
| 4 | S | CR₄R₂ | COR |
| 4 | S | SO₂NR | COOH |
| 4 | S | SO₂NR | I |
| 4 | S | SO₂NR | F |
| 4 | S | SO₂NR | COR |
| 4 | S | NRCNHNR | OH |
| 4 | S | NRCNHNR | I |
| 4 | S | NRCNHNR | F |
| 4 | S | C≡C | SH |
| 4 | NR | O | OH |
| 4 | NR | O | SH |
| 4 | NR | O | NH₂ |
| 4 | NR | NR | SO₂H |
| 4 | NR | NR | Cl |
| 4 | NR | NR | NHR |
| 4 | NR | NR | COR |
| 4 | NR | CONR | OH |
| 4 | NR | CONR | NH₂ |
| 4 | NR | CONR | NHR |
| 4 | NR | NRCONR | I |
| 4 | NR | NRCONR | F |
| 4 | NR | NRCOO | OH |
| 4 | NR | NRCOO | CONH₂ |
| 4 | NR | CH═CH | NH₂ |
| 4 | NR | CH═CH | NHR |
| 4 | NR | CH═CH | COR |
| 4 | CR₄R₂ | S | OH |
| 4 | CR₄R₂ | S | Br |
| 4 | CR₄R₂ | CR₄R₂ | SO₂H |
| 4 | CR₄R₂ | CR₄R₂ | CH═CH₂ |
| 4 | CR₄R₂ | CR₄R₂ | C≡CH |
| 4 | CR₄R₂ | SO₂NR | F |
| 4 | CR₄R₂ | SO₂NR | CN |
| 4 | CR₄R₂ | SO₂NR | N₃ |
| 4 | CR₄R₂ | NRCNHNR | CONH₂ |
| 4 | CR₄R₂ | NRCNHNR | CH═CH₂ |
| 4 | CR₄R₂ | NRCNHNR | C≡CH |
| 4 | CR₄R₂ | C≡C | Cl |
| 4 | CR₄R₂ | C≡C | Br |
| 4 | CR₄R₂ | C≡C | I |
| 4 | CONR | O | COH |
| 4 | CONR | O | COR |
| 4 | CONR | NR | OH |
| 4 | CONR | NR | Br |
| 4 | CONR | NR | N₃ |
| 4 | CONR | CONR | Br |
| 4 | CONR | CONR | N₃ |
| 4 | CONR | CONR | C≡CH |
| 4 | CONR | NRCONR | OH |
| 4 | CONR | NRCONR | SH |
| 4 | CONR | NRCONR | COH |
| 4 | CONR | NRCOO | F |
| 4 | CONR | NRCOO | CN |
| 4 | CONR | NRCOO | COR |
| 4 | CONR | CH═CH | OH |
| 4 | CONR | CH═CH | CN |
| 4 | CONR | CH═CH | COR |
| 4 | SO₂NR | S | OH |
| 4 | SO₂NR | S | SH |
| 4 | SO₂NR | CR₄R₂ | N₃ |
| 4 | SO₂NR | CR₄R₂ | NHR |
| 4 | SO₂NR | CR₄R₂ | COH |
| 4 | SO₂NR | SO₂NR | COOH |
| 4 | SO₂NR | SO₂NR | NHR |
| 4 | SO₂NR | SO₂NR | COH |
| 4 | SO₂NR | NRCNHNR | SH |
| 4 | SO₂NR | NRCNHNR | COOH |
| 4 | SO₂NR | NRCNHNR | SO₂H |
| 4 | SO₂NR | NRCNHNR | Cl |
| 4 | SO₂NR | C≡C | I |
| 4 | SO₂NR | C≡C | F |
| 4 | SO₂NR | C≡C | CN |
| 4 | NRCONR | O | F |
| 4 | NRCONR | O | CN |
| 4 | NRCONR | O | N₃ |
| 4 | NRCONR | NR | CONH₂ |
| 4 | NRCONR | NR | CH═CH₂ |
| 4 | NRCONR | NR | C≡CH |
| 4 | NRCONR | CONR | SH |
| 4 | NRCONR | CONR | COOH |
| 4 | NRCONR | NRCONR | CH═CH₂ |
| 4 | NRCONR | NRCOO | SH |
| 4 | NRCONR | NRCOO | COOH |
| 4 | NRCONR | CH═CH | SO₂H |
| 4 | NRCONR | CH═CH | Cl |
| 4 | NRCNHNR | S | Br |
| 4 | NRCNHNR | S | I |
| 4 | NRCNHNR | CR₄R₂ | N₃ |
| 4 | NRCNHNR | CR₄R₂ | CONH₂ |
| 4 | NRCNHNR | SO₂NR | SO₂H |
| 4 | NRCNHNR | SO₂NR | Cl |
| 4 | NRCNHNR | SO₂NR | Br |
| 4 | NRCNHNR | NRCNHNR | COR |
| 4 | NRCNHNR | C≡C | Br |
| 4 | NRCOO | O | COH |
| 4 | NRCOO | O | COR |
| 4 | NRCOO | NR | OH |
| 4 | NRCOO | NR | COH |
| 4 | NRCOO | NR | COR |
| 4 | NRCOO | CONR | OH |
| 4 | NRCOO | CONR | SH |
| 4 | NRCOO | NRCONR | NH₂ |
| 4 | NRCOO | NRCOO | SH |
| 4 | NRCOO | NRCOO | COOH |
| 4 | NRCOO | CH═CH | COH |
| 4 | NRCOO | CH═CH | COR |
| 4 | C≡C | S | OH |
| 4 | C≡C | CR₄R₂ | COOH |
| 4 | C≡C | CR₄R₂ | SO₂H |
| 4 | C≡C | SO₂NR | SO₂H |
| 4 | C≡C | SO₂NR | COR |
| 4 | C≡C | NRCNHNR | OH |
| 4 | C≡C | NRCNHNR | SH |
| 4 | C≡C | C≡C | CONH₂ |
| 4 | C≡C | C≡C | COR |
| 4 | CH═CH | O | OH |
| 4 | CH═CH | O | NH₂ |
| 4 | CH═CH | O | COR |
| 4 | CH═CH | NR | OH |
| 4 | CH═CH | NR | COH |
| 4 | CH═CH | CONR | OH |
| 4 | CH═CH | CONR | CH═CH₂ |
| 4 | CH═CH | CONR | C≡CH |
| 4 | CH═CH | CONR | NH₂ |
| 4 | CH═CH | NRCONR | C≡CH |
| 4 | CH═CH | NRCONR | NH₂ |
| 4 | CH═CH | NRCOO | I |
| 4 | CH═CH | NRCOO | C≡CH |
| 4 | CH═CH | CH═CH | OH |
| 4 | CH═CH | CH═CH | SH |
| 4 | CH═CH | CH═CH | Br |
| 4 | O | S | OH |
| 4 | O | S | SH |
| 4 | O | S | CONH₂ |
| 4 | O | CR₄R₂ | SH |
| 4 | O | CR₄R₂ | Cl |
| 4 | O | CR₄R₂ | NHR |
| 4 | O | SO₂NR | F |
| 4 | O | SO₂NR | CH═CH₂ |
| 4 | O | SO₂NR | COR |
| 4 | O | NRCNHNR | OH |
| 4 | O | NRCNHNR | NHR |
| 4 | O | C≡C | CN |
| 4 | O | C≡C | NHR |
| 4 | S | O | Br |
| 4 | S | O | C≡CH |
| 4 | S | O | NH₂ |
| 4 | S | NR | Br |
| 4 | S | NR | N₃ |
| 4 | S | NR | NH₂ |
| 4 | S | NR | NHR |
| 4 | S | CONR | OH |
| 4 | S | CONR | COR |
| 4 | S | NRCONR | COOH |
| 4 | S | NRCONR | I |
| 4 | S | NRCONR | F |
| 4 | S | NRCONR | COR |
| 4 | S | NRCOO | OH |
| 4 | S | NRCOO | I |
| 4 | S | NRCOO | F |
| 4 | S | CH═CH | SH |
| 4 | NR | S | OH |
| 4 | NR | S | SH |
| 4 | NR | S | NH₂ |
| 4 | NR | CR₄R₂ | SO₂H |
| 4 | NR | CR₄R₂ | Cl |
| 4 | NR | CR₄R₂ | NHR |
| 4 | NR | CR₄R₂ | COR |
| 4 | NR | SO₂NR | OH |
| 4 | NR | SO₂NR | NH₂ |
| 4 | NR | SO₂NR | NHR |
| 4 | NR | NRCNHNR | I |
| 4 | NR | NRCNHNR | F |
| 4 | NR | C≡C | OH |
| 4 | NR | C≡C | CONH₂ |
| 4 | CR₄R₂ | OO | NH₂ |
| 4 | CR₄R₂ | O | NHR |
| 4 | CR₄R₂ | O | COR |
| 4 | CR₄R₂ | NR | OH |
| 4 | CR₄R₂ | NR | Br |
| 4 | CR₄R₂ | CONR | SO₂H |
| 4 | CR₄R₂ | CONR | CH═CH₂ |
| 4 | CR₄R₂ | CONR | C≡CH |
| 4 | CR₄R₂ | NRCONR | F |
| 4 | CR₄R₂ | NRCONR | CN |
| 4 | CR₄R₂ | NRCONR | N₃ |
| 4 | CR₄R₂ | NRCOO | CONH₂ |
| 4 | CR₄R₂ | NRCOO | CH═CH₂ |
| 4 | CR₄R₂ | NRCOO | C≡CH |
| 4 | CR₄R₂ | CH═CH | Cl |
| 4 | CR₄R₂ | CH═CH | Br |
| 4 | CR₄R₂ | CH═CH | I |
| 4 | CONR | S | COH |
| 4 | CONR | S | COR |
| 4 | CONR | CR₄R₂ | OH |
| 4 | CONR | CR₄R₂ | Br |
| 4 | CONR | CR₄R₂ | N₃ |
| 4 | CONR | SO₂NR | Br |
| 4 | CONR | SO₂NR | N₃ |
| 4 | CONR | SO₂NR | C≡CH |
| 4 | CONR | NRCNHNR | OH |
| 4 | CONR | NRCNHNR | SH |
| 4 | CONR | NRCNHNR | COH |
| 4 | CONR | C≡C | F |
| 4 | CONR | C≡C | CN |
| 4 | CONR | C≡C | COR |
| 4 | SO₂NR | O | OH |
| 4 | SO₂NR | O | CN |
| 4 | SO₂NR | O | COR |
| 4 | SO₂NR | NR | OH |
| 4 | SO₂NR | NR | SH |
| 4 | SO₂NR | CONR | N₃ |
| 4 | SO₂NR | CONR | NHR |
| 4 | SO₂NR | CONR | COH |
| 4 | SO₂NR | NRCONR | COOH |
| 4 | SO₂NR | NRCONR | NHR |
| 4 | SO₂NR | NRCONR | COH |
| 4 | SO₂NR | NRCOO | SH |
| 4 | SO₂NR | NRCOO | COOH |
| 4 | SO₂NR | NRCOO | SO₂H |
| 4 | SO₂NR | NRCOO | Cl |
| 4 | SO₂NR | CH═CH | I |
| 4 | SO₂NR | CH═CH | F |
| 4 | SO₂NR | CH═CH | CN |
| 4 | NRCONR | S | F |
| 4 | NRCONR | S | CN |
| 4 | NRCONR | S | N₃ |
| 4 | NRCONR | CR₄R₂ | CONH₂ |
| 4 | NRCONR | CR₄R₂ | CH═CH₂ |
| 4 | NRCONR | CR₄R₂ | C≡CH |
| 4 | NRCONR | SO₂NR | SH |
| 4 | NRCONR | SO₂NR | COOH |
| 4 | NRCONR | NRCNHNR | CH═CH₂ |
| 4 | NRCONR | C≡C | SH |
| 4 | NRCONR | C≡C | COOH |
| 4 | NRCNHNR | O | SO₂H |
| 4 | NRCNHNR | O | Cl |
| 4 | NRCNHNR | NR | Br |
| 4 | NRCNHNR | NR | I |
| 4 | NRCNHNR | CONR | N₃ |
| 4 | NRCNHNR | CONR | CONH₂ |
| 4 | NRCNHNR | NRCONR | SO₂H |
| 4 | NRCNHNR | NRCONR | Cl |
| 4 | NRCNHNR | NRCONR | Br |
| 4 | NRCNHNR | NRCOO | COR |
| 4 | NRCNHNR | CH═CH | Br |
| 4 | NRCOO | S | COH |
| 4 | NRCOO | S | COR |
| 4 | NRCOO | CR₄R₂ | OH |
| 4 | NRCOO | CR₄R₂ | COH |
| 4 | NRCOO | CR₄R₂ | COR |
| 4 | NRCOO | SO₂NR | OH |
| 4 | NRCOO | SO₂NR | SH |
| 4 | NRCOO | NRCNHNR | NH₂ |
| 4 | NRCOO | C≡C | SH |
| 4 | NRCOO | C≡C | COO |
| 4 | C≡C | O | COH |
| 4 | C≡C | O | COR |
| 4 | C≡C | NR | OH |
| 4 | C≡C | CONR | COOH |
| 4 | C≡C | CONR | SO₂H |
| 4 | C≡C | NRCONR | SO₂H |
| 4 | C≡C | NRCONR | COR |
| 4 | C≡C | NRCOO | OH |
| 4 | C≡C | NRCOO | SH |
| 4 | C≡C | CH═CH | CONH₂ |
| 4 | C≡C | CH═CH | COR |
| 4 | CH═CH | S | OH |
| 4 | CH═CH | S | NH₂ |
| 4 | CH═CH | S | COR |
| 4 | CH═CH | CR₄R₂ | OH |
| 4 | CH═CH | CR₄R₂ | COH |
| 4 | CH═CH | SO₂NR | OH |
| 4 | CH═CH | SO₂NR | CH═CH₂ |
| 4 | CH═CH | SO₂NR | C≡CH |
| 4 | CH═CH | SO₂NR | NH₂ |
| 4 | CH═CH | NRCNHNR | C≡CH |
| 4 | CH═CH | NRCNHNR | NH₂ |
| 4 | CH═CH | C≡C | I |
| 4 | CH═CH | C≡C | C≡CH |
| 4 | CH═CH | CH═CH | N₃ |
| 4 | CH═CH | CH═CH | CONH₂ |
| 4 | CH═CH | CH═CH | NHR |
| 5 | O | O | CN |
| 5 | O | O | N₃ |
| 5 | O | NH | Br |
| 5 | O | NR | I |
| 5 | O | CONR | CONH₂ |
| 5 | O | CONR | CH═CH₂ |
| 5 | O | NRCONR | NHR |
| 5 | O | NRCONR | COH |
| 5 | O | NRCOO | OH |
| 5 | O | NRCOO | COOH |
| 5 | O | CH═CH | OH |
| 5 | O | CH═CH | C≡CH |
| 5 | S | S | Cl |
| 5 | S | S | Br |
| 5 | S | S | I |
| 5 | S | S | NH₂ |
| 5 | S | CR₅R₂ | COOH |
| 5 | S | CR₅R₂ | NHR |
| 5 | S | CR₅R₂ | COH |
| 5 | S | CR₅R₂ | COR |
| 5 | S | SO₂NR | Cl |
| 5 | S | SO₂NR | CN |
| 5 | S | SO₂NR | N₃ |
| 5 | S | SO₂NR | COR |
| 5 | S | NRCNHNR | OH |
| 5 | S | NRCNHNR | COR |
| 5 | S | C≡C | OH |
| 5 | S | C≡C | SH |
| 5 | NR | O | SH |
| 5 | NR | O | COOH |
| 5 | NR | O | SO₂H |
| 5 | NR | NR | OH |
| 5 | NR | NR | SH |
| 5 | NR | CONR | OH |
| 5 | NR | CONR | COR |
| 5 | NR | NRCONR | OH |
| 5 | NR | NRCONR | SH |
| 5 | NR | NRCOO | NH₂ |
| 5 | NR | NRCOO | NHR |
| 5 | NR | CH═CH | COOH |
| 5 | NR | CH═CH | SO₂H |
| 5 | CR₅R₂ | S | SO₂H |
| 5 | CR₅R₂ | S | NH₂ |
| 5 | CR₅R₂ | S | NHR |
| 5 | CR₅R₂ | S | COH |
| 5 | CR₅R₂ | CR₅R₂ | COOH |
| 5 | CR₅R₂ | CR₅R₂ | F |
| 5 | CR₅R₂ | SO₂NR | NH₂ |
| 5 | CR₅R₂ | SO₂NR | NHR |
| 5 | CR₅R₂ | SO₂NR | COH |
| 5 | CR₅R₂ | NRCNHNR | COH |
| 5 | CR₅R₂ | NRCNHNR | COR |
| 5 | CR₅R₂ | C≡C | OH |
| 5 | CR₅R₂ | C≡C | Cl |
| 5 | CONR | O | N₃ |
| 5 | CONR | O | COH |
| 5 | CONR | O | COR |
| 5 | CONR | NR | OH |
| 5 | CONR | NR | NHR |
| 5 | CONR | CONR | COOH |
| 5 | CONR | CONR | NHR |
| 5 | CONR | NRCONR | F |
| 5 | CONR | NRCONR | CN |
| 5 | CONR | NRCOO | OH |
| 5 | CONR | NRCOO | COH |
| 5 | CONR | CH═CH | I |
| 5 | CONR | CH═CH | F |
| 5 | CONR | CH═CH | COR |
| 5 | SO₂NR | S | OH |
| 5 | SO₂NR | S | SO₂H |
| 5 | SO₂NR | S | Cl |
| 5 | SO₂NR | CR₅R₂ | F |
| 5 | SO₂NR | CR₅R₂ | NHR |
| 5 | SO₂NR | SO₂NR | COOH |
| 5 | SO₂NR | SO₂NR | SO₂H |
| 5 | SO₂NR | SO₂NR | Cl |
| 5 | SO₂NR | SO₂NR | Br |
| 5 | SO₂NR | NRCNHNR | NH₂ |
| 5 | SO₂NR | NRCNHNR | NHR |
| 5 | SO₂NR | C≡C | COOH |
| 5 | SO₂NR | C≡C | COH |
| 5 | SO₂NR | C≡C | COR |
| 5 | NRCONR | O | OH |
| 5 | NRCONR | O | SH |
| 5 | NRCONR | O | COOH |
| 5 | NRCONR | O | CONH₂ |
| 5 | NRCONR | NR | CN |
| 5 | NRCONR | NR | NHR |
| 5 | NRCONR | NR | COH |
| 5 | NRCONR | CONR | CONH₂ |
| 5 | NRCONR | CONR | COH |
| 5 | NRCONR | CONR | COR |
| 5 | NRCONR | NRCONR | OH |
| 5 | NRCONR | NRCONR | SH |
| 5 | NRCONR | NRCONR | COOH |
| 5 | NRCONR | NRCOO | F |
| 5 | NRCONR | NRCOO | CN |
| 5 | NRCONR | CH═CH | Cl |
| 5 | NRCONR | CH═CH | Br |
| 5 | NRCONR | CH═CH | NH₂ |
| 5 | NRCNHNR | S | CONH₂ |
| 5 | NRCNHNR | S | CH═CH₂ |
| 5 | NRCNHNR | S | C≡CH |
| 5 | NRCNHNR | S | NH₂ |
| 5 | NRCNHNR | S | NHR |
| 5 | NRCNHNR | S | COH |
| 5 | NRCNHNR | CR₅R₂ | SO₂H |
| 5 | NRCNHNR | CR₅R₂ | Cl |
| 5 | NRCNHNR | SO₂NR | SO₂H |
| 5 | NRCNHNR | SO₂NR | Cl |
| 5 | NRCNHNR | SO₂NR | Br |
| 5 | NRCNHNR | SO₂NR | I |
| 5 | NRCNHNR | SO₂NR | F |
| 5 | NRCNHNR | SO₂NR | CN |
| 5 | NRCNHNR | NRCNHNR | NH₂ |
| 5 | NRCNHNR | NRCNHNR | NHR |
| 5 | NRCNHNR | NRCNHNR | COH |
| 5 | NRCNHNR | NRCNHNR | COR |
| 5 | NRCNHNR | C≡C | OH |
| 5 | NRCNHNR | C≡C | SH |
| 5 | NRCNHNR | C≡C | I |
| 5 | NRCNHNR | C≡C | NHR |
| 5 | NRCOO | O | COOH |
| 5 | NRCOO | O | SO₂H |
| 5 | NRCOO | O | NHR |
| 5 | NRCOO | O | COH |
| 5 | NRCOO | O | COR |
| 5 | NRCOO | NR | OH |
| 5 | NRCOO | NR | SH |
| 5 | NRCOO | NR | COOH |
| 5 | NRCOO | NR | SO₂H |
| 5 | NRCOO | CONR | NHR |
| 5 | NRCOO | CONR | COH |
| 5 | NRCOO | CONR | COR |
| 5 | NRCOO | NRCONR | OH |
| 5 | NRCOO | NRCONR | SH |
| 5 | NRCOO | NRCONR | COOH |
| 5 | NRCOO | NRCONR | COR |
| 5 | NRCOO | NRCOO | OH |
| 5 | NRCOO | NRCOO | SH |
| 5 | NRCOO | NRCOO | COH |
| 5 | NRCOO | NRCOO | COR |
| 5 | NRCOO | CH═CH | N₃ |
| 5 | NRCOO | CH═CH | CONH₂ |
| 5 | NRCOO | CH═CH | COH |
| 5 | NRCOO | CH═CH | COR |
| 5 | C≡C | S | OH |
| 5 | C≡C | S | SH |
| 5 | C≡C | S | COOH |
| 5 | C≡C | S | NH₂ |
| 5 | C≡C | CR₅R₂ | SH |
| 5 | C≡C | CR₅R₂ | SO₂H |
| 5 | C≡C | CR₅R₂ | N₃ |
| 5 | C≡C | CR₅R₂ | COR |
| 5 | C≡C | SO₂NR | NHR |
| 5 | C≡C | SO₂NR | COH |
| 5 | C≡C | SO₂NR | COR |
| 5 | C≡C | NRCNHNR | CN |
| 5 | C≡C | NRCNHNR | CH═CH₂ |
| 5 | C≡C | NRCNHNR | C≡CH |
| 5 | C≡C | C≡C | COOH |
| 5 | CH═CH | O | OH |
| 5 | CH═CH | O | C≡CH |
| 5 | CH═CH | O | NH₂ |
| 5 | CH═CH | O | NHR |
| 5 | CH═CH | NR | NHR |
| 5 | CH═CH | NR | COH |
| 5 | CH═CH | NR | COR |
| 5 | CH═CH | CONR | Br |
| 5 | CH═CH | CONR | COR |
| 5 | CH═CH | NRCONR | Br |
| 5 | CH═CH | NRCOO | OH |
| 5 | CH═CH | CH═CH | COOH |
| 5 | CH═CH | CH═CH | SO₂H |
| 5 | O | S | CN |
| 5 | O | S | N₃ |
| 5 | O | CR₅R₂ | Br |
| 5 | O | CR₅R₂ | I |
| 5 | O | SO₂NR | CONH₂ |
| 5 | O | SO₂NR | CH═CH₂ |
| 5 | O | NRCNHNR | NHR |
| 5 | O | NRCNHNR | COH |
| 5 | O | C≡C | OH |
| 5 | O | C≡C | COOH |
| 5 | S | O | OH |
| 5 | S | O | C≡CH |
| 5 | S | NR | Cl |
| 5 | S | NR | Br |
| 5 | S | NR | I |
| 5 | S | NR | NH₂ |
| 5 | S | CONR | COOH |
| 5 | S | CONR | NHR |
| 5 | S | CONR | COH |
| 5 | S | CONR | COR |
| 5 | S | NRCONR | Cl |
| 5 | S | NRCONR | CN |
| 5 | S | NRCONR | N₃ |
| 5 | S | NRCONR | COR |
| 5 | S | NRCOO | OH |
| 5 | S | NRCOO | COR |
| 5 | S | CH═CH | OH |
| 5 | S | CH═CH | SH |
| 5 | NR | S | SH |
| 5 | NR | S | COOH |
| 5 | NR | S | SO₂H |
| 5 | NR | CR₅R₂ | OH |
| 5 | NR | CR₅R₂ | SH |
| 5 | NR | SO₂NR | OH |
| 5 | NR | SO₂NR | COR |
| 5 | NR | NRCNHNR | OH |
| 5 | NR | NRCNHNR | SH |
| 5 | NR | C≡C | NH₂ |
| 5 | NR | C≡C | NHR |
| 5 | CR₅R₂ | O | COOH |
| 5 | CR₅R₂ | O | SO₂H |
| 5 | CR₅R₂ | NR | SO₂H |
| 5 | CR₅R₂ | NR | NH₂ |
| 5 | CR₅R₂ | NR | NHR |
| 5 | CR₅R₂ | NR | COH |
| 5 | CR₅R₂ | CONR | COOH |
| 5 | CR₅R₂ | CONR | F |
| 5 | CR₅R₂ | NRCONR | NH₂ |
| 5 | CR₅R₂ | NRCONR | NHR |
| 5 | CR₅R₂ | NRCONR | COH |
| 5 | CR₅R₂ | NRCOO | COH |
| 5 | CR₅R₂ | NRCOO | COR |
| 5 | CR₅R₂ | CH═CH | OH |
| 5 | CR₅R₂ | CH═CH | Cl |
| 5 | CONR | S | N₃ |
| 5 | CONR | S | COH |
| 5 | CONR | S | COR |
| 5 | CONR | CR₅R₂ | OH |
| 5 | CONR | CR₅R₂ | NHR |
| 5 | CONR | SO₂NR | COOH |
| 5 | CONR | SO₂NR | NHR |
| 5 | CONR | NRCNHNR | F |
| 5 | CONR | NRCNHNR | CN |
| 5 | CONR | C≡C | OH |
| 5 | CONR | C≡C | COH |
| 5 | SO₂NR | O | I |
| 5 | SO₂NR | O | F |
| 5 | SO₂NR | O | COR |
| 5 | SO₂NR | NR | OH |
| 5 | SO₂NR | NR | SO₂H |
| 5 | SO₂NR | NR | Cl |
| 5 | SO₂NR | CONR | F |
| 5 | SO₂NR | CONR | NHR |
| 5 | SO₂NR | NRCONR | COOH |
| 5 | SO₂NR | NRCONR | SO₂H |
| 5 | SO₂NR | NRCONR | Cl |
| 5 | SO₂NR | NRCONR | Br |
| 5 | SO₂NR | NRCOO | NH₂ |
| 5 | SO₂NR | NRCOO | NHR |
| 5 | SO₂NR | CH═CH | COOH |
| 5 | SO₂NR | CH═CH | COH |
| 5 | SO₂NR | CH═CH | COR |
| 5 | NRCONR | S | OH |
| 5 | NRCONR | S | SH |
| 5 | NRCONR | S | COOH |
| 5 | NRCONR | S | CONH₂ |
| 5 | NRCONR | CR₅R₂ | CN |
| 5 | NRCONR | CR₅R₂ | NHR |
| 5 | NRCONR | CR₅R₂ | COH |
| 5 | NRCONR | SO₂NR | CONH₂ |
| 5 | NRCONR | SO₂NR | COH |
| 5 | NRCONR | SO₂NR | COR |
| 5 | NRCONR | NRCNHNR | OH |
| 5 | NRCONR | NRCNHNR | SH |
| 5 | NRCONR | NRCNHNR | COOH |
| 5 | NRCONR | C≡C | F |
| 5 | NRCONR | C≡C | CN |
| 5 | NRCNHNR | O | Cl |
| 5 | NRCNHNR | O | Br |
| 5 | NRCNHNR | OO | NH₂ |
| 5 | NRCNHNR | NR | CONH₂ |
| 5 | NRCNHNR | NR | CH═CH₂ |
| 5 | NRCNHNR | NR | C≡CH |
| 5 | NRCNHNR | NR | NH₂ |
| 5 | NRCNHNR | NR | NHR |
| 5 | NRCNHNR | NR | COH |
| 5 | NRCNHNR | CONR | SO₂H |
| 5 | NRCNHNR | CONR | Cl |
| 5 | NRCNHNR | NRCONR | SO₂H |
| 5 | NRCNHNR | NRCONR | Cl |
| 5 | NRCNHNR | NRCONR | Br |
| 5 | NRCNHNR | NRCONR | I |
| 5 | NRCNHNR | NRCONR | F |
| 5 | NRCNHNR | NRCONR | CN |
| 5 | NRCNHNR | NRCOO | NH₂ |
| 5 | NRCNHNR | NRCOO | NHR |
| 5 | NRCNHNR | NRCOO | COH |
| 5 | NRCNHNR | NRCOO | COR |
| 5 | NRCNHNR | CH═CH | OH |
| 5 | NRCNHNR | CH═CH | SH |
| 5 | NRCHNHR | CH═CH | I |
| 5 | NRCNHNR | CH═CH | NHR |
| 5 | NRCOO | S | COOH |
| 5 | NRCOO | S | SO₂H |
| 5 | NRCOO | S | NHR |
| 5 | NRCOO | S | COH |
| 5 | NRCOO | S | COR |
| 5 | NRCOO | CR₅R₂ | OH |
| 5 | NRCOO | CR₅R₂ | SH |
| 5 | NRCOO | CR₅R₂ | COOH |
| 5 | NRCOO | CR₅R₂ | SO₂H |
| 5 | NRCOO | SO₂NR | NHR |
| 5 | NRCOO | SO₂NR | COH |
| 5 | NRCOO | SO₂NR | COR |
| 5 | NRCOO | NRCNHNR | OH |
| 5 | NRCOO | NRCNHNR | SH |
| 5 | NRCOO | NRCNHNR | COOH |
| 5 | NRCOO | NRCNHNR | COR |
| 5 | NRCOO | C≡C | OH |
| 5 | NRCOO | C≡C | SH |
| 5 | NRCOO | C≡C | COH |
| 5 | NRCOO | C≡C | COR |
| 5 | C≡C | O | N₃ |
| 5 | C≡C | O | CONH₂ |
| 5 | C≡C | O | COH |
| 5 | C≡C | O | COR |
| 5 | C≡C | NR | OH |
| 5 | C≡C | NR | SH |
| 5 | C≡C | NR | COOH |
| 5 | C≡C | NR | NH₂ |
| 5 | C≡C | CONR | SH |
| 5 | C≡C | CONR | SO₂H |
| 5 | C≡C | CONR | N₃ |
| 5 | C≡C | CONR | COR |
| 5 | C≡C | NRCONR | NHR |
| 5 | C≡C | NRCONR | COH |
| 5 | C≡C | NRCONR | COR |
| 5 | C≡C | NRCOO | CN |
| 5 | C≡C | NRCOO | CH═CH₂ |
| 5 | C≡C | NRCOO | C≡CH |
| 5 | C≡C | CH═CH | COOH |
| 5 | CH═CH | S | OH |
| 5 | CH═CH | S | C≡CH |
| 5 | CH═CH | S | NH₂ |
| 5 | CH═CH | S | NHR |
| 5 | CH═CH | CR₅R₂ | NHR |
| 5 | CH═CH | CR₅R₂ | COH |
| 5 | CH═CH | CR₅R₂ | COR |
| 5 | CH═CH | SO₂NR | Br |
| 5 | CH═CH | SO₂NR | COR |
| 5 | CH═CH | NRCNHNR | Br |
| 5 | CH═CH | C≡C | OH |
| 5 | CH═CH | CH═CH | CH═CH₂ |
| 5 | CH═CH | CH═CH | C≡CH |

R₁, and R₂=hydrogen, alkyl, alkenyl, alkynyl, aryl, and terocyclic

TABLE 7

| | |
|---|---|
| 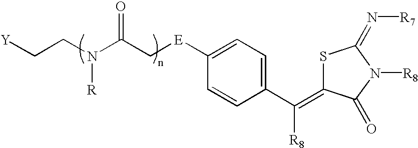 | 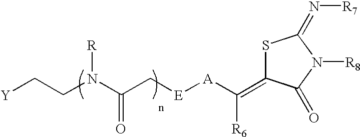 |
| 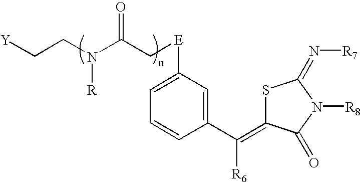 | 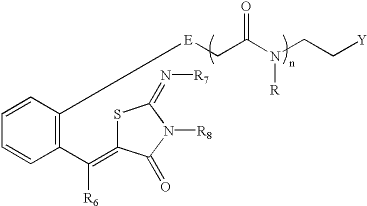 |
| 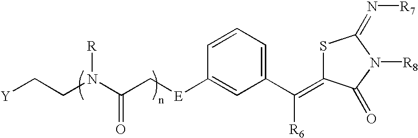 | 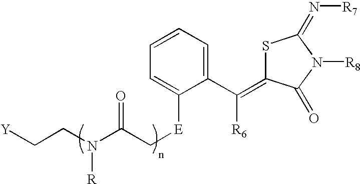 |
| 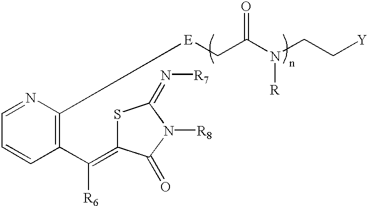 | 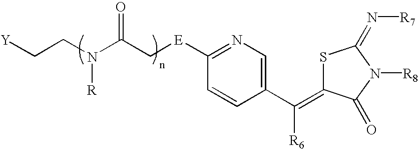 |
| 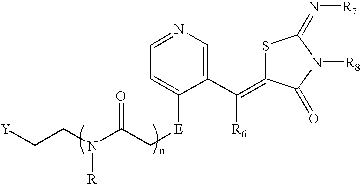 | 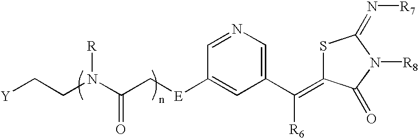 |

The variables E, Y, and n can have the values provided in Table 5 above. R in the compounds is alkyls, alkenyl, alkynyl, aromatic, or heterocyclic.

TABLE 8

| | |
|---|---|
| 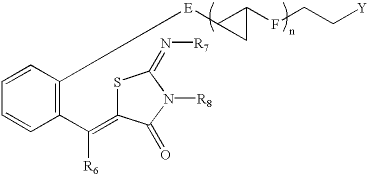 | 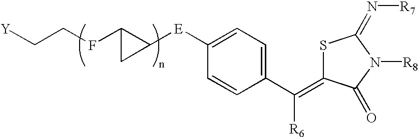 |

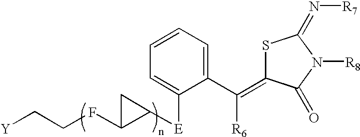

| | |
|---|---|
| 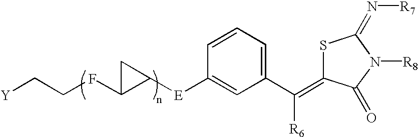 | 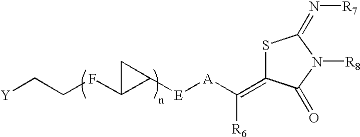 |
| 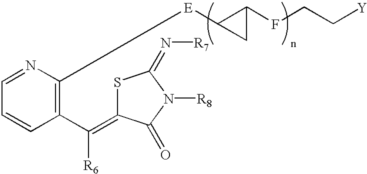 | 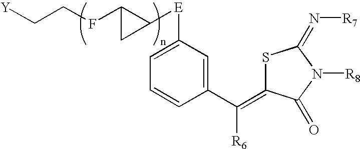 |

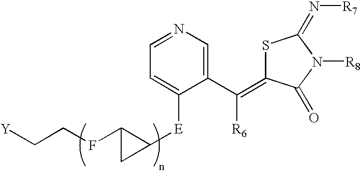

| | |
|---|---|
| 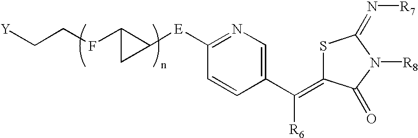 | 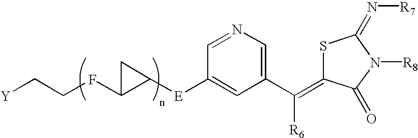 |

The variables E, F, Y, and n can have the values provided in Table 6 above.

TABLE 9

| | |
|---|---|
| 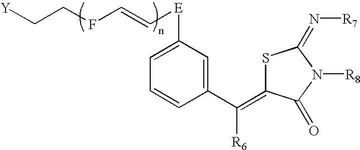 | 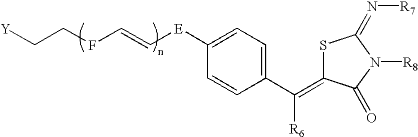 |

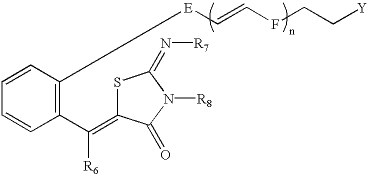

| | |
|---|---|
| 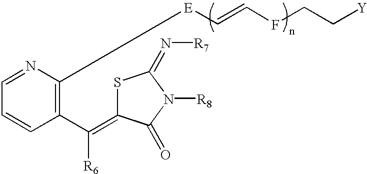 | 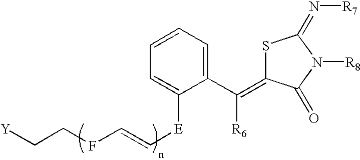 |
| 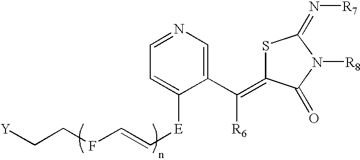 | 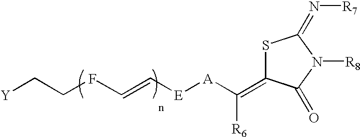 |
| 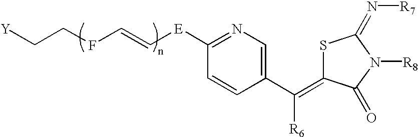 | 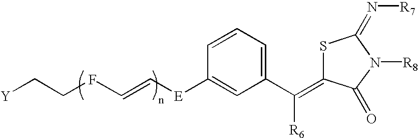 |

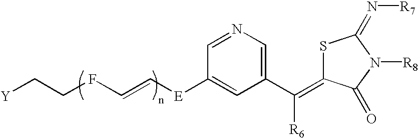

The variables E, F, Y, and n can have the values provided in Table 6 above.

TABLE 10

| | |
|---|---|
| 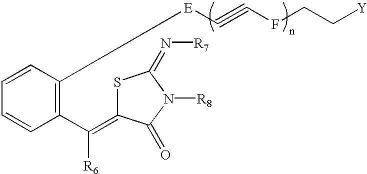 | 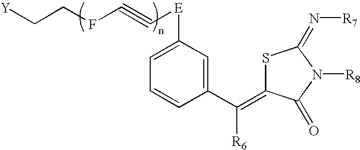 |

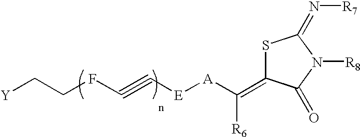

| | |
|---|---|
| 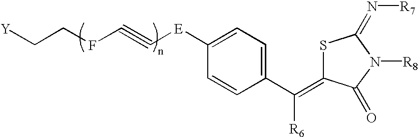 | 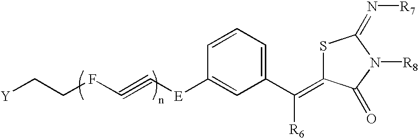 |
| 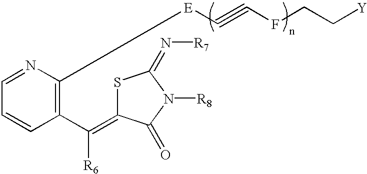 | 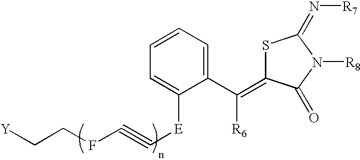 |
| 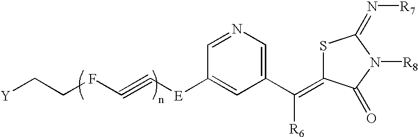 | 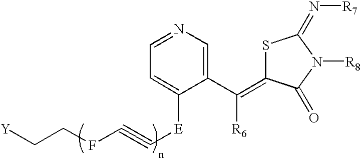 |

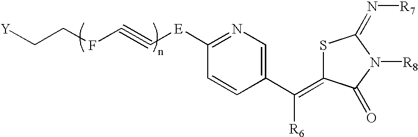

The variables E, F, Y, and n can have the values provided in Table 6 above.

### Example 20

Preparation of Bi-Ligand Libraries of the Present Invention

This example provides a general procedure for preparing bi-ligand libraries from common ligand mimics of the invention according to the reaction scheme presented in FIG. 4a. Compound numbers correspond to the numbers in the figure.

HOBt resin is in dry DMF. The resin then is added to a solution of compound 10 dissolved in a mixture of dry DMF and DIC (N,N′-diisopropylcarbodiimide). The solution is shaken at room temperature for a period of about 2 to 20 hours and then washed three times with dry DMF and three times with dry THF.

The resin is added to a solution of the amine dissolved in a mixture of dry THF/DMF (8:2). The mixture is again shaken at room temperature for a period of 2 to 20 hours. The resin is filtered and washed once with dry DMF. The filtrate is collected and vacuum dried to provide compound 11. Amines that can be used for the development of bi-ligand libraries of the invention using this reaction are provided in Table 1.

### Example 21

Preparation of Bi-Ligand Libraries of the Present Invention

This example provides a general procedure for preparing bi-ligand libraries from common ligand mimics of the invention according to the reaction scheme presented in FIG. 4b. Compound numbers correspond to the numbers in the figure.

HOBt resin is swelled in dry DMF. The resin is added to a solution of carboxylic acid (1-naphthalene acetic acid) dissolved in a mixture of dry DMF and DIC. The solution is shaken at room temperature overnight and washed with 3× dry DMF and 1× dry THF.

The resin is added to a solution of compound 12 dissolved in a mixture of dry THF/DMF. The solution is again shaken at room temperature overnight. The resin is filtered and washed once with dry DMF. The filtrate is collected and vacuum dried to provide compound 13. Carboxylic acids that can be used for the development of bi-ligand libraries of the invention using this reaction are provided in Table 2.

### Example 22

Preparation of Bi-Ligand Libraries of the Present Invention

This example provides a general procedure for preparing bi-ligand libraries from common ligand mimics of the invention according to the reaction scheme presented in FIG. 4c. Compound numbers correspond to the numbers in the figure.

Three equivalents of an isocyanate is added to a solution of compound 12 in DMSO. The reaction is allowed to proceed overnight. Then, aminomethylated polystyrene Resin (NovaBiochem, Cat. No. 01-64-0383) is added to the solution. The mixture is shaken for several hours at room temperature. The resin is filtered off, and the solution is dried under reduced pressure to yield compound 14. Isocyanates that can be used for the development of bi-ligand libraries of the invention using this reaction are provided in Table 3.

### Example 23

Screening of Selected Pseudothiohydantoins for Binding to Dehydrogenases and Oxidoreductases

This example describes the screening of three pseudothiohydantoincommon ligand mimics for binding activity to a variety of dehydrogenases and oxidoreductases.

The pseudothiohydantoin compounds: 5-(4-hydroxy-3-nitro-benzylidene)-2-imino-thiazolidin-4-one; 4-(2-imino-4-oxo-thiazolidin-5-ylidenemethyl)-benzoic acid; 5-(4-hydroxy-3-methoxy-benzylidene)-2-imino-thiazolidin-4-one were produced following the method of Example 1. The compounds were screened for binding to the following enzymes: dihydrodipicolinate reductase (DHPR), inosine-5′-monophosphate dehydrogenase (IMPDH), HMG CoA reductase (HMGCoAR), dihydrofolate reductase (DHFR), 1-deoxy-D-xylulose-5-phosphate reductase (DOXPR), aldose reductase (AR), 3-isopropylmalate (IPMDH), alcohol dehydrogenase (ADH), lactate dehydrogenase (LDH), and glyceraldehyde-3-phosphate dehydrogenase (GAPDH).

DHPR

For DHPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DHPR was diluted in 10 mM HEPES at a pH of 7.4. DHPS (dihydrodipicolinate synthase) was not diluted and was stored in eppindorf tubes.

| | Stock | Final | Volume needed |
|---|---|---|---|
| ddH₂O | | | 798 μl |
| HEPES (pH 7.8) | 1 M | 0.1 M | 100 μl |
| Pyruvate | 50 mM | 1 mM | 20 μl |
| NADPH | 1 mM | 6 μM | 6 μl |
| L-ASA | 28.8 mM | 40 μM | 13.9 μl |
| DHPS | 1 mg/ml | | 7 μl |
| DHPR | 1:1000 dilution of | | 5 μl |
| | 1 mg/ml stock | | |
| Inhibitor | 15 mM | 100 μM | 6.7 μl |
| | | | (0.67 DMSO) |
| DMSO | 100% | 5% | 43.3 μl |
| Total Assay volume = | | | 1000 μl |

The L-ASA (L-aspartate semialdehyde) solution was prepared in the following manner. 180 μM stock solution of ASA was prepared. 100 μl of the ASA stock solution was mixed with 150 μl of concentrated NaHCO₃ and 375 μl of H₂O. For use in the assay, 28.8 mM L-ASA was equal to 625 μl of the solution. The L-ASA stock solution was kept at a temperature of −20° C. After dilution, the pH of the 28.8 mM solution was checked and maintained between 1 and 2.

The DHPS reaction was monitored at 340 nm prior to and after addition of the inhibitor to detect background reaction with the inhibitor. The solution for background detection was a 945 μl solution containing 0.1 HEPES (pH 7.8), 1 mM pyruvate, 6 μM NADPH, 40 μM L-ASA, and 7 μl of 1 mg/ml DHPS at 25° C. in the volumes provided above. The sample solution was then mixed and incubated

| | Stock | Final | Volume needed |
|---|---|---|---|
| ddH₂O | | | 780 μl |
| HEPES (pH 7.4) | 1 M | 0.1 M | 100 μl |
| Pyruvate | 50 mM | 2.5 mM | 50 μl |
| NADH | 1 mM | 10 μM | 10 μl |
| LDH | 1:2000 dilution of | | 10 μl |
| | 1 mg/ml stock | | |
| Inhibitor | 15 mM | 100 μM | 6.7 μl |
| | | | (0.67% |
| | | | DMSO) |
| DMSO | 100% | 5% | 43.3 μl |
| Total Assay volume = | | | 1000 μl |

The LDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 10 μM NADH, and 2.5 mM of pyruvate. The reaction was then initiated with 10 μl of LDH from Rabbit Muscle (0.5 μg/ml; 1:2000 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.3 μM was substituted for inhibitor to yield 50 to 70% inhibition. The substrate was kept at a level at least 10 times the Km.

ADH

For ADH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| DdH₂O | | | 787 μl |
| HEPES (pH 8.0) | 1 M | 0.1 M | 100 μl |
| EtOH | 10 M | 130 mM | 13 μl |
| NAD+ | 2 mM | 80 μM | 40 μl |
| ADH | 1:400 dilution of | | 10 μl |
| | 1 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The ADH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a for 10 minutes. Next, 500 nM solutions of the inhibitors and enough DMSO to provide a final DMSO concentration of 5% of the total assay volume were added. The solution was mixed and incubated for an additional 6 minutes.

In DHPR samples, 5 μl of the diluted DHPR enzyme were added. The sample was mixed for 20 seconds and then the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 μM was substituted for inhibitor to yield 70 to 80% inhibition. The substrate was kept at a level at least 10 times the Km. The final concentration of L-ASA was about 1 mM.

LDH

For LDH analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below. 990 μl of a solution containing 0.1 M HEPES, pH 8.0, 80 μM NAD+, and 130 mM of ethanol. The reaction was then initiated with 10 μl of ADH from Bakers Yeast (3.3 μg/ml; 1:400 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 15.5 μM was substituted for inhibitor to yield 50 to 60% inhibition. The substrate was kept at a level at least 10 times the Km. The final concentration of pyruvate was about 2.5 mM.

Where only a simple read was desired, as in the case of NAD+ concentration determination, 13 μl (10 M stock) of ethanol was used to drive the reaction, and 10 μl of pure enzyme (1 mg/ml) was used. NAD+ was soluble at 2 mM, which allowed the concentration determination step to be skipped. In this situation, the procedure was as follows. All of the ingredients except for the enzyme were mixed together. The solution was mixed well and the absorbance at 340 nm read. The enzyme was added and read again at OD 340 after the absorbance stopped changing, generally 10 to 15 minutes after the enzyme was added.

DHFR

For DHFR analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below. H₂ folate was dissolved in DMSO to about 10 mM and then diluted with water to a concentration of 0.1 mM.

| | Stock | Final | Volume needed |
|---|---|---|---|
| ddH₂O | | | 616 μl |
| Tris-HCl (pH 7.0) | 1 M | 0.1 M | 100 μl |
| KCl | 1 mM | 0.15 M | 150 μl |
| H₂ Folate | 0.1 mM | 5 μM | 50 μl |
| NADPH | 2 mM | 52 μM | 26 μl |
| DHFR | 1:85 dilution of | | 8 μl |
| | 4 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The DHFR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 992 μl of a solution containing 0.1 M Tris-HCl, pH 7.0, 150 mM KCl, 5 μM H₂ folate, and 52 μM NADH. The oxidation reaction was then initiated with 8 μl of DHFR (0.047 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 always contained the control reaction (no inhibitor), and cuvette #2 always contained the positive control reaction in which Cibacron Blue at 3 μM was substituted for inhibitor to yield 50 to 70% inhibition. The substrate was kept at a level at least 10 times the Km.

DOXPR

For DOXPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DOXPR was diluted in 10 mM HEPES at a pH of 7.4.

| | Stock | Final | Volume needed |
|---|---|---|---|
| ddH₂O | | | 707 μl |
| HEPES (pH 7.4) | 1 M | 0.1 M | 100 μl |
| DOXP | 10 mM | 1.15 mM | 115 μl |
| NADPH | 1 mM | 8 μM | 8 μl |
| MnCl₂ | 100 mM | 1 mM | 10 μl |
| DOXPR | 1:200 dilution of | | 10 μl |
| | 2 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The DOXPR reaction was monitored at 340 nm prior to and after addition of the inhibitor to detect background reaction with the inhibitor. Solutions of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 1 mM MnCl₂ 1.15 mM DOXP, and 8 μM NADPH. The oxidation reaction was then initiated with 10 μl of DOXP reductoisomerase (10 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.32 μM was substituted for inhibitor to yield 70 to 80% inhibition. The substrate was kept at a level at least 10 times the Km.

GAPDH

For GAPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 739 μl |
| Triethanolamine | 1 M | 25 mM | 125 μl |
| (pH 7.5) | | | |
| GAP | 50 mM | 145 μM | 3 μl |
| NAD+ | 5 mM | 0.211 mM | 42 μl |
| Sodium Arsenate | 200 mM | 5 mM | 25 μl |
| 2-BME | 500 mM | 3 mM | 6 μl |
| GAPDH | 1:200 dilution of | | 10 μl |
| | 1 mg/ml stock | | |
| Inhibitor | 12.5 mM | 100 μM | 8 μl |
| | | | (total |
| | | | 5% DMSO) |
| DMSO | 100% | 5% | 42 μl |
| Total Assay volume = | | | 1000 μl |

The GAPDH reaction was monitored at 340 nm prior to and after addition of the inhibitor to detect background reaction with the inhibitor. Solutions of 100 μl of the inhibitors incubated for 6 minutes at 25° C. in a 990 μl of a solution containing 125 mM triethanolamine, pH 7.5, 145 μM glyceraldehyde 3-phosphate (GAP), 0.211 mM NAD, 5 mM sodium arsenate, and 3 mM β-metcaptoethanol (2-BME). The reaction was then initiated with 10 μl of E. coli GAPDH (1:200 dilution of 1.0 mg/ml). After the enzyme was added, the solution was mixed for 20 seconds, and the reaction was run for 10minutes. After a 50 second lag, the samples were read in a Cary spectrophotometer at 340 nm. Reading of the samples was continued until 300 seconds. The final concentration of DMSO in a cuvette was about 5% of the total assay volume. Cuvette #1 contained the control reaction (no inhibitor).

GAP for use in this experiment was deprotected from the diethyl acetal in the following manner. Water was boiled in recrystallizing dish. Dowex (1.5 mg) and GAP (200 mg; SIGMA G-5376) were weighed and placed in a 15 ml conical tube. The Dowex and GAP were resuspended in 2 ml dH₂O, followed by shaking of the tube until the GAP dissolved. The tube was then immersed, while shaking, in the boiling water for 3 minutes. Next, the tube was placed in an ice bath to cool for 5 minutes. As the sample cooled, a resin settled to the bottom of the test tube, allowing removal of the supernatant with a pasteur pipette. The supernatant was filtered through a 0.45 or 0.2 μM cellulose acetate syringe filter.

The filtered supernatant was retained, and another 1 ml of dH₂O was added to the resin tube. The tube was then shaken and centrifuged for 5 minutes at 3,000 rpm. The supernatant was again removed with a pasteur pipette and passed through a 0.45 or 0.2 μM cellulose acetate syringe filter. The two supernatant aliquots were then pooled to provide a total GAP concentration of about 50 mM. The GAP was then divided into 100 μl aliquots and stored at −20° C. until use.

IMPDH

For IMPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 447 μl |
| Tris-HCl (pH 8.0) | 1 M | 0.1 M | 100 μl |
| KCl | 1 M | 0.25 M | 250 μl |
| NAD+ | 2 mM | 30 μM | 15 μl |
| IMP | 6 mM | 600 μM | 100 μl |
| Glycerol | 10% | 0.3% | 30 μl |
| IMPDH | 0.75 mg/ml, | | 8 μl |
| | undiluted | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The IMPDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 37° C. in a 992 μl of a solution containing 0.1 M Tris-HCl, pH 8.0, 0.25 M KCl, 0.3% glycerol, 30 μM NAD+, and 600 μM IMP (inosine monophosphate). The reaction was then initiated with 8 μl of IMPDH (0.75 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue was substituted for inhibitor. The substrate was kept at a level at least 10 times the Km.

HMGCoAR

For HMGCoAR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. The enzyme was diluted in 1 M NaCl. To prepare the dilution buffer, 10 μl of HMGCoAR (1 mg/ml) was mixed with 133 μl of 3 M NaCl solution and 257 μl of 25 mM KH₂PO₄ buffer (pH 7.5; containing 50 mM NaCl, μl mM EDTA (ethylenediaminetetraacetic acid), and 5 mM DTT (dithiothreitol).

| | | | | | Volume | |
|---|---|---|---|---|---|---|
| | Stock | | Final | | needed | |
| ddH₂O | | | | | 841 | μl |
| KH₂PO₄ (pH 7.5) | 1 | M | 25 | mM | 25 | μl |
| HMGCoA | 10 | mM | 160 | mM | 16 | μl |
| NADPH | 1 | mM | 13 | μM | 13 | μl |
| NaCl | 1 | M | 50 | mM | 50 | μl |
| EDTA | 50 | mM | 1 | mM | 20 | μl |
| DTT | 500 | mM | 5 | mM | 10 | μl |
| HMGCoAR | 1:40 dilution of | | | | 5 | μl |
| | 0.65 mg/ml stock | | | | | |
| Inhibitor | 10 | mM | 100 | μM | 10 | μl |
| DMSO | 100% | | 2% | | 10 | μl |
| Total Assay volume = 1000 μl | | | | | | |

The HMGCoAR reaction was monitored at 340 nm prior to and after addition of the inhibitor to detect background reaction with the inhibitor. Solutions of 100 μM of the inhibitors in DMSO were prepared to provide a final DMSO concentration of 2% of the total assay volume. These solutions were incubated for 6 minutes at 25° C. in a 994 μl of a solution containing 25 mM KH₂PO₄, pH 7.5, 160 μM HMGCoA, 13 μM NADPH, 50 mM NaCl, 1 mM EDTA, and 5 mM DTT. The reaction was then initiated with 5 μl of HMGCoAR enzyme (1:40 dilution of 0.65 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.05 μM was substituted for inhibitor to yield 50 to 70% inhibition. The substrate was kept at a level at least 10 times the Km.

IPMDH

For IPMDH analysis, the compounds were screened using a kinetic protocol that spectrophotometrically evaluates reduction of NAD.

Stock solutions of each of the reagents were prepared in the following concentrations. Dilutions of the stock solutions were prepared prior to running the assay in the concentrations indicated below.

| | | | | | Volume | |
|---|---|---|---|---|---|---|
| | Stock | | Final | | needed | |
| ddH₂O | | | | | 407 | μl |
| KH₂PO₄ (pH 7.6) | 1 | M | 20 | mM | 20 | μl |
| KCl | 1 | M | 0.3 | M | 300 | μl |
| MNCl₂ | 20 | mM | 0.2 | mM | 10 | μl |
| NAD | 3.3 | mM | 109 | μM | 33 | μl |
| IPM | 2 | mM | 340 | μM | 170 | μl |
| E. coli IPMDH | 1:300 dilution of | | | | 10 | μl |
| | 2.57 mg/ml stock | | | | | |
| Inhibitor | 16 | mM | 200 | μM | 12.5 | μl |
| DMSO | 100% | | 5% | | 37.5 | μl |
| Total Assay volume = 1000 μl | | | | | | |

The IPMDH reaction was monitored at 340 nm prior to and after addition of the inhibitor to detect background reaction with the inhibitor. Inhibitor was incubated for 5 minutes at 37° C. in a 990 μl of a solution containing 20 mM potassium phosphate, pH 7.6, 0.3 M potassium chloride, 0.2 mM manganese chloride, 109 μM NAD, and 340 μM DL-threo-3-isopropylmalic acid (IPM). The reaction was then initiated with 10 μl of E. coli isopropylmalate dehydrogenase (1:300 dilution of 2.57 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in the cuvette was 5% of the total assay volume. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

AR

For AR analysis, the compounds were screened using a kinetic protocol that spectrophotometrically measures enzyme activity.

Stock solutions of each of the reagents were prepared in the following concentrations. Dilutions of the stock solutions were prepared prior to running the assay in the concentrations indicated below.

| | | | | | Volume | |
|---|---|---|---|---|---|---|
| | Stock | | Final | | needed | |
| ddH₂O | | | | | 565.5 | μl |
| KH₂PO₄ (pH 7.5) | 1 | M | 100 | mM | 100 | μl |
| Ammonium Sulfate | 1 | M | 0.3 | M | 300 | μl |
| EDTA | 500 | mM | 1 | mM | 2 | μl |
| NADPH | 1 | mM | 3.8 | μM | 3.8 | μl |
| Glyceraldehyde | 100 | mM | 171 | μM | 1.7 | μl |
| DTT | 100 | mM | 0.1 | mM | 1 | μl |
| Human ALDR | 1:5 dilution of | | | | 10 | μl |
| | 0.55 mg/ml stock | | | | | |
| Inhibitor | 12.5 | mM | 200 | μM | 16 | μl |
| Total Assay volume = 1000 μl | | | | | | |

The AR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 5 minutes at 25° C. in a 990 μl of a solution containing 100 mM potassium phosphate, pH 7.5, 0.3 M ammonium sulfate, 1.0 mM ethylenediaminetetraacetic acid (EDTA), 3.8 μM B-Nicotinamide adenine dinucleotide phosphate (NADPH), 171 μM DL-glyceraldehyde and 0.1 mM DL-dithiothreitol. The reaction was then initiated with 10 μl of Human Aldose Reductase (1:5 dilution of 0.55 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final DMSO concentration in the cuvette was 5%. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

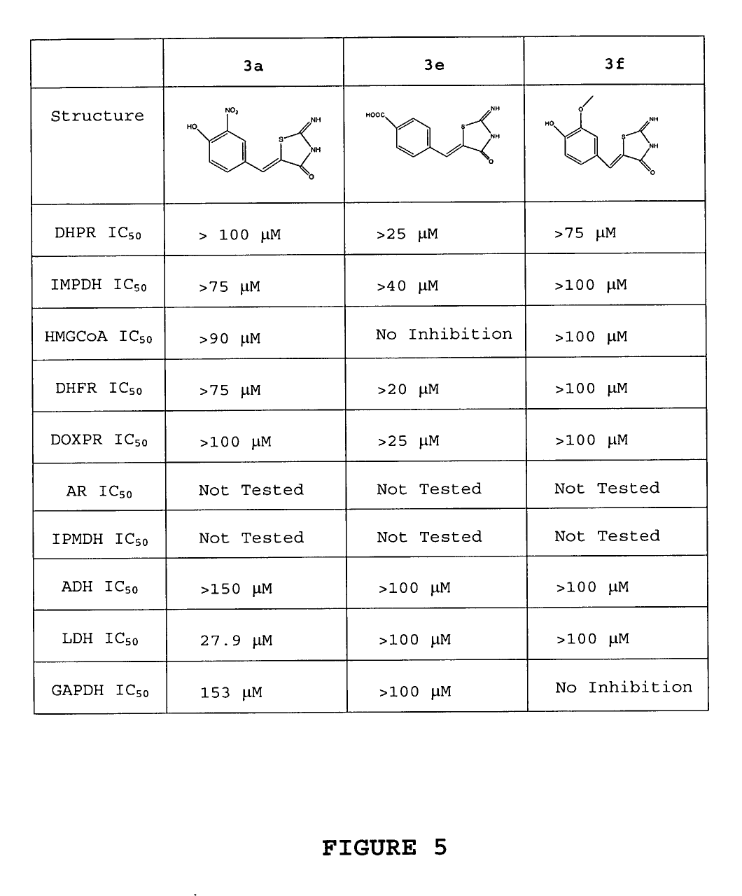

IC₅₀ data for these compounds are presented in FIG. 5. The compound 5-(4-hydroxy-3-nitro-benzylidene)-2-imino-thiazolidin-4-one exhibited IC₅₀ values of 27.9 μM for LDH and 153 μM for GAPDH. DOXPR and DHPR each exhibited IC₅₀ values greater than 100 μM. IMPDH and DHFR each exhibited IC₅₀ values greater than 75 μM. IC₅₀ values for ADH and HMGCoAR were greater than 150 μM and greater than 90 μM, respectively.

The compound 4-(2-imino-4-oxo-thiazolidin-5-ylidenemethyl)-benzoic acid exhibited IC₅₀ values greater than 100 μM for LDH, ADH, and GAPDH. The compound exhibited IC₅₀ values greater than 25 μM for DHPR and DOXPR. The IC₅₀ values for IMPDH and DHFR were greater than 40 μM and greater than 20 μM, respectively.

The compound 5-(4-hydroxy-3-methoxy-benzylidene)-2-imino-thiazolidin-4-one exhibited IC₅₀ values for DHFR, ADH, IMPDH, HMGCoAR, DOXPR, LDH of greater than 100 μM. The compound exhibited an IC₅₀ value greater than 75 μM for DHPR.

### Example 24

Screening of Selected Pseudothiohydantoins for Binding to Dehydrogenases and Oxidoreductases

This example describes the screening of pseudothiohydantoincommon ligand mimics for binding activity to a variety of dehydrogenases and oxidoreductases.

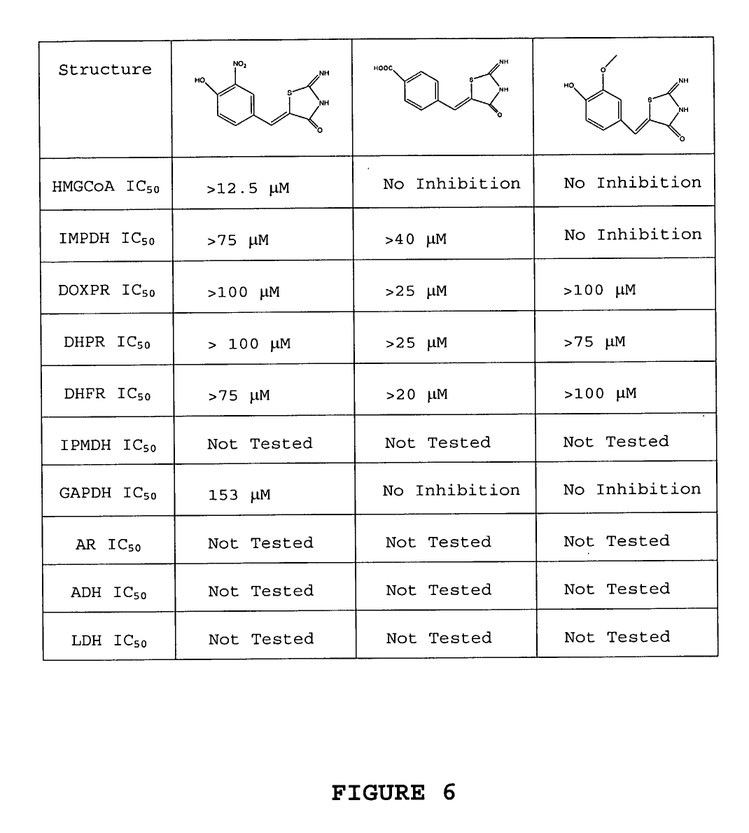

The following compounds were produced by the method of Example 1: 5-(4-hydroxy-3-nitro-benzylidene)-2-imino-thiazolidin-4-one; 4-(2-imino-4-oxo-thiazolidin-5-ylidenemethyl)-benzoic acid; and5-(4-hydroxy-2-methoxy-benzylidene)-2-imino-thiazolidin-4-one [Please verify the compound names with the structures in FIG. 6]. The compounds were screened for binding to the following enzymes using the sreening methods described in Example 23: HMG CoA reductase (HMGCoAR), inosine-5′-monophosphate dehydrogenase (IMPDH), 1-deoxy-D-xylulose-5-phosphate reductase (DOXPR), dihydrodipicolinate reductase (DHPR), dihydrofolate reductase (DHFR), 3-isopropylmalate (IPMDH), glyceraldehyde-3-phosphate dehydrogenase (GAPDH), aldose reductase (AR), alcohol dehydrogenase (ADH), and lactate dehydrogenase (LDH).

IC₅₀ data for these compounds are presented in FIG. 6. The compound 5-(4-hydroxy-3-nitro-benzylidene)-2-imino-thiazolidin-4-one demonstrated an IC₅₀ value of 153 μM for GAPDH and 27.9 μM for LDH. The compound exhibited IC₅₀ values greater than 100 μM for DOXPR and DHPR and greater than 75 μM for IMPDH and DHFR. IC₅₀ values for ADH and HMGCoAR were greater than 150 μM and 90 μM, respectively.

The compound 4-(2-imino-4-oxo-thiazolidin-5-ylidenemethyl)-benzoic acid exhibited IC₅₀ values greater than 25 μM for DOXPR and DHPR. The compound exhibited IC₅₀ values for LDH, IMPDH, and DHFR greater than 100 μM, greater than 40 μM, and greater than 20 μM, respectively. The compound showed no inhibition of GAPDH or ADH.

The compound 5-(4-hydroxy-2-methoxy-benzylidene)-2-imino-thiazolidin-4-one exhibited IC₅₀ values greater than 100 μM for DOXPR and DHFR. The IC 50 value for DHPR was greater than 75 μM. The compound showed no inhibition for HMGCoAR, IMPDH and GAPDH.

### Example 25

Screening of Biligands for Binding to Dihydrodipicolinate Reductase (DHPR)

This example describes the screening of bi-ligands having common ligand mimics for binding activity to dihydrodipicolinate reductase (DHPR).

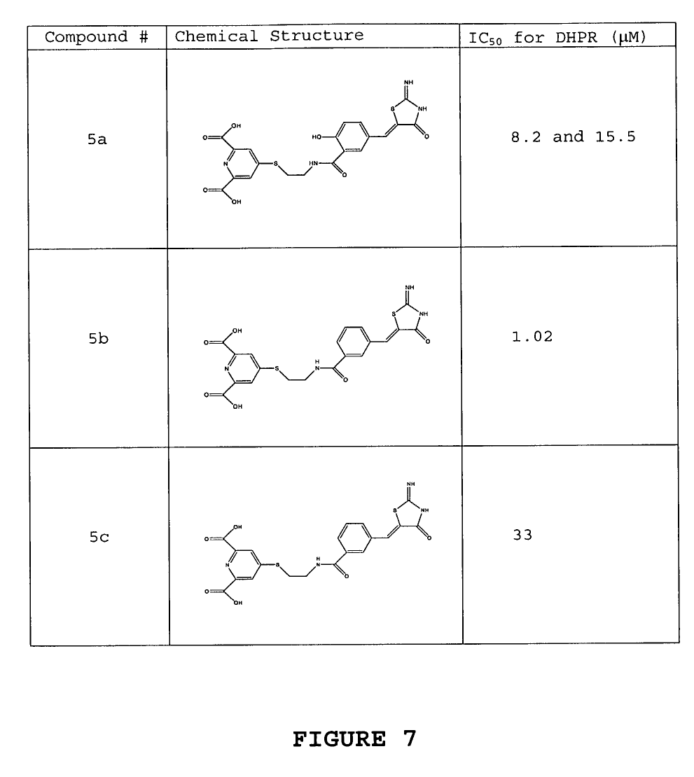

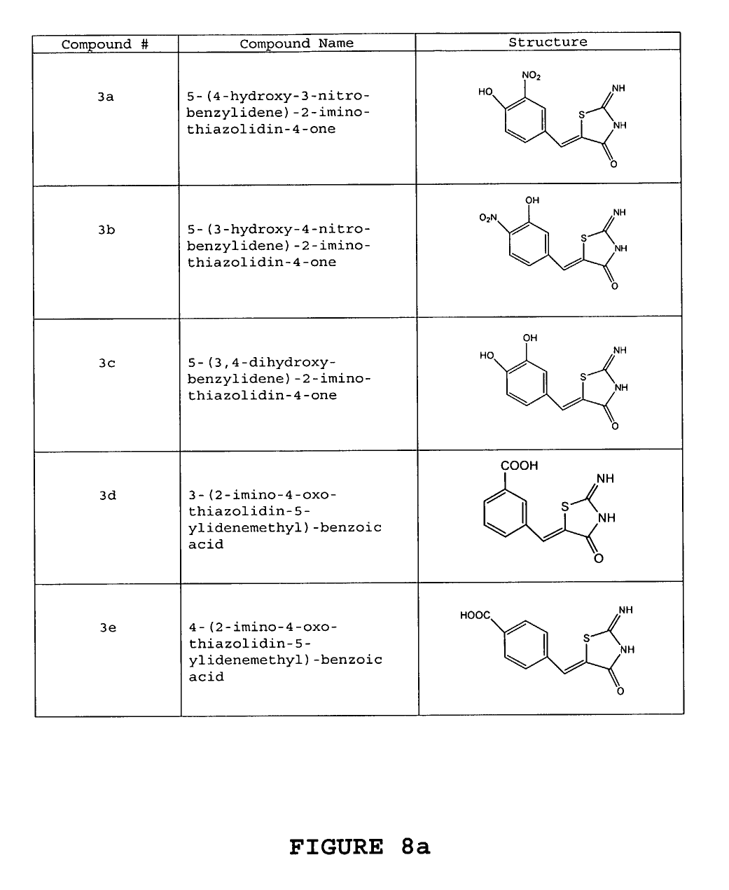

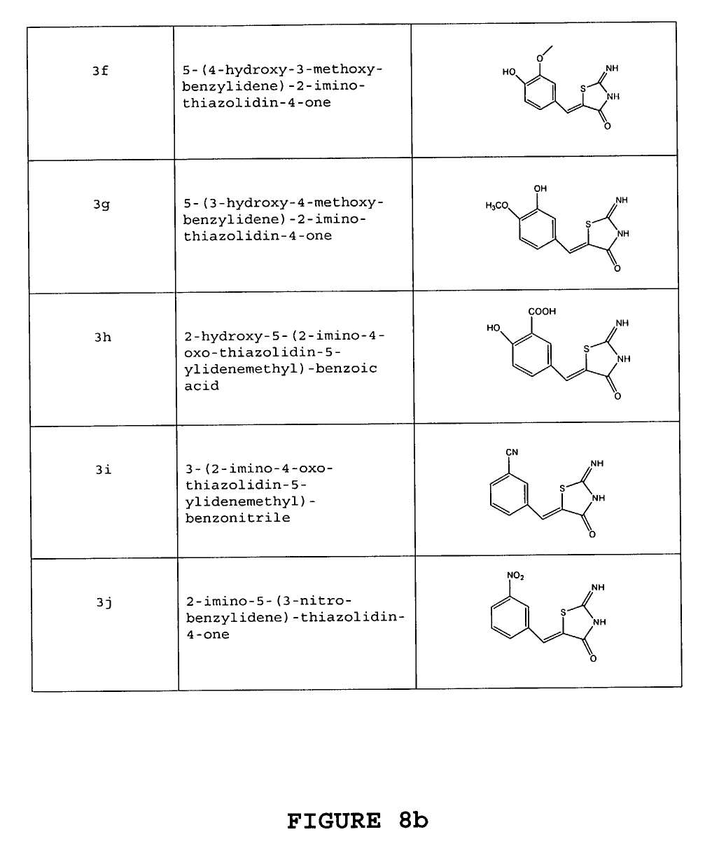

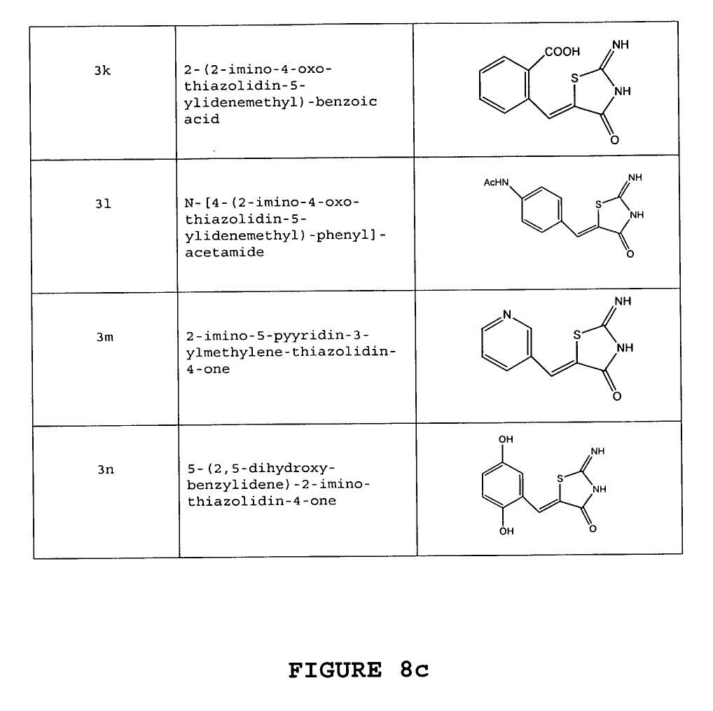

Bi-ligands were produced by the methods of Examples 16 to 18. The bi-ligands were screened for binding to DHPR. IC₅₀ data for these compounds are presented in FIG. 7.

Stock solutions of each of the reagents were prepared in the following concentrations. Dilutions of the stock solutions were prepared prior to running the assay in the concentrations indicated below. Dilution of DHPR was prepared in 10 mM HEPES at a pH of 7.4. DHPS was not diluted and was stored in eppindorf tubes.

| | | | | | Volume | |
|---|---|---|---|---|---|---|
| | Stock | | Final | | needed | |
| ddH₂O | | | | | 798 | μl |
| HEPES (pH 7.8) | 1 | M | 0.1 | M | 100 | μl |
| Pyruvate | 50 | mM | 1 | mM | 20 | μl |
| NADPH | 1 | mM | 6 | μM | 6 | μl |
| L-ASA | 28.8 | mM | 40 | μM | 3.9 | μl |
| DHPS | 1 | mg/ml | 7 | μl | | |
| DHPR | 1:1000 dilution of | | | | 5 | μl |
| | 1 mg/ml stock | | | | | |
| Inhibitor | 10 | mM | 500 | μM | 50 | μl |
| DMSO | 100% | | 5% | | 0 | μl |
| Total Assay volume = 1000 μl | | | | | | |

The L-ASA solution was prepared in the following manner. 180 μM stock solution of ASA was prepared. 100 μl of the ASA stock was mixed with 150 μl of concentrated NaHCO₃ and 375 μl of H₂I. For use in the assay, 28.8 mM L-ASA equal 625 μl of the solution. The L-ASA stock solution was kept at a temperature of −20° C. After dilution, the pH of the 28.8 mM solution was checked and maintained between 1 and 2.

First, the DHPS reaction was monitored at 340 nm prior to and after addition of the inhibitor to detect background reaction with the inhibitor. The solution for background detection was a 945 μl solution containing 0.1 HEPES (pH 7.8), 1 mM pyruvate, 6 μM NADPH, 40 μM L-ASA, and 7 μl of 1 mg/ml DHPS at 25° C. in the volumes provided above. The sample solution was then mixed and incubated for 10 minutes. Next, 500 nM solutions of the inhibitors and enough DMSO to provide a final DMSO concentration of 5% were added. The solution was mixed and incubated for an additional 6 minutes.

In DHPR samples, 5 μl of the diluted DHPR enzyme were added. The sample was mixed for 20 seconds and then the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 μM was substituted for inhibitor to yield 70 to 80% inhibition. The substrate and NADPH or NAHD were kept near their Kₘ values.

IC₅₀ data for these compounds are presented in FIG. 7. The pseudothiohydantoinderivative bi-ligands 5a, 5b, and 5c displayed IC₅₀ values for dihydrodipicolinate reductase (DHPR) of about 8.2 μM (and 15.5 μM), 1.02 μM, and 33 μM, respectively.

We claim:

1. A compound comprising the formula: 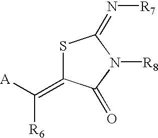
wherein
A is an aromatic carbocyclic or heterocyclic ring having 5, 6, or 7 members and from 0 to 3 heterocyclic atoms selected from the group consisting of oxygen, nitrogen, and sulfur, optionally substituted with from one to five substituents each independently selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring,
with the proviso that at least one of R₁ to R₆ is other than hydrogen.

2. The compound of claim 1, wherein A is substituted with one substituent.

3. The compound of claim 1, wherein A is substituted with an acid group.

4. The compound of claim 1, wherein A is substituted with a hydroxy group.

5. The compound of claim 1, wherein A is substituted with a nitrile group.

6. The compound of claim 1, wherein A is substituted with a nitro group.

7. The compound of claim 1, wherein A is substituted with an NHAc group.

8. The compound of claim 1, wherein A is substituted with two substituents.

9. The compound of claim 1, wherein A is substituted with two hydroxy groups.

10. The compound of claim 1, wherein A is substituted with a hydroxy group and a nitro group.

11. The compound of claim 1, wherein A is substituted with a hydroxy group and a methoxy group.

12. The compound of claim 1, wherein A is substituted with an acid group and a hydroxy group.

13. The compound of claim 1, wherein A is substituted with three or more substituents.

14. The compound of claim 1, wherein A is an aromatic carbocyclic ring.

15. The compound of claim 1, wherein A is an aromatic heterocyclic ring.

16. The compound of claim 1, wherein A is a five membered ring.

17. The compound of claim 1, wherein A is a six membered ring.

18. The compound of claim 1, wherein A is a seven membered ring.

19. The compound of claim 1, having the formula 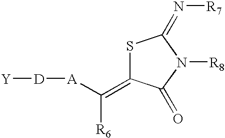
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

20. The compound of claim 1, having the formula 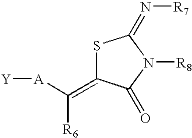
wherein Y is OH, NHR₁₁, SH, COOH, SO₂OH, X, CN, N₃, CONH₂, CONHR₁₁, C≡CH, or CH═CH₂.

21. The compound of claim 1, having the formula 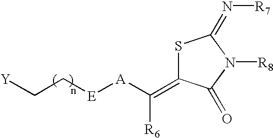
wherein
E present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

22. The compound of claim 1, having the formula 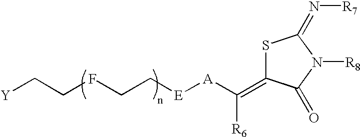
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

23. The compound of claim 1, having the formula 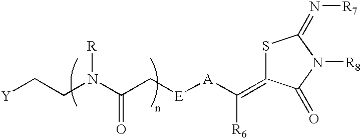
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

24. The compound of claim 1, having the formula 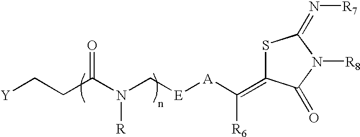
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

25. The compound of claim 1, having the formula 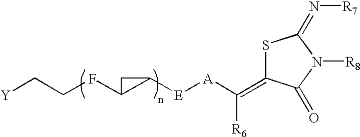
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

26. The compound of claim 1, having the formula 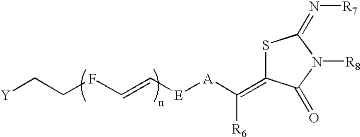
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

27. The compound of claim 1, having the formula 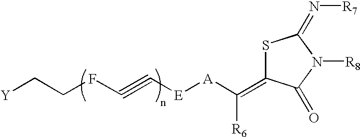
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

28. The compound of claim 1, having the formula 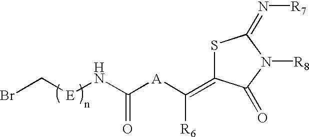
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

29. The compound of claim 1, having the formula 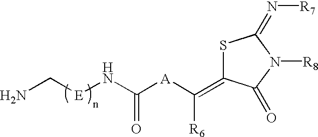
wherein
E is present or absent and when present is CH₂, CH₂CH₂OCH or CH₂CH₂SCH and n is an integer between 1 and 10, inclusive.

30. The compound of claim 29, wherein n is greater than 4 and E is CH₂CH₂OCH or CH₂CH₂SCH.

31. The compound of claim 1, having the formula

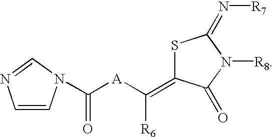

32. A compound comprising the formula: 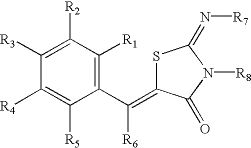
wherein
R₁ to R₆ each independently is selected from the group consisting of H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁, each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring,
with the proviso that at least one of R₁ to R₆ is other than hydrogen.

33. The compound of claim 32, wherein at least one of R₁ to R₆ is an acid group.

34. The compound of claim 32, wherein wherein at least one of R₁ to R₆ is a hydroxy group.

35. The compound of claim 32, wherein wherein at least one of R₁ to R₆ is a nitrile group.

36. The compound of claim 32, wherein wherein at least one of R₁ to R₆ is a nitro group.

37. The compound of claim 32, wherein wherein at least one of R₁ to R₆ is an NHAc group.

38. The compound of claim 32, wherein two or more of R₁ to R₆ are substituted.

39. The compound of claim 32, wherein at least two of R₁ to R₆ are hydroxy groups.

40. The compound of claim 32, wherein at least two of R₁ to R₆ independently are an acid group and a hydroxy group.

41. The compound of claim 32, wherein at least two of R₁ to R₆ independently are a hydroxy group and a nitro group.

42. The compound of claim 32, wherein at least two of R₁ to R₆ independently are a hydroxy group and a methoxy group.

43. The compound of claim 32, having the formula:

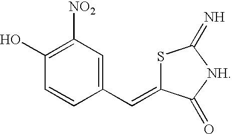

44. The compound of claim 32, having the formula:

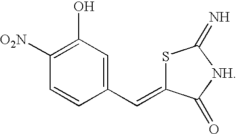

45. The compound of claim 32, having the formula:

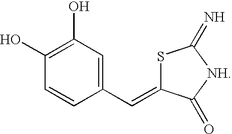

46. The compound of claim 32, having the formula:

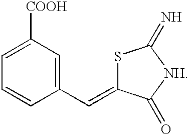

47. The compound of claim 32, having the formula:

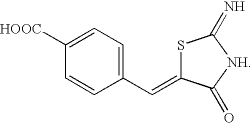

48. The compound of claim 32, having the formula:

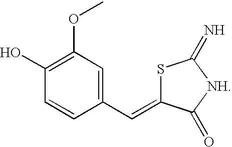

49. The compound of claim 32, having the formula:

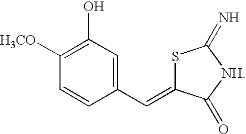

50. The compound of claim 32, having the formula:

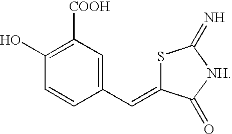

51. The compound of claim 32, having the formula:

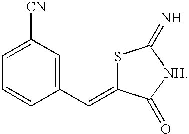

52. The compound of claim 32, having the formula:

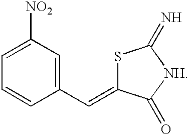

53. The compound of claim 32, having the formula:

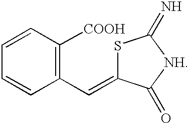

54. The compound of claim 32, having the formula:

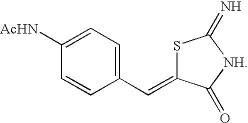

55. The compound of claim 32, having the formula:

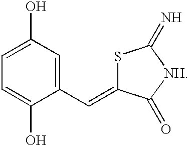

56. The compound of claim 32, having the formula 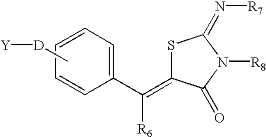
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

57. The compound of claim 32, having the formula 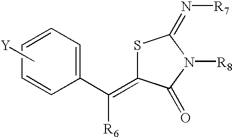
wherein Y is OH, NHR₁₁, SH, COOH, SO₂OH, X, CN, N₃, CONH₂, CONHR₁₁, C≡CH, or CH═CH₂.

58. The compound of claim 32, having the formula 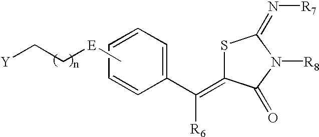
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

59. The compound of claim 32, having the formula 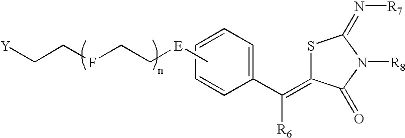
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

60. The compound of claim 32, having the formula 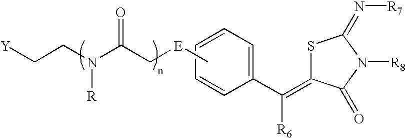
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

61. The compound of claim 32, having the formula 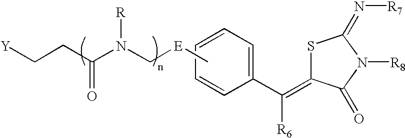
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH2, CONHR₁₁, C≡CH, or CH═CH₂;
R is hydrogen, alkyl, alkenyl, alkynyl, aryl, or heterocycle; and
n is an integer between 0 and 5, inclusive.

62. The compound of claim 32, having the formula 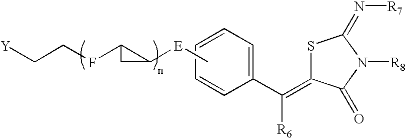
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

63. The compound of claim 32, having the formula 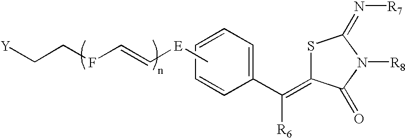
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

64. The compound of claim 32, having the formula 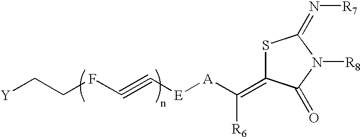
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

65. The compound of claim 32, having the formula 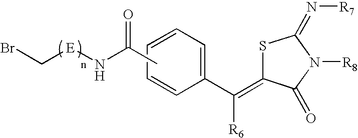
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

66. The compound of claim 32, having the formula 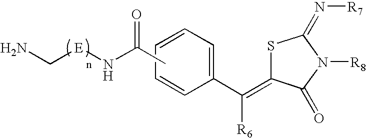
wherein
E present or absent and when present is CH₂, CH₂CH₂OCH or CH₂CH₂SCH and n is an integer between 1 and 10, inclusive.

67. The compound of claim 66, wherein n is greater than 4 and E is CH₂CH₂OCH or CH₂CH₂SCH.

68. The compound of claim 32, having the formula

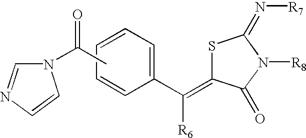

69. A compound comprising the formula: 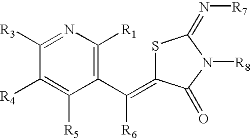
wherein
R₁, R₃, R₄, R₅, and R₆ each independently is selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁, each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring,
with the proviso that at least one of R₁ to R₆ is other than hydrogen.

70. The compound of claim 69, wherein at least one of R₁, R₃, R₄, R₅, or R₆ is COOH.

71. The compound of claim 69, wherein at least one of R₁, R₃, R₄, R₅, or R₆ is OH.

72. The compound of claim 69, wherein at least one of R₁, R₃, R₄, R₅s or R₆ is NO₂.

73. The compound of claim 69, wherein at least one of R₁, R₃, R₄, R₅, or R₆ is CN.

74. The compound of claim 69, wherein at least one of R₁, R₃, R₄, R₅, or R₆ is OAlkyl.

75. The compound of claim 69, wherein at least one of R₁, R₃, R₄, R₅, or R₆ is COOAlkyl.

76. The compound of claim 69, wherein at least one of R₁, R₃, R₄, R₅, or R₆ is NHAc.

77. The compound of claim 69, having the formula:

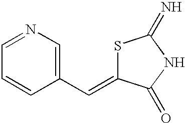

78. The compound of claim 69, having the formula 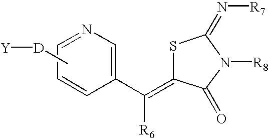
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

79. The compound of claim 69, having the formula 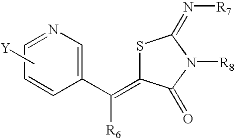
wherein Y is OH, NHR₁₁, SH, COOH, SO₂OH, X, CN, N₃, CONH₂, CONHR₁₁, C≡CH, or CH═CH₂.

80. The compound of claim 69, having the formula 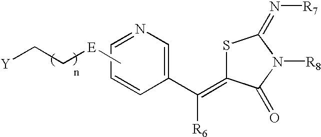
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

81. The compound of claim 69, having the formula 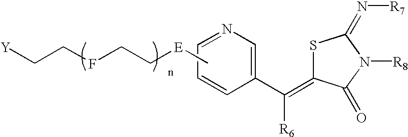
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

82. The compound of claim 69, having the formula 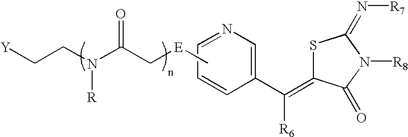
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

83. The compound of claim 69, having the formula 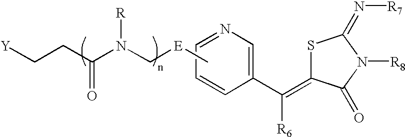
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

84. The compound of claim 69, having the formula 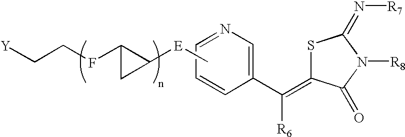
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

85. The compound of claim 69, having the formula 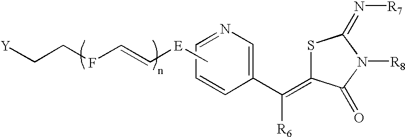
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

86. The compound of claim 69, having the formula 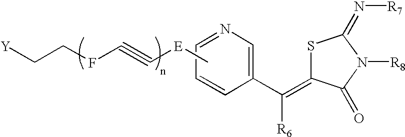
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

87. The compound of claim 69, having the formula 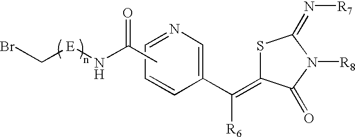
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

88. The compound of claim 69, having the formula 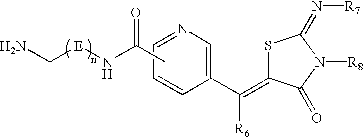
wherein
E is present or absent and when present is CH₂, CH₂CH₂OCH or CH₂CH₂SCH and n is an integer between 1 and 10, inclusive.

89. The compound of claim 88, wherein n is greater than 4 and E is CH₂CH₂OCH or CH₂CH₂SCH.

90. The compound of claim 69, having the formula

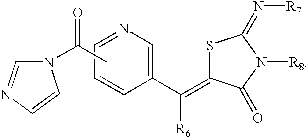

91. A combinatorial library of two or more compounds comprising a common ligand variant of a compound of the formula: 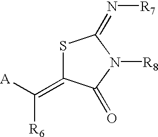
wherein
A is an aromatic carbocyclic or heterocyclic ring having 5, 6, or 7 members and from 0 to 3 heterocyclic atoms selected from the group consisting of oxygen, nitrogen, and sulfur, optionally substituted with from one to five substituents each independently selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

92. The combinatorial library of claim 91, wherein A is substituted with one substituent.

93. The combinatorial library of claim 91, wherein A is substituted with an acid group.

94. The combinatorial library of claim 91, wherein A is substituted with a hydroxy group.

95. The combinatorial library of claim 91, wherein A is substituted with a nitrile group.

96. The combinatorial library of claim 91, wherein A is substituted with a nitro group.

97. The combinatorial library of claim 91, wherein A is substituted with an NHAc group.

98. The combinatorial library of claim 91, wherein A is substituted with two substituents.

99. The combinatorial library of claim 91, wherein A is substituted with two hydroxy groups.

100. The combinatorial library of claim 91, wherein A is substituted with a hydroxy group and a nitro group.

101. The combinatorial library of claim 91, wherein A is substituted with a hydroxy group and a methoxy group.

102. The combinatorial library of claim 91, wherein A is substituted with an acid group and a hydroxy group.

103. The combinatorial library of claim 91, wherein A is substituted with three or more substituents.

104. The combinatorial library of claim 91, having the formula 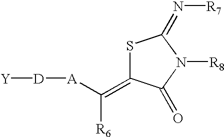
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

105. The combinatorial library of claim 91, having the formula 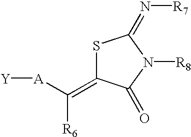
wherein Y is OH, NHR₁₁, SH, COOH, SO₂OH, X, CN, N₃, CONH₂, CONHR₁₁, C≡CH, or CH═CH₂.

106. The combinatorial library of claim 91, having the formula 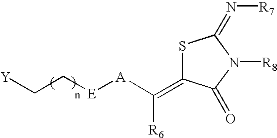
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

107. The combinatorial library of claim 91, having the formula 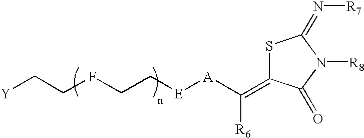
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

108. The combinatorial library of claim 91, having the formula 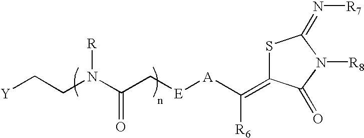
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

109. The combinatorial library of claim 91, having the formula 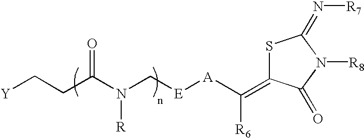
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

110. The combinatorial library of claim 91, having the formula 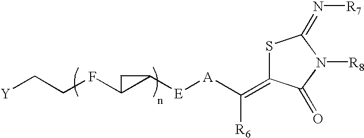
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

111. The combinatorial library of claim 91, having the formula 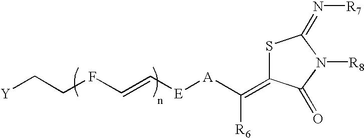
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

112. The combinatorial library of claim 91, having the formula 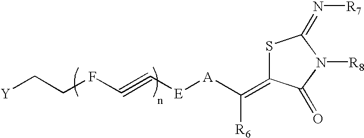
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

113. The combinatorial library of claim 91, having the formula 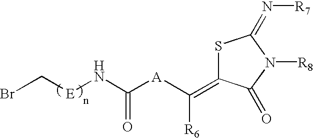
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

114. The combinatorial library of claim 91, having the formula 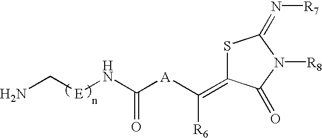
wherein
E is present or absent and when present is CH₂, CH₂CH₂OCH or CH₂CH₂SCH and n is an integer between 1 and 10, inclusive.

115. The combinatorial library of claim 114, where n is greater than 4 and E is CH₂CH₂OCH or CH₂CH₂SCH.

116. The combinatorial library of claim 91, having the formula

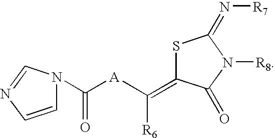

117. A combinatorial library of two or more compounds comprising a common ligand variant of a compound of the formula: 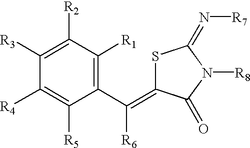
wherein
R₁ to R₆ each independently is selected from the group consisting of H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

118. The combinatorial library of claim 117, wherein at least one of R₁ to R₆ is an acid group.

119. The combinatorial library of claim 117, wherein wherein at least one of R₁ to R₆ is a hydroxy group.

120. The combinatorial library of claim 117, wherein wherein at least one of R₁ to R₆ is a nitrile group.

121. The combinatorial library of claim 117, wherein wherein at least one of R₁ to R₆ is a nitro group.

122. The combinatorial library of claim 117, wherein wherein at least one of R₁ to R₆ is an NHAc group.

123. The combinatorial library of claim 117, wherein two or more of R₁ to R₆ are substituted.

124. The combinatorial library of claim 117, wherein at least two of R₁ to R₆ are hydroxy groups.

125. The combinatorial library of claim 117, wherein at least two of R₁ to R₆ independently are an acid group and a hydroxy group.

126. The combinatorial library of claim 117, wherein at least two of R₁ to R₆ independently are a hydroxy group and a nitro group.

127. The combinatorial library of claim 117, wherein at least two of R₁ to R₆ independently are a hydroxy group and a methoxy group.

128. The combinatorial library of claim 117, having the formula:

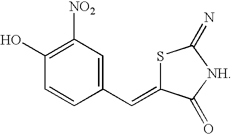

129. The combinatorial library pound of claim 117, having the formula:

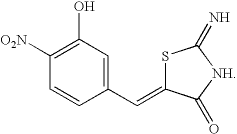

130. The combinatorial library of claim 117, having the formula:

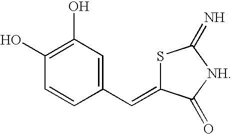

131. The combinatorial library of claim 117, having the formula:

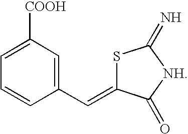

132. The combinatorial library of claim 117, having the formula:

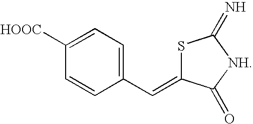

133. The combinatorial library of claim 117, having the formula:

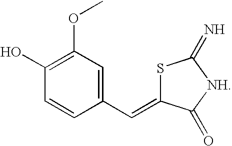

134. The combinatorial library of claim 117, having the formula:

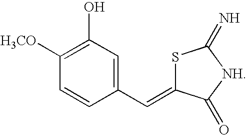

135. The combinatorial library of claim 117, having the formula:

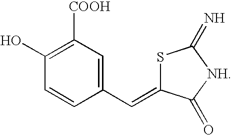

136. The combinatorial library of claim 117, having the formula:

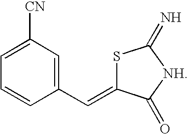

137. The combinatorial library of claim 117, having the formula:

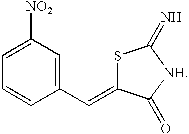

138. The combinatorial library of claim 117, having the formula:

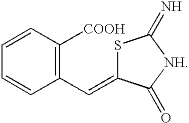

139. The combinatorial library of claim 117, having the formula:

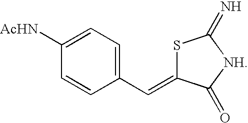

140. The combinatorial library of claim 117, having the formula:

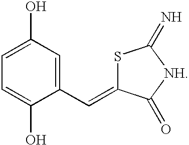

141. The combinatorial library of claim 117, having the formula 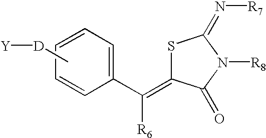
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

142. The combinatorial library of claim 117, having the formula 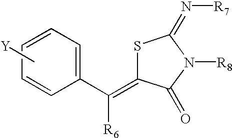
wherein Y is OH, NHR₁₁, SH, COOH, SO₂OH, X, CN, N₃, CONH₂, CONHR₁₁, C≡CH, or CH═CH₂.

143. The combinatorial library of claim 117, having the formula 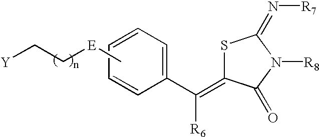
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

144. The combinatorial library of claim 117, having the formula 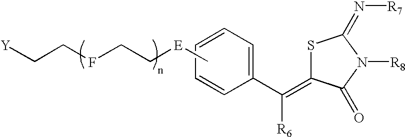
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

145. The combinatorial library of claim 117, having the formula 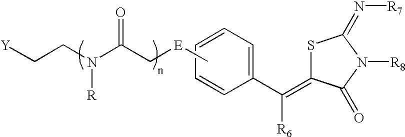
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

146. The combinatorial library of claim 117, having the formula 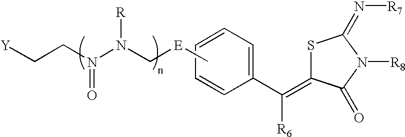
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

147. The combinatorial library of claim 117, having the formula 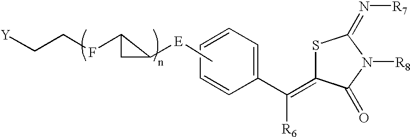
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

148. The combinatorial library of claim 117, having the formula 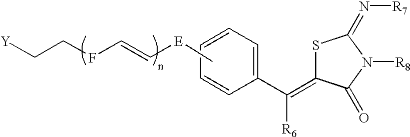
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

149. The combinatorial library of claim 117, having the formula 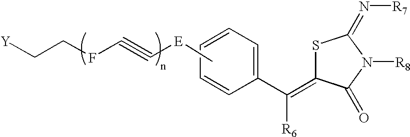
wherein
E is present and absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

150. The combinatorial library of claim 117, having the formula 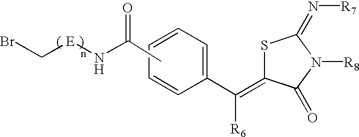
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

151. The combinatorial library of claim 117, having the formula 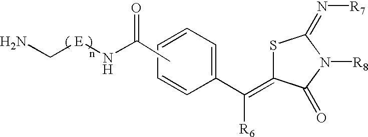
wherein
E present or absent and when present is CH₂, CH₂CH₂OCH or CH₂CH₂SCH and n is an integer between 1 and 10, inclusive.

152. The combinatorial library of claim 151, where n is greater than 4 and E is CH₂CH₂OCH or CH₂CH₂SCH.

153. The combinatorial library of claim 117, having the formula

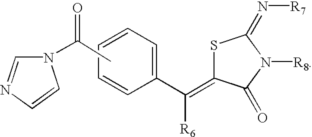

154. A combinatorial library of two or more compounds comprising a common ligand variant of a compound of the formula: 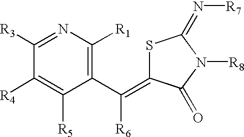
wherein
R₁, R₃, R₄, R₅, and R₆ each independently is selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR_{1, NR}₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

155. The combinatorial library of claim 154, wherein at least one of R₁, R₃, R₄, R₅, or R₆ is COOH.

156. The combinatorial library of claim 154, wherein at least one of R₁, R₃, R₄, R₅s or R₆ is OH.

157. The combinatorial library of claim 154, wherein at least one of R₁, R₃, R₄, R₅, or R₆ is NO₂.

158. The combinatorial library of claim 154, wherein at least one of R₁, R₃, R₄, R₅, or R₆ is CN.

159. The combinatorial library of claim 154, wherein at least one of R₁, R₃, R₄, R₅, or R₆ is OAlkyl.

160. The combinatorial library of claim 154, wherein at least one of R₁, R₃, R₄, R₅, or R₆ is COOAlkyl.

161. The combinatorial library of claim 154, wherein at least one of R₁, R₃, R₄, R₅, or R₆ is NHAc.

162. The combinatorial library of claim 154, having the formula:

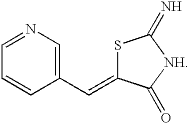

163. The combinatorial library of claim 154, having the formula 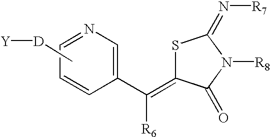
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

164. The combinatorial library of claim 154, having the formula 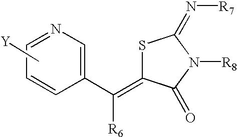
wherein Y is OH, NHR₁₁, SH, COOH, SO₂OH, X, CN, N₃, CONH₂, CONHR₁₁, C≡CH, or CH═CH₂.

165. The combinatorial library of claim 154, having the formula 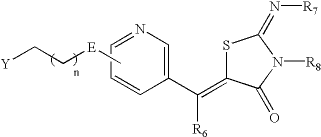
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

166. The combinatorial library of claim 154, having the formula 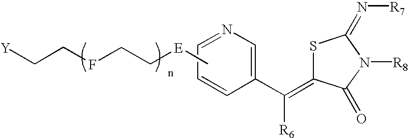
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

167. The combinatorial library of claim 154, having the formula 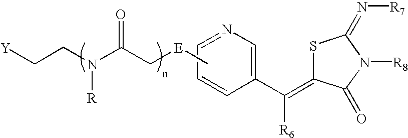
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

168. The combinatorial library of claim 154, having the formula 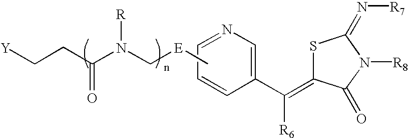
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

169. The combinatorial library of claim 154, having the formula 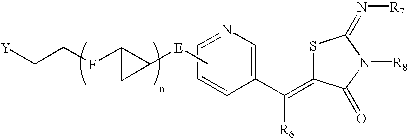
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

170. The combinatorial library of claim 154, having the formula 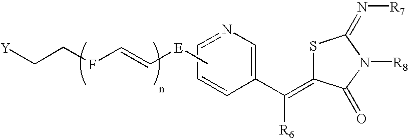
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

171. The combinatorial library of claim 154, having the formula 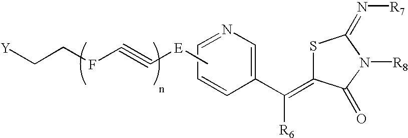
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

172. The combinatorial library of claim 154, having the formula 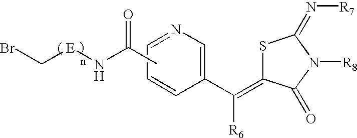
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

173. The combinatorial library of claim 154, having the formula 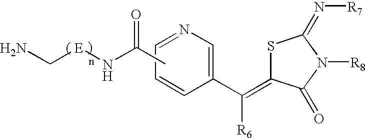
wherein
E is present or absent and when present is CH₂, CH₂CH₂OCH or CH₂CH₂SCH and n is an integer between 1 and 10, inclusive.

174. The combinatorial library of claim 173, where n is greater than 4 and E is CH₂CH₂OCH or CH₂CH₂SCH.

175. The combinatorial library of claim 154, having the formula

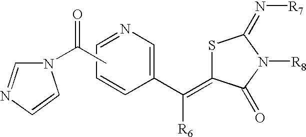

176. A combinatorial library of two or more-bi-ligands comprising the reaction product of a specificity ligand and a common ligand mimic having the formula: 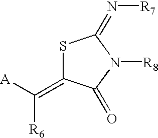
wherein
A is an aromatic carbocyclic or heterocyclic ring having 5, 6, or 7 members and from 0 to 3 heterocyclic atoms selected from the group consisting of oxygen, nitrogen, and sulfur, optionally substituted with from one to five substituents each independently selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S (O) R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

177. The combinatorial library of claim 176, wherein A is substituted with one substituent.

178. The combinatorial library of claim 176, wherein A is substituted with an acid group.

179. The combinatorial library of claim 176, wherein A is substituted with a hydroxy group.

180. The combinatorial library of claim 176, wherein A is substituted with a nitrile group.

181. The combinatorial library of claim 176, wherein A is substituted with a nitro group.

182. The combinatorial library of claim 176, wherein A is substituted with an NHAc group.

183. The combinatorial library of claim 176, wherein A is substituted with two substituents.

184. The combinatorial library of claim 176, wherein A is substituted with two hydroxy groups.

185. The combinatorial library of claim 176, wherein A is substituted with a hydroxy group and a nitro group.

186. The combinatorial library of claim 176, wherein A is substituted with a hydroxy group and a methoxy group.

187. The combinatorial library of claim 176, wherein A is substituted with an acid group and a hydroxy group.

188. The combinatorial library of claim 176, wherein A is substituted with three or more substituents.

189. The combinatorial library of claim 176, wherein the common ligand mimic comprises a compound of the formula: 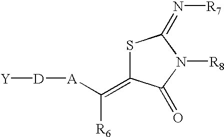
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

190. The combinatorial library of claim 176, wherein the common ligand mimic comprises a compound of the formula: 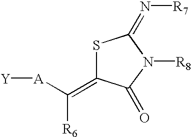
wherein Y is OH, NHR₁₁, SH, COOH, SO₂OH, X, CN, N₃, CONH₂, CONHR₁₁, C≡CH, or CH═CH₂.

191. The combinatorial library of claim 176, wherein the common ligand mimic comprises a compound of the formula: 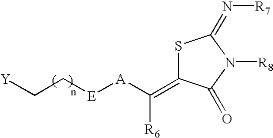
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

192. The combinatorial library of claim 176, wherein the common ligand mimic comprises a compound of the formula: 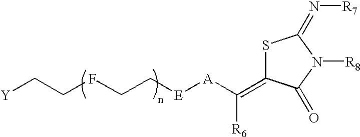
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

193. The combinatorial library of claim 176, wherein the common ligand mimic comprises a compound of the formula: 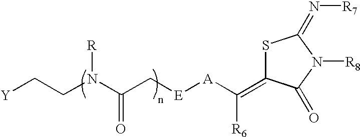
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

194. The combinatorial library of claim 176, wherein the common ligand mimic comprises a compound of the formula: 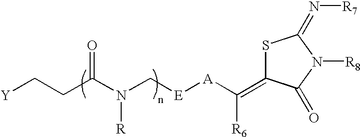
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

195. The combinatorial library of claim 176, wherein the common ligand mimic comprises a compound of the formula: 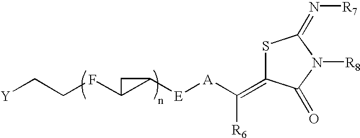
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

196. The combinatorial library of claim 176, wherein the common ligand mimic comprises a compound of the formula: 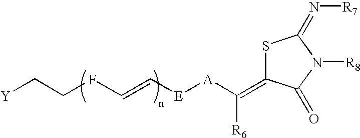
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

197. The combinatorial library of claim 176, wherein the common ligand mimic comprises a compound of the formula: 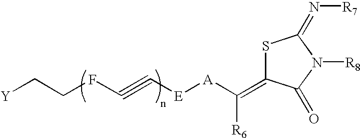
wherein
E present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

198. The combinatorial library of claim 176, wherein the common ligand mimic comprises a compound of the formula: 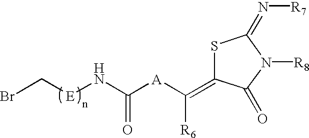
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

199. The combinatorial library of claim 176, wherein the common ligand mimic comprises a compound of the formula: 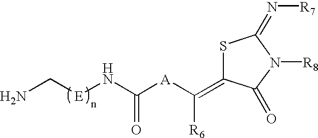
wherein
E is present or absent and when present is CH₂, CH₂CH₂OCH or CH₂CH₂SCH and n is an integer between 1 and 10, inclusive.

200. The combinatorial library of claim 199, where n is greater than 4 and E is CH₂CH₂OCH or CH₂CH₂SCH.

201. The combinatorial library of claim 176, wherein the common ligand mimic comprises a compound of the formula:

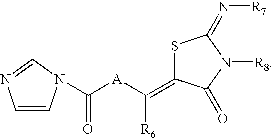

202. A combinatorial library of two or more bi-ligands comprising the reaction product of a specificity ligand and a common ligand mimic having the formula: 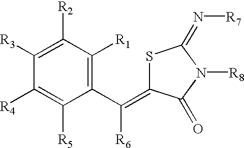
wherein
R₁ to R₆ each independently is selected from the group consisting of H, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R₇ and R₈ each independently is. selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

203. The combinatorial library of claim 202, wherein at least one of R₁ to R₆ is an acid group.

204. The combinatorial library of claim 202, wherein wherein at least one of R₁ to R₆ is a hydroxy group.

205. The combinatorial library of claim 202, wherein wherein at least one of R₁ to R₆ is a nitrile group.

206. The combinatorial library of claim 202, wherein wherein at least one of R₁ to R₆ is a nitro group.

207. The combinatorial library of claim 202, wherein wherein at least one of R₁ to R₆ is an NHAc group.

208. The combinatorial library of claim 202, wherein two or more of R₁ to R₆ are substituted.

209. The combinatorial library of claim 202, wherein at least two of R₁ to R₆ are hydroxy groups.

210. The combinatorial library of claim 202, wherein at least two of R₁ to R₆ independently are an acid group and a hydroxy group.

211. The combinatorial library of claim 202, wherein at least two of R₁ to R₆ independently are a hydroxy group and a nitro group.

212. The combinatorial library of claim 202, wherein at least two of R₁ to R₆ independently are a hydroxy group and a methoxy group.

213. The combinatorial library of claim 202, wherein the common ligand mimic comprises a compound of the formula:

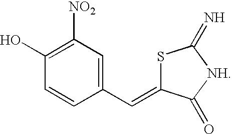

214. The combinatorial library of claim 202, wherein the common ligand mimic comprises a compound of the formula:

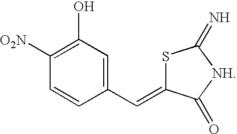

215. The combinatorial library of claim 202, wherein the common ligand mimic comprises a compound of the formula:

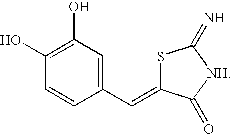

216. The combinatorial library of claim 202, wherein the common ligand mimic comprises a compound of the formula:

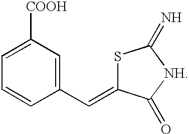

217. The combinatorial library of claim 202, wherein the common ligand mimic comprises a compound of the formula:

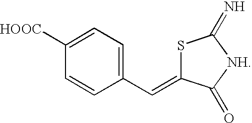

218. The combinatorial library of claim 202, wherein the common ligand mimic comprises a compound of the formula:

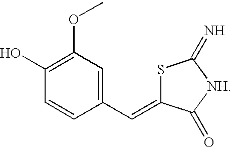

219. The combinatorial library of claim 202, wherein the common ligand mimic comprises a compound of the formula:

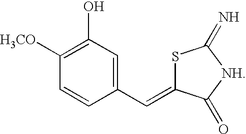

220. The combinatorial library of claim 202, wherein the common ligand mimic comprises a compound of the formula:

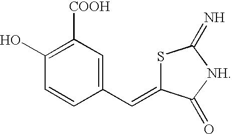

221. The combinatorial library of claim 202, wherein the common ligand mimic comprises a compound of the formula:

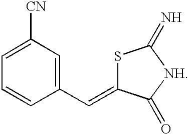

222. The combinatorial library of claim 202, wherein the common ligand mimic comprises a compound of the formula:

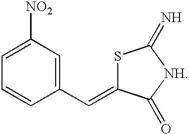

223. The combinatorial library of claim 202, wherein the common ligand mimic comprises a compound of the formula:

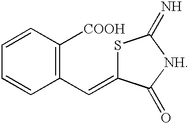

224. The combinatorial library of claim 202, wherein the common ligand mimic comprises a compound of the formula:

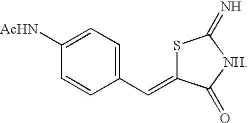

225. The combinatorial library of claim 202, wherein the common ligand mimic comprises a compound of the formula:

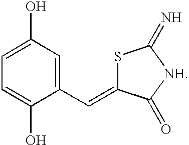

226. The combinatorial library of claim 202, wherein the common ligand mimic comprises a compound of the formula: 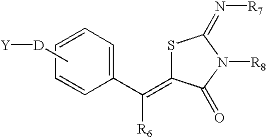
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

227. The combinatorial library of claim 202, wherein the common ligand mimic comprises a compound of the formula: 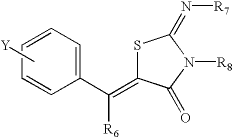
wherein Y is OH, NHR₁₁, SH, COOH, SO₂OH, X, CN, N₃, CONH₂, CONHR₁₁, C≡CH, or CH═CH₂.

228. The combinatorial library of claim 202, wherein the common ligand mimic comprises a compound of the formula: 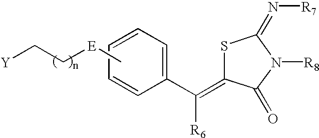
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

229. The combinatorial library of claim 202, wherein the common ligand mimic comprises a compound of the formula: 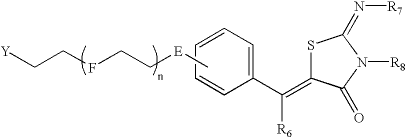
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

230. The combinatorial library of claim 202, wherein the common ligand mimic comprises a compound of the formula: 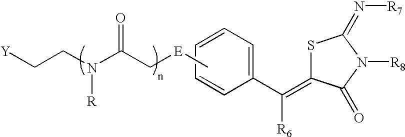
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

231. The combinatorial library of claim 202, wherein the common ligand mimic comprises a compound of the formula: 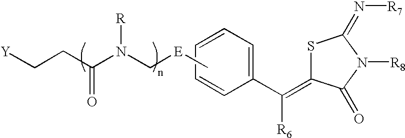
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

232. The combinatorial library of claim 202, wherein the common ligand mimic comprises a compound of the formula: 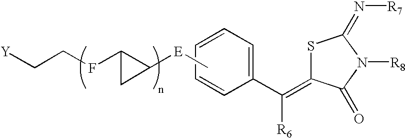
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

233. The combinatorial library of claim 202, wherein the common ligand mimic comprises a compound of the formula: 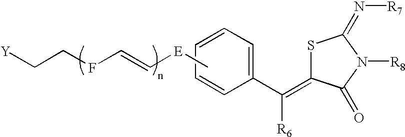
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

234. The combinatorial library of claim 202, wherein the common ligand mimic comprises a compound of the formula: 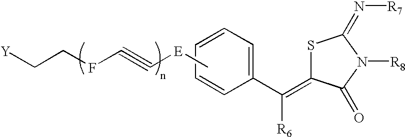
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

235. The combinatorial library of claim 202, wherein the common ligand mimic comprises a compound of the formula: 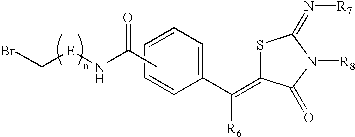
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

236. The combinatorial library of claim 202, wherein the common ligand mimic comprises a compound of the formula: 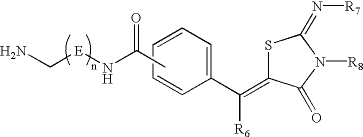
wherein
E is present or absent and when present is CH₂, CH₂CH₂OCH or CH₂CH₂SCH and n is an integer between 1 and 10, inclusive.

237. The combinatorial library of claim 236, where n is greater than 4 and E is CH₂CH₂OCH or CH₂CH₂SCH.

238. The combinatorial library of claim 202, wherein the common ligand mimic comprises a compound of the formula:

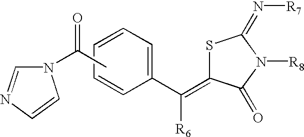

239. A combinatorial library of two or more bi-ligands comprising the reaction product of a specificity ligand and a common ligand mimic having the formula: 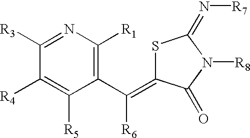
wherein
R₁, R₃, R₄, R₅, and R₆ each independently is selected from the group consisting of hydrogen, alkyl, alkenyl, alkynyl, aryl, heterocycle, COOH, COOAlkyl, CONR₉R₁₀, C(O)R₁₁, OH, OAlkyl, OAc, SH, SR₁₁, SO₃H, S(O)R₁₁, SO₂NR₉R₁₀, S(O)₂R₁₁, NH₂, NHR₁₁, NR₉R₁₀, NHCOR₁₁, NR₁₀COR₁₁, N₃, NO₂, PH₃, PH₂R₁₁, PO₄H₂, H₂PO₃, H₂PO₂, HPO₄R₁₁, PO₂R₁₀R₁₁, CN, and X;
R⁷ and R⁸ each independently is selected from the group consisting of hydrogen, OH, NH₂, alkyl, alkenyl, alkynyl, aryl, and heterocycle, or R₇ and R₈ can be attached indirectly through an alkylene, alkenylene, or alkynylene chain to form a heterocyclic ring fused to the thiohydantoin ring; and
R₉, R₁₀, and R₁₁ each independently is selected from the group consisting of hydrogen, alkyl, alkenyl, alkynyl, aryl, and heterocycle, or R₉ and R₁₀ together with the nitrogen atom to which they are attached can be joined to form a heterocyclic ring.

240. The combinatorial library of claim 239, wherein at least one of R₁, R₃, R₄, R₅ or R₆ is COOH.

241. The combinatorial library of claim 239, wherein at least one of R₁, R₃, R₄, R₅, or R₆ is OH.

242. The combinatorial library of claim 239, wherein at least one of R₁, R₃, R₄, R₅, or R₆ is NO₂.

243. The combinatorial library of claim 239, wherein at least one of R₁, R₃, R₄, R₅ or R₆ is CN.

244. The combinatorial library of claim 239, wherein at least one of R₁, R₃, R₄, R₅, or R₆ is OAlkyl.

245. The combinatorial library of claim 239, wherein at least one of R₁, R₃, R₄, R₅, or R₆ is COOAlkyl.

246. The combinatorial library of claim 239, wherein at least one of R₁, R₃, R₄, R₅, or R₆ is NHAc.

247. The combinatorial library of claim 239, having the formula:

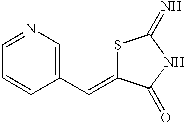

248. The combinatorial library of claim 239, wherein the common ligand mimic comprises a compound of the formula: 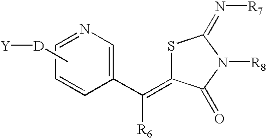
wherein
D is alkylene, alkenylene, alkynylene, aryl, or heterocycle; and
Y is OH, NHR₁₁, SH, COOH, SO₂OH, X, CN, N₃, CONH₂, CONHR₁₁, C≡CH, or CH═CH₂.

249. The combinatorial library of claim 239, wherein the common ligand mimic comprises a compound of the formula: 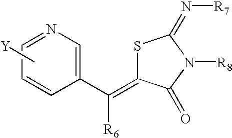
wherein Y is OH, NHR₁₁, SH, COOH, SO₂OH, X, CN, N₃, CONH₂, CONHR₁₁, C≡CH, or CH═CH₂.

250. The combinatorial library of claim 239, wherein the common ligand mimic comprises a compound of the formula: 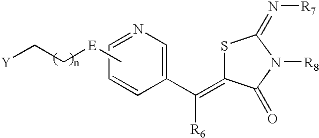
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

251. The combinatorial library of claim 239, wherein the common ligand mimic comprises a compound of the formula: 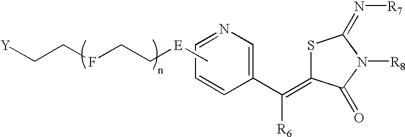
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

252. The combinatorial library of claim 239, wherein the common ligand mimic comprises a compound of the formula: 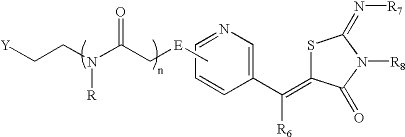
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

253. The combinatorial library of claim 239, wherein the common ligand mimic comprises a compound of the formula: 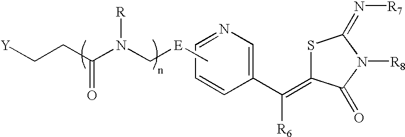
wherein
E is present or absent and when present is O, S, NH, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
Y is OH, NHR₁₁, SH, COOH, SO₂OH, X, CN, N₃, CONH₂, CONHR₁₁, C≡CH, or CH═CH₂;
R is hydrogen, alkyl, alkenyl, alkynyl, aryl, or heterocycle; and
n is an integer between 0 and 5, inclusive.

254. The combinatorial library of claim 239, wherein the common ligand mimic comprises a compound of the formula: 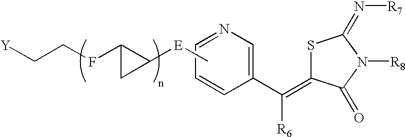
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of O, S, NR₁₁, CR₁₀C₁₁, CONR₁₁, SO₂NR₁₁, NR₁₀CONR₁₁, NR₁₀CNHNR₁₁, NR₁₁COO, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

255. The combinatorial library of claim 239, wherein the common ligand mimic comprises a compound of the formula: 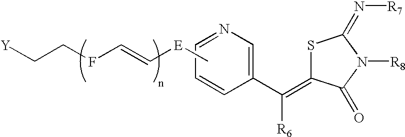
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

256. The combinatorial library of claim 239, wherein the common ligand mimic comprises a compound of the formula: 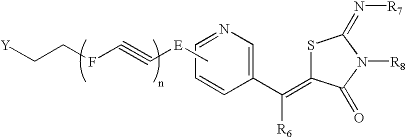
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH;
F each independently is selected from the group consisting of CR₁₀C₁₁, CONR₁₁, C≡C, and CH═CH;
Y is OH, NHR₁₁, SH, COOH, SO₂OH, X, CN, N₃, CONH₂, CONHR₁₁, C≡CH, or CH═CH₂; and
n is an integer between 0 and 5, inclusive.

257. The combinatorial library of claim 239, wherein the common ligand mimic comprises a compound of the formula: 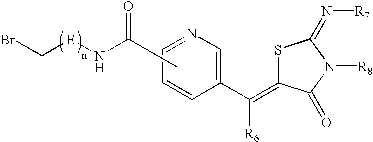
wherein
E is present or absent and when present is O, S, NR₁₁, CR₁₀C₁₁, CONR₁₁, SO₂NR₁₁, NR₁₀CONR₁₁, NR₁₀CNHNR₁₁, NR₁₁COO, C≡C, or CH═CH; and
n is an integer between 0 and 5, inclusive.

258. The combinatorial library of claim 239, wherein the common ligand mimic comprises a compound of the formula: 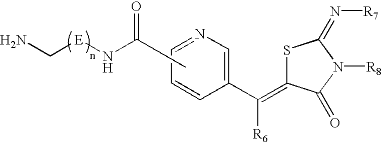
wherein
E present or absent and when present is CH₂, CH₂CH₂OCH or CH₂CH₂SCH and n is an integer between 1 and 10, inclusive.

259. The combinatorial library of claim 258, where n is greater than 4 and E is CH₂CH₂OCH or CH₂CH₂SCH.

260. The combinatorial library of claim 239, wherein the common ligand mimic comprises a compound of the formula: [chemical structure]
